(19) United States
(12) Patent Application Publication
(10) Pub. No.: US 2009/0264431 A1
(43) Pub. Date: Oct. 22, 2009

(54) NOVEL CATHEPSIN C INHIBITORS AND THEIR USE

(76) Inventors: Michael R. Palovich, King of Prussia, PA (US); Haibo Xie, Collegeville, PA (US); Jianghe Deng, Collegeville, PA (US); Dramane Ibrahim Laine, King of Prussia, PA (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 12/193,128

(22) Filed: Aug. 18, 2008

Related U.S. Application Data
(60) Provisional application No. 60/956,744, filed on Aug. 20, 2007.

(51) Int. Cl. C07D 413/02; C07D 207/14; A61K 31/5377; A61K 31/40

(52) U.S. Cl. 514/237.2; 548/557; 514/426; 544/141

(57) ABSTRACT

The invention is directed to compounds according to Formula I:

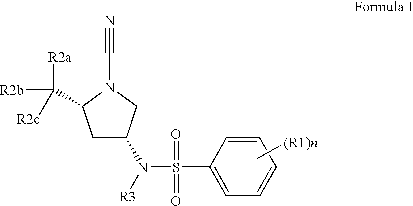

wherein R1, R2a, R2b, R2c, R3, and n are defined below, and to pharmaceutically-acceptable salts thereof. They are cathepsin C inhibitors and can be used in the treatment of diseases mediated by the cathepsin C enzyme, such as COPD.

### FIELD OF THE INVENTION

The invention is directed to novel cathepsin C inhibitors and their use in the treatment of diseases mediated by the cathepsin C enzyme.

### BACKGROUND OF THE INVENTION

Cathepsins are a family of enzymes included in the papain superfamily of cysteine proteases. Cathepsins B, C, F, H, K, L, O, S, V, W, and X have been described in the scientific literature. Cathepsin C is also known in the literature as Dipeptidyl Peptidase I or “DPPI.”

A number of published studies have begun to describe the role cathepsin C plays in certain inflammatory processes. See E.g. Methot et al., The Journal of Biological Chemistry, 282 (29): 20836-46 (2007); Pagano et al., Proc Natl Acad Sci USA 104 (8): 2855-60 (2007); Xuchu Que et al., The Journal of Biological. Chemistry, 282 (7): 4994-5003 (2007); Adkison et al., The Journal of Clinical Investigation 109: 363-371 (2002); Tran et al., Archives of Biochemistry and Biophysics 403: 160-170 (2002); Thiele et al., The Journal of Immunology 158: 5200-5210 (1997); Bidere et al., The Journal of Biological Chemistry 277: 32339-32347 (2002); Mabee et al., The Journal of Immunology 160: 5880-5885; McGuire et al., The Journal of Biological Chemistry, 268: 2458-2467; and Paris et al., FEBS Letters 369: 326-330 (1995). From these studies, it appears that cathepsin C is co-expressed with certain serine proteases, which are released from inflammatory cells recruited to sites of inflammation, and acts as a physiological activator of these proteases. Once activated, these proteases are capable of degrading various extracellular matrix components, which can lead to tissue damage and chronic inflammation.

For example, Chronic Obstructive Pulmonary Disease (“COPD”) is a chronic inflammatory disease where cathepsin C is believed to play a role. The American Thoracic Society defines COPD as “a disease characterized by the presence of airflow obstruction due to chronic bronchitis or emphysema; the airflow obstruction is generally progressive, may be accompanied by airway hyperreactivity, and may be partially reversible.” American Journal of Respiratory and Critical Care Medicine 152: S77-S120 (1995). Chronic bronchitis is generally characterized by a chronic productive cough, whereas emphysema is generally characterized by permanent enlargement of the airspaces distal to the terminal bronchioles and airway wall destruction. Chronic bronchitis and emphysema usually occur together in COPD patients.

Cigarette smoking is a significant risk factor for developing COPD. Exposure to cigarette smoke and/or other noxious particles and gases may result in chronic inflammation of the lung. In response to such exposure, inflammatory cells such as CD8+ Tcells, macrophages, and neutrophils are recruited to the area. These recruited inflammatory cells release proteases, which are believed to play a major role in the disease etiology by degrading airway walls. Proteases believed to be involved in this process include the serine proteases neutrophil elastase (“NE”), chymase (“CY”), cathepsin G, proteinase 3 and granzymes A and B. Cathepsin C appears to be involved in activating these enzymes.

Rheumatoid arthritis (“RA”) is another chronic inflammatory disease where cathepsin C is believed to play a role. Arthritis and Rheumatism. 52: 2553-8 (2005). Neutrophils are recruited to the site of joint inflammation and release cathepsin G, NE, and proteinase 3, which are believed to be responsible for cartilage destruction associated with RA. Cathepsin C appears also to be involved in activating these enzymes.

Other conditions where cathepsin C is believed to play a role include abdominal or thoracic aortic aneurism, adult respiratory distress syndrome, acute lung injury, osteoarthritis, asthma, multiple sclerosis, sepsis, and toxoplasmosis. See E.g. Moraes, T. J., Chow, C-W., Downey, G. P. Proteases and lung injury Critical Care Medicine 31 (suppl.): S189-S194 (2003); Okayama N., Kakihana Y., Setoguchi D., Matsui K. Yuyama N. Akaiwa M. Yoshida N L. Maeda M. Sugita Y. Izuhara K., Identification of an alternative splicing variant of cathepsin C/dipeptidyl-peptidase I, Gene 293 (1-2): 1-7 (2002); Wolters P J. Laig-Webster M. Caughey G H., Dipeptidyl peptidase I cleaves matrix-associated proteins and is expressed mainly by mast cells in normal dog airways, American Journal of Respiratory Cell & Molecular Biology 22 (2): 183-90 (2000); Mallen-St Clair J. Pham C T. Villalta S A. Caughey G H. Wolters P J., Mast cell dipeptidyl peptidase I mediates survival from sepsis, Journal of Clinical Investigation 113: 628-34 (2004); Xuchu Que, Juan C. Engel, David Ferguson, Annette Wunderlich, Stanislas Tomavo, and Sharon L. Reed, Cathepsin Cs Are Key for the Intracellular Survival of the Protazoan Parasite, Toxoplasma gondii, The Journal of Biological Chemistry, 282 (7): 4994-5003 (2007).

One approach to treating these conditions is to inhibit the activity of the serine proteases involved in the inflammatory process, especially NE activity. See E.g., Ohbayashi, “Neutrophil elastase inhibitors as treatment for COPD”, Expert Opin. Investig. Drugs 11 (7): 965-980 (2002); Shapiro, “Neutrophil Elastase: Path Clearer, Pathogen Killer, or Just Pathologic?”, Am. J. Respir. Cell Mol. Biol. 26: 266-268 (2002); Imabayashi T., Omae T., Matsunaga A., Kanmura Y., Clinical effects of a neutrophil elastase inhibitor, sivelestat, in patients with acute respiratory distress syndrome Journal of Anesthesia 20: 6-10 (2006). In light of the role cathepsin C plays in activating certain serine proteases, especially NE, it is desirable to prepare compounds that inhibit its activity, which thereby inhibit serine protease activity. Thus, there is a need to identify compounds that inhibit cathepsin C, which can be used in the treatment of a variety of conditions mediated by the cathepsin C enzyme.

### SUMMARY OF THE INVENTION

This invention related to compounds of Formula I:

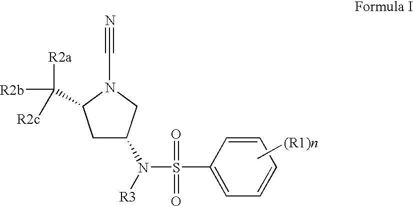

wherein:

each R1 is independently selected from the group consisting of: halo, C1-C4 alkyl, CF₃, CN, NO₂, —ORa, —OCF₃, —C(O)NHRa, —C(O)ORa, —NRaRa, —NHC(O)Ra, or —NHC(O)NHRa;

n is an integer from 0 to 4;

R2a is H, halo, —C(O)Rx, —C(O)ORy, —C(O)NRaRy, —OC(O)Rx, —OC(O)NRaRy, —NRaRy, —NRaC(O)Rx, NRaC(O)R22, —NRaC(O)ORy, —NRaC(O)NRaRy, R20, R21, R22, R23, R24, —OH, —OR20, —OR21, —OR22, —OR23, or —OR24; —CN

R2b is H or C1-C4 alkyl;

or R2a and R2b taken together with the carbon atom to which they are attached form a C3-C7 cycloalkyl group;

R2c is H or C1-C4 alkyl;

R20 is C1-C4 alkyl;

wherein said R20 is optionally substituted with one or more substituents independently selected from the group consisting of: halo, CF₃, CN, NO₂, R21, R22, R23, R24, —ORy, —C(O)Rx, —C(O)ORy, —C(O)NRaRy, —OC(O)Rx, —OC(O)NRaRy, —NRaRy, —NRaC(O)Rx, —NRaC(O)ORy, —NRaC(O)NRaRy;

R21 is C3-C6 cycloalkyl;

wherein said R21 is optionally substituted with one or more substituents independently selected from the group consisting of: CF₃, Rc, —ORa, —OCF₃, and —NRaRa;

R22 is heterocycloalkyl;

wherein said R22 is optionally substituted with one or more substituents independently selected from the group consisting of: CF₃, Rc, —ORa, —OCF₃, and —NRaRa;

R23 is phenyl;

wherein said R23 is optionally substituted with one or more substituents independently selected from the group consisting of: halo, CF₃, CN, NO₂, Rc, —ORa, —OCF₃, —C(O)Rb, —C(O)ORa, —C(O)NRaRa, —OC(O)Rb, —OC(O)NRaRa, —NRaRa, —NRaC(O)Rb, —NRaC(O)ORa, —NRaC(O)NRaRa;

R24 is monocyclic heteroaryl;

wherein said R24 is optionally substituted with one or more substituents independently selected from the group consisting of: halo, CF₃, CN, NO₂, Rc, —ORa, —OCF₃, —C(O)Rb, —C(O)ORa, —C(O)NRaRa, —OC(O)Rb, —OC(O)NRaRa, —NRaRa, —NRaC(O)Rb, —NRaC(O)ORa, —NRaC(O)NRaRa;

R3 is H, R30, or R31;

R30 is C1-C4 alkyl, C2-C4 alkenyl, or C2-C4 alkynyl;

wherein said R30 is optionally substituted with one or more substituents independently selected from the group consisting of: CF₃, Re, Rf, Rg, CN, —ORa, —OCF₃, —ORf, —ORg, —OR31, and —NRaRa;

R31 is C3-C6 cycloalkyl;

wherein said R31 is optionally substituted with one or more substituents independently selected from the group consisting of: Rb, —ORa, —OCF₃, and —NRaRa;

each Ra is independently H or C1-C4 alkyl;

each Rb is independently C1-C4 alkyl;

each Rc is independently C1-C4 alkyl; wherein said C1-C4 alkyl is optionally substituted with one or more substituents independently selected from the group consisting of: CF₃, —ORa, OCF₃, and —NRaRa;

each Rd is independently C1-C4 alkyl; wherein said C1-C4 alkyl is optionally substituted with one or more substituents independently selected from the group consisting of: CF₃, —ORa, OCF₃, —NRaRa, Re, and Rf;

each Re is independently phenyl or heteroaryl optionally substituted with one or more substituents independently selected from the group consisting of: halo, NO₂, CF₃, Rb, R23, R24, —ORa, OCF₃, and —NRaRa;

each Rf is independently monocyclic heteroaryl optionally substituted with one or more substituents independently selected from the group consisting of: halo, CF₃, Rb, R23, R24, —ORa, OCF₃, and —NRaRa;

each R9 is independently napthyl optionally substituted with one or more substituents independently selected from the group consisting of: halo, CF₃, Rb, —ORa, OCF₃, and —NRaRa;

each Rh is independently C3-C6 cycloalkyl optionally substituted with one or more substituents independently selected from the group consisting of: Rb, —ORa, —OCF₃, and —NRaRa;

each Rx is Rd, Re, Rf, Rg, or Rh; and

each Ry is H, Rd, Re, Rf, Rg, or Rh; or

a salt thereof.

The compounds of the invention are cathepsin C inhibitors and can be used in the treatment of diseases mediated by the cathepsin C enzyme, such as COPD. Accordingly, the invention is further directed to pharmaceutical compositions comprising a compound of the invention, or pharmaceutically acceptable salt of it. The invention is still further directed to methods of inhibiting cathepsin C and treatment of conditions associated therewith using a compound of the invention or a pharmaceutical composition comprising a compound of the invention, or a pharmaceutically acceptable salt it.

### DETAILED DESCRIPTION OF THE INVENTION

In describing the invention, chemical elements are identified in accordance with the Periodic Table of the Elements. Abbreviations and symbols utilized herein are in accordance with the common usage of such abbreviations and symbols by those skilled in the chemical and biological arts. For example, the following abbreviations are used herein:

“aq” is an abbreviation for aqueous

“AcCN” is an abbreviation for acetonitrile

“BOC” or “boc” is an abbreviation for tert-butyloxycarbonyl

“° C.” is an abbreviation for degrees Celsius

“Cbz” is an abbreviation for carbobenzyloxy

“CDI” is an abbreviation for carbodiimidazole

“DCM/CH₂Cl₂” is an abbreviation for dichloromethane

“DEAD” is an abbreviation for diethylazodicarboxylate

“DMAP” is an abbreviation for dimethylaminopyridine

“DIPEA” or “DIEA” is an abbreviation for di-isopropylethylamine

“DMF” is an abbreviation for dimethylformamide

“DPPA” is an abbreviation for diphenylphosphoryl azide

“EA” or “EtAc” is an abbreviation for ethyl acetate

“ESI” is an abbreviation for electrospray ionization

“eq” is an abbreviation for equivalent

“HPLC” is an abbreviation for High Pressure Liquid Chromatography

“g” is an abbreviation for grams

“h” or “hr” is an abbreviation for hour

“L” is an abbreviation for liters

“LC-MS” or “LC/MS” is an abbreviation for Liquid chromatography-Mass spectrometry

“Me” is an abbreviation for methyl

“Ms” is an abbreviation for methanesulfonyl

“mL” is an abbreviation for milliliters

“min” is an abbreviation for minute or minutes

“mmol” is an abbreviation for millimole or millimolar

“N” is an abbreviation for Normal and refers to the number of equivalents of reagent per liter of solution

“PE” is an abbreviation for petroleum ether

“Ph” is an abbreviation for phenyl

“PS” is an abbreviation for polymer-supported

“sat” is an abbreviation for saturated

“Si” is an abbreviation for silica

“SPE” is an abbreviation for solid phase extraction

“TBAF” is an abbreviation for tetra-butylammonium fluoride

“TBS” is an abbreviation for t-butyldimethyl silyl

“TBS-Cl” is an abbreviation for t-butyldimethyl silyl chloride

“TBTU” is an abbreviation for O-(benzotriazol-1-yl)-N,N,N′,N′-tetramethyluronium tetrafluoroborate

“TEA” is an abbreviation for triethylamine

“TEMPO” is an abbreviation for 2,2,6,6,-tetramethylpiperidine 1-oxyl

“TFA” is an abbreviation for trifluoroacetic acid

“THF” is an abbreviation for tetrahydrofuran

“UV” is an abbreviation for ultraviolet.

“Alkyl” refers to a saturated hydrocarbon chain having the specified number of carbons. For example, C1-C8 alkyl refers to an alkyl group having from 1 to 8 carbons. Alkyl groups may be optionally substituted with one or more substituents as defined herein. Alkyl groups may be straight or branched. Representative branched alkyl groups have one, two, or three branches. Alkyl includes methyl, ethyl, propyl (n-propyl and isopropyl), butyl (n-butyl, isobutyl, and t-butyl), pentyl (n-pentyl, isopentyl, and neopentyl), and hexyl.

“Alkenyl” refers to an unsaturated hydrocarbon chain having the specified number of carbons and having one at least one carbon-carbon double bond. For example, C2-C6 alkenyl refers to an alkenyl group having from 2 to 6 carbons. Alkenyl groups may be optionally substituted with one or more substituent as defined herein. Alkenyl groups may be straight or branched. They, may have a cis or trans configuration. Representative branched alkenyl groups have one, two, or three branches. Alkenyl includes ethylenyl, propenyl, butenyl, pentenyl, and hexenyl.

“Alkynyl” refers to an unsaturated hydrocarbon chain having the specified number carbons and having one at least one carbon-carbon triple bond. For example, C2-C6 alkynyl refers to a group having from 2 to 6 carbons. Alkynyl groups may be optionally substituted with one or more substituent as defined herein. Alkynyl groups may be straight or branched. Alkynyl includes ethylynyl, propynyl, butynyl, pentynyl, and hexynyl.

“Aryl” refers to a monovalent aromatic hydrocarbon ring. Aryl groups are monocyclic ring systems or bicyclic ring systems. Monocyclic aryl ring refers to phenyl. Bicyclic aryl ring refers to napthyl, biphenyl, and to rings wherein phenyl is fused to a cycloalkyl or cycloalkenyl ring having 5, 6, or 7 member atoms. Aryl groups may be optionally substituted with one or more substituents as defined herein.

“Cycloalkyl” or “cycloalkenyl” refers to a saturated or unsaturated hydrocarbon ring having the specified number of carbons. For example, C3-C6 cycloalkyl refers to a cycloalkyl group having from 3 to 6 carbons. And C4-C6 cycloalkyenyl refers to a ring that has 4 to 6 carbons and at least 1 double bond. These rings are not aromatic. Either group may be optionally substituted with one or more substituents as defined herein. Cycloalkyl includes cyclopropyl, cyclobutyl, cyclopentyl, and cyclohexyl. Cycloalkenyl includes cyclobutenyl and cyclohexenyl, for example.

“Enantiomerically enriched” refers to products whose enantiomeric excess is greater than zero. For example, enantiomerically enriched refers to products whose enantiomeric excess is greater than 50% ee, greater than 75% ee, and greater than 90% ee.

“Enantiomeric excess” or “ee” is the excess of one enantiomer over the other expressed as a percentage. As a result, since both enantiomers are present in equal amounts in a racemic mixture, the enantiomeric excess is zero (0% ee). However, if one enantiomer was enriched such that it constitutes 95% of the product, then the enantiomeric excess would be 90% ee (the amount of the enriched enantiomer, 95%, minus the amount of the other enantiomer, 5%).

“Enantiomerically pure” refers to products whose enantiomeric excess is 99% ee or greater.

“Half-life” (or “half-lives”) refers to the time required for half of a quantity of a substance to be converted to another chemically distinct specie in vitro or in vivo.

“Halo” refers to the halogen radical fluoro, chloro, bromo, or iodo.

“Haloalkyl” refers to an alkyl group that is substituted with one or more halo substituents. Haloalkyl includes trifluoromethyl.

“Heteroaryl” refers to an aromatic ring containing from 1 to 4 heteroatoms in the ring. Heteroaryl groups containing more than one heteroatom may contain different heteroatoms. Heteroaryl groups may be optionally substituted with one or more substituents as defined herein. Unless otherwise specified, heteroaryl groups are monocyclic ring systems or are fused, spiro, or bridged bicyclic ring systems. Monocyclic heteroaryl rings have 5 or 6 atoms. Bicyclic heteroaryl rings have from 7 to 11 atoms. Bicyclic heteroaryl rings include those rings wherein phenyl and a monocyclic heterocycloalkyl ring are attached forming a fused, spiro, or bridged bicyclic ring system, and those rings wherein a monocyclic heteroaryl ring and a monocyclic cycloalkyl, cycloalkenyl, heterocycloalkyl, or heteroaryl ring are attached forming a fused, spiro, or bridged bicyclic ring system. Heteroaryl includes pyrrolyl, pyrazolyl, imidazolyl, oxazolyl, isoxazolyl, oxadiazolyl, thiazolyl, isothiazolyl, thiadiazolyl, furanyl, furazanyl, thienyl, triazolyl, pyridinyl, pyrimidinyl, pyridazinyl, pyrazinyl, triazinyl, tetrazinyl, tetrazolyl, indolyl, isoindolyl, indolizinyl, indazolyl, purinyl, quinolinyl, isoquinolinyl, quinoxalinyl, quinazolinyl, pteridinyl, cinnolinyl, benzimidazolyl, benzopyranyl, benzoxazolyl, benzisoxazolyl, benzofuranyl, isobenzofuranyl, benzothiazolyl, benzisothiazolyl, benzothienyl, furopyridinyl, and napthyridinyl.

“Heteroatom” refers to a nitrogen, sulphur, or oxygen atom.

“Heterocycloalkyl” or “heterocycloalkenyl” refers to a saturated or unsaturated ring containing from 1 to 4 heteroatoms atoms in the ring. These rings are not aromatic. A ring containing more than one heteroatom may contain different heteroatoms. A ring may be optionally substituted with one or more substituent as defined herein, either on a carbon or on the heteroatom. Unless otherwise specified, these rings are monocyclic or may be fused, spiro, or a bridged bicyclic ring system. Monocyclic rings have from 5 to 7 member atoms. Bicyclic rings have from 7 to 11 member atoms. These rings include, for example, pyrrolidinyl, tetrahydrofuranyl, dihydrofuranyl, pyranyl, tetrahydropyranyl, dihydropyranyl, tetrahydrothienyl, pyrazolidinyl, oxazolidinyl, thiazolidinyl, piperidinyl, homopiperidinyl, piperazinyl, morpholinyl, thiamorpholinyl, azepinyl, 1,3-dioxolanyl, 1,3-dioxanyl, 1,4-dioxanyl, 1,3-oxathiolanyl, 1,3-oxathianyl, 1,3-dithianyl, azetidinyl, azabicylo[3.2.1]octyl, azabicylo[3.3.1]nonyl, azabicylo[4.3.0]nonyl, oxabicylo[2.2.1]heptyl, and

“Optionally substituted” indicates that a group, such as alkyl, alkenyl, alkynyl, aryl, cycloalkyl, cycloalkenyl, heterocycloalkyl, or heteroaryl, may be unsubstituted or substituted with one or more substituents as defined herein. “Substituted” in reference to a group indicates that a hydrogen atom attached to a member atom within a group is replaced. It should be understood that the term “substituted” includes the implicit provision that such substitution be in accordance with the permitted valence of the substituted atom and the substituent and that the substitution results in a stable compound (i.e. one that does not spontaneously undergo transformation such as by rearrangement, cyclization, or elimination). In certain embodiments, a single atom may be substituted with more than one substituent as long as such substitution is in accordance with the permitted valence of the atom. Suitable substituents are defined herein for each substituted or optionally substituted group.

“Pharmaceutically acceptable” refers to those compounds, materials, compositions, and dosage forms which are, within the scope of sound medical judgment, suitable for use in contact with the tissues of human beings and animals without excessive toxicity, irritation, or other problem or complication, commensurate with a reasonable benefit/risk ratio.

### Compounds

Compounds of particular interest herein include, in collectively or independently, the following groups:

Compounds of Formula I where

R1 is independently selected from the group consisting of: halo, C1-C4 alkyl, CF₃, CN, NO₂, —ORa, and —OCF₃;

Ra is H or C1-C4alkyl;

R2a is H, halo, —C(O)Rx, —C(O)ORy, or —C(O)NraRy; or

R2a is —NRaRy, —NRaC(O)Rx, —NRaC(O)ORy, or —NRaC(O)NraRy; or

R2a is R20, R21, R22, R23, or R24; or

R2a is —OH, —OR20, —OR21, —OR22, —OR23, or —OR24;

R2b is H;

R2c is H;

R3 is H;

Rx is Rd; and

Ry is phenyl optionally substituted with one or more substituents independently selected from the group consisting of: CF₃, Rb, and —ORa.

The compounds according to Formula I may contain one or more asymmetric centers (also referred to as a chiral center) and may, therefore, exist as individual enantiomers, diastereomers, or other stereoisomeric forms, or as mixtures thereof. Chiral centers may also be present in a substituent such as an alkyl group. Where the stereochemistry of a chiral center present in Formula I, or in any chemical structure illustrated herein, is not specified the structure is intended to encompass any stereoisomer and all mixtures thereof. Thus, compounds according to Formula I containing one or more chiral center may be used as racemic mixtures, enantiomerically enriched mixtures, or as enantiomerically pure individual stereoisomers.

Individual stereoisomers of a compound according to Formula I which contain one or more asymmetric center may be resolved by methods known to those skilled in the art. For example, such resolution may be carried out (1) by formation of diastereoisomeric salts, complexes or other derivatives; (2) by selective reaction with a stereoisomer-specific reagent, for example by enzamatic oxidation or reduction; or (3) by gas-liquid or liquid chromatography in a chiral environment, for example, on a chiral support such as silica with a bound chiral ligand or in the presence of a chiral solvent. The skilled artisan will appreciate that where the desired stereoisomer is converted into another chemical entity by one of the separation procedures described above, a further step is required to liberate the desired form. Alternatively, specific stereoisomers may be synthesized by asymmetric synthesis using optically active reagents, substrates, catalysts or solvents, or by converting one enantiomer to the other by asymmetric transformation.

The compounds according to Formula I may also contain double bonds or other centers of geometric asymmetry. Where the stereochemistry of a center of geometric asymmetry present in Formula I, or in any chemical structure illustrated herein, is not specified, the structure is intended to encompass the trans (E) geometric isomer, the cis (Z) geometric isomer, and all mixtures thereof. If there is a cycloalkyl or cycloalkenyl group present, some substituent patterns may result in and axial or an equatorial configuration. Both forms are included, unless specified otherwise.

All tautomeric forms are also included in Formula I whether such tautomers exist in equilibrium or predominately in one form.

In certain embodiments, compounds according to Formula I may contain an acidic functional group and are therefore capable of forming pharmaceutically-acceptable base addition salts by treatment with a suitable base. In certain other embodiments, compounds according to Formula I may contain a basic functional group and are therefore capable of forming pharmaceutically-acceptable acid addition salts by treatment with a suitable acid. Thus, the skilled artisan will appreciate that pharmaceutically-acceptable salts of the compounds according to Formula I may be prepared. Indeed, in certain embodiments of the invention, pharmaceutically-acceptable salts of the compounds according to Formula I may be preferred over the respective free base or free acid because such salts impart greater stability or solubility to the molecule thereby facilitating formulation into a dosage form. Accordingly, the invention is further directed to pharmaceutically-acceptable salts of the compounds according to Formula.

As used herein, the term “pharmaceutically-acceptable salts” refers to salts that retain the desired biological activity of the subject compound and exhibit minimal undesired toxicological effects. These pharmaceutically-acceptable salts may be prepared in situ during the final isolation and purification of the compound, or by separately reacting the purified compound in its free acid or free base form with a suitable base or acid, respectively.

As used herein, the term “compounds of the invention” means both the compounds according to Formula I and the pharmaceutically-acceptable salts thereof. The term “a compound of the invention” also appears herein and refers to both a compound according to Formula I and its pharmaceutically-acceptable salts.

In the solid state, compounds of the invention can exist in crystalline, semi-crystalline and amorphous forms, as well as mixtures thereof. The skilled artisan will appreciate that pharmaceutically-acceptable solvates of a compound of the invention may be formed wherein solvent molecules are incorporated into the solid-state structure during crystallization. Solvates may involve water or nonaqueous solvents, or mixtures thereof. In addition, the solvent content of such solvates can vary in response to environment and upon storage. For example, water may displace another solvent over time depending on relative humidity and temperature.

Solvates wherein water is the solvent that is incorporated into the solid-state structure are typically referred to as “hydrates.” Solvates wherein more than one solvent is incorporated into the solid-state structure are typically referred to as “mixed solvates”. Solvates include “stoichiometric solvates” as well as compositions containing variable amounts of solvent (referred to as “non-stoichiometric solvates”). Stoichiometric solvates wherein water is the solvent that is incorporated into the solid-state structure are typically referred to as “stoichiometric hydrates”, and non-stoichiometric solvates wherein water is the solvent that is incorporated into the solid-state structure are typically referred to as “non-stoichiometric hydrates”. The invention includes both stoichiometric and non-stoichiometric solvates.

In addition, crystalline forms of a compound of the invention, including solvates thereof, may contain solvent molecules, which are not incorporated into the solid-state structure. For example, solvent molecules may become trapped in the crystals upon isolation. In addition, solvent molecules may be retained on the surface of the crystals. The invention includes such forms.

The skilled artisan will further appreciate that compounds of the invention, including solvates thereof, may exhibit polymorphism (i.e. the capacity to occur in different crystalline packing arrangements). These different crystalline forms are typically known as “polymorphs.” The invention includes all such polymorphs. Polymorphs have the same chemical composition but differ in packing, geometrical arrangement, and other descriptive properties of the crystalline solid state. Polymorphs, therefore, may have different physical properties such as shape, density, hardness, deformability, stability, and dissolution properties. Polymorphs typically exhibit different IR spectra and X-ray powder diffraction patterns, which may be used for identification. Polymorphs may also exhibit different melting points, which may be used for identification. The skilled artisan will appreciate that different polymorphs may be produced, for example, by changing or adjusting the reaction conditions or reagents, used in making the compound. For example, changes in temperature, pressure, or solvent may result in the production of different polymorphs. In addition, one polymorph may spontaneously convert to another polymorph under certain conditions.

### Methods of Use

The compounds of the invention inhibit the cathepsin C enzyme and can be useful in the treatment of conditions wherein the underlying pathology is (at least in part) attributable to cathepsin C involvement or in conditions wherein cathepsin C inhibition offers some clinical benefit even though the underlying pathology is not (even in part) attributable to cathepsin C involvement. Examples of such conditions include COPD, and rheumatoid arthritis. Accordingly, in another aspect the invention is directed to methods of treating such conditions.

The methods of treatment of the invention comprise administering a safe and effective amount of a compound of the invention to a patient in need thereof. In one embodiment, the invention is directed to method for treating COPD. In another embodiment, the invention is directed to a method for treating rheumatoid arthritis. In yet another embodiment, the invention is directed to a method for treating abdominal or thoracic aneurism. In yet another embodiment, the invention is directed to a method for treating adult respiratory distress syndrome. In yet another embodiment, the invention is directed to a method for treating acute lung injury. In yet another embodiment, the invention is directed to a method for treating asthma. In yet another embodiment, the invention is directed to a method for treating osteoarthritis. In yet another embodiment, the invention is directed to a method for treating multiple sclerosis. In yet another embodiment, the invention is directed to a method for treating sepsis. In yet another embodiment, the invention is directed to a method for treating taxoplasmosis.

As used herein, “treatment” in reference to a condition means: (1) the amelioration or prevention of the condition being treated or one or more of the biological manifestations of the condition being treated, (2) the interference with (a) one or more points in the biological cascade that leads to or is responsible for the condition being treated or (b) one or more of the biological manifestations of the condition being treated, or (3) the alleviation of one or more of the symptoms or effects associated with the condition being treated.

As indicated above, “treatment” of a condition includes prevention of the condition. The skilled artisan will appreciate that “prevention” is not an absolute term. In medicine, “prevention” is understood to refer to the prophylactic administration of a drug to substantially diminish the likelihood or severity of a condition or biological manifestation thereof, or to delay the onset of such condition or biological manifestation thereof.

As used herein, “safe and effective amount” in reference to a compound of the invention or other pharmaceutically-active agent means an amount of the compound sufficient to significantly induce a positive modification in the condition to be treated but low enough to avoid serious side effects (at a reasonable benefit/risk ratio) within the scope of sound medical judgment. A safe and effective amount of a compound of the invention will vary with the particular compound chosen (e.g. consider the potency, efficacy, and half-life of the compound); the route of administration chosen; the condition being treated; the severity of the condition being treated; the age, size, weight, and physical condition of the patient being treated; the medical history of the patient being treated; the duration of the treatment; the nature of concurrent therapy; the desired therapeutic effect; and like factors, but can nevertheless be determined by the skilled artisan.

As used herein, “patient” refers to a human or other animal.

The compounds of the invention may be administered by any suitable route of administration, including both systemic administration and topical administration. Systemic administration includes oral administration, parenteral administration, transdermal administration, rectal administration, and administration by inhalation. Parenteral administration refers to routes of administration other than enteral, transdermal, or by inhalation, and is typically by injection or infusion. Parenteral administration includes intravenous, intramuscular, and subcutaneous injection or infusion. Inhalation refers to administration into the patient's lungs whether inhaled through the mouth or through the nasal passages. Topical administration includes application to the skin as well as intraocular, otic, intravaginal, and intranasal administration.

The compounds of the invention may be administered once or according to a dosing regimen wherein a number of doses are administered at varying intervals of time for a given period of time. For example, doses may be administered one, two, three, or four times per day. Doses may be administered until the desired therapeutic effect is achieved or indefinitely to maintain the desired therapeutic effect. Suitable dosing regimens for a compound of the invention depend on the pharmacokinetic properties of that compound, such as absorption, distribution, and half-life, which can be determined by the skilled artisan. In addition, suitable dosing regimens, including the amount administered and the duration such regimens are administered, for a compound of the invention depend on the condition being treated, the severity of the condition being treated, the age and physical condition of the patient being treated, the medical history of the patient to be treated, the nature of concurrent therapy, the particular route of administration chosen, the desired therapeutic effect, and like factors within the knowledge and expertise of the skilled artisan. It will be further understood by such skilled artisans that suitable dosing regimens may require adjustment given an individual patient's response to the dosing regimen or over time as individual patient needs change. Typical daily dosages range from 1 mg to 1000 mg.

Additionally, the compounds of the invention may be administered as prodrugs. As used herein, a “prodrug” of a compound of the invention is a functional derivative of the compound which, upon administration to a patient, eventually liberates the compound of the invention in vivo. Administration of a compound of the invention as a prodrug may enable the skilled artisan to do one or more of the following: (a) modify the onset of the compound in vivo; (b) modify the duration of action of the compound in vivo; (C) modify the transportation or distribution of the compound in vivo; (d) modify the solubility of the compound in vivo; and (e) overcome or overcome a side effect or other difficulty encountered with the compound. Typical functional derivatives used to prepare prodrugs include modifications of the compound that are chemically or enzymatically cleaved in vivo. Such modifications, which include the preparation of phosphates, amides, esters, thioesters, carbonates, and carbamates, are well known to those skilled in the art.

### Compositions

The compounds of the invention will normally, but not necessarily, be formulated into a pharmaceutical composition prior to administration to a patient. Accordingly, in another aspect the invention is directed to pharmaceutical compositions comprising a compound of the invention and a pharmaceutically-acceptable excipient.

The pharmaceutical compositions of the invention may be prepared and packaged in bulk form wherein a safe and effective amount of a compound of the invention can be extracted and then given to the patient such as with powders, syrups, and solutions for injection. Alternatively, the pharmaceutical compositions of the invention may be prepared and packaged in unit dosage form wherein each physically discrete unit contains a safe and effective amount of a compound of the invention. When prepared in unit dosage form, the pharmaceutical compositions of the invention typically contain from 1 mg to 1000 mg.

The pharmaceutical compositions of the invention typically contain one compound of the invention. However, in certain embodiments, the pharmaceutical compositions of the invention contain more than one compound of the invention. For example, in certain embodiments the pharmaceutical compositions of the invention contain two compounds of the invention. In addition, the pharmaceutical compositions of the invention may optionally further comprise one or more additional pharmaceutically active compounds. Conversely, the pharmaceutical compositions of the invention typically contain more than one pharmaceutically-acceptable excipient. However, in certain embodiments, the pharmaceutical compositions of the invention contain one pharmaceutically-acceptable excipient.

As used herein, “pharmaceutically-acceptable excipient” means a pharmaceutically acceptable material, composition or vehicle involved in giving form or consistency to the pharmaceutical composition. Each excipient must be compatible with the other ingredients of the pharmaceutical composition when commingled such that interactions which would substantially reduce the efficacy of the compound of the invention when administered to a patient and interactions which would result in pharmaceutical compositions that are not pharmaceutically acceptable are avoided. In addition, each excipient must of course be of sufficiently high purity to render it pharmaceutically-acceptable.

The compound of the invention and the pharmaceutically-acceptable excipient or excepients will typically be formulated into a dosage form adapted for administration to the patient by the desired route of administration. For example, dosage forms include those adapted for (1) oral administration such as tablets, capsules, caplets, pills, troches, powders, syrups, elixers, suspensions, solutions, emulsions, sachets, and cachets; (2) parenteral administration such as sterile solutions, suspensions, and powders for reconstitution; (3) transdermal administration such as transdermal patches; (4) rectal administration such as suppositories; (5) inhalation such as aerosols and solutions; and (6) topical administration such as creams, ointments, lotions, solutions, pastes, sprays, foams, and gels.

Suitable pharmaceutically-acceptable excipients will vary depending upon the particular dosage form chosen. In addition, suitable pharmaceutically-acceptable excipients may be chosen for a particular function that they may serve in the composition. For example, certain pharmaceutically-acceptable excipients may be chosen for their ability to facilitate the production of uniform dosage forms. Certain pharmaceutically-acceptable excipients may be chosen for their ability to facilitate the production of stable dosage forms. Certain pharmaceutically-acceptable excipients may be chosen for their ability to facilitate the carrying or transporting the compound or compounds of the invention once administered to the patient from one organ, or portion of the body, to another organ, or portion of the body. Certain pharmaceutically-acceptable excipients may be chosen for their ability to enhance patient compliance.

Suitable pharmaceutically-acceptable excipients include the following types of excipients: Diluents, fillers, binders, disintegrants, lubricants, glidants, granulating agents, coating agents, wetting agents, solvents, co-solvents, suspending agents, emulsifiers, sweetners, flavoring agents, flavor masking agents, coloring agents, anticaking agents, hemectants, chelating agents, plasticizers, viscosity increasing agents, antioxidants, preservatives, stabilizers, surfactants, and buffering agents. The skilled artisan will appreciate that certain pharmaceutically-acceptable excipients may serve more than one function and may serve alternative functions depending on how much of the excipient is present in the formulation and what other ingredients are present in the formulation.

Skilled artisans possess the knowledge and skill in the art to enable them to select suitable pharmaceutically-acceptable excipients in appropriate amounts for use in the invention. In addition, there are a number of resources that are available to the skilled artisan which describe pharmaceutically-acceptable excipients and may be useful in selecting suitable pharmaceutically-acceptable excipients. Examples include Remington's Pharmaceutical Sciences (Mack Publishing Company), The Handbook of Pharmaceutical Additives (Gower Publishing Limited), and The Handbook of Pharmaceutical Excipients (the American Pharmaceutical Association and the Pharmaceutical Press).

The pharmaceutical compositions of the invention are prepared using techniques and methods known to those skilled in the art. Some of the methods commonly used in the art are described in Remington's Pharmaceutical Sciences (Mack Publishing Company).

In one aspect, the invention is directed to a solid oral dosage form such as a tablet or capsule comprising a safe and effective amount of a compound of the invention and a diluent or filler. Suitable diluents and fillers include lactose, sucrose, dextrose, mannitol, sorbitol, starch (e.g. corn starch, potato starch, and pre-gelatinized starch), cellulose and its derivatives (e.g. microcrystalline cellulose), calcium sulfate, and dibasic calcium phosphate. The oral solid dosage form may further comprise a binder. Suitable binders include starch (e.g. corn starch, potato starch, and pre-gelatinized starch), gelatin, acacia, sodium alginate, alginic acid, tragacanth, guar gum, povidone, and cellulose and its derivatives (e.g. microcrystalline cellulose). The oral solid dosage form may further comprise a disintegrant. Suitable disintegrants include crospovidone, sodium starch glycolate, croscarmelose, alginic acid, and sodium carboxymethyl cellulose. The oral solid dosage form may further comprise a lubricant. Suitable lubricants include stearic acid, magnesuim stearate, calcium stearate, and talc.

In another aspect, the invention is directed to a dosage form adapted for administration to a patient by inhalation. For example, the compound of the invention may be inhaled into the lungs as a dry powder, an aerosol, a suspension, or a solution.

Dry powder compositions for delivery to the lung by inhalation typically comprise a compound of the invention as a finely divided powder together with one or more pharmaceutically-acceptable excipients as finely divided powders. Pharmaceutically-acceptable excipients particularly suited for use in dry powders are known to those skilled in the art and include lactose, starch, mannitol, and mono-, di-, and polysaccharides.

The dry powder may be administered to the patient via a reservoir dry powder inhaler (RDPI) having a reservoir suitable for storing multiple (un-metered doses) of medicament in dry powder form. RDPIs typically include a means for metering each medicament dose from the reservoir to a delivery position. For example, the metering means may comprise a metering cup, which is movable from a first position where the cup may be filled with medicament from the reservoir to a second position where the metered medicament dose is made available to the patient for inhalation.

Alternatively, the dry powder may be presented in capsules (e.g. gelatin or plastic), cartridges, or blister packs for use in a multi-dose dry powder inhaler (MDPI). MDPIs are inhalers wherein the medicament is comprised within a multi-dose pack containing (or otherwise carrying) multiple defined doses (or parts thereof) of medicament. When the dry powder is presented as a blister pack, it comprises multiple blisters for containment of the medicament in dry powder form. The blisters are typically arranged in regular fashion for ease of release of the medicament therefrom. For example, the blisters may be arranged in a generally circular fashion on a disc-form blister pack, or the blisters may be elongate in form, for example comprising a strip or a tape. Each capsule, cartridge, or blister may, for example, contain between 20 μg-10 mg of the compound of the invention.

Aerosols may be formed by suspending or dissolving a compound of the invention in a liquified propellant. Suitable propellants include halocarbons, hydrocarbons, and other liquified gases. Representative propellants include: trichlorofluoromethane (propellant 11), dichlorofluoromethane (propellant 12), dichlorotetrafluoroethane (propellant 114), tetrafluoroethane (HFA-134a), 1,1-difluoroethane (HFA-152a), difluoromethane (HFA-32), pentafluoroethane (HFA-12), heptafluoropropane (HFA-227a), perfluoropropane, perfluorobutane, perfluoropentane, butane, isobutane, and pentane. Aerosols comprising a compound of the invention will typically be administered to a patient via a metered dose inhaler (MDI). Such devices are known to those skilled in the art.

The aerosol may contain additional pharmaceutically-acceptable excipients typically used with MDIs such as surfactants, lubricants, cosolvents and other excipients to improve the physical stability of the formulation, to improve valve performance, to improve solubility, or to improve taste.

Suspensions and solutions comprising a compound of the invention may also be administered to a patient via a nebulizer. The solvent or suspension agent utilized for nebulization may be any pharmaceutically-acceptable liquid such as water, aqueous saline, alcohols or glycols, e.g., ethanol, isopropylalcohol, glycerol, propylene glycol, polyethylene glycol, etc. or mixtures thereof. Saline solutions utilize salts which display little or no pharmacological activity after administration. Both organic salts, such as alkali metal or ammonium halogen salts, e.g., sodium chloride, potassium chloride or organic salts, such as potassium, sodium and ammonium salts or organic acids, e.g., ascorbic acid, citric acid, acetic acid, tartaric acid, etc. may be used for this purpose.

Other pharmaceutically-acceptable excipients may be added to the suspension or solution. The compound of the invention may be stabilized by the addition of an inorganic acid, e.g., hydrochloric acid, nitric acid, sulphuric acid and/or phosphoric acid; an organic acid, e.g., ascorbic acid, citric acid, acetic acid, and tartaric acid, etc., a complexing agent such as EDTA or citric acid and salts thereof; or an antioxidant such as antioxidant such as vitamin E or ascorbic acid. These may be used alone or together to stabilize the compound of the invention. Preservatives may be added such as benzalkonium chloride or benzoic acid and salts thereof. Surfactant may be added particularly to improve the physical stability of suspensions. These include lecithin, disodium dioctylsulphosuccinate, oleic acid and sorbitan esters.

Compound Preparation The compounds according to Formula I are prepared using conventional organic syntheses. Suitable synthetic routes are depicted below in the following general reaction schemes. Starting materials and reagents depicted below in the general reaction schemes are commercially available or can be made from commercially available starting materials using methods known by those skilled in the art.

The skilled artisan will appreciate that if a substituent described herein is not compatible with the synthetic methods described herein, the substituent may be protected with a suitable protecting group that is stable to the reaction conditions. The protecting group may be removed at a suitable point in the reaction sequence to provide a desired intermediate or target compound. Suitable protecting groups and methods for protecting and de-protecting different substituents using such suitable protecting groups are well known to those skilled in the art; examples of which may be found in T. Greene and P. Wuts, Protecting Groups in Chemical Synthesis (3rd ed.), John Wiley & Sons, NY (1999). In some instances, a substituent may be specifically selected to be reactive under the reaction conditions used. Under these circumstances, the reaction conditions convert the selected substituent into another substituent that is either useful as an intermediate compound or is a desired substituent in a target compound.

As used herein, “BrCN” refers to a 3 N BrCN solution in DCM, and PS-trisamine refers to a tris-(2-aminoethyl)amine polystyrene resin.

LC-MS of Intermediates and Examples were carried out using the following equipment and conditions:

### Liquid Chromatograph:

| | |
|---|---|
| System: | Shimadzu LC system with SCL-10A Controller and dual |
| | UV detector |
| Autosampler: | Leap CTC with a Valco six port injector |
| Column: | Aquasil/Aquasil (C18 40 × 1 mm) |
| Inj. Volume: | 2.0 μL |
| Solvent A: | H₂O, 0.02% TFA |
| Solvent B: | MeCN, 0.018% TFA |
| Gradient: | linear |
| Channel A: | UV 214 nm |
| Channel B: | ELS |

| Step | Time (min) | Dura.(min) | Flow (μL/min) | Sol. A | Sol. B |
|---|---|---|---|---|---|
| 0 | 0.00 | 0.00 | 300.00 | 95.00 | 5.00 |
| 1 | 0.00 | 0.01 | 300.00 | 95.00 | 5.00 |
| 2 | 0.01 | 3.20 | 300.00 | 10.00 | 90.00 |
| 3 | 3.21 | 1.00 | 300.00 | 10.00 | 90.00 |
| 4 | 4.21 | 0.10 | 300.00 | 95.00 | 5.00 |
| 5 | 4.31 | 0.40 | 300.00 | 95.00 | 5.00 |

### Mass Spectrometer:

| | |
|---|---|
| Instrument: | PE Sciex Single Quadrupole LC/MS API-150 |
| Polarity: | Positive |
| Acquisition mode: | Profile |

Automated preparatory HPLC purifications were conducted using a Gilson® semi-preparative HPLC system under the following conditions:

Column: 75×33 mm I. D., S-5 um, 12 nm

Flow rate: 30 mL/min

Injection Volume: 0.800 mL

Room temperature

The eluent was a mixture composed of solvents A and B:
Solvent A: 0.1% trifluoroacetic acid in water
Solvent B: 0.1% trifluoroacetic acid in acetonitrile

Automated flash chromatography purifications were conducted using a CombiFlash® Companion® personal flash chromatography system under the following conditions:

Silica cartridge:
Size, 4, 12, 40, 80 or 120 g depending on the amount of material to be purified

Flow rate: Between 4 and 85 mL/min

Room temperature

The eluent was a mixture composed of solvents A and B:
Solvent A: Hexane
Solvent B: Ethyl acetate

Methods for making compounds are illustrated in the following schemes.

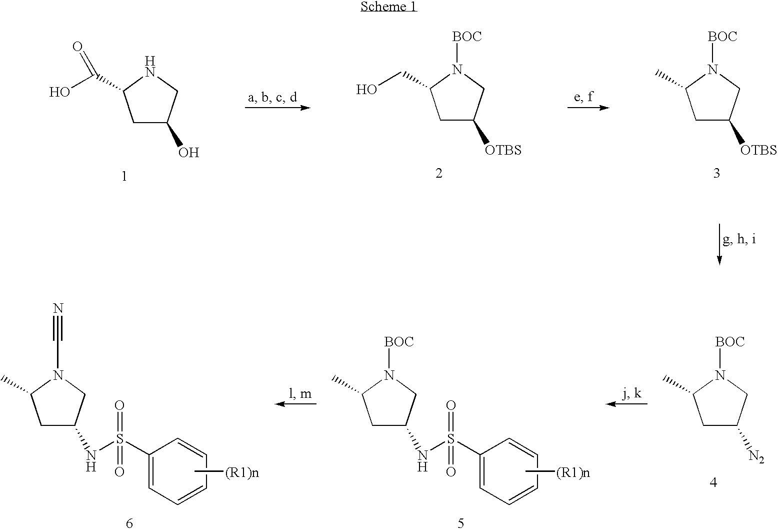

Scheme 1 reagents and conditions: a) MeOH, AcCl; b) (BOC)₂O, TEA; c) TBSCl, imidazole; d) LiBH₄; e) MsCl, TEA; f) LiEt₃BH; g) TBAF, THF; h) same as e); i) tBu₄N⁺N₃⁻, CH₃CN; j) H₂, Pd/C, MeOH; k) (R1)nPhSO₂Cl, TEA; l) 4 N HCl, Dioxane; m) BrCN, DCM.

Scheme 1 represents a general reaction scheme for preparing compounds according to Formula I wherein R2a, R2b, R2c, and R3 are each H. R1 and n are as defined above unless otherwise defined. As shown in Scheme 1, the (5S)-methyl substituted compounds of Formula (I) can be prepared in a multi-step sequence from the commercially available (4S)-4-hydroxy-D-proline 1. Esterification of 1 in acidic methanol, followed by successive conversion of the amine to the corresponding BOC derivative then of the secondary alcohol to a TBS ether using standard functional group protection procedures well know in the art, gives, after reduction of the methyl ester moiety, the primary alcohol 2. Subsequent conversion of 2 to the corresponding methanesulfonate ether followed by treatment with lithium triethyl borohydride produces the (5S)-methyl derivative 3. Further desilylation of compound 3 following standard deprotection procedures well known in the art such as treatment with TBAF gives a secondary alcohol intermediate, which is transformed to the azide 4 with inversion of configuration at C-3 by activation of the alcohol as methanesulfonate ether and treatment with an azide reagent such as tetra-t-butyl ammonium azide. Palladium-catalyzed reduction of 4 with hydrogen followed by treatment with a suitable sulfonyl chloride R₁SO₂Cl gives the derivative 5. Subsequent removal of the BOC protecting group with an acid reagent such as 4N HCl in dioxane followed by treatment with cyanogen bromide results in the formation of the desired final compound 6.

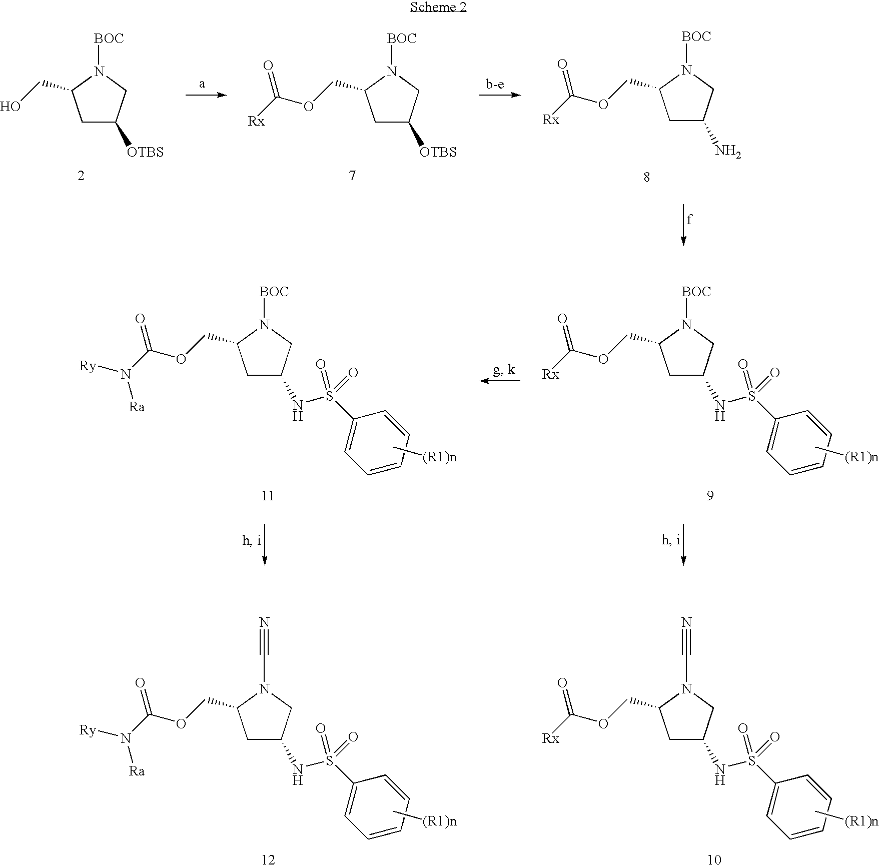

Scheme 2 reagents and conditions: a) RxCOCl, base; b) TBAF, THF; c) MsCl, TEA; d) tBu₄N⁺N₃⁻, CH₃CN; e) H₂, Pd/C; f) (R1)nPhSO₂Cl, TEA; g) Hydrolysis; h) H⁺; i) BrCN, DCM; k) RyN═C═O (where Ry is not H and Ra is H) TEA or RyRaNC(O)Cl (where Ry and Ra are not H), TEA or CDI, DCM then RyRaNH (where Ry and Ra are not H).

Scheme 2 represents a general reaction scheme for preparing compounds according to Formula I wherein R2a is —OC(O)Rx or —OC(O)NRaRy and R2b, R2c, and R3 are each H. R1 and n are as defined above unless otherwise defined. As depicted in Scheme 2, compound 2 can be treated with a suitable acyl chloride to produce the corresponding ester 7. Conversion of 7 to the primary amine 8 is carried out following a sequence of reactions analogous to that shown in Scheme 1. Treatment of 8 with a suitable sulfonyl chloride produces compound 9 that can be further elaborated to the ester derivative 10 of Formula (I) following a two-step cyanation procedure analogous to that depicted in Scheme 1. Alternatively, intermediate 9 can be hydrolysed to a primary alcohol using standard conditions such as LiOH in methanol and water and subsequently treated with a suitable reagent to produce the carbamate derivative 11. Transformation to the corresponding cyanamide 12 of Formula (I) can be achieved following a two-step cyanation procedure analogous to that depicted in Scheme 1.

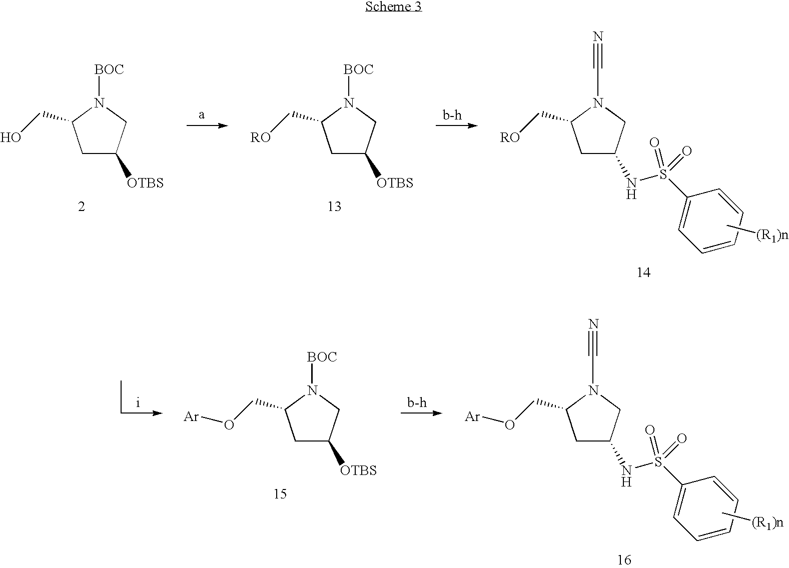

Scheme 3 reagents and conditions: a) NaH, RX, DMF; b) TBAF, THF; c) MsCl, TEA; d) tBu₄N⁺N₃⁻, CH₃CN; e) H₂, Pd/C; f) (R1)nPhSO₂Cl, TEA; g) H⁺; h) BrCN, DCM; i) PPh₃, DEAD, DCM, ArOH (where Ar is R23 or R24).

Scheme 3 represents a general reaction scheme for preparing compounds according to Formula I wherein R2a is an ether functional group (—OR20, —OR21, —OR22, —OR23, or —OR24) and R2b, R2c, and R3 are each H. R1 and n are as defined above unless otherwise defined. As depicted in Scheme 3, compound 2 can be treated with a suitable alkylating reagent in the presence of a base such as sodium hydride and an organic solvent such as DMF to give the corresponding ether intermediate 13 (R=R20, R21, or R22). Alternatively, a Mitsunobu reaction between 2 and a suitable hydroxy aryl derivative following procedures well known in the art affords the corresponding phenoxy or heteroaryloxy substituted compound 15 (Ar=R23 or R24). Both intermediates 13 and 15 can be further elaborated respectively to the cyanamide 14 or 16 of Formula (I) following a sequence of reactions analogous to that depicted in Scheme 1.

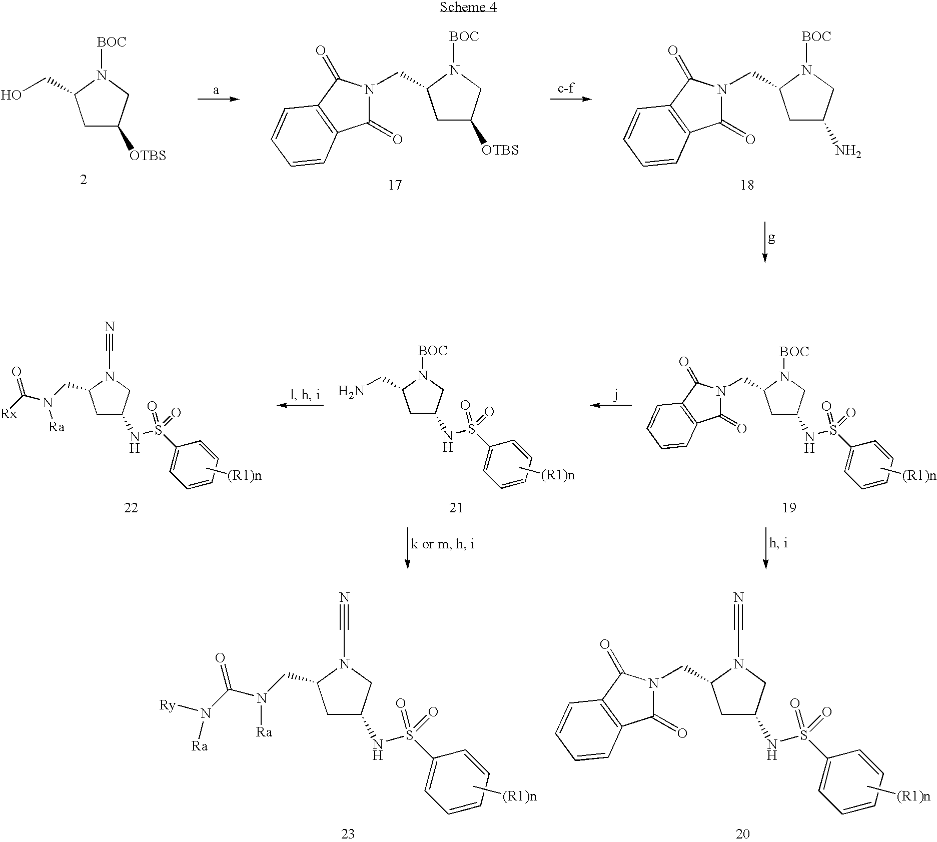

Scheme 4 reagents and conditions: a) phthalimide, PPh₃, DEAD; c) TBAF, THF; d) MsCl, TEA, DMAP; e) tBu₄N⁺N₃⁻, CH₃CN; f) H₂, Pd/C; g) (R1)nPhSO₂Cl, TEA; h) H⁺; i) BrCN, DCM; j) hydrazine, EtOH k) RyN═C═O (where Ry is not H and each Ra is H), DCM; l) RxC(O)Cl, TEA; m) Phenyl chloroformate, TEA then RyNHRa

Scheme 4 represents a general reaction scheme for preparing compounds according to Formula I wherein R2a is —NRaC(O)Rx, —NRaC(O)NRaRy, NRaC(O)R22, or pthalimidyl, and each Ra is H. R2b, R2c, and R3 are each H. R1 and n are as defined above unless otherwise defined. As depicted in Scheme 4, compound 2 can be transformed to the phthalimide derivative 17, by conversion to the corresponding methanesulfonate ether followed by treatment with phthalimide in the presence of a base following procedures well known in the art. Intermediate 17 can be further processed to the primary amine 18 following a four-step sequence analogous to that depicted in Scheme 1. Treatment of 18 with a suitable sulfonyl chloride produces compound 19 that can be further elaborated to the phthalimide derivative 20 of Formula (I) following a two-step cyanation procedure analogous to that depicted in Scheme 1. Alternatively, removal of the phthalimide group by reaction with a nucleophile such as methylamine or hydrazine leads to the amine intermediate 21. Coupling of compound 21 with a suitable reagent such as an acyl chloride or an isocyanate (either bought from a commercial source or prepared from a carboxylic acid via the Curtius reaction) followed by a two-step cyanation procedure analogous to that depicted in Scheme 1 produces respectively the amide 22 or the urea 23. Alternatively 23 can be prepared by reacting 21 with phenylchoroformate followed by treatment with an amine and a two-step cyanation procedure analogous to that depicted in Scheme 1.

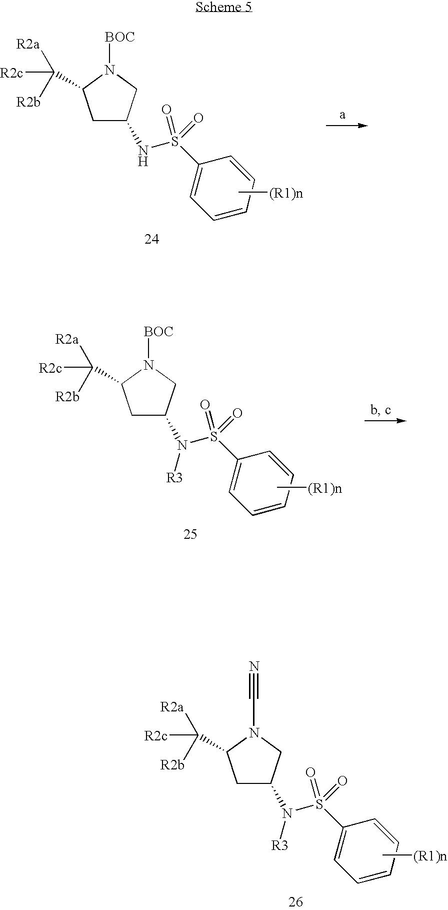

Scheme 5 reagents and conditions: a) R3X, NaH; b) H⁺ c) BrCN, DCM

Scheme 5 represents a general reaction scheme for preparing compounds according to Formula I wherein R3 is a functional group as defined other than H. R2a, R2b, R2c, R1 and n are as defined above unless otherwise defined. As depicted in Scheme 5, a suitably protected intermediate 24, which can prepared following procedures analogous to those depicted in Schemes 1-4, can be treated with an alkylating reagent R3X in the presence of a base such as NaH to give the N-alkylated derivative 25. Subsequent removal of the BOC protecting group of 25 with an acid reagent such as 4N HCl in dioxane followed by treatment with cyanogen bromide results in the formation of the desired compounds 26.

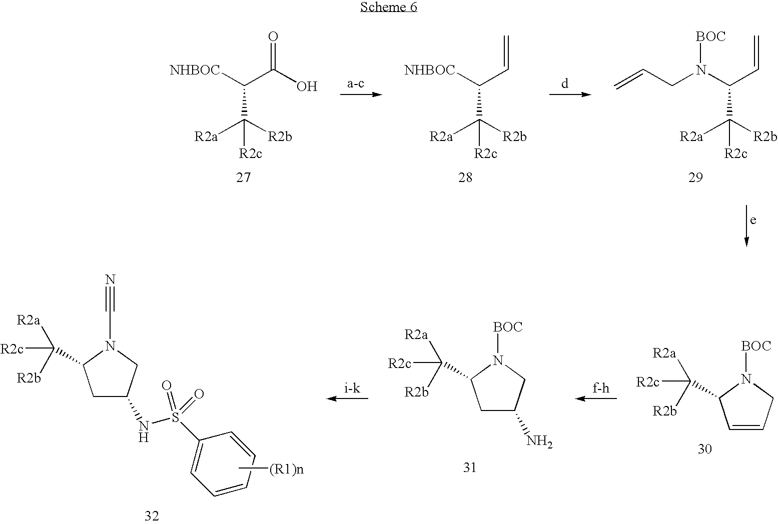

Scheme 6 reagents and conditions: a) NHOMe; b) LiAlH₄; c) (PhP)₃P⁺Br⁻, base; d) Allyl bromide, NaH; e) Grubbs catalyst, DCM; f) BH₃, H₂O₂; g) DPPA, DEAD or Ms₂O then NaN₃; h) H₂, Pd/C; i) (R1)nPhSO₂Cl, TEA; j) H⁺; k) BrCN, DCM.

Scheme 6 represents another general reaction scheme for preparing compounds according to Formula I wherein R3 is H. R2a, R2b, R2c, R1 and n are as defined above unless otherwise defined. As depicted in Scheme 6, a suitable BOC-protected amino-acid 27 is transformed to the corresponding alkene derivative 28 by first conversion to the corresponding Weinreb amide, followed by reduction with LiAlH₄ and subsequent Wittig reaction with tri-phenyl phosphonium bromide in the presence of a strong base following procedures well known to those skilled in the art. Treatment of 28 with allyl bromide in the presence of a base such as NaH leads to the formation of the disubstituted derivative 29. Compound 29 can undergo a ring closing metathesis reaction using Grubbs' catalyst to give the pyrroline derivative 30. Oxidative hydroboration of 30, followed by treatment of the resulting alcohol with DPPA or a mesylation/azide displacement reaction sequence then hydrogenation of the azide intermediate yields amine 31. Treatment of compound 31 with a suitable sulfonyl chloride followed by a two-step cyanation procedure analogous to that depicted in Scheme 1, produces compound 32.

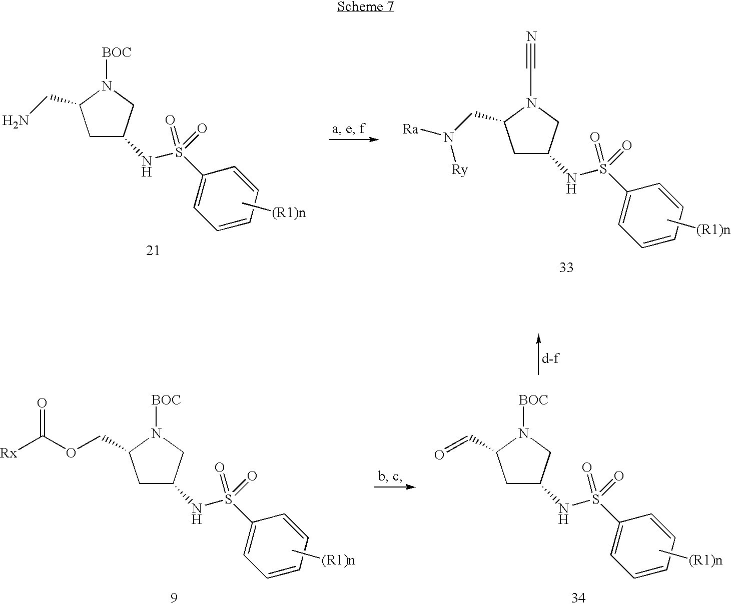

Scheme 7 reagents and conditions: a) aldehyde, NaBH₄; b) MeONa, MeOH or LiOH, MeOH, H₂O; c) NaBr, TEMPO, DCM; d) RaRyNH, PS-(OAc)₃BH; e) H⁺; f) BrCN, DCM.

Scheme 7 represents general reaction schemes for preparing compounds according to Formula I wherein R2a is an amine functional group (—NRaRy) and R2b, R2c, and R3 are each H. R1 and n are as defined above unless otherwise defined. As depicted in Scheme 7, compound 21 can be treated with a suitable aldehyde in the presence of sodium borohydride to give an intermediate which can subsequently undergo a two-step cyanation procedure analogous to that depicted in Scheme 1 to give compound 33 of Formula (I). Alternatively, compound 9 can be hydrolysed or methanolysed then re-oxidized with a suitable reagent such as TEMPO to give an aldehyde intermediate 34. Compound 34 can undergo a reductive amination with a suitable amine in the presence of a polymer-supported triacetoxy borohydride reagent then a two-step cyanation procedure analogous to that depicted in Scheme 1 to produce compound 33.

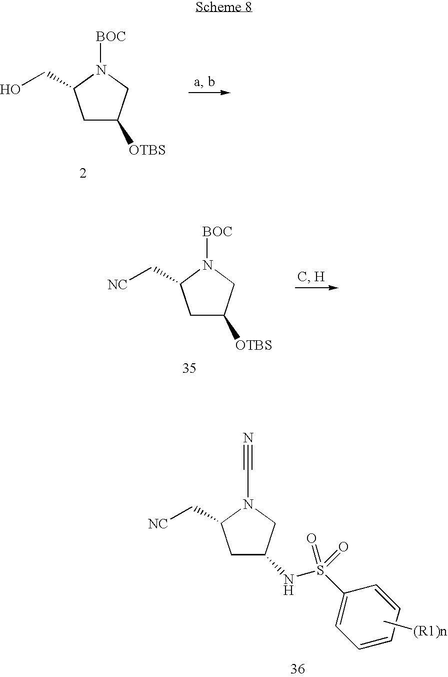

Scheme 8 reagents and conditions: a) MsCl, TEA; b) tBuN⁺CN⁻, AcCN; c) TBAF, THF then MsCl, TEA; d) tBuN⁺N₃⁻, CH₃CN; e) H₂, Pd/C; f) (R1)nPhSO₂Cl, TEA; g) H⁺; h) BrCN, DCM.

Scheme 8 represents a general reaction scheme for preparing compounds according to Formula I wherein R2a is a nitrile functional group (—CN) and R2b, R2c, and R3 are each H. R1 and n are as defined above unless otherwise defined. As depicted in Scheme 8, compound 2 can be mesylated then treated with a cyanide reagent such as tetrabutylammonium cyanide to give the nitrile derivative 35. Compound 35 can then be transformed into compound 36 of Formula I following a sequence of reactions analogous to that depicted in Scheme 3 for the preparations of the ether derivatives 14 and 16 from the intermediates 13 and 15.

### INTERMEDIATES

The following Intermediate compounds are useful for preparing compounds of Formula I.

### Intermediate 1

### methyl (4S)-4-hydroxy-D-prolinate

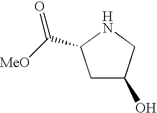

In a three necked flask under argon atmosphere equipped with a magnetic stir bar and a reflux condenser was placed MeOH (7 mL). To the system was added acetyl chloride (0.898 g, 11.4 mmol) in an ice bath followed by (4S)-4-hydroxy-D-proline (1 g, 7.6 mmol). The resultant mixture was heated at reflux for 12 hours and cooled down to room temperature. Ether was added to cause precipitation and the resulting brown powder was filtered off to afford the title compound. LC-MS: m/z, 146 (M+H).

### Intermediate 2

### 1-(1,1-dimethylethyl) 2-methyl (2R,4S)-4-hydroxy-1,2-pyrrolidinedicarboxylate

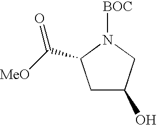

To methyl (4S)-4-hydroxy-D-prolinate (1.1147 g, 7.6 mmol) in DCM (8 mL) was added triethylamine (3.19 mL, 22.9 mmol) while the bath cooled down to 0° C. To the resultant mixture, di-tert-butyl dicarbonate (1.8318 g, 8.4 mmol) was added, stirred from 0° C. to room temperature over the weekend. The resultant mixture was washed with 1 N HCl and NaHCO₃ solution, dried over MgSO₄ and evaporated to afford the title compound as a yellow oil (1.4406 g). LC-MS: m/z, 246 (M+H).

### Intermediate 3

### 1-(1,1-dimethylethyl) 2-methyl (2R,4S)-4-{[(1,1-dimethylethyl)(dimethyl)silyl]oxy}-1,2-pyrrolidinedicarboxylate

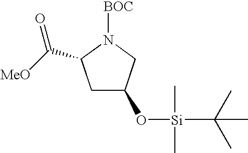

To 1-(1,1-dimethylethyl) 2-methyl (2R,4S)-4-hydroxy-1,2-pyrrolidinedicarboxylate (1.4406 g, 5.9 mmol) in DMF (˜4 mL) was added imidazole (0.4399 g, 6.46 mmol) and TBSCl (1.8512 g, 12.3 mmol). The resultant mixture was stirred at room temperature for 1.5 hours and diluted in ether and water. The organic layers were washed with 1 N HCl and NaHCO₃ solution, dried on MgSO₄ and the ether removed under vacuum to afford the title compound as a yellow liquid (2.3591 g). LC-MS: m/z, 360 (M+H).

### Intermediate 4

### 1,1-dimethylethyl (2R,4S)-4-{[(1,1-dimethylethyl)(dimethyl)silyl]oxy}-2-(hydroxymethyl)-1-pyrrolidinecarboxylate

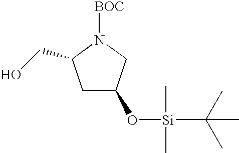

1-(1,1-dimethylethyl) 2-methyl (2R,4S)-4-{[(1,1-dimethylethyl)(dimethyl)silyl]oxy}-1,2-pyrrolidinedicarboxylate (2.3591 g, 6.56 mmol) was diluted in THF (30 mL) and the mixture was cooled down to 0° C. under argon. To the resultant mixture a solution of LiBH₄ in THF (15 mL) was added dropwise over 30 minutes, and stirred from 0° C. to room temperature overnight. The reaction mixture was cooled down to 0° C., neutralized with 1 N HCl, and water (50 mL) and ethyl acetate (50 mL) were added. The layers were separated and the aqueous layer was extracted with ethyl acetate (˜30 mL). The organic layers were combined and washed with NaHCO₃ solution (3×20 mL), dried on MgSO₄ and evaporated under vacuum to afford the title compound as a colorless liquid (2.02 g). LC-MS: m/z, 332 (M+H).

### Intermediate 5

### 1,1-dimethylethyl (2R,4S)-4-{[(1,1-dimethylethyl)(dimethyl)silyl]oxy}-2-{[(methylsulfonyl)oxy]methyl}-1-pyrrolidinecarboxylate

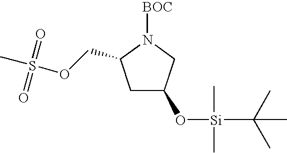

To 1,1-dimethylethyl (2R,4S)-4-{[(1,1-dimethylethyl)(dimethyl)silyl]oxy}-2-(hydroxymethyl)-1-pyrrolidinecarboxylate (2.02 g, 6.09 mmol) in DCM (˜12 mL) was added triethylamine (1.69 mL, 12.19 mmol). The resultant mixture was placed under argon and cooled down to −10° C. To the resultant mixture methanesulfonyl chloride (0.71 mL, 9.14 mmol) was added and stirred from −10° C. to room temperature overnight. The reaction mixture was diluted in DCM (˜50 mL) and water (˜50 mL) and the layers were separated. The organic layer was washed with 1 N HCl (2×20 mL), neutralized with NaHCO₃ solution, dried on MgSO₄ and the DCM was evaporated under vacuum to afford the title compound as a colorless liquid (1.9462 g). LC-MS: m/z, 410 (M+H).

### Intermediate 6

### 1,1-dimethylethyl (2S,4S)-4-{[(1,1-dimethylethyl)(dimethyl)silyl]oxy}-2-methyl-1-pyrrolidinecarboxylate

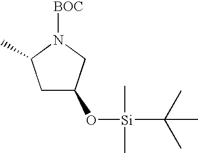

1,1-dimethylethyl (2R,4S)-4-{[(1,1-dimethylethyl)(dimethyl)silyl]oxy}-2-{[(methylsulfonyl)oxy]methyl}-1-pyrrolidinecarboxylate (0.1 g, 0.24 mmol) was diluted in THF (1 mL) and the mixture was cooled down to 0° C. under argon. To the resultant mixture LiEt₃BH (superhydride), (2.44 mL, 2.44 mmol) was added dropwise and stirred at room temperature for 2 hours. The reaction mixture was diluted in water (40 mL) and ethyl acetate (60 mL), the layers were separated and the aqueous layer was extracted with ethyl acetate. The organic layers were combined, washed with 1 N HCl and NaHCO₃ solution, dried on MgSO₄ and the solvent was evaporated to afford the title compound as an orange oil (0.0824 g). LC-MS: m/z, 316 (M+H).

### Intermediate 7

### 1,1-dimethylethyl-(2S,4S)-4-hydroxy-2-methyl-1-pyrrolidinecarboxylate

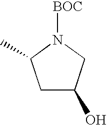

To 1,1-dimethylethyl (2S,4S)-4-{[(1,1-dimethylethyl)(dimethyl)silyl]oxy}-2-methyl-1-pyrrolidinecarboxylate (0.19201 g, 6.08 mmol) in THF (˜1 mL) was added TBAF (12 mL, 12.17 mmol) under argon. The resultant mixture was stirred at room temperature for 3 hours and purified by flash chromatography (silica gel) with gradient ethyl acetate:hexanes (1:2) to (2:1) to afford the title compound (1.0326 g). LC-MS: m/z, 202 (M+H).

### Intermediate 8

### 1,1-dimethylethyl (2S,4S)-2-methyl-4-[(methylsulfonyl)oxy]-1-pyrrolidinecarboxylate

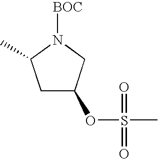

1,1-dimethylethyl (2S,4S)-4-hydroxy-2-methyl-1-pyrrolidinecarboxylate (1.0326 g, 5.13 mmol) in DCM (˜2 mL) was placed under argon and cooled to 0° C. To the reaction mixture was added triethylamine (1.2973 g, 12.8 mmol) and methanesulfonyl chloride (1.1753 g, 10.3 mmol). The resultant mixture was stirred for 16 hours and diluted in ether (˜100 mL) and water (˜60 mL). The aqueous layer was extracted with ethyl acetate (×2). The organic layers were combined, washed with 1 N HCl and NaHCO₃ solution, dried on MgSO₄ and the solvent evaporated under vacuum to afford the title compound as a yellow liquid (0.6395 g). LC-MS: m/z, 280 (M+H).

### Intermediate 9

### 1,2-dimethylethyl-(2S,2R)-4-azido-2-methyl-1-pyrrolidinecarboxylate

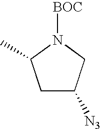

To 1,1-dimethylethyl (2S,4S)-2-methyl-4-[(methylsulfonyl)oxy]-1-pyrrolidinecarboxylate (1.55 g, 5.55 mmol) in acetonitrile (15 mL) was added tertabutylammonium azide (1.77 g, 6.22 mmol) in acetonitrile (10 ml). The resultant mixture was heated at reflux overnight. The solvent was removed under vacuum and the resulting crude material was diluted in ethyl acetate (˜300 ml) and water (˜100 ml). Both layers were separated and the aqueous layer was extracted again with ethyl acetate (×2). The organic layers were combined, dried over MgSO₄ then concentrated under vacuum to give a crude material, which was purified by automated flash chromatography (40 g silica cartridge) to afford the title compound as a colorless liquid (0.966 g). LC-MS: m/z, 227 (M+H).

### Intermediate 10

### 1,1-dimethylethyl (2S,4R)-4-amino-2-methyl-1-pyrrolidinecarboxylate

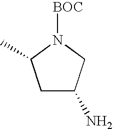

1,1-dimethylethyl (2S,4R)-4-azido-2-methyl-1-pyrrolidinecarboxylate (0.966 g, 4.26 mmol) in MeOH (˜10 mL) was hydrogenated over 10% Pd/C in a H-Cube™ reactor (ThalesNano Nanotechnology Inc., H1031 Budapest, Zahony u. 7., Graphisoft Park Hungary, http://www.thalesnano.com/products/h-cube-midi). The solvent was evaporated under vacuum to afford the title compound as a colorless liquid (0.898 g). LC-MS: m/z, 201 (M+H).

### Intermediate 11

### 1,1-dimethylethyl (2S,4R)-4-{[(2,5-dibromophenyl)sulfonyl]amino}-2-methyl-1-pyrrolidinecarboxylate

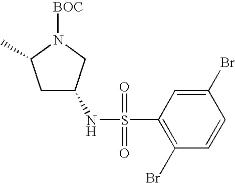

To 1,1-dimethylethyl (2S,4R)-4-amino-2-methyl-1-pyrrolidinecarboxylate (0.080 g, 0.40 mmol) in DCM (˜3 mL) was added DIPEA (0.12 mL, 0.70 mmol) and 2,5-dibromobenzenesulfonyl chloride (0.117 g, 0.35 mmol). The resultant mixture was stirred at room temperature for 3 hours; the solvent was evaporated under vacuum and loaded onto a 5 g silica SPE cartridge, eluting sequentially with dichloromethane and ethyl acetate. The ethyl acetate fractions were combined and evaporated to afford the title compound (0.1627 g). LC-MS: m/z, 500 (M+H).

### Intermediate 12

### 1,1-dimethylethyl (2S,4R)-4-{[(2,5-dichlorophenyl)sulfonyl]amino}-2-methyl-1-pyrrolidinecarboxylate

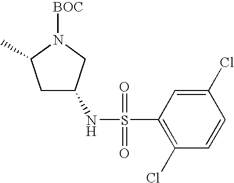

To 1,1-dimethylethyl (2S,4R)-4-amino-2-methyl-1-pyrrolidinecarboxylate (0.0208 g, 0.10 mmol) in DCM (˜1 mL) was added DIPEA (0.04 mL, 0.20 mmol) and 2,5-dichlorobenzenesulfonyl chloride (0.026 g, 0.10 mmol). The resultant mixture was stirred at room temperature for 16 hours, the solvent evaporated under vacuum to yield a brown oil which was loaded onto a 2 g silica SPE cartridge, eluting sequentially with dichloromethane and ethyl acetate. The DCM fractions were combined and evaporated to afford the title compound as a colorless liquid (0.0149 g). LC-MS: m/z, 410 (M+H).

### Intermediate 13

### 1,1-dimethylethyl (2S,4R)-4-({[2,5-bis(methyloxy)phenyl]sulfonyl}amino)-2-methyl-1-pyrrolidinecarboxylate

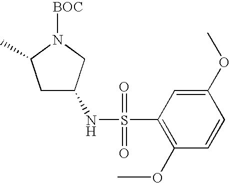

To 1,1-dimethylethyl (2S,4R)-4-amino-2-methyl-1-pyrrolidinecarboxylate (0.100 g, 0.50 mmol) in DCM (˜5 mL) was added DIPEA (0.176 mL, 1 mmol) and 2,5-bis(methyloxy)benzenesulfonyl chloride (0.118 g, 0.50 mmol). The resultant mixture was stirred at room temperature overnight, the solvent evaporated under vacuum and the resulting crude diluted in DCM. The DCM layer was isolated and loaded onto a silica SPE cartridge, eluting sequentially with DCM and ethyl acetate. The relevant fractions were combined and evaporated to afford the title compound (0.0615 g). LC-MS: m/z, 401 (M+H).

### Intermediate 14

### 1,1-dimethylethyl (2S,4R)-4-{[(3-bromophenyl)sulfonyl]amino}-2-methyl-1-pyrrolidinecarboxylate

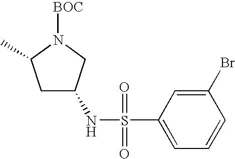

Intermediate 14 was prepared using the general procedure described above for Intermediate 13, replacing 2,5-bis(methyloxy)benzenesulfonyl chloride (0.118 g, 0.50 mmol) with 3-bromobenzenesulfonyl chloride (0.128 g, 0.50 mmol) to afford the title compound (0.1351 g). LC-MS: m/z, 421 (M+H).

### Intermediate 15

### 1,1-dimethylethyl (2S,4R)-4-({[2-bromo-5-(trifluoromethyl)phenyl]sulfonyl}amino)-2-methyl-1-pyrrolidinecarboxylate

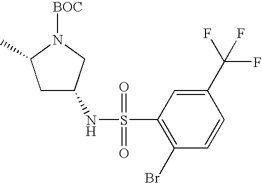

Intermediate 15 was prepared using the general procedure described above for Intermediate 13, replacing 2,5-bis(methyloxy)benzenesulfonyl chloride (0.118 g, 0.50 mmol) with 2-bromo-5-(trifluoromethyl)benzenesulfonyl chloride (0.162 g, 0.50 mmol) to afford the title compound (0.1206 g). LC-MS: m/z, 487 (M+H).

### Intermediate 16

### 1,1-dimethylethyl (2S,4R)-4-({[2-chloro-5-(trifluoromethyl)phenyl]sulfonyl}amino)-2-methyl-1-pyrrolidinecarboxylate

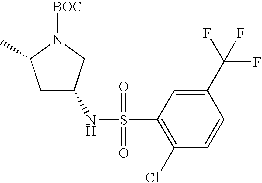

Intermediate 16 was prepared using the general procedure described above for Intermediate 13, replacing 2,5-bis(methyloxy)benzenesulfonyl chloride (0.118 g, 0.50 mmol) with 2-chloro-5-(trifluoromethyl)benzenesulfonyl chloride (0.139 g, 0.50 mmol) to afford the title compound (0.2066 g). LC-MS: m/z, 443 (M+H).

### Intermediate 17

### 1,1-dimethylethyl (2S,4R)-4-({[5-bromo-2-(methyloxy)phenyl]sulfonyl}amino)-2-methyl-1-pyrrolidinecarboxylate

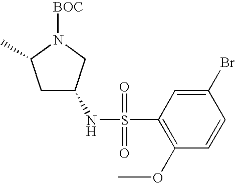

Intermediate 17 was prepared using the general procedure described above for Intermediate 13, replacing 2,5-bis(methyloxy)benzenesulfonyl chloride (0.118 g, 0.50 mmol) with 5-bromo-2-(methyloxy)benzenesulfonyl chloride (0.143 g, 0.50 mmol) to afford the title compound (0.1285 g). LC-MS: m/z, 450 (M+H).

### Intermediate 18

### 1,1-dimethylethyl (2S,4R)-4-{[(5-bromo-2,4-difluorophenyl)sulfonyl]amino}-2-methyl-1-pyrrolidinecarboxylate

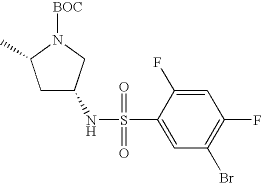

To 1,1-dimethylethyl (2S,4R)-4-amino-2-methyl-1-pyrrolidinecarboxylate (0.0835 g, 0.42 mmol) in DCM (˜5 mL) was added a solution of 5-bromo-2,4-difluorobenzenesulfonyl chloride (0.122 g, 0.42 mmol) in DCM (˜2 mL) and DIPEA (0.146 mL, 0.84 mmol). The resultant mixture was stirred at room temperature overnight, evaporated and loaded onto a 5 g silica SPE cartridge, eluting sequentially with DCM and ethyl acetate. The ethyl acetate fractions were combined and evaporated to afford the title compound (0.140 g).

LC-MS: m/z, 456 (M+H).

### Intermediate 19

### 1,1-dimethylethyl (2S,4R)-2-methyl-4-{[(2,4,5-trifluorophenyl)sulfonyl]amino}-1-pyrrolidinecarboxylate

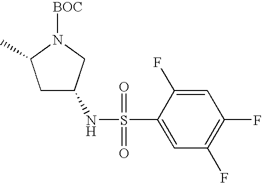

To 1,1-dimethylethyl (2S,4R)-4-amino-2-methyl-1-pyrrolidinecarboxylate (0.075 g, 0.38 mmol) in DCM (˜5 mL) was added a solution of 2,4,5-trifluorobenzenesulfonyl chloride (0.075 g, 0.38 mmol) in DCM (˜5 mL) and DIPEA (0.13 mL, 0.75 mmol). The resultant mixture was stirred at room temperature overnight, evaporated and loaded onto a 5 g silica SPE cartridge, eluting sequentially with DCM and ethyl acetate. The ethyl acetate fractions were combined and evaporated to afford the title compound (0.0942 g). LC-MS: m/z, 395 (M+H).

### Intermediate 20

### 1,1-dimethylethyl (2R,4S)-4-{[(1,1-dimethylethyl)(dimethyl)silyl]oxy}-2-[(methyloxy)methyl]-1-pyrrolidinecarboxylate

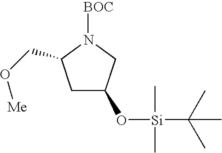

1,1-dimethylethyl (2R,4S)-4-{[(1,1-dimethylethyl)(dimethyl)silyl]oxy}-2-(hydroxymethyl)-1-pyrrolidinecarboxylate (0.170 g, 0.51 mmol) in DMF (˜2 mL) was cooled down to 0° C. and placed under argon atmosphere. NaH (0.049 g, 2.0 mmol) and methyl iodide (0.32 mL, 5.13 mmol) were added and the resultant mixture stirred from 0° C. to room temperature for 5 hours. The reaction mixture was quenched with MeOH and diluted in ethyl acetate (˜50 mL). The organic layer was washed with 0.1 N HCl and NaHCO₃ solution, dried over MgSO₄ and concentrated to afford the title compound (0.0848 g). LC-MS: m/z, 346 (M+H).

### Intermediate 21

### 1,1-dimethylethyl (2R,4S)-4-hydroxy-2-[(methyloxy)methyl]-1-pyrrolidinecarboxylate

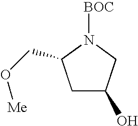

To 1,1-dimethylethyl (2R,4S)-4-{[(1,1-dimethylethyl)(dimethyl)silyl]oxy}-2-[(methyloxy)methyl]-1-pyrrolidinecarboxylate (0.698 g, 2.02 mmol) in THF (˜10 mL) was added TBAF (4 mL, 4.02 mmol) under argon atmosphere and stirred at room temperature for 4 hours. The reaction mixture was concentrated and diluted in ethyl acetate. The organic layer was washed with water (2×10 mL). The aqueous layers were combined and washed again with EA (2×20 mL). The organic layers were dried over MgSO₄ and concentrated to afford the title compound as a yellow liquid (0.426 g). LC-MS: m/z, 232 (M+H).

### Intermediate 22

### 1,1-dimethylethyl (2R,4S)-2-[(methyloxy)methyl]-4-[(methylsulfonyl)oxy]-1-pyrrolidinecarboxylate

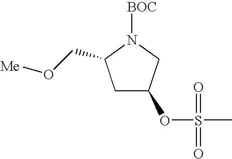

Under argon atmosphere at 0° C. 1,1-dimethylethyl (2R,4S)-4-hydroxy-2-[(methyloxy)methyl]-1-pyrrolidinecarboxylate (0.426 g, 1.8 mmol) was diluted in DCM (˜5 mL). To the resultant mixture triethylamine (0.64 mL, 4.60 mmol) and methanesulfonyl chloride (0.29 mL) were added and stirred from 0° C. to room temperature for 4.5 hours. The reaction mixture was concentrated and diluted in ethyl acetate and water. Both layers were separated and the organic layer was washed with 0.1 N HCl and NaHCO₃ solution, dried over MgSO₄ and concentrated to afford the title compound (0.5302 g). LC-MS: m/z, 310 (M+H).

### Intermediate 23

### 1,1-dimethylethyl (2R,4R)-4-azido-2-[(methyloxy)methyl]-1-pyrrolidinecarboxylate

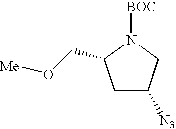

To 1,1-dimethylethyl (2R,4S)-2-[(methyloxy)methyl]-4-[(methylsulfonyl)oxy]-1-pyrrolidinecarboxylate (0.530 g, 1.7 mmol) in DMF (8 mL) was added NaN₃ (0.445 g, 6.8 mmol) and microwaved for 1 hour at 125° C. DMF was removed under vacuum and the crude was diluted in ethyl acetate and water. Both layers were separated and the organic layer was washed with 0.1 N HCl and NaHCO₃ solution, dried over MgSO₄, concentrated and purified by automated flash chromatography (12 g silica cartridge) to afford the title compound as a colorless liquid (0.3013 g). LC-MS: m/z, 257 (M+H).

### Intermediate 24

### 1,1-dimethylethyl (2R,4R)-4-amino-2-[(methyloxy)methyl]-1-pyrrolidinecarboxylate

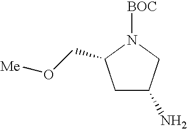

1,1-dimethylethyl (2R,4R)-4-azido-2-[(methyloxy)methyl]-1-pyrrolidinecarboxylate (0.3013 g, 1.2 mmol) in MeOH (˜10 mL) was hydrogenated over 10% Pd/C in a H-Cube™ reactor. The product was concentrated to afford the title compound as a colorless oil (0.230 g). LC-MS: m/z, 231 (M+H).

### Intermediate 25

### 1,1-dimethylethyl (2R,4R)-4-{[(2,5-dibromophenyl)sulfonyl]amino}-2-[(methyloxy)methyl]-1-pyrrolidinecarboxylate

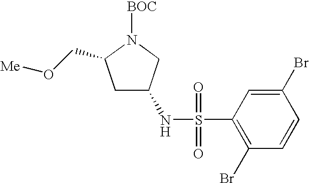

To 1,1-dimethylethyl (2R,4R)-4-amino-2-[(methyloxy)methyl]-1-pyrrolidinecarboxylate (0.077 g, 0.33 mmol) in DCM (˜3 mL) was added DIPEA (0.115 mL, 0.66 mmol) and 2,5-dibromobenzenesulfonyl chloride (0.110 g, 0.33 mmol). The resultant mixture was stirred at room temperature overnight. The solvent was removed under vacuum and loaded onto a 5 g silica SPE cartridge, eluting sequentially with DCM and ethyl acetate. The ethyl acetate fractions were combined and evaporated to afford the title compound as a colorless oil (0.159 g). LC-MS: m/z, 529 (M+H).

### Intermediate 26

### 1,1-dimethylethyl (2R,4R)-4-{[(2,5-dichlorophenyl)sulfonyl]amino}-2-[(methyloxy)methyl]-1-pyrrolidinecarboxylate

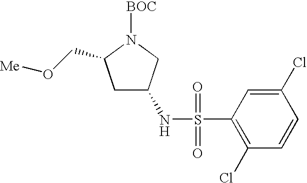

Intermediate 26 was prepared using the general procedure described above for Intermediate 25, replacing 2,5-dibromobenzenesulfonyl chloride (0.110 g, 0.33 mmol) with 2,5-dichlorobenzenesulfonyl chloride (0.081 g, 0.33 mmol) to afford the title compound as a colorless oil (0.144 g). LC-MS: m/z, 440 (M+H).

### Intermediate 27

### 1,1-dimethylethyl (2R,4R)-4-({[2-bromo-5-(trifluoromethyl)phenyl]sulfonyl}amino)-2-[(methyloxy)methyl]-1-pyrrolidinecarboxylate

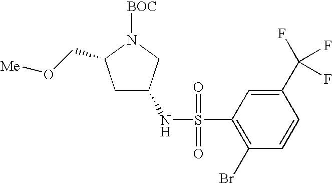

Intermediate 27 was prepared using the general procedure described above for Intermediate 25, replacing 2,5-dibromobenzenesulfonyl chloride (0.110 g, 0.33 mmol) with 2-bromo-5-(trifluoromethyl)benzenesulfonyl chloride (0.107 g, 0.33 mmol) to afford the title compound as a colorless oil (0.129 g). LC-MS: m/z, 517 (M+H).

### Intermediate 28

### 1,1-dimethylethyl (2R,4S)-4-{[(1,1-dimethylethyl)(dimethyl)silyl]oxy}-2-[(phenyloxy)methyl]-1-pyrrolidinecarboxylate

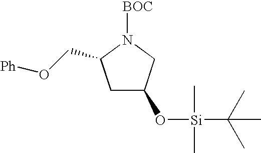

PPh₃ (0.030 g, 0.11 mmol) was mixed with a solution of diethylazodicarboxylate (0.020 g, 0.11 mmol) in CH₂Cl₂ (˜0.5 mL) and stirred at 0° C. for 5 minutes. To the resultant mixture a solution of 1,1-dimethylethyl (2R,4S)-4-{[(1,1-dimethylethyl)(dimethyl)silyl]oxy}-2-(hydroxymethyl)-1-pyrrolidinecarboxylate (0.025 g, 0.07 mmol, 1 eq), phenol (0.007 g, 0.07 mmol) and triethylamine (0.0114 g, 0.11 mmol) in CH₂Cl₂ (˜0.5 mL) was added. The resultant mixture was stirred at room temperature overnight. To this mixture diethylazodicarboxylate (1 eq), PPh₃, triethylamine and phenol (0.5 eq) were added and stirred for an additional 4 hours. The crude was concentrated and loaded onto a 5 g silica SPE cartridge, then eluted sequentially with hexane, DCM and ethyl acetate. The relevant DCM fraction was evaporated to afford the title compound (0.0084 g). LC-MS: m/z, 408 (M+H).

### Intermediate 29

### 1,1-dimethylethyl (2R,4S)-4-hydroxy-2-[(phenyloxy)methyl]-1-pyrrolidinecarboxylate

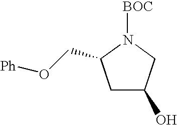

Under argon atmosphere in a solution of 1,1-dimethylethyl (2R,4S)-4-{[(1,1-dimethylethyl)(dimethyl)silyl]oxy}-2-[(phenyloxy)methyl]-1-pyrrolidinecarboxylate (0.3955 g, 0.95 mmol) in THF (˜5 mL) was added TBAF (1.9 mL, 1.90 mmol) and stirred at room temperature for 3 hours. The crude was concentrated, diluted in ethyl acetate and the organic layer was washed with water. The aqueous layer was extracted with ethyl acetate (×2); the organic layers were combined, dried over MgSO₄ and concentrated to afford the title compound (0.390 g). LC-MS: m/z, 294 (M+H).

### Intermediate 30

### 1,1-dimethylethyl (2R,4S)-4-[(methylsulfonyl)oxy]-2-[(phenyloxy)methyl]-1-pyrrolidinecarboxylate

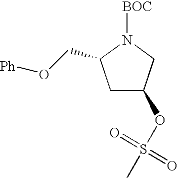

Under argon atmosphere at 0° C., 1,1-dimethylethyl (2R,4S)-4-hydroxy-2-[(phenyloxy)methyl]-1-pyrrolidinecarboxylate (0.390 g, 1.3 mmol, 1 eq.) was diluted in DCM (˜5 mL). To the resulting solution was added triethylamine (0.46 mL, 3.32 mmol) and methanesulfonyl chloride (0.2 mL, 2.66 mmol). The resultant mixture was stirred from 0° C. to room temperature for 5 hours. To this mixture triethylamine (1 eq.) and methanesulfonyl chloride (1 eq.) were added at 0° C. and the mixture was stirred from 0° C. to room temperature overnight. To the resultant mixture, DMAP (1 eq.) was added at 0° C. followed by methanesulfonyl chloride (1 eq.) which was stirred for 3 hours at room temperature. To the mixture triethylamine (0.368 mL, 2.6 mmol) and methanesulfonyl chloride (0.2 mL, 2.58 mmol) were added at 0° C. and stirred overnight to room temperature. The crude was concentrated, diluted in EA (˜100 mL) and washed with 0.1 N HCl (2×100 mL) and NaHCO₃ solution (2×100 mL). The mixture was dried over MgSO₄ and concentrated under vacuum to afford the title compound (0.5267 g). LC-MS: m/z, 372 (M+H).

### Intermediate 31

### 1,1-dimethylethyl (2R,4R)-4-azido-2-[(phenyloxy)methyl]-1-pyrrolidinecarboxylate

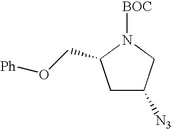

Under argon atmosphere, 1,1-dimethylethyl (2R,4S)-4-[(methylsulfonyl)oxy]-2-[(phenyloxy)methyl]-1-pyrrolidinecarboxylate (0.5267 g, 1.4 mmol) was diluted in acetonitrile (˜7 mL). After addition of tertbutylamino azide (0.50 g, 1.7 mmol) the resultant mixture was stirred at room temperature overnight, heated at reflux for 3 hours and stirred at room temperature over the weekend. The solvent was removed under vacuum and the resulting orange solid was diluted in ethyl acetate, washed with 0.1 N HCl and a sat. aq. NaHCO₃ solution and dried over MgSO₄. The organic layer was concentrated and purified by automated flash chromatography (6 g silica cartridge) to afford the title compound as (0.931 g). LC-MS: m/z, 319 (M+H).

### Intermediate 32

### 1,1-dimethylethyl (2R,4R)-4-amino-2-[(phenyloxy)methyl]-1-pyrrolidinecarboxylate

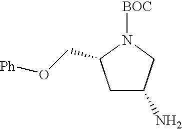

1,1-dimethylethyl (2R)-4-azido-2-[(phenyloxy)methyl]-1-pyrrolidinecarboxylate (0.093 g, 0.29 mmol) in MeOH (˜10 mL) was hydrogenated over 10% Pd/C in a H-Cube™ reactor. The product was concentrated to afford the title compound as a white solid (0.0686 g). LC-MS: m/z, 293 (M+H).

### Intermediate 33

### 1,1-dimethylethyl (2R,4R)-4-{[(2,5-dichlorophenyl)sulfonyl]amino}-2-[(phenyloxy)methyl]-1-pyrrolidinecarboxylate

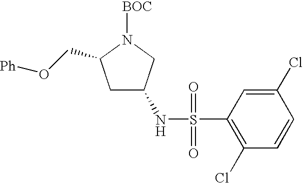

To 1,1-dimethylethyl (2R)-4-amino-2-[(phenyloxy)methyl]-1-pyrrolidinecarboxylate (0.0343 g, 0.12 mmol) in DCM (˜1 mL) was added DIPEA (0.041 mL, 0.24 mmol) and 2,5-dichlorobenzenesulfonyl chloride (0.029 g, 0.12 mmol). The resultant mixture was stirred at room temperature overnight. The crude material was concentrated under vacuum and loaded onto a 2 g silica SPE cartridge, then eluting sequentially with DCM and ethyl acetate. The DCM fractions were combined and evaporated to afford the title compound as a colorless liquid (0.0545 g). LC-MS: m/z, 501 (M+H).

### Intermediate 34

### 1,1-dimethylethyl (2R,4R)-4-{[(2,5-dibromophenyl)sulfonyl]amino}-2-[(phenyloxy)methyl]-1-pyrrolidinecarboxylate

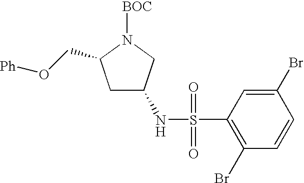

Intermediate 34 was prepared using the general procedure described above for Intermediate 33, replacing 2,5-dichlorobenzenesulfonyl chloride (0.029 g, 0.12 mmol) with 2,5-dibromobenzenesulfonyl chloride (0.039 g, 0.12 mmol) to afford the title compound as a colorless liquid (0.063 g). LC-MS: m/z, 590 (M+H).

### Intermediate 35

### 1,1-dimethylethyl (2R,4S)-4-{[(1,1-dimethylethyl)(dimethyl)silyl]oxy}-2-{[(2,2-dimethylpropanoyl)oxy]methyl}-1-pyrrolidinecarboxylate

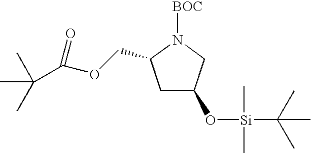

To 1,1-dimethylethyl (2R,4S)-4-{[(1,1-dimethylethyl)(dimethyl)silyl]oxy}-2-(hydroxymethyl)-1-pyrrolidinecarboxylate (2.0 g, 6.03 mmol, 1 eq.) in THF (˜50 mL) was added triethylamine (1.25 mL, 9.0 mmol) and 2,2-dimethylpropanoyl chloride (0.89 mL, 7.2 mmol). The flask was fitted with a drying flask and the reaction mixture was stirred at room temperature overnight. Triethylamine (1.5 eq.) and 2,2-dimethylpropanoyl chloride (1.2 eq) were added and the mixture was stirred at room temperature for 1 week. The crude material was concentrated under vacuum and the resulting orange solid was diluted in ethyl acetate, washed with 0.1 N HCl and a sat. aq. NaHCO₃ solution dried over MgSO₄ and concentrated under vacuum to afford the title compound as an orange oil (2.7732 g). LC-MS: m/z, 416 (M+H).

### Intermediate 36

### 1,1-dimethylethyl (2R,4S)-2-{[(2,2-dimethylpropanoyl)oxy]methyl}-4-hydroxy-1-pyrrolidinecarboxylate

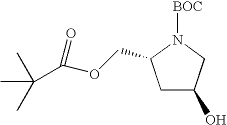

To a solution of 1,1-dimethylethyl (2R,4S)-4-{[(1,1-dimethylethyl)(dimethyl)silyl]oxy}-2-{[(2,2-dimethylpropanoyl)oxy]methyl}-1-pyrrolidinecarboxylate (2.5232 g, 6.07 mmol) in THF (˜10 mL) under argon atmosphere was added TBAF (12 mL, 12 mmol). The reaction mixture was stirred at room temperature for 2.45 hours. The crude was concentrated and the resulting oil was diluted in ethyl acetate and extracted with water (3×). The organic layer was concentrated under vacuum to afford the title compound as a brown liquid (1.8895 g). LC-MS: m/z, 302 (M+H).

### Intermediate 37

### 1,1-dimethylethyl (2R,4S)-2-{[(2,2-dimethylpropanoyl)oxy]methyl}-4-[(methylsulfonyl)oxy]-1-pyrrolidinecarboxylate

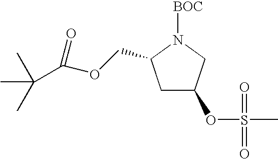

To a solution of 1,1-dimethylethyl (2R,4S)-2-{[(2,2-dimethylpropanoyl)oxy]methyl}-4-hydroxy-1-pyrrolidinecarboxylate (2.1522 g, 7.1 mmol) in DCM (35 mL) under argon atmosphere at 0° C. was added triethylamine (2.49 mL, 17.9 mmol) and methanesulfonyl chloride (1.1 mL, 14.3 mmol). The reaction mixture was stirred from 0° C. to room temperature for 16 hours. The solvent was removed under vacuum. The resulting orange solid was diluted in ethyl acetate, washed with 0.1 N HCl (2×) and NaHCO₃ solution. The aqueous layer was dried on MgSO₄, filtered and concentrated in vacuum to afford the title compound as an orange oil (2.09 g). LC-MS: m/z, 380 (M+H).

### Intermediate 38

### 1,1-dimethylethyl (2R,4R)-4-azido-2-{[(2,2-dimethylpropanoyl)oxy]methyl}-1-pyrrolidinecarboxylate

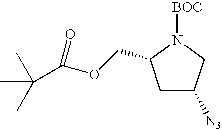

To a solution of 1,1-dimethylethyl (2R,4S)-2-{[(2,2-dimethylpropanoyl)oxy]methyl}-4-[(methylsulfonyl)oxy]-1-pyrrolidinecarboxylate (1 g, 2.6 mmol, 1 eq.) in acetonitrile (19 mL) under argon atmosphere was added tertbutylamino azide (1.01 g, 3.5 mmol). The resultant mixture was heated at reflux for 2 hours and stirred at room temperature overnight. The mixture was heated at reflux for an additional 1 hour and stirred at room temperature over the weekend. The solvent was removed under vacuum and the crude was diluted in ethyl acetate, washed with 0.1 N HCl and NaHCO₃ solution, dried over MgSO₄ and concentrated in vacuum. Some starting material was left. To the mixture tertbutylamino azide (0.78 g, 1 eq.) in acetonitrile (10 mL) was added and the resultant mixture was stirred at reflux for 6 hours then at room temperature overnight. The crude was processed as previously described above to afford the title compound (0.823 g). LC-MS: m/z, 327 (M+H).

### Intermediate 39

### 1,1-dimethylethyl (2R,4R)-4-amino-2-{[(2,2-dimethylpropanoyl)oxy]methyl}-1-pyrrolidinecarboxylate

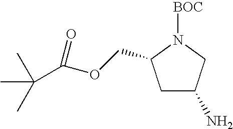

A solution of 1,1-dimethylethyl (2R,4R)-4-azido-2-{[(2,2-dimethylpropanoyl)oxy]methyl}-1-pyrrolidinecarboxylate (0.823 g, 2.5 mmol) in MeOH (˜20 mL) was hydrogenated over 10% Pd/C in a H-Cube™ reactor. The crude material was concentrated under vacuum to afford the title compound (0.5659 g). LC-MS: m/z, 301 (M+H).

### Intermediate 40

### 1,1-dimethylethyl (2R,4R)-4-{[(2,5-dibromophenyl)sulfonyl]amino}-2-{[(2,2-dimethylpropanoyl)oxy]methyl}-1-pyrrolidinecarboxylate

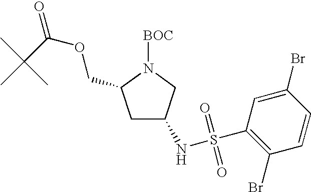

To 1,1-dimethylethyl (2R,4R)-4-amino-2-{[(2,2-dimethylpropanoyl)oxy]methyl}-1-pyrrolidinecarboxylate (0.200 g, 0.66 mmol) in DCM (10 mL) was added DIPEA (0.23 mL, 1.33 mmol) and 2,5-dibromobenzenesulfonyl chloride (0.022 g, 0.66 mmol). The resultant mixture was stirred at room temperature for 5 hours. The crude material was concentrated under vacuum then loaded onto a 10 g silica SPE cartridge and eluted sequentially with DCM and ethyl acetate. The ethyl acetate fractions were combined and evaporated to afford a crude residue which was diluted in ethyl acetate, washed with 0.1 N HCl and NaHCO₃ solution and concentrated under vacuum to afford the title compound (0.245 g). LC-MS: m/z, 599 (M+H).

### Intermediate 41

### 1,1-dimethylethyl (2R,4R)-4-{[(2,5-dibromophenyl)sulfonyl]amino}-2-(hydroxymethyl)-1-pyrrolidinecarboxylate

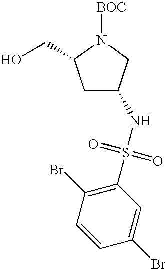

### Method A:

To 1,1-dimethylethyl (2R,4R)-4-{[(2,5-dibromophenyl)sulfonyl]amino}-2-{[(2,2-dimethylpropanoyl)oxy]methyl}-1-pyrrolidinecarboxylate (0.150 g, 0.25 mmol) in MeOH (1 ml) was added a 5% solution of sodium methoxide in methanol (6 ml). The resultant mixture was stirred at room temperature overnight, and then the solvent was removed under vacuum. The resulting residue was diluted with ethyl acetate then washed with 0.1N HCl, a sat. aq. NaHCO₃ solution, dried over MgSO₄ and concentrated under vacuum to afford the title compound (45 mg). LC-MS: m/z, 515 (M+H).

### Method B:

To 1,1-dimethylethyl (2R,4R)-4-{[(2,5-dibromophenyl)sulfonyl]amino}-2-{[(2,2-dimethylpropanoyl)oxy]methyl}-1-pyrrolidinecarboxylate (4.09 g, 6.84 mmol) in suspension in MeOH (30 ml) and water (15 ml) was added lithium hydroxide monohydrate (1.43 g, 34.2 mmol). The resultant mixture was stirred at room temperature over the week-end. The methanol was then removed under vacuum and the resulting aqueous layer was diluted in ethyl acetate. The organic layer was separated then washed with 0.1N HCl, a sat. aq. NaHCO₃ solution, dried over MgSO₄ and concentrated under vacuum to afford the title compound (3.52 g). LC-MS: m/z, 515 (M+H).

### Intermediate 42

### 1,1-dimethylethyl (2R,4R)-4-{[(2,5-dichlorophenyl)sulfonyl]amino}-2-{[(2,2-dimethylpropanoyl)oxy]methyl}-1-pyrrolidinecarboxylate

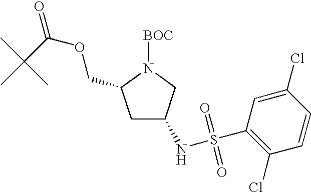

To 1,1-dimethylethyl (2R,4R)-4-amino-2-{[(2,2-dimethylpropanoyl)oxy]methyl}-1-pyrrolidinecarboxylate (0.100 g, 0.33 mmol) in DCM (3 mL) was added DIEA (0.12 mL, 0.66 mmol) and 2,5-dichlorobenzenesulfonyl chloride (0.082 g, 0.33 mmol). The resultant mixture was stirred at room temperature for 3 hours. The crude was washed with 0.1 N HCl and NaHCO₃ solution and concentrated under vacuum to afford the title compound (0.157 g). LC-MS: m/z, 510 (M+H).

### Intermediate 43

### 1,1-dimethylethyl (2R,4R)-4-({[5-chloro-2-(methyloxy)phenyl]sulfonyl}amino)-2-{[(2,2-dimethylpropanoyl)oxy]methyl}-1-pyrrolidinecarboxylate

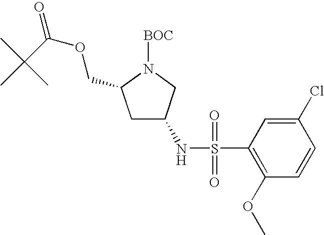

Intermediate 43 was prepared using the general procedure described above for Intermediate 42, replacing 2,5-dichlorobenzenesulfonyl chloride (0.082 g, 0.33 mmol) with 5-chloro-2-(methyloxy)benzenesulfonyl chloride (0.080 g, 0.33 mmol) to afford the title compound (0.133 g). LC-MS: m/z, 506 (M+H).

### Intermediate 44

### 1,1-dimethylethyl (2R,4R)-4-{[(2,5-dibromophenyl)sulfonyl]amino}-2-({[(phenylamino)carbonyl]oxy}methyl)-1-pyrrolidinecarboxylate

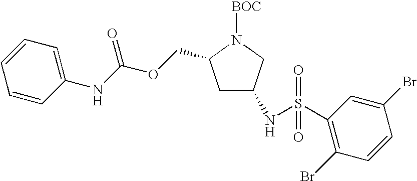

To 1,1-dimethylethyl (2R,4R)-4-{[(2,5-dibromophenyl)sulfonyl]amino}-2-(hydroxymethyl)-1-pyrrolidinecarboxylate (0.075 g, 0.146 mmol) in DCM (1 mL) was added NEt₃ (21 μL, 0.15 mmol) and isocyanatobenzene (17 μL, 0.16 mmol). The resultant mixture was stirred at room temperature for 2 hours then diluted in DCM, washed with water, 0.1 N HCl, and a sat. aq. NaHCO₃ solution and concentrated under vacuum to afford the title compound (0.060 g). LC-MS: m/z, 633 (M+H).

### Intermediate 45

### 1,1-dimethylethyl (2R,4R)-4-{[(2,5-dibromophenyl)sulfonyl]amino}-2-[({[(phenyl methyl)amino]carbonyl}oxy)methyl]-1-pyrrolidinecarboxylate

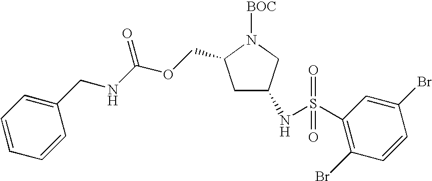

To 1,1-dimethylethyl (2R,4R)-4-{[(2,5-dibromophenyl)sulfonyl]amino}-2-(hydroxymethyl)-1-pyrrolidinecarboxylate (0.075 g, 0.146 mmol, 1 eq.) in DCM (1 mL) was added NEt₃ (0.0203 mL, 0.146 mmol). To the resultant mixture was added (isocyanatomethyl)benzene (0.0214 g, 0.16 mmol) in DCM (1.5 mL) and it was stirred at room temperature for 4 hours. To this mixture NEt₃ (1 eq.) and (isocyanatomethyl)benzene (1 eq.) were added and it was stirred at room temperature overnight. The crude was diluted in DCM, and then washed with water, 0.1 N HCl and a sat. aq. NaHCO₃ solution. Both layers were separated using a hydrophobic frit and the organic layer was concentrated under vacuum. The resulting crude material was loaded onto a 12 g silica SPE cartridge, and eluted sequentially with DCM ether and ethyl acetate. The relevant fractions were evaporated to afford the title compound (0.0723 g). LC-MS: m/z, 648 (M+H).

### Intermediate 46

### 1,1-dimethylethyl (2R,4R)-4-{[(2,5-dibromophenyl)sulfonyl]amino}-2-({[({[2-(methyloxy)phenyl]methyl}amino)carbonyl]oxy}methyl)-1-pyrrolidinecarboxylate

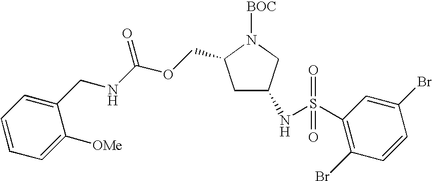

Intermediate 46 was prepared using the general procedure described above for Intermediate 45, replacing (isocyanatomethyl)benzene (0.0214 g, 0.16 mmol) with 1-(isocyanatomethyl)-2-(methyloxy)benzene (0.0262 g, 0.16 mmol) to afford the title compound (0.0846 g). LC-MS: m/z, 678 (M+H).

### Intermediate 47

### 1,1-dimethylethyl (2R,4R)-4-{[(2,5-dibromophenyl)sulfonyl]amino}-2-({[({[4-(methyloxy)phenyl]methyl}amino)carbonyl]oxy}methyl)-1-pyrrolidinecarboxylate

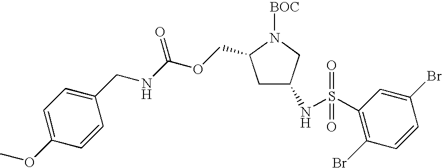

Intermediate 47 was prepared using the general procedure described above for Intermediate 45, replacing (isocyanatomethyl)benzene (0.0214 g, 0.16 mmol) with 1-(isocyanatomethyl)-4-(methyloxy)benzene (0.0229 mL, 0.16 mmol) to afford the title compound (0.0788 g). LC-MS: m/z, 678 (M+H).

### Intermediate 48

### 1,1-dimethylethyl (2R,4R)-4-{[(2,5-dibromophenyl)sulfonyl]amino}-2-{[({[2-(methyloxy)phenyl]amino}carbonyl)oxy]methyl}-1-pyrrolidinecarboxylate

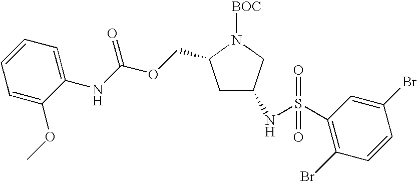

Intermediate 48 was prepared using the general procedure described above for Intermediate 45, replacing (isocyanatomethyl)benzene (0.0214 g, 0.16 mmol) with 1-isocyanato-2-(methyloxy)benzene (0.0226 mL, 0.16 mmol) to afford the title compound (0.0919 g). LC-MS: m/z, 664 (M+H).

### Intermediate 49

### 1,1-dimethylethyl (2R,4R)-4-{[(2,5-dibromophenyl)sulfonyl]amino}-2-{[({[3-(methyloxy)phenyl]amino}carbonyl)oxy]methyl}-1-pyrrolidinecarboxylate

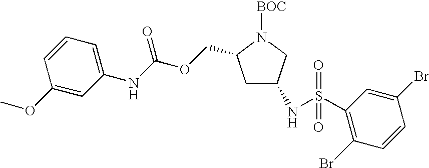

Intermediate 49 was prepared using the general procedure described above for Intermediate 45, replacing (isocyanatomethyl)benzene (0.0214 g, 0.16 mmol) with 1-isocyanato-3-(methyloxy)benzene (0.0211 mL, 0.16) to afford the title compound (0.1028 g). LC-MS: m/z, 664 (M+H).

### Intermediate 50

### 1,1-dimethylethyl (2R,4R)-4-{[(2,5-dibromophenyl)sulfonyl]amino}-2-{[({[4-(methyloxy)phenyl]amino}carbonyl)oxy]methyl}-1-pyrrolidinecarboxylate

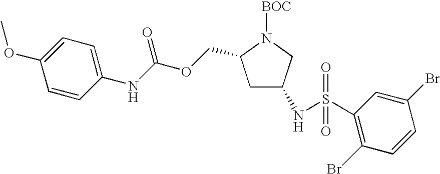

Intermediate 50 was prepared using the general procedure described above for Intermediate 45, replacing (isocyanatomethyl)benzene (0.0214 g, 0.16 mmol) with 1-isocyanato-4-(methyloxy)benzene (0.0209 mL, 0.16 mmol) to afford the title compound (0.0729 g). LC-MS: m/z, 664 (M+H).

### Intermediate 51

### 1,1-dimethylethyl (2R,4R)-4-{[(2,5-dibromophenyl)sulfonyl]amino}-2-{[({[2-(trifluoromethyl)phenyl]amino}carbonyl)oxy]methyl}-1-pyrrolidinecarboxylate

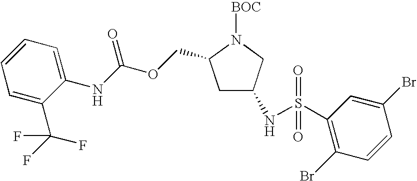

Intermediate 51 was prepared using the general procedure described above for Intermediate 45, replacing (isocyanatomethyl)benzene (0.0214 g, 0.16 mmol) with 1-isocyanato-2-(trifluoromethyl)benzene (0.0300 g, 0.16 mmol) to afford the title compound (0.0919 g). LC-MS: m/z, 702 (M+H).

### Intermediate 52

### 1,1-dimethylethyl (2R,4R)-4-{[(2,5-dibromophenyl)sulfonyl]amino}-2-{[({[3-(trifluoromethyl)phenyl]amino}carbonyl)oxy]methyl}-1-pyrrolidinecarboxylate

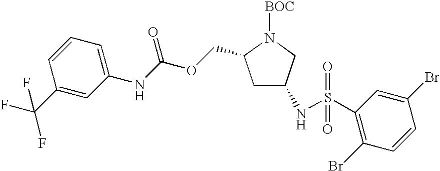

Intermediate 52 was prepared using the general procedure described above for Intermediate 45, replacing (isocyanatomethyl)benzene (0.0214 g, 0.16 mmol) with 1-isocyanato-3-(trifluoromethyl)benzene (0.0300 g, 0.16 mmol) to afford the title compound (0.1214 g). LC-MS: m/z, 702 (M+H).

### Intermediate 53

### 1,1-dimethylethyl (2R,4R)-4-{[(2,5-dibromophenyl)sulfonyl]amino}-2-{[({[4-(trifluoromethyl)phenyl]amino}carbonyl)oxy]methyl}-1-pyrrolidinecarboxylate

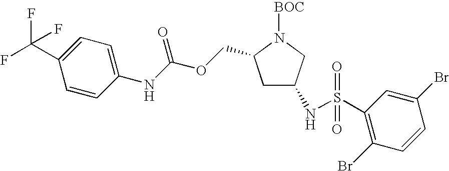

To 1,1-dimethylethyl (2R,4R)-4-{[(2,5-dibromophenyl)sulfonyl]amino}-2-(hydroxymethyl)-1-pyrrolidinecarboxylate (0.1 g, 0.194 mmol) in DCM (1 mL) was added NEt₃ (0.0271 mL, 0.194 mmol). To the resultant mixture was added 1-isocyanato-4-(trifluoromethyl)benzene (0.0400 g, 0.21 mmol) in DCM (1.5 mL). The reaction mixture was stirred at room temperature overnight then it was loaded onto a 2 g silica SPE cartridge, and eluted sequentially with DCM ether and ethyl acetate. The relevant fractions were evaporated to afford the title compound (0.1404 g). LC-MS: m/z, 702 (M+H).

### Intermediate 54

### 1,1-dimethylethyl (2R,4R)-4-{[(2,5-dibromophenyl)sulfonyl]amino}-2-[({[(2-methylphenyl)amino]carbonyl}oxy)methyl]-1-pyrrolidinecarboxylate

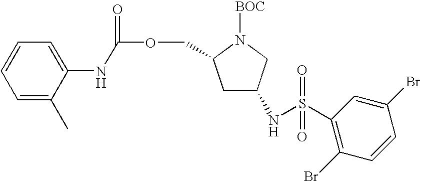

Intermediate 54 was prepared using the general procedure described above for Intermediate 53, replacing 1-isocyanato-4-(trifluoromethyl)benzene (0.0400 g, 0.21 mmol) with 1-isocyanato-2-methylbenzene (0.0265 mL, 0.214 mmol) to afford the title compound (0.0178 g). LC-MS: m/z, 648 (M+H).

### Intermediate 55

### 1,1-dimethylethyl (2R,4R)-4-{[(2,5-dibromophenyl)sulfonyl]amino}-2-[({[(3-methyl phenyl)amino]carbonyl}oxy)methyl]-1-pyrrolidinecarboxylate

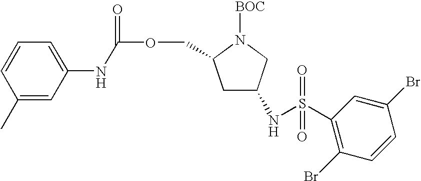

Intermediate 55 was prepared using the general procedure described above for Intermediate 53, replacing 1-isocyanato-4-(trifluoromethyl)benzene (0.0400 g, 0.21 mmol) with 1-isocyanato-3-methylbenzene (0.0285 g, 0.21 mmol) to afford the title compound (0.1203 g). LC-MS: m/z, 648 (M+H).

### Intermediate 56

### 1,1-dimethylethyl (2R,4R)-4-{[(2,5-dibromophenyl)sulfonyl]amino}-2-({[(1-naphthalenylamino)carbonyl]oxy}methyl)-1-pyrrolidinecarboxylate

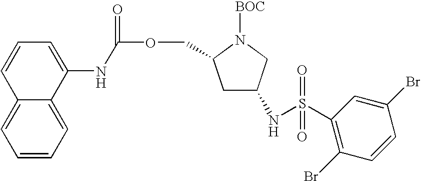

Intermediate 56 was prepared using the general procedure described above for Intermediate 53, replacing 1-isocyanato-4-(trifluoromethyl)benzene (0.0400 g, 0.21 mmol) with 1-isocyanatonaphthalene (0.0362 g, 0.21 mmol) to afford the title compound (0.1203 g). LC-MS: m/z, 684 (M+H).

### Intermediate 57

### 1,1-dimethylethyl (2R,4R)-4-{[(2,5-dibromophenyl)sulfonyl]amino}-2-({[(2-naphthalenylamino)carbonyl]oxy}methyl)-1-pyrrolidinecarboxylate

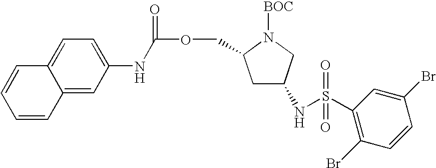

Intermediate 57 was prepared using the general procedure described above for Intermediate 53, replacing 1-isocyanato-4-(trifluoromethyl)benzene (0.0400 g, 0.21 mmol) with 2-isocyanatonaphthalene (0.0362 g, 0.21 mmol) to afford the title compound (0.1179 g). LC-MS: m/z, 684 (M+H).

### Intermediate 58

### 1,1-dimethylethyl (2R,4R)-4-{[(2,5-dibromophenyl)sulfonyl]amino}-2-({[(ethylamino)carbonyl]oxy}methyl)-1-pyrrolidinecarboxylate

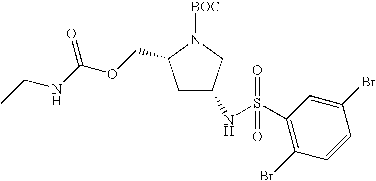

Intermediate 58 was prepared using the general procedure described above for Intermediate 53, replacing 1-isocyanato-4-(trifluoromethyl)benzene (0.0400 g, 0.21 mmol) with isocyanatoethane (0.0152 g, 0.21 mmol) to afford the title compound (0.1132 g). LC-MS: m/z, 586 (M+H).

### Intermediate 59

### 1,1-dimethylethyl (2R,4R)-4-{[(2,5-dibromophenyl)sulfonyl]amino}-2-[({[(1,1-dimethylethyl)amino]carbonyl}oxy)methyl]-1-pyrrolidinecarboxylate

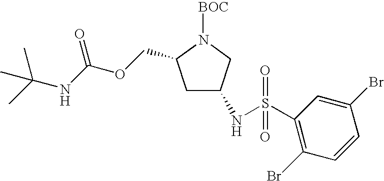

To 1,1-dimethylethyl (2R,4R)-4-{[(2,5-dibromophenyl)sulfonyl]amino}-2-(hydroxymethyl)-1-pyrrolidinecarboxylate (0.1 g, 0.194 mmol, 1 eq) in DCM (1 mL) was added NEt₃ (0.0271 mL, 0.194 mmol). To the resultant mixture was added 1-isocyanato-4-(trifluoromethyl)benzene (0.0400 g, 0.21 mmol) in DCM (1.5 mL) and the mixture was stirred at room temperature overnight. NEt₃ (4 eq) were added and the reaction was heated at 40° C. for 2 h. The reaction mixture was then concentrated under vacuum and was loaded onto a 2 g silica SPE cartridge, eluting sequentially with DCM ether and ethyl acetate. The relevant fractions were evaporated to afford the title compound (0.1116 g). LC-MS: m/z, 614 (M+H).

### Intermediate 60

### 1,1-dimethylethyl (2R,4R)-2-({[(cyclohexylamino)carbonyl]oxy}methyl)-4-{[(2,5-dibromophenyl)sulfonyl]amino}-1-pyrrolidinecarboxylate

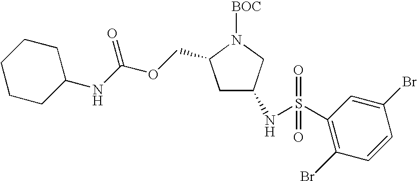

Intermediate 60 was prepared using the general procedure described above for Intermediate 53, replacing 1-isocyanato-4-(trifluoromethyl)benzene (0.0400 g, 0.21 mmol) with isocyanatocyclohexane (0.0268 g, 0.21 mmol) to afford the title compound (0.1013 g). LC-MS: m/z, 640 (M+H).

### Intermediate 61

### ((2R,4R)-4-{[(2,5-dibromophenyl)sulfonyl]amino}-2-pyrrolidinyl)methyl {[2-(methyl oxy)phenyl]methyl}carbamate

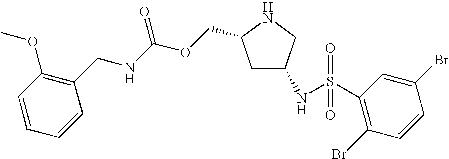

To Intermediate 46 (0.0846 g, 0.125 mmol) in dioxane (0.5 mL) was added 4 N HCl (0.70 mL) for 2 hours. The solvent was removed under vacuum to afford the title compound (0.0752 g). LC-MS: m/z, 578 (M+H).

### Intermediate 62

### ((2R,4R)-4-{[(2,5-dibromophenyl)sulfonyl]amino}-2-pyrrolidinyl)methyl {[3-(methyloxy)phenyl]methyl}carbamate

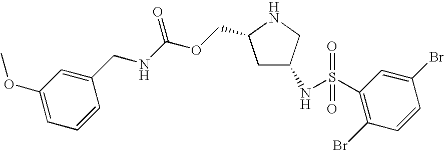

To 1,1-dimethylethyl (2R,4R)-4-{[(2,5-dibromophenyl)sulfonyl]amino}-2-(hydroxymethyl)-1-pyrrolidinecarboxylate (0.075 g, 0.146 mmol, 1 eq.) in DCM (1 mL) was added NEt₃ (0.0203 mL, 0.146 mmol). To the resultant mixture was added 1-(isocyanatomethyl)-3-(methyloxy)benzene (0.0262 g, 0.16 mmol) in DCM (1.5 mL) and stirred at room temperature for 4 hours. To this mixture NEt₃ (1 eq.) and (isocyanatomethyl)benzene (1 eq.) were added and stirred at room temperature overnight. The reaction mixture was diluted in DCM, washed with water, 0.1 N HCl, then a sat. aq. NaHCO₃ solution. Both layers were separated using a hydrophobic frit and the organic layer was concentrated under vacuum. To the resulting crude residue in dioxane (0.5 mL) was added 4 N HCl (0.70 mL). After stirring the reaction mixture for two hours, the solvent was removed under vacuum to afford the title compound (0.08 g). LC-MS: m/z, 578 (M+H).

### Intermediate 63

### ((2R,4R)-4-{[(2,5-dibromophenyl)sulfonyl]amino}-2-pyrrolidinyl)methyl {[4-(methyloxy)phenyl]methyl}carbamate

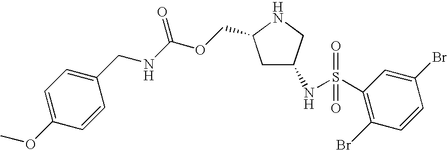

To Intermediate 47 (0.0788 g, 0.116 mmol) in dioxane (0.5 mL) was added 4 N HCl (0.70 mL) for 2 hours. The solvent was removed under vacuum to afford the title compound (0.0556 g). LC-MS: m/z, 578 (M+H).

### Intermediate 64

### ((2R,4R)-4-{[(2,5-dibromophenyl)sulfonyl]amino}-2-pyrrolidinyl)methyl [2-(methyloxy)phenyl]carbamate

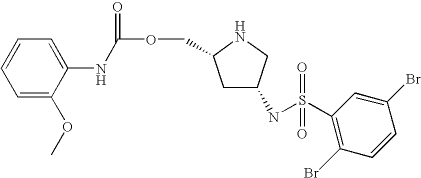

To Intermediate 48 (0.0919 g, 0.139 mmol) in dioxane (0.5 mL) was added 4 N HCl (0.70 mL) for 2 hours. The solvent was removed under vacuum and was loaded onto a 2 g silica SPE cartridge, then eluted sequentially with DCM ether and EA. The relevant fractions were combined and evaporated to afford the title compound (0.0304 g). LC-MS: m/z, 564 (M+H).

### Intermediate 65

### ((2R,4R)-4-{[(2,5-dibromophenyl)sulfonyl]amino}-2-pyrrolidinyl)methyl [3-(methyloxy)phenyl]carbamate

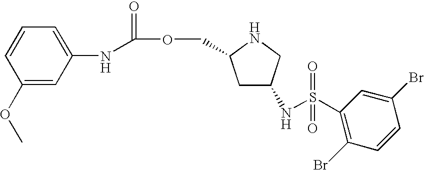

To Intermediate 49 (0.1028 g, 0.155 mmol) in dioxane (0.5 mL) was added 4 N HCl (0.70 mL) for 2 hours. The solvent was removed under vacuum and was loaded onto a 2 g silica SPE cartridge, then eluted sequentially with DCM ether and ethyl acetate. The relevant fractions were evaporated to afford the title compound (0.0496 g). LC-MS: m/z, 564 (M+H).

### Intermediate 66

### ((2R,4R)-4-{[(2,5-dibromophenyl)sulfonyl]amino}-2-pyrrolidinyl)methyl [4-(methyloxy)phenyl]carbamate

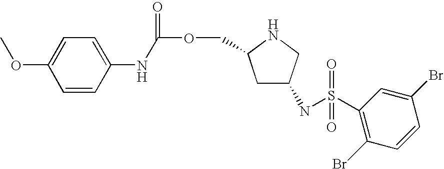

To Intermediate 50 (0.0729 g, 0.11 mmol) in dioxane (0.5 mL) was added 4 N HCl (0.70 mL) and the resulting mixture was stirred for 2 hours. The solvent was removed under vacuum to afford the title compound (0.0395 g). LC-MS: m/z, 564 (M+H).

### Intermediate 67

### ((2R,4R)-4-{[(2,5-dibromophenyl)sulfonyl]amino}-2-pyrrolidinyl)methyl [2-(trifluoromethyl)phenyl]carbamate

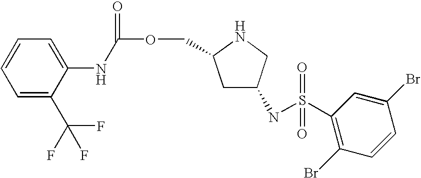

To Intermediate 51 (0.0919 g, 0.131 mmol) in dioxane (0.5 mL) was added 4 N HCl (0.70 mL) for 2 hours. The solvent was removed under vacuum to afford the title compound (0.069 g). LC-MS: m/z, 602 (M+H).

### Intermediate 68

### ((2R,4R)-4-{[(2,5-dibromophenyl)sulfonyl]amino}-2-pyrrolidinyl)methyl [3-(trifluoromethyl)phenyl]carbamate

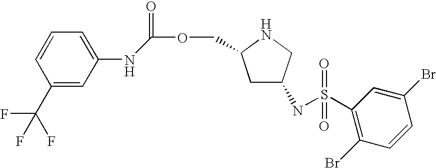

To Intermediate 52 (0.1214 g, 0.173 mmol) in dioxane (0.5 mL) was added 4 N HCl (0.70 mL) and the resulting mixture was stirred for 2 hours. The solvent was removed under vacuum to afford the title compound (0.0746 g). LC-MS: m/z, 602 (M+H).

### Intermediate 69

### ((2R,4R)-4-{[(2,5-dibromophenyl)sulfonyl]amino}-2-pyrrolidinyl)methyl [4-(trifluoromethyl)phenyl]carbamate

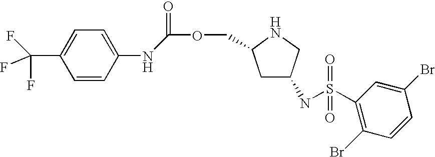

To Intermediate 54 (0.1404 g, 0.2 mmol) in dioxane (0.5 mL) was added 4 N HCl (0.9 mL) and the resulting mixture was stirred for 2 hours. The solvent was removed under vacuum to afford the title compound (0.0829 g). LC-MS: m/z, 602 (M+H).

### Intermediate 70

### ((2R,4R)-4-{[(2,5-dibromophenyl)sulfonyl]amino}-2-pyrrolidinyl)methyl (3-methylphenyl)carbamate

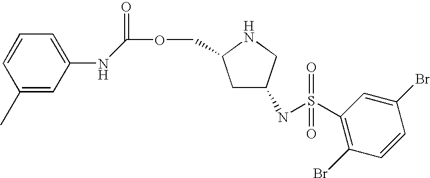

To Intermediate 55 (0.1363 g, 0.211 mmol) in dioxane (0.5 mL) was added 4 N HCl (0.9 mL) and the resulting mixture was stirred for 2 hours. The solvent was removed under vacuum to afford the title compound (0.0697 g). LC-MS: m/z, 548 (M+H).

### Intermediate 71

### ((2R,4R)-4-{[(2,5-dibromophenyl)sulfonyl]amino}-2-pyrrolidinyl)methyl 1-naphthalenylcarbamate

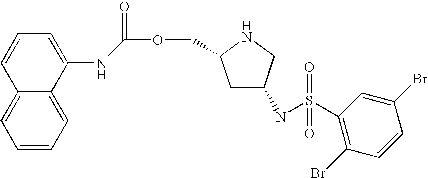

To Intermediate 56 (0.1203 g, 0.176 mmol) in dioxane (0.5 mL) was added 4 N HCl (0.9 mL) and the resulting mixture was stirred for 2 hours. The solvent was removed under vacuum to afford the title compound (0.1157 g). LC-MS: m/z, 584 (M+H).

### Intermediate 72

### ((2R,4R)-4-{[(2,5-dibromophenyl)sulfonyl]amino}-2-pyrrolidinyl)methyl 2-naphthalenylcarbamate

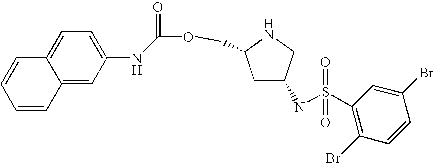

To Intermediate 57 (0.1179 g, 0.173 mmol) in dioxane (0.5 mL) was added 4 N HCl (0.9 mL) and the resulting mixture was stirred for 2 hours. The solvent was removed under vacuum to afford the title compound (0.0879 g). LC-MS: m/z, 584 (M+H).

### Intermediate 73

### ((2R,4R)-4-{[(2,5-dibromophenyl)sulfonyl]amino}-2-pyrrolidinyl)methyl ethylcarbamate

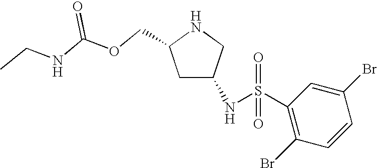

To Intermediate 58 (0.1132 g, 0.193 mmol) in dioxane (0.5 mL) was added 4 N HCl (0.9 mL) and the resulting mixture was stirred for 2 hours. The solvent was removed under vacuum to afford the title compound (0.0871 g). LC-MS: m/z, 486 (M+H).

### Intermediate 74

### ((2R,4R)-4-{[(2,5-Dibromophenyl)sulfonyl]amino}-2-pyrrolidinyl)methyl (1,1-dimethylethyl)carbamate

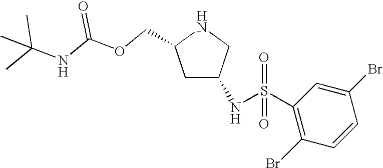

To Intermediate 59 (0.1116 g, 0.182 mmol) in dioxane (0.5 mL) was added 4 N HCl (0.9 mL) and the resulting mixture was stirred for 2 hours. The solvent was removed under vacuum to afford the title compound (0.253 g). LC-MS: m/z, 514 (M+H).

### Intermediate 75

### ((2R,4R)-4-{[(2,5-dibromophenyl)sulfonyl]amino}-2-pyrrolidinyl)methyl cyclohexylcarbamate

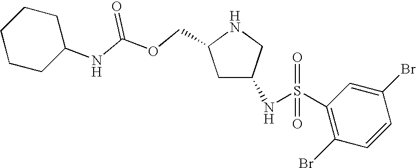

To Intermediate 60 (0.1013 g, 0.158 mmol) in dioxane (0.5 mL) was added 4 N HCl (0.9 mL) and the resulting mixture was stirred for 2 hours. The solvent was removed under vacuum to afford the title compound (0.0843 g). LC-MS: m/z, 540 (M+H).

### Intermediate 76

### 1,1-dimethylethyl (2S,4R)-4-{[(2,5-dibromo-3,6-difluorophenyl)sulfonyl]amino}-2-methyl-1-pyrrolidinecarboxylate

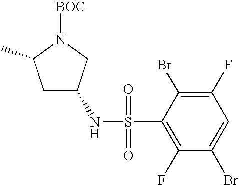

To 1,1-dimethylethyl (2S,4R)-4-amino-2-methyl-1-pyrrolidinecarboxylate (100 mg, 0.5 mmol) in DCM (4 mL) was added 2,5-dibromo-3,6-difluorobenzenesulfonyl chloride (222 mg, 0.6 mmol) and DIEA (129 mg, 1 mmol). The reaction mixture was stirred at room temperature over the weekend and washed with 1 N HCl and saturated NaHCO₃. The DCM layer was isolated and loaded onto a 20 g amino SPE cartridge then eluted sequentially with DCM and ethyl acetate. The ethyl acetate fractions were combined and evaporated to afford the title compound as a pale yellow oil (263 mg). LC-MS: m/z, 533 (M+H).

### Intermediate 77

### 1,1-dimethylethyl (2R,4S)-4-{[(1,1-dimethylethyl)(dimethyl)silyl]oxy}-2-[(1,3-dioxo-1,3-dihydro-2H-isoindol-2-yl)methyl]-1-pyrrolidinecarboxylate

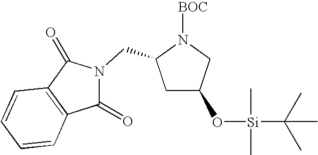

To 1,1-dimethylethyl (2R,4S)-4-{[(1,1-dimethylethyl)(dimethyl)silyl]oxy}-2-(hydroxymethyl)-1-pyrrolidinecarboxylate (331 mg, 1.0 mmol) in THF (5 mL) was added under argon 1H-isoindole-1,3(2H)-dione (aka phthalimide, 164 mg, 1.1 mmol) and triphenylphosphane (288 mg, 1.1 mmol). The reaction mixture was stirred for 5 minutes followed by the dropwise addition of diethyl azodicarboxylate (191 mg, 1.1 mmol). The resultant mixture was stirred at room temperature overnight, quenched with brine (10 mL) and diluted with diethyl ether. The ether layer was dried (Na₂SO₄), evaporated and purified by automated flash chromatography (12 g silica cartridge) to afford the title compound as a colorless oil (˜380 mg). LC-MS: m/z, 461 (M+H).

### Intermediate 78

### 1,1-dimethylethyl (2R,4S)-2-[(1,3-dioxo-1,3-dihydro-2H-isoindol-2-yl)methyl]-4-hydroxy-1-pyrrolidinecarboxylate

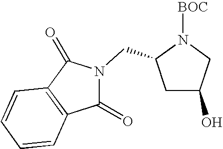

To 1,1-dimethylethyl (2R,4S)-4-{[(1,1-dimethylethyl)(dimethyl)silyl]oxy}-2-[(1,3-dioxo-1,3-dihydro-2H-isoindol-2-yl)methyl]-1-pyrrolidinecarboxylate (2.11 g, 4.59 mmol) in THF (50 mL) at 0° C. was added a tetra-n-butylammonium fluoride solution in THF, (1 M, 7 mL, 7 mmol) dropwise. The reaction mixture was stirred at 0° C. for 1 hour followed by addition of water. The organic layer was separated and washed with brine. The aqueous layers were combined and extracted with ethyl acetate. The combined organic layers were dried over Na₂SO₄ and evaporated to give a crude material, which was purified by loading it onto a SPE cartridge (20 g silica cartridge) and eluting sequentially with DCM and ethyl acetate. The relevant fractions were combined and evaporated to afford the title compound as a white solid (˜1.58 g).

### Intermediate 79

### 1,1-dimethylethyl (2R,4S)-2-[(1,3-dioxo-1,3-dihydro-2H-isoindol-2-yl)methyl]-4-[(methylsulfonyl)oxy]-1-pyrrolidinecarboxylate

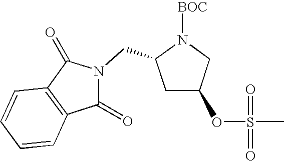

To 11,1-dimethylethyl (2R,4S)-2-[(1,3-dioxo-1,3-dihydro-2H-isoindol-2-yl)methyl]-4-hydroxy-1-pyrrolidinecarboxylate (1.58 g, 4.56 mmol) in DCM (20 mL) were added triethylamine (2.0 mL, 14.4 mmol) and 4-(dimethylamino)pyridine (840 mg, 6.9 mmol). To the resultant mixture was added dropwise methanesulfonyl chloride (540 μL, 6.9 mmol) at 0° C. while stirring. The resultant mixture was stirred under argon overnight, then diluted with ethyl acetate, and washed sequentially with 1 N HCl and a sat. aq. NaHCO₃ solution. After drying over Na₂SO₄, the solution was evaporated to afford the title compound as a white solid (˜1.96 g). LC-MS: m/z, 425 (M+H).

### Intermediate 80

### 1,1-dimethylethyl (2R,4R)-4-azido-2-[(1,3-dioxo-1,3-dihydro-2H-isoindol-2-yl)methyl]-1-pyrrolidinecarboxylate

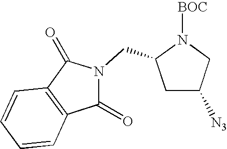

To 1,1-dimethylethyl (2R,4S)-2-[(1,3-dioxo-1,3-dihydro-2H-isoindol-2-yl)methyl]-4-[(methylsulfonyl)oxy]-1-pyrrolidinecarboxylate (1.96 g, 4.62 mmol) in CH₃CN (30 mL) was added tetrabutylammonium azide (1.97 g, 6.93 mmol) in CH₃CN (2 mL) while stirring. The reaction mixture was stirred at room temperature overnight and refluxed for 3 hours. The mixture was refluxed for an additional 1 hour, evaporated and purified by automated flash chromatography (40 g silica cartridge) to afford the title compound as a white solid (1.37 g).

### Intermediate 81

### 1,1-dimethylethyl (2R,4R)-4-amino-2-[(1,3-dioxo-1,3-dihydro-2H-isoindol-2-yl)methyl]-1-pyrrolidinecarboxylate

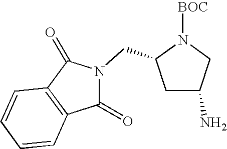

To 1,1-dimethylethyl (2R,4R)-4-azido-2-[(1,3-dioxo-1,3-dihydro-2H-isoindol-2-yl)methyl]-1-pyrrolidinecarboxylate (1.37 g, 3.7 mmol) in MeOH (75 mL) was hydrogenated in a H-Cube™ reactor (10% Pd/C cartridge) at room temperature to afford the title compound as a white solid (˜1.15 g).

### Intermediate 82

### 1,1-dimethylethyl (2R,4R)-4-{[(2,5-dibromophenyl)sulfonyl]amino}-2-[(1,3-dioxo-1,3-dihydro-2H-isoindol-2-yl)methyl]-1-pyrrolidinecarboxylate

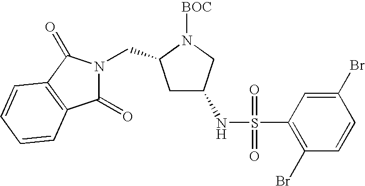

To 1,1-dimethylethyl (2R,4R)-4-amino-2-[(1,3-dioxo-1,3-dihydro-2H-isoindol-2-yl)methyl]-1-pyrrolidinecarboxylate (1.15 g, 3.33 mmol) in DCM (4 mL) was added 2,5-dibromo-3,6-difluorobenzenesulfonyl chloride (1.11 g, 3.33 mmol) and DIEA (859 mg, 6.66 mmol). The reaction mixture was stirred at room temperature over the weekend and washed with 1 N HCl then a sat. aq. NaHCO₃ solution. The DCM layer was isolated and evaporated under vacuum. The residue was purified by automated flash chromatography (40 g silica catridge) to afford the title compound as a white solid (˜1.73 g). LC-MS: m/z, 644 (M+H).

### Intermediate 83

### 1,1-dimethylethyl (2R,4R)-2-(aminomethyl)-4-{[(2,5-dibromophenyl)sulfonyl]amino}-1-pyrrolidinecarboxylate

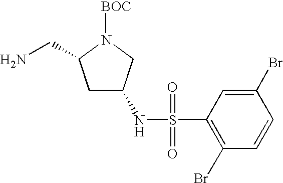

To 1,1-dimethylethyl (2R,4R)-4-{[(2,5-dibromophenyl)sulfonyl]amino}-2-[(1,3-dioxo-1,3-dihydro-2H-isoindol-2-yl)methyl]-1-pyrrolidinecarboxylate (1.57 g, 2.44 mmol) in EtOH (40 mL) was added hydrazine hydrate (500 mg, 10 mmol). The reaction mixture was refluxed for 2 hours. The by-product was filtered off, and the filtrated was concentrated and purified by preparatory HPLC (without TFA) to afford the title compound as a white solid (˜853 mg). LC-MS: m/z, 514 (M+H).

### Intermediate 84

### 1,1-dimethylethyl (2R,4R)-4-{[(2,5-dibromophenyl)sulfonyl]amino}-2-{[(phenylcarbonyl)amino]methyl}-1-pyrrolidinecarboxylate

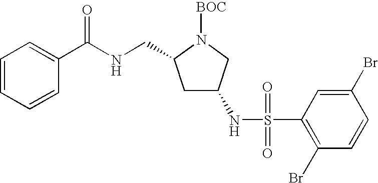

To 1 1,1-dimethylethyl (2R,4R)-2-(aminomethyl)-4-{[(2,5-dibromophenyl)sulfonyl]amino}-1-pyrrolidinecarboxylate (50 mg, 0.1 mmol) in DCM (3 mL) was added TEA (20 mg, 0.2 mmol) and benzoyl chloride (15 mg, 0.1 mmol) at room temperature while stirring. The reaction mixture was stirred at room temperature for 1 hour, washed with 1 N HCl and the DCM layer was isolated and evaporated to afford the title compound.

### Intermediate 85

### 1,1-dimethylethyl (2R,4R)-4-{[(2,5-dibromophenyl)sulfonyl]amino}-2-{[(2,2-dimethylpropanoyl)amino]methyl}-1-pyrrolidinecarboxylate

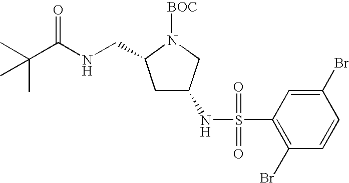

To 1 1,1-dimethylethyl (2R,4R)-2-(aminomethyl)-4-{[(2,5-dibromophenyl)sulfonyl]amino}-1-pyrrolidinecarboxylate (50 mg, 0.1 mmol) in DCM (3 mL) was added TEA (20 mg, 0.2 mmol) and 2,2-dimethylpropanoyl chloride (12 mg, 0.1 mmol) at room temperature while stirring. The reaction mixture was stirred at room temperature for 1 hour, washed with 1 N HCl and the DCM layer was isolated and evaporated to afford the title compound.

### Intermediate 86

### 1,1-dimethylethyl (2R,4R)-4-{[(2,5-dibromophenyl)sulfonyl]amino}-2-{[(phenylacetyl)amino]methyl}-1-pyrrolidinecarboxylate

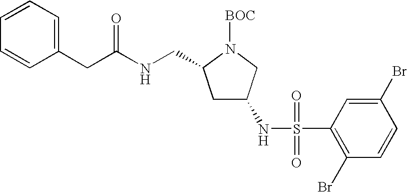

Intermediate 86 was prepared using the general procedure described above for Intermediate 84, replacing benzoyl chloride (15 mg, 0.1 mmol) with phenylacetyl chloride (31 mg, 0.2 mmol) to afford the title compound.

### Intermediate 87

### 1,1-dimethylethyl (2R,4R)-4-{[(2,5-dibromophenyl)sulfonyl]amino}-2-({[(3-methyl phenyl)carbonyl]amino}methyl)-1-pyrrolidinecarboxylate

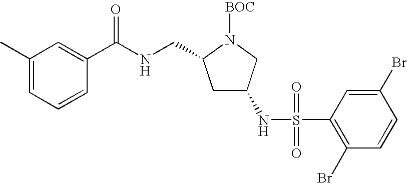

Intermediate 87 was prepared using the general procedure described above for Intermediate 84, replacing benzoyl chloride (15 mg, 0.1 mmol) with 3-methylbenzoyl chloride (31 mg, 0.2 mmol) to afford the title compound.

### Intermediate 88

### 1,1-dimethylethyl (2R,4R)-4-{[(2,5-dibromophenyl)sulfonyl]amino}-2-[({[2-(methyloxy)phenyl]carbonyl}amino)methyl]-1-pyrrolidinecarboxylate

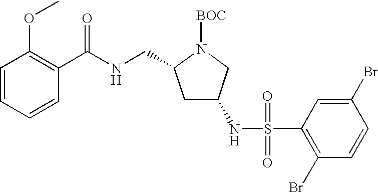

Intermediate 88 was prepared using the general procedure described above for Intermediate 84, replacing benzoyl chloride (15 mg, 0.1 mmol) with 2-(methyloxy)benzoyl chloride (34 mg, 0.2 mmol) to afford the title compound.

### Intermediate 89

### 1,1-dimethylethyl formate-N-[((2R,4R)-4-{[(2,5-dibromophenyl)sulfonyl]amino}-1-methyl-2-pyrrolidinyl)methyl]cyclohexanecarboxamide (1:1)

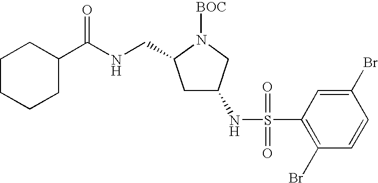

Intermediate 89 was prepared using the general procedure described above for Intermediate 84, replacing benzoyl chloride (15 mg, 0.1 mmol) with cyclohexanecarbonyl chloride (29 mg, 0.2 mmol) to afford the title compound.

### Intermediate 90

### 1,1-dimethylethyl (2R,4R)-4-{[(2,5-dibromophenyl)sulfonyl]amino}-2-{[(2-thienylcarbonyl)amino]methyl}-1-pyrrolidinecarboxylate

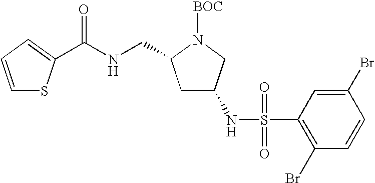

Intermediate 90 was prepared using the general procedure described above for Intermediate 84, replacing benzoyl chloride (15 mg, 0.1 mmol) with 2-thiophenecarbonyl chloride (29 mg, 0.2 mmol) to afford the title compound.

### Intermediate 91

### 1,1-dimethylethyl (2S,4R)-4-({[5-chloro-2-(methyloxy)phenyl]sulfonyl}amino)-2-methyl-1-pyrrolidinecarboxylate

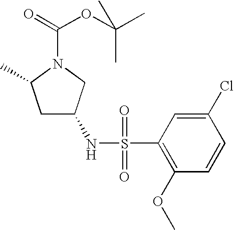

To 1,1-dimethylethyl (2S,4R)-4-amino-2-methyl-1-pyrrolidinecarboxylate (30 mg, 0.15 mmol) in DCM (20 mL) was added triethylamine (0.1 mL). The reaction mixture was chilled down to 0° C., then 5-chloro-2-(methyloxy)benzenesulfonyl chloride (40 mg, 0.16 mmol) was added. After stirring overnight, the reaction mixture was treated with water. After stirring for 5 minutes, the organic phase was isolated through a phase separator then concentrated under vacuum to afford the title compound (93.7 mg). LC-MS: m/z, 405.1 (M+H), rt 2.15 min.

### Intermediate 92

### 1,1-dimethylethyl (2S,4R)-4-{[(2,5-dimethylphenyl)sulfonyl]amino}-2-methyl-1-pyrrolidinecarboxylate

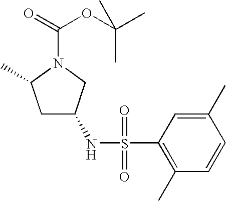

To 1,1-dimethylethyl (2S,4R)-4-amino-2-methyl-1-pyrrolidinecarboxylate (30 mg, 0.15 mmol) in DCM (20 mL) was added triethylamine (0.1 mL). The resulting mixture was chilled down to 0° C. then 2,5-dimethylbenzenesulfonyl chloride (34 mg, 0.17 mmol) was added. After stirring for 3 hours the reaction mixture was treated with water. After stirring for 5 minutes, the organic phase was isolated through a phase separator then concentrated under vacuum to afford the title compound (110.7 mg). LC-MS: m/z, 369 (M+H), rt 2.16 min.

### Intermediate 93

### 1,1-dimethylethyl (2S,4R)-2-methyl-4-({[3-(trifluoromethyl)phenyl]sulfonyl}amino)-1-pyrrolidinecarboxylate

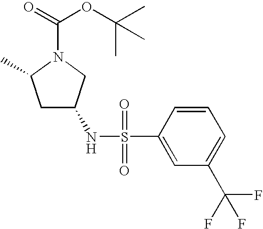

To 1,1-dimethylethyl (2S,4R)-4-amino-2-methyl-1-pyrrolidinecarboxylate (30 mg, 0.15 mmol) in DCM (20 mL) was added triethylamine (0.1 mL). The reaction mixture was chilled down to 0° C. then 3-(trifluoromethyl)benzenesulfonyl chloride (40 mg, 0.16 mmol) was added. After stirring for 3 hours the reaction mixture was treated with water. After stirring for 5 minutes, the organic phase was isolated through a phase separator then concentrated under vacuum to afford the title compound (104 mg). LC-MS: m/z, 409 (M+H), rt 2.17 min.

### Intermediate 94

### 2,5-dibromo-4-fluoro-N-[(3R,5S)-5-methyl-3-pyrrolidinyl]benzenesulfonamide

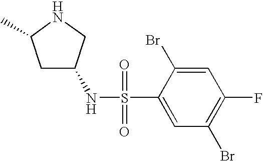

1,4-dibromo-2-fluorobenzene (5 g, 19.7 mmol) was added portionwise to a solution of CISO₃H (4 mL, 20 mmol) in 1,2 dichloroethane (9 mL) at 0° C. The reaction mixture was warmed to room temperature and stirred for 4 hours. The resultant mixture was cooled to 0° C., diluted with hexane (20 mL) and water (10 mL) was added with caution. The organic layer was separated, washed with brine, dried and evaporated to afford the title compound as a crude oil (3.10 g).

To the crude oil previously prepared in DCM (5 mL) and TEA (208 μL) was added 1,1-dimethylethyl (2S,4R)-4-amino-2-methyl-1-pyrrolidinecarboxylate (100 mg, 0.50 mmol) at room temperature. The solvent was evaporated under vacuum to give a crude oil that was treated with 4 N HCl in dioxane (6 ml). The residue was redissolved in water (100 mL), washed with ether (100 mL×2) and basified to pH 13 with 6N NaOH. The resultant mixture was extracted with EA (3×70 mL), dried over MgSO₄ and evaporated to afford the title compound as a brown solid (260 mg). LC-MS: m/z, 417 (M+H), rt 1.33 min

### Intermediate 95

### 1,1-Dimethylethyl (2R,4R)-4-{[(2,5-dichlorophenyl)sulfonyl]amino}-2-[(1,3-dioxo-1,3-dihydro-2H-isoindol-2-yl)methyl]-1-pyrrolidinecarboxylate

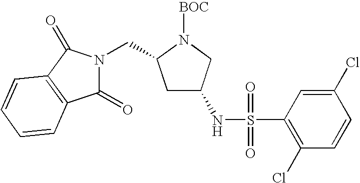

To 1,1-dimethylethyl (2R,4R)-4-amino-2-[(1,3-dioxo-1,3-dihydro-2H-isoindol-2-yl)methyl]-1-pyrrolidinecarboxylate (600 mg, 1.74 mmol) in DCM (15 mL) was added 2,5-dichlorobenzenesulfonyl chloride (448 mg, 1.83 mmol) and DIEA (0.6 ml, 3.5 mmol). The reaction mixture was stirred at room temperature for 2 h and washed with 1 N HCl. The DCM layer was isolated, dried (MgSO₄) and evaporated. The residue was purified by automated flash chromatography (40 g silica cartridge) to afford the title compound as a white solid (˜0.8 g). LC-MS: m/z, 556 (M+H), rt 2.30 min.

### Intermediate 96

### 1,1-Dimethylethyl (2R,4R)-4-({[5-chloro-2-(methyloxy)phenyl]sulfonyl}amino)-2-[(1,3-dioxo-1,3-dihydro-2H-isoindol-2-yl)methyl]-1-pyrrolidinecarboxylate

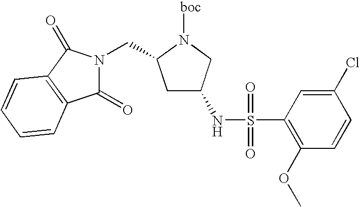

To 1,1-dimethylethyl (2R,4R)-4-amino-2-[(1,3-dioxo-1,3-dihydro-2H-isoindol-2-yl)methyl]-1-pyrrolidinecarboxylate (300 mg, 1.74 mmol) in DCM (5 mL) was added and TEA (0.242 mL, 1.737 mmol) followed by 2-methoxy-5-chlorobenzenesulfonyl chloride (209 mg, 0.869 mmol). The reaction mixture was stirred at room temperature overnight. The mixture was diluted with DCM and washed with 1 N HCl and saturated NaHCO₃. The DCM layer was isolated, dried (Na₂SO₄) and evaporated. The residue was purified by automated flash chromatography (12 g silica cartridge) to afford the title compound (400 mg). LC-MS: m/z, 550 (M+H), rt 1.15 min.

### Intermediate 97

### 1,1-Dimethylethyl (2R,4R)-4-({[5-bromo-2-(methyloxy)phenyl]sulfonyl}amino)-2-[(1,3-dioxo-1,3-dihydro-2H-isoindol-2-yl)methyl]-1-pyrrolidinecarboxylate

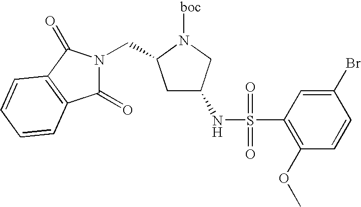

To 1,1-dimethylethyl (2R,4R)-4-amino-2-[(1,3-dioxo-1,3-dihydro-2H-isoindol-2-yl)methyl]-1-pyrrolidinecarboxylate (300 mg, 1.74 mmol) in DCM (5 mL) was added and TEA (0.242 mL, 1.737 mmol) followed by 2-methoxy-5-bromobenzenesulfonyl chloride (248 mg, 0.869 mmol). The reaction mixture was stirred at room temperature overnight. The mixture was diluted with DCM and washed with 1 N HCl and saturated NaHCO₃. The DCM layer was isolated, dried (Na₂SO₄) and evaporated. The residue was purified by automated flash chromatography (12 g silica cartridge) to afford the title compound (420 mg). LC-MS: m/z, 596 (M+H), rt 1.17 min.

### Intermediate 98

### 1,1-Dimethylethyl (2R,4R)-4-({[2,5-bis(methyloxy)phenyl]sulfonyl}amino)-2-[(1,3-dioxo-1,3-dihydro-2H-isoindol-2-yl)methyl]-1-pyrrolidinecarboxylate

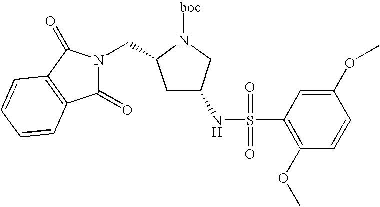

To 1,1-dimethylethyl (2R,4R)-4-amino-2-[(1,3-dioxo-1,3-dihydro-2H-isoindol-2-yl)methyl]-1-pyrrolidinecarboxylate (300 mg, 1.74 mmol) in DCM (5 mL) was added and TEA (0.242 mL, 1.737 mmol) followed by 2,5-dimethoxybenzenesulfonyl chloride (206 mg, 0.869 mmol). The reaction mixture was stirred at room temperature overnight. The mixture was diluted with DCM and washed with 1 N HCl and saturated NaHCO₃. The DCM layer was isolated, dried (MgSO₄) and evaporated. The residue was purified by automated flash chromatography (12 g silica cartridge) to afford the title compound (388 mg). LC-MS: m/z, 546 (M+H), rt 1.05 min.

### Intermediate 99

### 1,1-Dimethylethyl (2R,4R)-4-[{[5-bromo-2-(methyloxy)phenyl]sulfonyl}(phenylmethyl)amino]-2-[(1,3-dioxo-1,3-dihydro-2H-isoindol-2-yl)methyl]-1-pyrrolidinecarboxylate

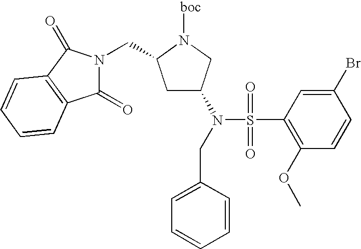

To a solution of 1,1-dimethylethyl (2R,4R)-4-({[5-bromo-2-(methyloxy)phenyl]sulfonyl}amino)-2-[(1,3-dioxo-1,3-dihydro-2H-isoindol-2-yl)methyl]-1-pyrrolidinecarboxylate (241 mg, 0.405 mmol) in DMF (5 ml), was added cesium carbonate (264 mg, 0.811 mmol) followed by benzyl bromide (0.072 ml, 0.608 mmol). The reaction mixture was stirred at room temperature overnight. The solvent was removed under vacuum and the residue was dissolved in ethyl acetate and water. The ethyl acetate layer was dried and concentrated to give crude as yellow oil, which was purified by automated flash chromatograph (12 g Silica cartridge) to afford the title compound (244 mg, 88%). LC-MS: m/z, 684 (M+H), rt 1.34 min.

### Intermediate 100

### 1,1-Dimethylethyl (2R,4R)-4-[{[2,5-bis(methyloxy)phenyl]sulfonyl}(phenylmethyl)amino]-2-[(1,3-dioxo-1,3-dihydro-2H-isoindol-2-yl)methyl]-1-pyrrolidinecarboxylate

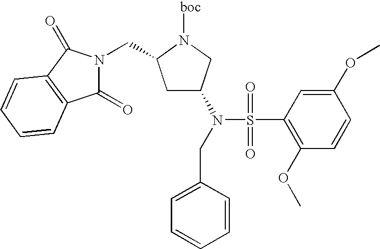

To a solution of 1,1-dimethylethyl (2R,4R)-4-({[2,5-bis(methyloxy)phenyl]sulfonyl}amino)-2-[(1,3-dioxo-1,3-dihydro-2H-isoindol-2-yl)methyl]-1-pyrrolidinecarboxylate (340 mg, 0.623 mmol) in DMF (5 ml), was added cesium carbonate (406 mg, 1.246 mmol) followed by benzyl bromide (0.111 ml, 0.935 mmol). The reaction mixture was stirred at room temperature overnight. Remove solvent under vacuum and the residue was dissolved in ethyl acetate and water. The ethyl acetate layer was dried and concentrated to give crude as yellow oil, which was purified by automated flash chromatograph (12 g Si cartridge) to afford the title compound (340 mg, 86%). LC-MS: m/z, 537 (M+H), rt 1.32 min.

### Intermediate 101

### 1,1-Dimethylethyl (2R,4R)-2-(aminomethyl)-4-{[(2,5-dichlorophenyl)sulfonyl]amino}-1-pyrrolidinecarboxylate

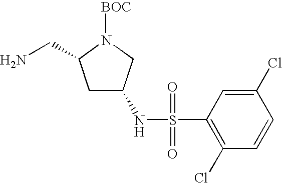

To 1,1-dimethylethyl (2R,4R)-4-{[(2,5-dichlorphenyl)sulfonyl]amino}-2-[(1,3-dioxo-1,3-dihydro-2H-isoindol-2-yl)methyl]-1-pyrrolidinecarboxylate (0.8 g, 1.44 mmol) in EtOH (20 mL) was added hydrazine hydrate (295 mg, 5.9 mmol). The reaction mixture was heated at reflux for 2 hours. The by-product was filtered off, and the filtrate was concentrated and purified by preparatory HPLC (without TFA) to afford the title compound as a white solid (390 mg, 64%). LC-MS: m/z, 426 (M+H), rt 1.58 min.

### Intermediate 102

### 1,1-Dimethylethyl (2R,4R)-2-(aminomethyl)-4-({[5-chloro-2-(methyloxy)phenyl]sulfonyl}amino)-1-pyrrolidinecarboxylate

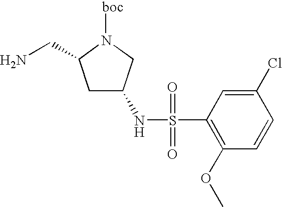

To 1,1-dimethylethyl (2R,4R)-4-({[5-chloro-2-(methyloxy)phenyl]sulfonyl}amino)-2-[(1,3-dioxo-1,3-dihydro-2H-isoindol-2-yl)methyl]-1-pyrrolidinecarboxylate (400 mg, 0.727 mmol) in EtOH (10 mL) was added hydrazine hydrate (0.178 mL, 3.64 mmol). The reaction mixture was heated at reflux for 1 hour. After cooling down, the mixture was filtered off, and the filtrate was concentrated to afford the title compound as a white solid (280 mg, 84%). LC-MS: m/z, 420 (M+H), rt 0.80 min.

### Intermediate 103

### 1,1-Dimethylethyl (2R,4R)-2-(aminomethyl)-4-({[5-bromo-2-(methyloxy)phenyl]sulfonyl}amino)-1-pyrrolidinecarboxylate

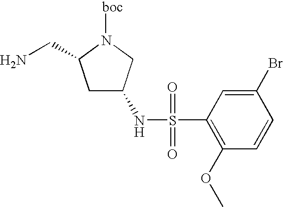

To 1,1-dimethylethyl (2R,4R)-4-({[5-bromo-2-(methyloxy)phenyl]sulfonyl}amino)-2-[(1,3-dioxo-1,3-dihydro-2H-isoindol-2-yl)methyl]-1-pyrrolidinecarboxylate (420 mg, 0.707 mmol) in EtOH (10 mL) was added hydrazine hydrate (0.173 mL, 3.53 mmol). The reaction mixture was refluxed for 1 hour. After cooling down, the mixture was filtered off, and the filtrate was concentrated to afford the title compound as a white solid (330 mg, 101%). LC-MS: m/z, 464 (M+H), rt 0.86 min.

### Intermediate 104

### 1,1-Dimethylethyl (2R,4R)-2-(aminomethyl)-4-({[2,5-bis(methyloxy)phenyl]sulfonyl}amino)-1-pyrrolidinecarboxylate

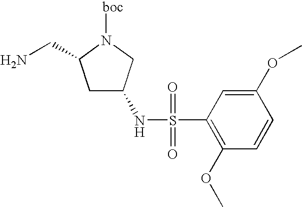

To 1,1-dimethylethyl (2R,4R)-4-({[2,5-bis(methyloxy)phenyl]sulfonyl}amino)-2-[(1,3-dioxo-1,3-dihydro-2H-isoindol-2-yl)methyl]-1-pyrrolidinecarboxylate (380 mg, 0.696 mmol) in EtOH (10 mL) was added hydrazine hydrate (0.171 mL, 3.48 mmol). The reaction mixture was refluxed for 1 hour. After cooling down, the mixture was filtered off, and the filtrate was concentrated to afford the title compound as a white solid (280 mg, 97%). LC-MS: m/z, 416 (M+H), rt 0.74 min.

### Intermediate 105

### 1,1-Dimethylethyl (2R,4R)-2-(aminomethyl)-4-[{[5-bromo-2-(methyloxy)phenyl]sulfonyl}(phenylmethyl)amino]-1-pyrrolidinecarboxylate

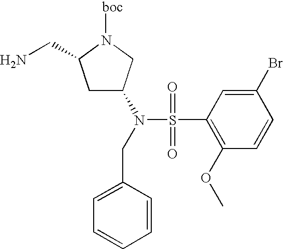

To 1,1-dimethylethyl (2R,4R)-4-[{[5-bromo-2-(methyloxy)phenyl]sulfonyl}(phenyl methyl)amino]-2-[(1,3-dioxo-1,3-dihydro-2H-isoindol-2-yl)methyl]-1-pyrrolidinecarboxylate (360 mg, 0.526 mmol) in EtOH (10 mL) was added hydrazine hydrate (0.132 mL, 2.63 mmol). The reaction mixture was refluxed for 1 hour. After cooling down, the mixture was filtered off, and the filtrate was concentrated to afford the title compound as a white solid (280 mg, 96%). LC-MS: m/z, 555 (M+H), rt 0.99 min.

### Intermediate 106

### 1,1-Dimethylethyl (2R,4R)-2-(aminomethyl)-4-[{[2,5-bis(methyloxy)phenyl]sulfonyl}(phenylmethyl)amino]-1-pyrrolidinecarboxylate

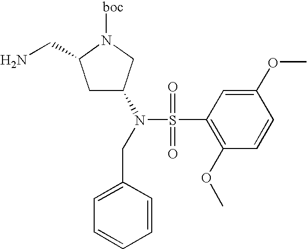

To 1,1-dimethylethyl (2R,4R)-4-[{[2,5-bis(methyloxy)phenyl]sulfonyl}(phenylmethyl)amino]-2-[(1,3-dioxo-1,3-dihydro-2H-isoindol-2-yl)methyl]-1-pyrrolidinecarboxylate (340 mg, 0.535 mmol) in EtOH (10 mL) was added hydrazine hydrate (0.131 mL, 2.67 mmol). The reaction mixture was refluxed for 1 hour. After cooling down, the mixture was filtered off, and the filtrate was concentrated to afford the title compound as a white solid (270 mg, 96%). LC-MS: m/z, 506 (M+H), rt 0.95 min.

### Intermediate 107

### 1,1-Dimethylethyl (2R,4S)-4-{[(1,1-dimethylethyl) (dimethyl)silyl]oxy}-2-({[4-(methyloxy)phenyl]oxy}methyl)-1-pyrrolidinecarboxylate

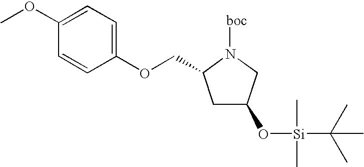

To a solution of 1,1-dimethylethyl (2R,4S)-4-{[(1,1-dimethylethyl)(dimethyl)silyl]oxy}-2-(hydroxymethyl)-1-pyrrolidinecarboxylate (510 mg, 1.54 mmol), 4-(methyloxy)phenol (573 mg, 4.62 mmol) and PPh₃ (524 mg, 2 mmol) in 6 ml dry THF, was added diethylazodicarboxylate (355 mg, 2.04 mmol) at room temperature. The resultant mixture was refluxed for 20 min., and then cooled to room temperature with stirring overnight. The reaction mixture was diluted with EA and washed with 1 N NaOH. The ethyl acetate layer was dried (MgSO₄) and evaporated. The residue was purified by automated flash chromatography (40 g silica cartridge) to afford the title compound as a colorless oil. (480 mg, 71%). LC-MS: m/z, 438 (M+H), rt 3.14 min.

### Intermediate 108

### 1,1-Dimethylethyl (2R,4S)-4-{[(1,1-dimethylethyl)(dimethyl)silyl]oxy}-2-{[(4-fluorophenyl)oxy]methyl}-1-pyrrolidinecarboxylate

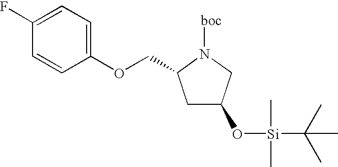

To a solution of 1,1-dimethylethyl (2R,4S)-4-{[(1,1-dimethylethyl)(dimethyl)silyl]oxy}-2-(hydroxymethyl)-1-pyrrolidinecarboxylate (558 mg, 1.68 mmol), 4-fluorophenol (566 mg, 5.05 mmol) and PPh₃ (572 mg, 2.18 mmol) in 6 ml dry THF, was added diethylazodicarboxylate (379 mg, 2.18 mmol) at room temperature. The resultant mixture was refluxed for 20 min., and then cooled to room temperature with stirring overnight. The reaction mixture was diluted with ethyl acetate and washed with 1 N NaOH. The ethyl acetate layer was dried (MgSO₄) and evaporated. The residue was purified by automated flash chromatography (40 g silica cartridge) to afford the title compound as a colorless oil. (540 mg, 76%). LC-MS: m/z, 428 (M+H), rt 3.21 min.

### Intermediate 109

### 1,1-Dimethylethyl (2R,4S)-4-{[(1,1-dimethylethyl) (dimethyl)silyl]oxy}-2-{[(phenylmethyl)oxy]methyl}-1-pyrrolidinecarboxylate

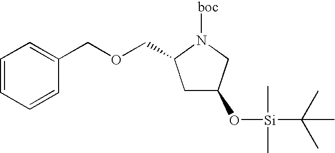

In a 10 mL round-bottomed flask was diluted 1,1-dimethylethyl (2R,4S)-4-{[(1,1-dimethylethyl)(dimethyl)silyl]oxy}-2-(hydroxymethyl)-1-pyrrolidinecarboxylate (0.5851 g, 1.765 mmol) in 1,2-dichloroethane (14 ml). Dudley reagent (0.9173 g, 2.63 mmol) and magnesium oxide (0.138 g, 3.42 mmol) were then added (suspension). The mixture was heated at reflux (83° C.) for 16 h, filtered through a pad of Celite, and the solvent was evaporated under vacuum to give a crude residue. The residue was loaded onto a 2 g silica SPE cartridge, and eluted sequentially with hexane, DCM and ethyl acetate. The DCM fractions were combined and condensed under vacuum to afford the title compound. (447 mg, 60%). LC-MS: m/z, 422 (M+H), rt 1.66 min.

### Intermediate 110

### 1,1-Dimethylethyl (2R,4S)-4-{[(1,1-dimethylethyl) (dimethyl)silyl]oxy}-2-({[3-(phenyloxy)propyl]oxy}methyl)-1-pyrrolidinecarboxylate

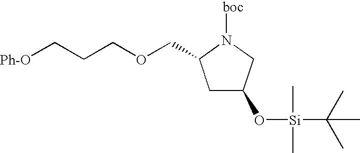

1,1-Dimethylethyl (2R,4S)-4-{[(1,1-dimethylethyl)(dimethyl)silyl]oxy}-2-(hydroxymethyl)-1-pyrrolidinecarboxylate (0.6 g, 1.810 mmol) was diluted in THF (1 ml) to give a colorless solution. The resulting mixture was cooled down to 0° C. under argon. Then sodium hydride (0.217 g, 9.05 mmol) in THF (2 mL) was added to give a white suspension followed by [(3-bromopropyl)oxy]benzene 3-bromopropyl phenyl ether (0.779 g, 3.62 mmol) in THF (1 ml). The resulting mixture was stirred at rt over the week end (96 h). The reaction was then quenched by addition of water at 0° C. The THF was removed in vacuo and the resulting crude was diluted in DCM and washed with 0.1M HCl, sat NaHCO₃ using a hydrophobic frit. The organic layer was then concentrated in vacuo to yield a colorless oil. This resulting oil was loaded onto a 5 g silica SPE cartridge, then eluted sequentially with hexane DCM and EA. The corresponding fractions were combined and evaporated to afford the title compound (0.45 g, 53%). LC-MS: m/z, 466 (M+H), rt 1.67 min.

### Intermediate 111

### 1,1-Dimethylethyl (2R,4S)-4-hydroxy-2-({[4-(methyloxy)phenyl]oxy}methyl)-1-pyrrolidinecarboxylate

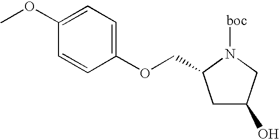

Under argon atmosphere in a solution of 1,1-dimethylethyl (2R,4S)-4-{[(1,1-dimethylethyl)(dimethyl)silyl]oxy}-2-{[(4-fluorophenyl)oxy]methyl}-1-pyrrolidinecarboxylate (480 mg, 1.1 mmol) in THF (˜5 mL) was added TBAF (1.6 mL, 1.60 mmol) and stirred at room temperature for 3 hours. The crude was concentrated, diluted in ethyl acetate and the organic layer was washed with water. The aqueous layer was extracted with ethyl acetate (×2); the organic layers were combined, dried over MgSO₄ and concentrated to afford the title compound as a crude oil (470 mg). LC-MS: m/z, 324 (M+H), rt 1.76 min.

### Intermediate 112

### 1,1-Dimethylethyl (2R,4S)-2-{[(4-fluorophenyl)oxy]methyl}-4-hydroxy-1-pyrrolidinecarboxylate

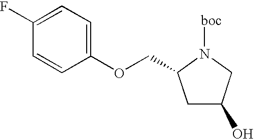

Under argon atmosphere in a solution of 1,1-dimethylethyl (2R,4S)-4-{[(1,1-dimethylethyl)(dimethyl)silyl]oxy}-2-{[(4-fluorophenyl)oxy]methyl}-1-pyrrolidinecarboxylate (280 mg, 0.66 mmol) in THF (˜5 mL) was added TBAF (1.0 mL, 1.0 mmol) and stirred at room temperature for 3 hours. The crude was concentrated, diluted in ethyl acetate and the organic layer was washed with water. The aqueous layer was extracted with ethyl acetate (×2); the organic layers were combined, dried over MgSO₄ and concentrated to afford the title compound as a crude oil (240 mg). LC-MS: m/z, 312 (M+H), rt 1.89 min.

### Intermediate 113

### 1,1-Dimethylethyl (2R,4S)-4-hydroxy-2-{[(phenylmethyl) oxy]methyl}-1-pyrrolidinecarboxylate

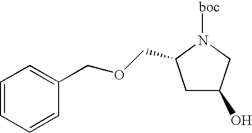

In a flask 1,1-dimethylethyl (2R,4S)-4-{[(1,1-dimethylethyl)(dimethyl)silyl]oxy}-2-{[(phenylmethyl)oxy]methyl}-1-pyrrolidinecarboxylate (0.4474 g, 1.061 mmol) was diluted in THF. TBAF (2.122 ml, 2.122 mmol) was added and the resulting mixture was stirred for 1.5 hr at rt. The THF was then removed in vacuo. The reaction mixture was diluted with DCM and washed with 0.1M HCl, sat NaHCO₃, and sat NaCl. The layers were separated using an hydrophobic frit and the organic layer was then concentrated in vacuo to yield the title compound as an oil. (0.293 g, 90%). LC-MS: m/z, 308 (M+H), rt 0.94 min.

### Intermediate 114

### 1,1-Dimethylethyl (2R,4S)-4-hydroxy-2-({[3-(phenyloxy) propyl]oxy}methyl)-1-pyrrolidinecarboxylate

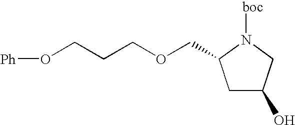

To a solution of 1,1-dimethylethyl (2R,4S)-4-{[(1,1-dimethylethyl)(dimethyl)silyl]oxy}-2-({[3-(phenyloxy)propyl]oxy}methyl)-1-pyrrolidinecarboxylate (0.45 g, 0.966 mmol) in THF was added TBAF (1.933 ml, 1.933 mmol, 1 M in THF). The resulting mixture was stirred for 2 h at room temperature. THF was then removed in vacuo and the reaction mixture was diluted with ethyl acetate and washed with 0.1M HCl, sat NaHCO₃, and sat. NaCl using a hydrophobic frit. The resulting crude was then concentrated in vacuo to yield the title compound as an oil. (0.204 g, 48%). LC-MS: m/z, 352 (M+H), rt 1.02 min.

### Intermediate 115

### 1,1-Dimethylethyl (2R,4R)-4-azido-2-({[4-(methyloxy)phenyl]oxy}methyl)-1-pyrrolidinecarboxylate

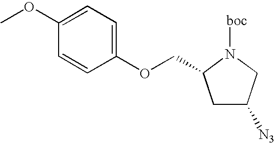

To a solution of 1,1-dimethylethyl (2R,4S)-4-hydroxy-2-({[4-(methyloxy)phenyl]oxy}methyl)-1-pyrrolidinecarboxylate (0.356 g, 1.1 mmol) in DCM (15 ml) was added TEA (0.460 ml, 3.30 mmol) and DMAP (0.202 g, 1.650 mmol) followed by dropwise adding mesyl chloride (0.129 ml, 1.650 mmol) at 0° C. with stirring. The reaction mixture was kept stirring under Argon for 2 h at room temperature. It was then diluted with DCM and washed with 1 N HCl and saturated NaHCO₃. The DCM layer was dried (Na₂SO₄) and evaporated to give the crude as a pale yellow oil, which was re-dissolved in acetonitrile (10 ml) followed by addition of tetrabutylammonium azide (469 mg, 1.650 mmol). The reaction mixture was refluxed under argon for 3 h. The solvent was removed under vacuum and the residue was purified by automated flash chromatography (12 g of silica) to afford the title compound as a pale yellow oil (340 mg, 68%). LC-MS: m/z, 349 (M+H), rt 2.31 min.

### Intermediate 116

### 1,1-Dimethylethyl (2R,4R)-4-azido-2-{[(4-fluorophenyl)oxy]methyl}-1-pyrrolidinecarboxylate

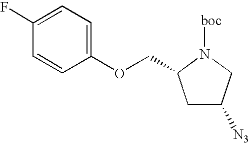

To a solution of 1,1-dimethylethyl (2R,4S)-2-{[(4-fluorophenyl)oxy]methyl}-4-hydroxy-1-pyrrolidinecarboxylate (0.205 g, 0.66 mmol) in DCM (10 ml) was added TEA (0.276 ml, 1.980 mmol) and DMAP (0.121 g, 0.990 mmol) followed by dropwise adding mesyl chloride (0.077 ml, 0.990 mmol) at 0° C. with stirring. The reaction mixture was kept stirring under argon for 2 h at room temperature. Dilute with DCM and washed with 1 N HCl and saturated NaHCO₃. The DCM layer was dried (Na₂SO₄) and evaporated to give the crude product as a pale yellow oil, which was re-dissolved in acetonitrile (10 ml) followed by addition of tetrabutylammonium azide (282 mg, 0.99 mmol). The reaction mixture was refluxed under argon for 1 h. The solvent was removed via vacuum and the residue was purified by automated flash chromatography (12 g of silica) to afford the title compound as a pale yellow oil (176 mg, 79%). LC-MS: m/z, 337 (M+H), rt 2.40 min.

### Intermediate 117

### 1,1-Dimethylethyl (2R,4R)-4-azido-2-{[(phenylmethyl)oxy]methyl}-1-pyrrolidinecarboxylate

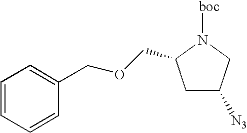

1,1-Dimethylethyl (2R,4S)-4-hydroxy-2-{[(phenylmethyl)oxy]methyl}-1-pyrrolidinecarboxylate (0.293 g, 0.952 mmol) was diluted in dichloromethane (DCM) (2 ml) to give a colorless solution at 0° C. Then triethylamine (0.332 ml, 2.381 mmol) and mesyl chloride (0.148 ml, 1.904 mmol) were added and the resulting mixture was stirred at room temperature for 48 hours. The crude was then washed with 0.1M HCl, sat NaHCO₃. The organic layer was separated and dried over MgSO₄, filtered and concentrated to yield a white solid (0.3112 g, 0.807 mmol). The solid was dissolved in acetonitrile (4 ml) under argon and was added tertabutylammonium azide (0.276 g, 0.969 mmol). The resulting mixture was then heated at reflux for 16 h. After cooling down, it was diluted with DCM and washed with H₂O, 0.1M HCl, and sat NaHCO₃. The organic layer was dried over MgSO₄, filtered and concentrated. The crude product was purified by automated flash chromatography (4 g of silica) to afford the title compound (0.22 g, 82%). LC-MS: m/z, 333 (M+H), rt 1.25 min.

### Intermediate 118

### 1,1-Dimethylethyl (2R,4R)-4-azido-2-({[3-(phenyloxy) propyl]oxy}methyl)-1-pyrrolidinecarboxylate

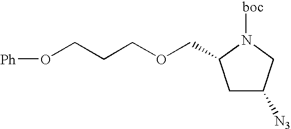

1,1-Dimethylethyl (2R,4S)-4-hydroxy-2-({[3-(phenyloxy)propyl]oxy}methyl)-1-pyrrolidinecarboxylate (0.2047 g, 0.582 mmol) was diluted in dichloromethane (DCM) (2 ml) to give a colorless solution at 0° C. Then triethylamine (0.203 ml, 1.456 mmol) and mesyl chloride (0.091 ml, 1.165 mmol) were added and the resulting mixture was stirred at room temperature for 16 h. The mixture was then washed with 0.1M HCl, sat NaHCO₃ and NaCl solution using an hydrophobic frit. The organic layer was then concentrated under vacuum. The residue was dissolved in acetonitrile (3 ml) under argon and to it was added tertabutylammonium azide (0.108 g, 0.381 mmol). The resulting mixture was then heated at reflux for 7 h. After cooling down, it was diluted in DCM and washed with H₂O, 0.1M HCl, saturated NaHCO₃ and brine using a hydrophobic frit. The organic layer was then concentrated in vacuo to afford the title compound (101 mg, 85%). LC-MS: m/z, 377 (M+H), rt 1.32 min.

### Intermediate 119

### 1,1-Dimethylethyl-(2R,4R)-4-amino-2-({[4-(methyloxy)phenyl]oxy}methyl)-1-pyrrolidinecarboxylate

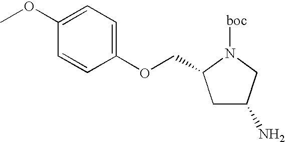

1,1-Dimethylethyl (2R,4R)-4-azido-2-({[4-(methyloxy)phenyl]oxy}methyl)-1-pyrrolidinecarboxylate (340 mg, 0.976 mmol) in methanol (20 ml) was hydrogenated over 10% Pd/C in a H-Cube™ reactor. The product was concentrated to afford the title compound as colorless oil (280 mg, 89%). LC-MS: m/z, 323 (M+H), rt 1.45 min.

### Intermediate 120

### 1,1-Dimethylethyl-(2R,4R)-4-amino-2-{[(4-fluorophenyl)oxy]methyl}-1-pyrrolidinecarboxylate

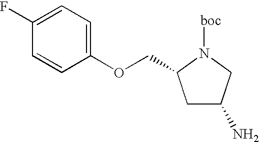

1,1-Dimethylethyl-(2R,4R)-4-azido-2-{[(4-fluorophenyl)oxy]methyl}-1-pyrrolidinecarboxylate (176 mg, 0.523 mmol) in Methanol (15 ml) was hydrogenated over 10% Pd/C in a H-Cube™ reactor. The product was concentrated to afford the title compound as colorless oil (140 mg, 86%). LC-MS: m/z, 323 (M+H), rt 0.94 min.

### Intermediate 121

### 1,1-Dimethylethyl (2R,4R)-4-amino-2-{[(phenylmethyl)oxy]methyl}-1-pyrrolidinecarboxylate

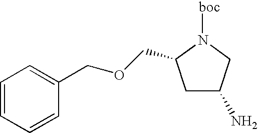

1,1-Dimethylethyl (2R,4R)-2-{[(phenylmethyl)oxy]methyl}-4-(2l5-1-triazen-2-yn-1-yl)-1-pyrrolidinecarboxylate (0.22 g, 0.662 mmol) in methanol (40 ml) was hydrogenated over 10% Pd/C in a H-Cube™ reactor. The product was concentrated to afford the title compound as colorless oil (0.192 g, 94%). LC-MS: m/z, 307 (M+H), rt 0.76 min.

### Intermediate 122

### 1,1-Dimethylethyl (2R,4R)-4-amino-2-({[3-(phenyloxy)propyl]oxy}methyl)-1-pyrrolidinecarboxylate

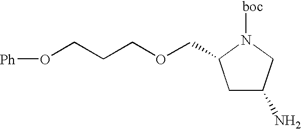

1,1-Dimethylethyl (2R,4R)-2-({[3-(phenyloxy)propyl]oxy}methyl)-4-(2l5-1-triazen-2-yn-1-yl)-1-pyrrolidinecarboxylate (0.1014 g, 0.269 mmol) in methanol (10 ml) was hydrogenated over 10% Pd/C in a H-Cube™ reactor. The product was concentrated to afford the title compound as colorless oil (0.192 g, 94%). LC-MS: m/z, 351 (M+H), rt 0.82 min.

### Intermediate 123

### 5-Bromo-2-chloro-4-fluorobenzenesulfonyl chloride

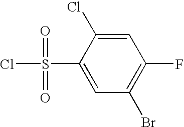

A mixture of 1-bromo-4-chloro-2-fluorobenzene (10 g, 47.7 mmol) and fuming sulfuric acid (18-24% SO₃, 40 ml) was heated at 110° C. over night. After cooling down to room temperature, the reaction mixture was carefully poured into ice (700 g). The resulting mixture was stirred at room temperature for 30 min followed by addition of ethyl acetate. The aqueous layer was isolated and concentrated under vacuum. The precipitate was filtered off to give 5-bromo-2-chloro-4-fluorobenzenesulfonic acid as white crystals (4.9 g, 35.4%).

The acid previously prepared (4.8 g, 16.58 mmol) and phosphorus pentachloride (10.36 g, 49.7 mmol) were heated at 110° C. over night. After cooling down to room temperature, the reaction mixture was carefully poured into ice (50 g). The resulting mixture was stirred at room temperature for 30 min followed by addition of ethyl acetate. The ethyl acetate layer was isolated and concentrated under vacuum to give the title compound as a yellow oil (5.1 g, 100%). ¹H NMR (CDCl₃) δ 8.40 (d, 1H), 7.46 (d, 1H).

### Intermediate 124

### 2,5-Dichloro-4-fluorobenzenesulfonyl chloride

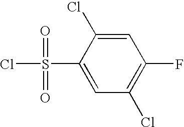

A mixture of 1,4-dichloro-2-fluorobenzene (5 g, 30.3 mmol) and fuming sulfuric acid (18-24% SO₃, 20 ml) was heated at 110° C. overnight. After cooling down to room temperature, the reaction mixture was carefully poured into ice (700 g). The resulting mixture was stirred at room temperature for 30 min followed by addition of ethyl acetate. The aqueous layer was isolated and concentrated under vacuum. The precipitate was filtered off to give 2,5-dichloro-4-fluorobenzenesulfonic acid as white crystals (3.33 g, 45%).

The acid previously prepared (2.0 g, 8.2 mmol) and phosphorus pentachloride (5 g, 24 mmol) were heated at 110° C. overnight. After cooling down to room temperature, the reaction mixture was carefully poured into ice (50 g) The resulting mixture was stirred at room temperature for 30 min followed by addition of ethyl acetate (100 mL). The ethyl acetate layer was isolated and concentrated under vacuum to give the title compound as yellow oil (1.29 g, 60%). ¹H NMR (d⁶-DMSO) δ 7.62 (d, 1H), 7.95 (d, 1H).

### Intermediate 125

### 2-Bromo-5-chloro-4-fluorobenzenesulfonyl chloride

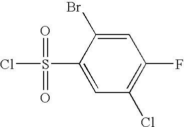

A mixture of 4-bromo-1-chloro-2-fluorobenzene (10 g, 47.7 mmol) and fuming sulfuric acid (18-24% SO₃, 40 ml) was heated at 110° C. over night. After cooling down to room temperature, the reaction mixture was carefully poured into ice (700 g). The resulting mixture was stirred at room temperature for 30 min followed by addition of EA. The aqueous layer was isolated and concentrated under vacuum. The precipitate was filtered off to give 2,5-dichloro-4-fluorobenzenesulfonic acid as white crystals (3.6 g, 26%).

The acid previously prepared (3.51 g, 11.4 mmol) and phosphorus pentachloride (7.77 g, 37.3 mmol) were heated at 110° C. overnight. After cooling down to room temperature, the reaction mixture was carefully poured into ice (50 g) The resulting mixture was stirred at room temperature for 30 min followed by addition of EA (100 mL). The ethyl acetate layer was isolated and concentrated under vacuum to give the title compound as a yellow oil (3.51 g, 92%). ¹H NMR (CDCl₃) δ 8.30 (d, 1H), 7.69 (d, 1H).

### Intermediate 126

### ((2R,4R)-4-{[(2,5-dibromophenyl)sulfonyl]amino}-1-{[(1,1-dimethylethyl)oxy]carbonyl}-2-pyrrolidinyl)methyl 1H-imidazole-1-carboxylate

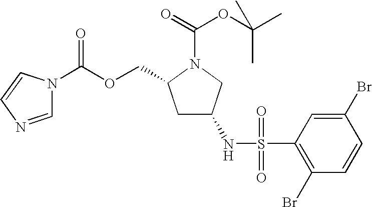

1,1-dimethylethyl (2R,4R)-4-{[(2,5-dibromophenyl)sulfonyl]amino}-2-(hydroxymethyl)-1-pyrrolidinecarboxylate (50 mg, 0.097 mmol) was diluted in DCM (1 mL) and mixed with carbodiimidazole at room temperature for 2.5 days. The reaction was then diluted with DCM and washed with brine. The organic layer was separated via a hydrophobic frit and concentrated under vacuum to give ((2R,4R)-4-{[(2,5-dibromophenyl)sulfonyl]amino}-1-{[(1,1-dimethylethyl)oxy]carbonyl}-2-pyrrolidinyl)methyl 1H-imidazole-1-carboxylate (59 mg, 100%). LC-MS: m/z, 440.8 (M+H), rt 2.04 min.

### Intermediate 127

### 1,1-dimethylethyl (2R,4R)-4-{[(2,5-dibromophenyl)sulfonyl]amino}-2-formyl-1-pyrrolidinecarboxylate

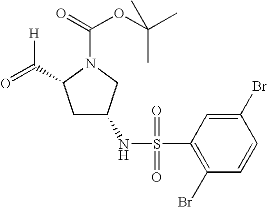

To 1,1-dimethylethyl (2R,4R)-4-{[(2,5-dibromophenyl)sulfonyl]amino}-2-(hydroxymethyl)-1-pyrrolidinecarboxylate (0.205 g, 0.399 mmol) in DCM (50 mL) was added NaBr (0.082 g, 0.797 mmol) and TEMPO (6.23 mg, 0.040 mmol) at 0° C. The solution became pale yellow, then 2 mL of a solution of NaHCO₃ (220 mg) and 10-13% aq NaClO (2.0 mL) in water (5.0 mL) was added. The solution turned brown very quickly and was immediately extracted with ether (10.0 mL) then washed with a aq. Na₂S₂O₃ solution (10.0 ml) then brine (10.0 mL). The organic layer was then dried over Mg₂SO₄ and concentrated to give a crude mixture containing 1,1-dimethylethyl (2R,4R)-4-{[(2,5-dibromophenyl)sulfonyl]amino}-2-formyl-1-pyrrolidinecarboxylate which was used as such for subsequent reactions.

### Intermediate 128

### 1,1-dimethylethyl (2R,4S)-4-{[(1,1-dimethylethyl)(dimethyl)silyl]oxy}-2-[(ethyloxy)methyl]-1-pyrrolidinecarboxylate

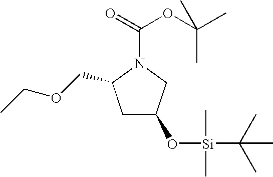

To 1,1-dimethylethyl (2R,4S)-4-{[(1,1-dimethylethyl)(dimethyl)silyl]oxy}-2-(hydroxymethyl)-1-pyrrolidinecarboxylate (1.0 g, 3.03 mmol) in THF (10 mL) were added ethyl bromide (3.29 g, 30.2 mmol) and KI (50 mg) at 0° C. under an argon atmosphere. NaH (0.725 g, 30.2 mmol) was added portionwise to the mixture, which was then allowed to stir from 0° C. to room temperature over 2.5 days. The reaction mixture was quenched with water then DCM was added. The organic layer was separated and washed with a 0.1 N HCl solution then a sat. aq NaHCO₃ solution, dried over MgSO₄ and concentrated to afford a crude material, which was shown to contain some unreacted 1,1-dimethylethyl (2R,4S)-4-{[(1,1-dimethylethyl)(dimethyl)silyl]oxy}-2-(hydroxymethyl)-1-pyrrolidinecarboxylate. The procedure (same amount of reagents, but a reaction time of 16 hours instead of 2.5 days) described above was repeated using the crude material instead of the pure alcohol starting material. The second crude material thus obtained was shown to still contain some alcohol starting material and was purified by automated flash chromatography (using a gradient of ethyl acetate in hexane) to give the title compound (0.4207 g). LC/MS: m/z, 259 (M−100(BOC)), rt 2.98 min.

### Intermediate 129

### 1,1-dimethylethyl (2R,4S)-2-[(ethyloxy)methyl]-4-hydroxy-1-pyrrolidinecarboxylate

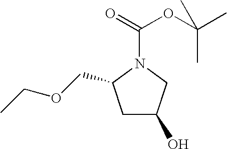

To 1,1-dimethylethyl (2R,4S)-4-{[(1,1-dimethylethyl)(dimethyl)silyl]oxy}-2-[(ethyloxy)methyl]-1-pyrrolidinecarboxylate (420.7 mg) in THF (11.7 mL) was added TBAF (2.34 mL, 2.34 mmol) and the resulting mixture was stirred at room temperature for 3 hours. THF was removed under vacuum then the residue was diluted with ethyl acetate. The mixture was washed with a 0.1 N HCl solution, a sat. aq NaHCO₃ solution, then a sat. aq. sodium chloride solution. The organic layer was dried over MgSO₄, filtered and concentrated to afford the title compound (0.257 g, trace of TBS-containing products still present). LC-MS: m/z, 245 (M+), rt 1.39 min.

### Intermediate 130

### 1,1-dimethylethyl (2R,4S)-2-[(ethyloxy)methyl]-4-[(methylsulfonyl)oxy]-1-pyrrolidinecarboxylate

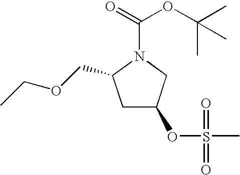

Under an argon atmosphere at 0° C., 1,1-dimethylethyl (2R,4S)-2-[(ethyloxy)methyl]-4-hydroxy-1-pyrrolidinecarboxylate (0.257 g, 1.048 mmol) was diluted in DCM (3 mL). To the resultant mixture, triethylamine (0.438 mL, 3.14 mmol), DMAP (0.192 g, 1.571 mmol) were added followed by methanesulfonyl chloride (0.122 ml, 1.571 mmol). The resulting mixture was stirred at room temperature for 2 hours. The reaction mixture was diluted with DCM (3 mL). The organic layer was washed with a 0.1 N HCl solution, a sat. aq. NaHCO₃ solution, then an aq. sat. NaCl solution. The organic layer was separated through a hydrophobic frit and concentrated to afford the title compound as an orange oil (0.315 g). LC-MS: m/z, 323.9 (M+H), rt 1.68 min.

### Intermediate 131

### 1,1-dimethylethyl (2R,4R)-4-azido-2-[(ethyloxy)methyl]-1-pyrrolidinecarboxylate

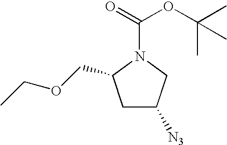

To an orange solution of 1,1-dimethylethyl (2R,4S)-2-[(ethyloxy)methyl]-4-[(methylsulfonyl)oxy]-1-pyrrolidinecarboxylate (0.3154 g, 0.975 mmol) in AcCN (4 mL) was added NaN₃ (0.333 g, 1.170 mmol) under an argon atmosphere. The reaction mixture was then heated at reflux for 5 hr then stirred at room temperature overnight. The reaction mixture was diluted with DCM and washed with water, a 0.1 N HCl solution and a sat.aq.NaHCO₃ solution then dried over MgSO₄. After concentration, a crude material containing the title compound and 15% of starting material was obtained. To this material in AcCN (2.0 mL) was added NaN₃ (0.166 g, 0.585 mmol). The resulting mixture was stirred at reflux for 3 hr, then diluted with DCM and washed with water, a 0.1 N HCl solution and a sat.aq.NaHCO₃ solution. It was then dried over MgSO₄ and concentrated to give a crude residue which was purified by automated flash chromatography (4 g Si cartridge, elution with a 0 to 50% gradient of ethyl acetate in hexane). The relevant fractions were collected, combined and concentrated to yield the title compound (0.1534 g,). LC-MS: m/z, 271 (M+H), rt 1.96 min.

### Intermediate 132

### 1,1-dimethylethyl (2R,4R)-4-amino-2-[(ethyloxy)methyl]-1-pyrrolidinecarboxylate

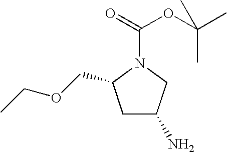

1,1-dimethylethyl (2R,4R)-4-azido-2-[(ethyloxy)methyl]-1-pyrrolidinecarboxylate (0.1534 g, 0.567 mmol) in MeOH (10 mL) was hydrogenated over 10% Pd/C in a H-Cube™ reactor. The reaction mixture was concentrated to afford the title compound (0.20 g, material purity is about 50% by LC/MS with an known impurity present). LC-MS: m/z, 245 (M+H), rt 1.31 min.

### Intermediate 133

### 1,1-dimethylethyl (2R,4R)-4-{[(2,5-dibromophenyl)sulfonyl]amino}-2-[(ethyloxy)methyl]-1-pyrrolidinecarboxylate

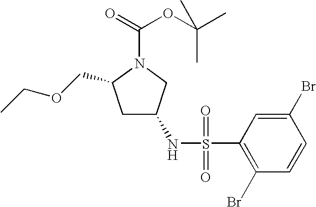

To 1,1-dimethylethyl (2R,4R)-4-amino-2-[(ethyloxy)methyl]-1-pyrrolidinecarboxylate (0.0834 g, 0.341 mmol) in DCM (1.5 mL) were added DIPEA (0.119 mL, 0.683 mmol) and 2,5-dibromobenzenesulfonyl chloride (0.114 g, 0.341 mmol). The resultant mixture was stirred at room temperature for 3 h. The reaction mixture was concentrated under vacuum and the residue was loaded onto a 1 g silica SPE cartridge, eluting sequentially with DCM and ethyl acetate. The ethyl acetate fractions were combined and concentrated to afford a residue containing the title compounds with some traces of DIPEA and an unknown product. This material was washed with water, a 0.1 N HCl solution then a sat. aq. NaHCO₃ solution. The organic layer was separated by passing the mixture through a hydrophobic frit. After concentration, the title compound was obtained (0.144 g, 0.266 mmol). LC-MS: m/z, 543 (M+H), rt 2.59 min.

### Intermediate 134

### 1,1-dimethylethyl (2S,4S)-2-(cyanomethyl)-4-{[(1,1-dimethylethyl)(dimethyl)silyl]oxy}-1-pyrrolidinecarboxylate

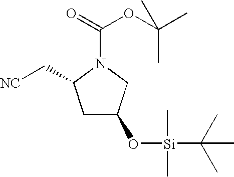

To a solution of 1,1-dimethylethyl (2R,4S)-4-{[(1,1-dimethylethyl)(dimethyl)silyl]oxy}-2-{[(methylsulfonyl)oxy]methyl}-1-pyrrolidinecarboxylate (3.66 g, 8.94 mmol) in acetonitrile (30 ml), tetrabutylammonium cyanide (3.60 g, 13.40 mmol) was added, then the mixture was heated to reflux for 4 h. After LC/MS showed the reaction was completed, it was cooled to room temperature. The solvent was removed and the residue was purified by flash automated chromatography (0-30% ethyl acetate gradient in hexane) to give the title compound as a colorless oil (2.31 g). LC/MS: m/z, 341 (M+H), rt 1.40 min

### Intermediate 135

### 1,1-dimethylethyl (2S,4S)-2-(cyanomethyl)-4-hydroxy-1-pyrrolidinecarboxylate

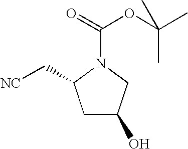

To 1,1-dimethylethyl (2S,4S)-2-(cyanomethyl)-4-{[(1,1-dimethylethyl)(dimethyl)silyl]oxy}-1-pyrrolidinecarboxylate (1.14 g, 3.35 mmol) in THF (10 mL), a TBAF (0.982 g, 3.68 mmol) solution in THF (15 ml) was added at room temperature and the resulting mixture was stirred for 2 h. LC/MS showed the reaction was completed. The solvent was removed under vacuum to give a residue, which was dissolved in EtOAc (20 ml), washed with water (10 ml) and brine (10 ml), and then dried over MgSO₄. The solvent was removed under vacuum, to give the title product as a colorless oil (1.02 g) which was used in the next step without purification. LC/MS: m/z, 227 (M+H), rt 1.31 min.

### Intermediate 136

### 1,1-dimethylethyl (2S,4S)-2-(cyanomethyl)-4-[(methylsulfonyl)oxy]-1-pyrrolidinecarboxylate

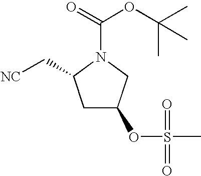

To a mixture of 1,1-dimethylethyl (2S,4S)-2-(cyanomethyl)-4-hydroxy-1-pyrrolidinecarboxylate (1.0 g, 3.35 mmol) and TEA (0.699 ml, 5.02 mmol) in DCM (15 ml), Ms-Cl (0.287 ml, 3.68 mmol) was added dropwise and the resulting reaction mixture was stirred at room temperature for 16 hours. LC/MS showed reaction was completed. The mixture was diluted with DCM (10 ml), washed with water (2×50 ml) and brine (5 ml), and then dried over MgSO₄. After filtration and concentration under vacuum, the crude product was purified by automated flash chromatography to give the title compound (0.848 g). LC/MS: m/z, 204.9 (M−100 (BOC)), rt 0.79 min.

### Intermediate 137

### 1,1-dimethylethyl (2R,4R)-4-azido-2-(cyanomethyl)-1-pyrrolidinecarboxylate

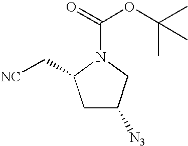

To a solution of 1,1-dimethylethyl (2S,4S)-2-(cyanomethyl)-4-[(methylsulfonyl)oxy]-1-pyrrolidinecarboxylate (0.848 g, 2.79 mmol) in acetonitrile (15 ml), tetrabutyl ammonium azide (1.189 g, 4.18 mmol) was added in several portions, then the mixture was heated at reflux for 4 hours. LC/MS showed that the reaction was completed. The mixture was cooled down to room temperature and the solvent was removed under reduced pressure. The resulting crude product was purified by automated flash chromatography to give the title compound (0.605 g). LC/MS: m/z, 241.9; rt 0.86 min.

### Intermediate 138

### 1,1-dimethylethyl (2R,4R)-4-amino-2-(cyanomethyl)-1-pyrrolidinecarboxylate

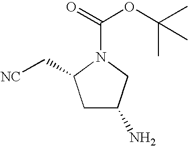

1,1-Dimethylethyl (2R,4R)-4-azido-2-(cyanomethyl)-1-pyrrolidinecarboxylate (510 mg, 2.030 mmol) in MeOH (50 mL) was hydrogenated over 10% Pd/C in a H-Cube™ reactor for 3 hr. The reaction mixture was concentrated to afford the title compound (449 mg). LC-MS: m/z, 225.9 (M), rt 0.78 min.

### Intermediate 139

### N²-{[(1,1-dimethylethyl)oxy]carbonyl}-N¹-methyl-N¹-(methyloxy)-L-valinamide

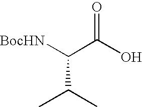

To a mixture of N-methoxymethanamine hydrochloride (49.4 g, 0.507 mol) in DCM (1.5 L) at 0° C. are added TEA (146 ml, 1.106 mol), N-{[(1,1-dimethylethyl)oxy]carbonyl}-L-valine (100 g, 0.461 mol) and TBTU (163 g, 0.507 mol). The reaction mixture is stirred for about 18 hr. Then water (500 ml) and DCM (500 ml) are added. The solid formed is removed by filtration, and the filtrate is placed for ten minutes. Then the organic phase is separated, washed with water (500 ml×3), dried over Na₂SO₄ and concentrated. The residue is purified by column chromatography on silica gel (PE:EA=4:1) to afford N²-{[(1,1-dimethylethyl)oxy]carbonyl}-N¹-methyl-N¹-(methyloxy)-L-valinamide (90 g, 75.0%). ¹H NMR (CDCl₃) δ 0.90 (q, 6H), 1.41 (s, 9H), 1.95 (m, 1H), 3.20 (s, 3H), 3.75 (s, 3H), 4.55 (s, 1H), 5.11 (d, 1H).

### Intermediate 140

### 1,1-dimethylethyl [(1S)-1-formyl-2-methylpropyl]carbamate

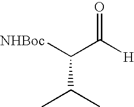

To a solution of N²-{[(1,1-dimethylethyl)oxy]carbonyl}-N¹-methyl-N¹-(methyloxy)-L-valinamide (90 g, 0.346 mol) in THF (1.5 L) at 0° C. is added LiAlH₄ (14.46 g, 0.381 mol) slowly. The reaction mixture is stirred for about 1 h. Then water (30 ml) is added dropwise, the solid formed is removed by filtration, and the filtrate is dried over Na₂SO₄ and concentrated to afford 1,1-dimethylethyl [(1S)-1-formyl-2-methylpropyl]carbamate (71 g) as a crude material. ¹H NMR (CDCl₃) δ 0.95 (q, 6H), 1.41 (s, 9H), 2.25 (s, 1H), 4.20 (s, 1H), 5.09 (s, 1H), 9.60 (s, 1H).

### Intermediate 141

### 1,1-dimethylethyl [(1S)-1-(1-methylethyl)-2-propen-1-yl]carbamate

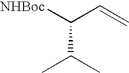

To a mixture of CH₃Ph₃PBr (28.4 g, 79.6 mmol) in THF (150 ml) at −78° C. is added n-BuLi (29.8 ml, 74.6 mmol) dropwise, the reaction mixture is allowed to warm to 0° C. and stirred for 0.5 h. Then a solution of 1,1-dimethylethyl [(1S)-1-formyl-2-methylpropyl]carbamate (10 g, 49.75 mmol) in THF (50 ml) is added dropwise at −78° C. The reaction is then allowed to warm to room temperature (28° C.) and stirred for 3 hr. Then water (100 ml) is added dropwise, the organic phase is separated, dried over Na₂SO₄ and concentrated. The residue is purified by column chromatography on silica gel to afford 1,1-dimethylethyl [(1S)-1-(1-methylethyl)-2-propen-1-yl]carbamate (3 g, 30.3%). ¹H NMR (CDCl₃) δ 0.85 (q, 6H), 1.40 (s, 9H), 1.70 (m, 1H), 3.91 (s, 1H), 4.45 (s, 1H), 5.05 (q, 2H), 5.69 (m, 1H).

### Intermediate 142

### 1,1-dimethylethyl [(1S)-1-(1-methylethyl)-2-propen-1-yl]2-propen-1-ylcarbamate

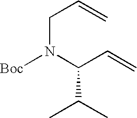

To a solution of 1,1-dimethylethyl [(1S)-1-(1-methylethyl)-2-propen-1-yl]carbamate (2 g, 10.035 mmol) in DMF (20 ml) at 0° C. is added NaH (1.6 g, 40.14 mmol) slowly. After being stirred for about half an hour, 3-bromoprop-1-ene (4.3 ml, 50.176 mmol) is added, the reaction is allowed to warm to room temperature (28° C.) and stirred for 18 hr. Then water (50 mL) is added slowly together with DCM (100 ml), the organic phase is separated and the water phase is extracted with DCM (30 ml×3). The combined organic layers were washed with aq.NH₄Cl solution three times, dried over Na₂SO₄ and concentrated. The residue is purified by column chromatography on silica gel (pet ether:ethyl acetate=140:1) to afford 1,1-dimethylethyl [(1S)-1-(1-methylethyl)-2-propen-1-yl]2-propen-1-ylcarbamate (1.2 g, 50.0%). ¹H NMR (CDCl₃) δ 0.80 (q, 6H), 1.39 (s, 9H), 1.88 (m, 1H), 3.30-4.00 (m, 3H), 5.05 (q, 4H), 5.78 (m, 2H).

### Intermediate 143

### [(1S)-1-(1-methylethyl)-2-propen-1-yl]2-propen-1-ylamine hydrochloride

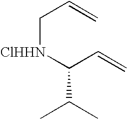

A solution of tert-butyl allyl(S)-4-methylpent-1-en-3-ylcarbamate (21 g, 87.866 mmol) in dioxane/HCl (100 ml) solution is stirred for about 2 hr. The resulting mixture is concentrated to afford [(1S)-1-(1-methylethyl)-2-propen-1-yl]2-propen-1-ylamine hydrochloride (15.37 g, 100%). ¹H NMR (CDCl₃) δ 1.05 (q, 6H), 2.38 (m, 1H), 3.31 (m, 1H), 3.41 (m, 1H), 3.85 (m, 1H), 5.40 (m, 4H), 5.85 (m, 1H), 6.11 (m, 1H), 9.40 (s, 1H), 9.75 (s, 1H).

### Intermediate 144

### phenylmethyl [(1S)-1-(1-methylethyl)-2-propen-1-yl]2-propen-1-ylcarbamate

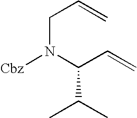

To a mixture of [(1S)-1-(1-methylethyl)-2-propen-1-yl]2-propen-1-ylamine hydrochloride (15.37 g, 87.83 mmol) in ethyl acetate (100 ml) and water (100 ml) is added K₂CO₃ (36.4 g, 263.48 mmol) and then carbobenzyloxyCl (22.5 ml, 131.74 mmol). The reaction mixture is stirred for 16 hr. Then the organic phase is separated and the water phase is extracted with ethyl acetate (50 ml×3). The combined organic layers were dried over Na₂SO₄ and concentrated. The residue is purified by column chromatography on silica gel (PE:EA=50:1) to afford phenylmethyl [(1S)-1-(1-methylethyl)-2-propen-1-yl]2-propen-1-ylcarbamate (23.7 g, 89.3%) as a yellow oil. ¹H NMR (CDCl₃) δ 0.85 (q, 6H), 1.95 (s, 1H), 3.19-4.00 (m, 3H), 5.11 (m, 6H), 5.83 (m, 2H), 7.32 (m, 5H).

### Intermediate 145

### phenylmethyl (2S)-2-(1-methylethyl)-2,5-dihydro-1H-pyrrole-1-carboxylate

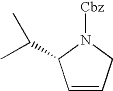

To a solution of phenylmethyl [(1S)-1-(1-methylethyl)-2-propen-1-yl]2-propen-1-ylcarbamate (10 g, 36.68 mmol) in DCM (100 ml) is added Grubbs Catalyst (1 g). The reaction mixture is stirred for 16 hr. The resulting mixture is concentrated and the residue is purified by column chromatography on silica gel (pet ether:ethyl acetate=50:1) to afford phenylmethyl (2S)-2-(1-methylethyl)-2,5-dihydro-1H-pyrrole-1-carboxylate (95.0% yield). ¹H NMR (CDCl₃) δ 0.70 (q, 3H), 0.91 (q, 3H), 2.20-2.50 (m, 1H), 4.05 (m, 1H), 4.26 (m, 1H), 4.55 (m, 1H), 5.12 (m, 2H), 5.11-5.88 (m, 2H), 7.35 (m, 5H).

### Intermediate 146

### phenylmethyl (2R,4S)-4-hydroxy-2-(1-methylethyl)-1-pyrrolidinecarboxylate

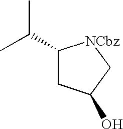

A solution of phenylmethyl (2S)-2-(1-methylethyl)-2,5-dihydro-1H-pyrrole-1-carboxylate (9 g, 36.690 mmol) in BH₃/THF (110 ml, 110.07 mmol) under N₂ atmosphere is heated to reflux for 16 hs. After cooling, NaOH (8.8 g, 220.14 mmol) solid is added. The reaction solution is then cooled to 0° C. And H₂O₂ (33 ml, 293.52 mmol) is added dropwise, the resulting mixture is heated to reflux for 2 hr. Then aq. Na₂SO₃ solution is added in portions. The organic phase is separated and the water phase is extracted with ethyl acetate (50 ml×3). The combined organic layers were dried over Na₂SO₄ and concentrated under reduced pressure. The residue is purified by column chromatography on silica gel (DCM:MeOH=100:1) to afford phenylmethyl (2R,4S)-4-hydroxy-2-(1-methylethyl)-1-pyrrolidinecarboxylate (5.3 g, 54.92%) of the pure compound. ¹H NMR (CDCl₃) δ 0.65-0.95 (m, 6H), 1.81 (m, 2H), 1.90-2.45 (m, 1H), 3.35 (m, 1H), 3.50-4.00 (m, 2H), 4.15-4.35 (m, 1H), 5.10 (s, 2H), 7.28 (m, 5H).

### Intermediate 147

### 1,1-dimethylethyl (2R,4S)-4-hydroxy-2-(1-methylethyl)-1-pyrrolidinecarboxylate

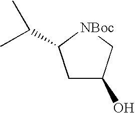

To a solution of (2R,4S)-benzyl 4-hydroxy-2-isopropylpyrrolidine-1-carboxylate (5.3 g, 20.152 mmol) and (Boc)₂O (4.8 ml, 22.167 mmol) in EtOH (100 ml) at H₂ (40 psi) atmosphere is added Pd/C (1 g) as a catalyst. The reaction is stirred for 16 hr. Then the mixture is filtered and the filtrate is condensed to afford 1,1-dimethylethyl (2R,4S)-4-hydroxy-2-(1-methylethyl)-1-pyrrolidinecarboxylate (4.8 g, 100%) as a yellow oil. ¹H NMR (CDCl₃) δ 0.85 (m, 6H), 1.45 (s, 9H), 1.83 (m, 2H), 1.92-2.40 (m, 1H), 3.30 (m, 1H), 3.50-4.00 (m, 2H), 4.19-4.40 (m, 1H).

### Intermediate 148

### 1,1-dimethylethyl (2R,4S)-2-(1-methylethyl)-4-[(methylsulfonyl)oxy]-1-pyrrolidinecarboxylate

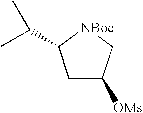

To a solution of 1,1-dimethylethyl (2R,4S)-4-hydroxy-2-(1-methylethyl)-1-pyrrolidinecarboxylate (5.0 g, 21.834 mmol) in DCM (100 mL) is added triethylamine (8.6 ml, 65.502 mmol). Then hypochlorous methanesulfonic anhydride (3.4 ml, 43.668 mmol) is added dropwise at 0° C. The mixture is stirred for 30 min at 25° C. The reaction is monitored via TLC. Then the mixture is washed with water and extracted with ethyl acetate. The organic phase is dried over Na₂SO₄ and evaporated under reduced pressure to afford crude 1,1-dimethylethyl (2R,4S)-2-(1-methylethyl)-4-[(methylsulfonyl)oxy]-1-pyrrolidinecarboxylate (7.5 g) as a yellow oil. ¹H NMR (CDCl₃) δ 0.70-1.00 (m, 6H), 1.45 (s, 9H), 1.70-2.40 (m, 3H), 3.01 (s, 3H), 3.30-4.10 (m, 3H), 5.02-5.19 (m, 1H).

### Intermediate 149

### 1,1-dimethylethyl (2R,4R)-4-azido-2-(1-methylethyl)-1-pyrrolidinecarboxylate

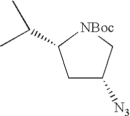

To a solution of 1,1-dimethylethyl (2R,4S)-2-(1-methylethyl)-4-[(methylsulfonyl)oxy]-1-pyrrolidinecarboxylate (7.5 g, 24.43 mmol) in N,N-dimethylformamide (70 mL) is added NaN₃ (6.4 g, 97.72 mmol). The mixture is stirred for 18 hr at 60° C. The mixture is washed with water and saturated NH₄Cl aqueous, then extracted with dichloromethane. The organic phase is dried over Na₂SO₄ and evaporated under reduced pressure. The residue is purified by column chromatography on silica gel (pet ether:ethyl acetate=5:1) to afford 1,1-dimethylethyl (2R,4R)-4-azido-2-(1-methylethyl)-1-pyrrolidinecarboxylate (1.4 g, 22.6%). ¹H NMR (CDCl₃) δ 0.85 (m, 6H), 1.45 (s, 9H), 1.70 (m, 1H), 2.19 (m, 2H), 2.98 (m, 1H), 3.65 (m, 1H), 3.95 (m, 2H).

### Intermediate 150

### 1,1-dimethylethyl (2R,4R)-4-amino-2-(1-methylethyl)-1-pyrrolidinecarboxylate

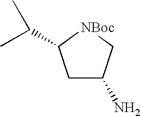

The mixture of 1,1-dimethylethyl (2R,4R)-4-azido-2-(1-methylethyl)-1-pyrrolidinecarboxylate (1.4 g, 5.512 mmol) and Pd/C (300 mg) in ethyl acetate (70 mL) is stirred for 2 h under hydrogen atmosphere. Then the mixture is filtered and the filtrate is condensed to afford 1,1-dimethylethyl (2R,4R)-4-amino-2-(1-methylethyl)-1-pyrrolidinecarboxylate (1.3 g, 100%) as a yellow solid. ¹H NMR (CDCl₃) δ 0.75 (s, 6H), 1.30 (m, 1H), 1.39 (s, 9H), 2.05 (m, 1H), 2.10-2.39 (m, H), 2.65 (m, 1H), 3.28 (m, 1H), 3.60-3.95 (m, 2H).

### Intermediate 151

### 1,1-dimethylethyl (2R,4R)-4-({[(9H-fluoren-9-ylmethyl)oxy]carbonyl}amino)-2-(1-methylethyl)-1-pyrrolidinecarboxylate

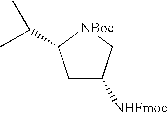

To a solution of (2R,4R)-4-amino-2-(1-methylethyl)-1-pyrrolidinecarboxylate (1.2 g, 5.217 mmol) in ethyl acetate-water (80 mL, 1:1) are added (9H-fluoren-9-yl)methyl carbonochloridate (1.6 g, 6.261 mmol) and potassium carbonate (1.4 g, 10.434 mmol). The mixture is stirred for 2 h at 30° C., and then partitioned between ethyl acetate and water. The organic phase is evaporated under reduced pressure, and the residue is purified by chromatograph on silica gel (pet ether:ethyl acetate=10:1 to 5:1) to afford 1,1-dimethylethyl (2R,4R)-4-({[(9H-fluoren-9-ylmethyl)oxy]carbonyl}amino)-2-(1-methylethyl)-1-pyrrolidinecarboxylate (1.9 g, 82.61%) as a white solid. ¹H NMR (CDCl₃) δ 0.80 (m, 6H), 1.45 (s, 9H), 2.30 (m, 2H), 2.80 (m, 1H), 3.79 (s, 1H), 4.05 (s, 2H), 4.23 (m, 1H), 4.45 (m, 1H), 4.80 (s, 1H), 7.30 (t, 2H), 7.40 (t, 2H), 7.56 (d, 2H), 7.75 (d, 2H).

### Intermediate 152

### 1,1-Dimethylethyl (2R,4R)-4-{[(2,5-dibromophenyl) sulfonyl]amino}-2-({[(phenyloxy) carbonyl]amino}methyl)-1-pyrrolidinecarboxylate

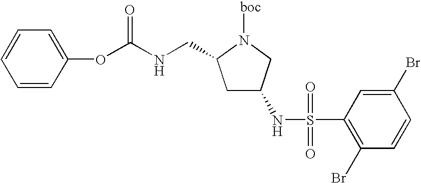

To a solution of 1,1-dimethylethyl (2R,4R)-2-(aminomethyl)-4-{[(2,5-dibromophenyl)sulfonyl]amino}-1-pyrrolidinecarboxylate (530 mg, 1.033 mmol) in THF (3 ml), was added TEA (0.288 ml, 2.065 mmol) followed by phenyl chloroformate (0.143 ml, 1.136 mmol). The reaction mixture was stirred at room temperature overnight. It was then diluted with DCM and washed with 1 N HCl and saturated NaHCO₃. The DCM layer was dried (Na₂SO₄) and evaporated to give the crude as a white solid (654 mg, 100%), which was used for next step without further purification

### Intermediate 153

### 1,1-Dimethylethyl (2R,4R)-4-({[5-chloro-2-(methyloxy)phenyl]sulfonyl}amino)-2-({[(phenyloxy)carbonyl]amino}methyl)-1-pyrrolidinecarboxylate

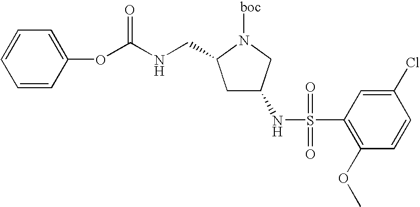

To a solution of 1,1-dimethylethyl (2R,4R)-2-(aminomethyl)-4-({[5-chloro-2-(methyloxy)phenyl]sulfonyl}amino)-1-pyrrolidinecarboxylate (490 mg, 1.033 mmol) in THF (3 ml), was added TEA (0.325 ml, 2.334 mmol) followed by phenyl chloroformate (0.161 mL 1.284 mmol). The reaction mixture was stirred at room temperature overnight. It was diluted with DCM and washed with 1 N HCl and saturated NaHCO₃. The DCM layer was dried (Na₂SO₄) and evaporated to give the crude as a white solid (630 mg, 100%), which was used for next step without further purification.

### Intermediate 154

### 1,1-Dimethylethyl (2R,4R)-4-({[2,5-bis(methyloxy)phenyl]sulfonyl}amino)-2-({[(phenyloxy)carbonyl]amino}methyl)-1-pyrrolidinecarboxylate

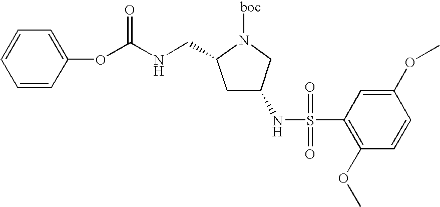

To a solution of 1,1-dimethylethyl (2R,4R)-2-(aminomethyl)-4-({[2,5-bis(methyloxy)phenyl]sulfonyl}amino)-1-pyrrolidinecarboxylate (473 mg, 1.138 mmol) in THF (15 ml), was added TEA (0.317 ml, 2.277 mmol) followed by phenyl chloroformate (0.157 mL 1.252 mmol). The reaction mixture was stirred at room temperature overnight, diluted with DCM and washed with 1 N HCl and saturated NaHCO₃. The DCM layer was dried (Na₂SO₄) and evaporated to give the crude as a white solid (458 mg, 75%), which was used for next step without further purification.

### EXAMPLE 1

### 2,5-Dibromo-N-[(3R,5S)-1-cyano-5-methyl-3-pyrrolidinyl]benzenesulfonamide

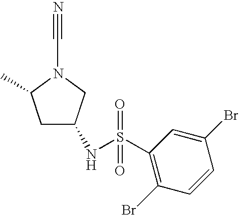

1,1-Dimethylethyl (2S,4R)-4-{[(2,5-dibromophenyl)sulfonyl]amino}-2-methyl-1-pyrrolidinecarboxylate (0.1627 g, 0.326 mmol, 1 eq.) in 4 N HCl (3 mL, 12 mmol) was mixed for 1 hour and the dioxane was evaporated under vacuum to yield a white solid which was diluted in DCM (˜3 mL), mixed with DIEA (0.228 mL, 1.31 mmol) and BrCN (0.327 mL, 0.98 mmol). The resultant mixture was stirred at room temperature overnight; PS-trisamine (0.3275 g, 4 eq.) was added and the mixture was stirred at room temperature for another 2 hours. The resin was filtered out, the solvent evaporated under vacuum and the residue purified by preparatory HPLC (without TFA) to afford the title compound (0.0805 g). LC-MS: m/z, 424 (M+H). 1H NMR (400 MHz, CHLOROFORM-d) δ ppm 8.29 (1H, d, J=2.26 Hz) 7.59-7.66 (1H, m) 7.63 (1H, q, J=8.45 Hz) 5.44 (1H, d, J=7.78 Hz) 3.89 (1H, dd, J=15.94, 7.91 Hz) 3.63 (1H, dt, J=9.10, 6.37 Hz) 3.51 (1H, dd, J=9.91, 7.40 Hz) 3.26 (1H, dd, J=9.91, 7.15 Hz) 2.29 (1H, dt, J=12.86, 6.49 Hz) 1.47-1.53 (1H, m) 1.38 (3H, d, J=6.27 Hz).

### EXAMPLE 2

### 2,5-Dichloro-N-[(3R,5S)-1-cyano-5-methyl-3-pyrrolidinyl]benzenesulfonamide

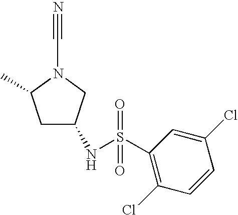

1,1-Dimethylethyl (2S,4R)-4-{[(2,5-dichlorophenyl)sulfonyl]amino}-2-methyl-1-pyrrolidinecarboxylate (0.0149 g, 0.036 mmol, 1 eq.) was mixed with 4 N HCl in dioxane (8 mL) and stirred at room temperature overnight. The solvent was evaporated under vacuum to yield a white solid which was diluted in DCM (˜1 mL), mixed with DIEA (0.025 mL, 0.15 mmol) and BrCN (0.36 mL, 0.11 mmol). The resultant mixture was stirred at room temperature for 6 hours, PS-trisamine (4 eq) was added and the mixture was stirred at room temperature overnight. The resin was filtered out, the solvent evaporated under vacuum, and the residue purified by preparatory HPLC (without TFA) to afford the title compound (0.0048 g). LC-MS: m/z, 334 (M+H). 1H NMR (400 MHz, CHLOROFORM-d) δ ppm 8.02 (1H, d, J=2.26 Hz) 7.41-7.49 (2H, m) 5.20-5.27 (1H, m) 3.79-3.86 (1H, m) 3.54 (1H, ddd, J=9.16, 6.27, 6.15 Hz) 3.43 (1H, dd, J=9.79, 7.28 Hz) 3.16 (1H, dd, J=9.91, 7.15 Hz) 2.20 (1H, dt, J=12.86, 6.49 Hz) 1.37-1.43 (1H, m) 1.29 (3H, d, J=6.27 Hz).

### EXAMPLE 3

### N-[(3R,5S)-1-Cyano-5-methyl-3-pyrrolidinyl]-2,5-bis(methyloxy)benzenesulfonamide

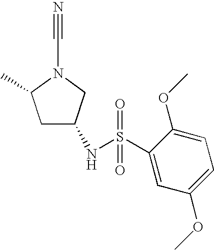

1,1-Dimethylethyl (2S,4R)-4-({[2,5-bis(methyloxy)phenyl]sulfonyl}amino)-2-methyl-1-pyrrolidinecarboxylate (0.0615 g, 0.15 mmol, 1 eq.) was mixed with 4 N HCl (3 mL). The reaction mixture was stirred at room temperature for 12 hours, the solvent evaporated and the resulting solid was dissolved in DCM (˜3 mL). To the resultant mixture was added DIEA (0.32 mL, 0.19 mmol) and BrCN (0.47 mL, 0.14 mmol). The resultant mixture was stirred for 4 hours at room temperature and then PS-trisamine (4 eq.) was added to quench the reaction. The reaction was stirred for another 2 hours at room temperature, filtered, concentrated and purified by preparatory HPLC (without TFA) to afford the title compound (0.0252 g). LC-MS: m/z, 326 (M+H). 1H NMR (400 MHz, CHLOROFORM-d) δ ppm 7.43 (1H, d, J=2.27 Hz) 7.12 (1H, dd, J=8.81, 2.27 Hz) 7.02 (1H, d, J=9.06 Hz) 5.37 (1H, d, J=7.81 Hz) 3.97 (3H, s) 3.85-3.89 (1H, m) 3.83 (3H, s) 3.54-3.61 (1H, m) 3.40 (1H, t, J=8.44 Hz) 3.17 (1H, t) 2.21 (1H, ddd, J=12.72, 6.55, 6.42 Hz) 1.42 (1H, q) 1.33 (3H, d, J=6.04 Hz)

### EXAMPLE 4

### 3-Bromo-N-[(3R,5S)-1-cyano-5-methyl-3-pyrrolidinyl]benzenesulfonamide

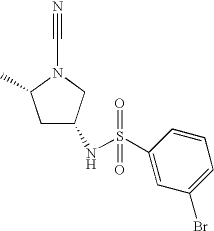

Example 4 was prepared using the general procedure described above for Example 3, replacing 1,1-dimethylethyl (2S,4R)-4-({[2,5-bis(methyloxy)phenyl]sulfonyl}amino)-2-methyl-1-pyrrolidinecarboxylate (0.0615 g, 0.15 mmol)) with 1,1-dimethylethyl (2S,4R)-4-{[(3-bromophenyl)sulfonyl]amino}-2-methyl-1-pyrrolidinecarboxylate (0.1351 g, 0.32 mmol) to afford the title compound (0.0465 g). LC-MS: m/z, 345 (M+H). 1H NMR (400 MHz, CHLOROFORM-d) δ ppm 8.02 (1H, s) 7.81 (1H, d, J=7.55 Hz) 7.75 (1H, d, J=8.06 Hz) 7.44 (1H, t, J=7.81 Hz) 5.88 (1H, d, J=6.80 Hz) 3.80-3.88 (1H, m) 3.58-3.68 (1H, m) 3.52 (1H, t, J=8.56 Hz) 3.23 (1H, t, J=8.44 Hz) 2.25 (1H, dt, J=12.59, 6.29 Hz) 1.46 (1H, q) 1.34 (3H, d, J=6.04 Hz)

### EXAMPLE 5

### 2-Bromo-N-[(3R,5S)-1-cyano-5-methyl-3-pyrrolidinyl]-5-(trifluoromethyl)benzenesulfonamide

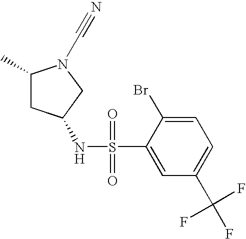

Example 5 was prepared using the general procedure described above for Example 3, replacing 1,1-dimethylethyl (2S,4R)-4-({[2,5-bis(methyloxy)phenyl]sulfonyl}amino)-2-methyl-1-pyrrolidinecarboxylate (0.0615 g, 0.15 mmol)) with 1,1-dimethylethyl (2S,4R)-4-({[2-bromo-5-(trifluoromethyl)phenyl]sulfonyl}amino)-2-methyl-1-pyrrolidinecarboxylate (0.1206 g, 0.25 mmol) to afford the title compound (0.0512 g). LC-MS: m/z, 413 (M+H). 1H NMR (400 MHz, CHLOROFORM-d) δ ppm 8.39 (s, 1H) 7.93 (d, J=8.31 Hz, 1H) 7.72 (d, J=8.06 Hz, 1H) 5.82 (d, J=7.05 Hz, 1H) 3.87-3.94 (m, 1H) 3.59-3.65 (m, 1H) 3.52 (t, J=8.56 Hz, 1H) 3.23-3.34 (m, 1H) 2.27 (ddd, J=12.72, 6.55, 6.42 Hz, 1H) 1.44-1.58 (m, 1H) 1.36 (d, J=6.04 Hz, 3H)

### EXAMPLE 6

### 2-Chloro-N-[(3R,5S)-1-cyano-5-methyl-3-pyrrolidinyl]-5-(trifluoromethyl)benzenesulfonamide

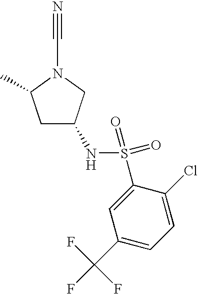

Example 6 was prepared using the general procedure described above for Example 3, replacing 1,1-dimethylethyl (2S,4R)-4-({[2,5-bis(methyloxy)phenyl]sulfonyl}amino)-2-methyl-1-pyrrolidinecarboxylate (0.0615 g, 0.15 mmol)) with 1,1-dimethylethyl (2S,4R)-4-({[2-chloro-5-(trifluoromethyl)phenyl]sulfonyl}amino)-2-methyl-1-pyrrolidinecarboxylate (0.2066 g, 0.47 mmol) to afford the title compound (0.0050 g). LC-MS: m/z, 368 (M+H). 1H NMR (400 MHz, CHLOROFORM-d) δ ppm 8.34 (br. s., 1H) 7.81 (d, J=8.31 Hz, 1H) 7.68-7.75 (m, 1H) 6.12 (br. s., 1H) 3.90 (qd, J=7.34, 7.18 Hz, 1H) 3.55-3.66 (m, 1H) 3.51 (t, J=8.44 Hz, 1H) 3.29 (t, J=8.44 Hz, 1H) 2.22 (dt, J=12.59, 6.29 Hz, 1H) 1.52 (t, J=9.95 Hz, 1H) 1.33 (d, J=6.04 Hz, 3H)

### EXAMPLE 7

### 5-Bromo-N-[(3R,5S)-1-cyano-5-methyl-3-pyrrolidinyl]-2-(methyloxy)benzenesulfonamide

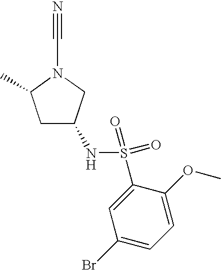

Example 7 was prepared using the general procedure described above for Example 3, replacing 1,1-dimethylethyl (2S,4R)-4-({[2,5-bis(methyloxy)phenyl]sulfonyl}amino)-2-methyl-1-pyrrolidinecarboxylate (0.0615 g, 0.15 mmol)) with 1,1-dimethylethyl (2S,4R)-4-({[5-bromo-2-(methyloxy)phenyl]sulfonyl}amino)-2-methyl-1-pyrrolidinecarboxylate (0.1285 g, 0.28 mmol) to afford the title compound (0.0773 g). LC-MS: m/z, 375 (M+H). 1H NMR (400 MHz, CHLOROFORM-d) δ ppm 8.02 (s, 1H) 7.69 (d, J=8.56 Hz, 1H) 6.97 (d, J=8.81 Hz, 1H) 5.53 (d, J=7.55 Hz, 1H) 4.00 (s, 3H) 3.82-3.89 (m, 1H) 3.56-3.63 (m, 1H) 3.44 (t, J=8.56 Hz, 1H) 3.18 (t, J=8.31 Hz, 1H) 2.20 (dt, J=12.78, 6.33 Hz, 1H) 1.37-1.49 (m, 1H) 1.33 (d, J=6.04 Hz, 3H)

### EXAMPLE 8

### 5-Bromo-N-[(3R,5S)-1-cyano-5-methyl-3-pyrrolidinyl]-2,4-difluorobenzenesulfonamide

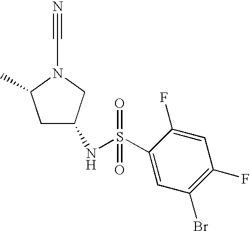

A mixture of 1,1-dimethylethyl (2S,4R)-4-{[(5-bromo-2,4-difluorophenyl)sulfonyl]amino}-2-methyl-1-pyrrolidinecarboxylate (0.140 g, 0.31 mmol, 1 eq.) in 4 N HCl in dioxane (3 mL) was stirred for 2 hours and concentrated under vacuum. The orange oil obtained was diluted in DCM (˜5 mL) and DIEA (0.22 mL, 1.24 mmol) was added. To the resultant mixture BrCN (0.31 mL, 0.93 mmol) was added and the mixture was stirred at room temperature overnight. To the resultant mixture PS-trisamine (4 eq) was added and stirred for another 2 hours, filtered, concentrated and purified by preparatory HPLC (without TFA) to afford the title compound (0.046 g). LC-MS: m/z, 381 (M+H). 1H NMR (400 MHz, CHLOROFORM-d) δ ppm 8.05 (t, J=7.40 Hz, 1H) 7.02 (dd, J=9.54, 7.78 Hz, 1H) 5.68 (br. s., 1H) 3.89 (br. s., 1H) 3.54-3.60 (m, J=6.27, 3.51, 3.14, 3.14 Hz, 1H) 3.51 (dd, J=9.79, 7.28 Hz, 1H) 3.21 (dd, J=9.91, 7.15 Hz, 1H) 2.22 (dt, J=12.86, 6.49 Hz, 1H) 1.42 (dt, J=12.80, 9.16 Hz, 1H) 1.29 (d, J=6.27 Hz, 3H)

### EXAMPLE 9

### N-[(3R,5S)-1-Cyano-5-methyl-3-pyrrolidinyl]-2,3,5-trifluorobenzenesulfonamide

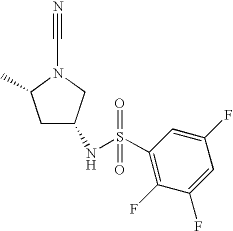

Example 9 was prepared using the general procedure described above for Example 8, replacing 1,1-dimethylethyl (2S,4R)-4-{[(5-bromo-2,4-difluorophenyl)sulfonyl]amino}-2-methyl-1-pyrrolidinecarboxylate (0.140 g, 0.31 mmol) with 1,1-dimethylethyl (2S,4R)-2-methyl-4-{[(2,4,5-trifluorophenyl)sulfonyl]amino}-1-pyrrolidinecarboxylate (0.0942 g, 0.24 mmol) to afford the title compound (9 mg). LC-MS: m/z, 320 (M+H). 1H NMR (400 MHz, CHLOROFORM-d) δ ppm 7.70 (td, J=8.66, 6.53 Hz, 1H) 7.07 (td, J=9.29, 5.77 Hz, 1H) 5.27 (d, J=6.78 Hz, 1H) 3.85-3.96 (m, 1H) 3.53-3.60 (m, J=6.27, 3.51, 3.14, 3.14 Hz, 1H) 3.51 (dd, J=9.91, 7.40 Hz, 1H) 3.20 (dd, J=9.79, 7.03 Hz, 1H) 2.24 (dt, J=12.86, 6.49 Hz, 1H) 1.40 (dt, J=12.80, 9.03 Hz, 1H) 1.30 (d, J=6.27 Hz, 3H)

### EXAMPLE 10

### 2,5-Di bromo-N-{(3R,5R)-1-cyano-5-[(methyloxy)methyl]-3-pyrrolidinyl}benzenesulfonamide

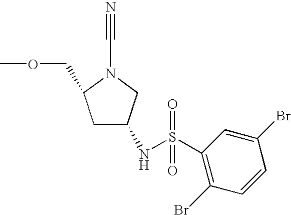

1,1-Dimethylethyl (2R,4R)-4-{[(2,5-dibromophenyl)sulfonyl]amino}-2-[(methyloxy)methyl]-1-pyrrolidinecarboxylate (0.159 g, 0.30 mmol, 1 eq.) was mixed for 1 hour at room temperature with 4 N HCl in dioxane (3 mL). The dioxane was removed under vacuum to yield a white solid which was diluted in DCM (˜15 mL) and DIEA (0.2 mL, 0.12 mmol). BrCN (0.3 mL, 0.90 mmol) was added and the resultant mixture was stirred at room temperature for 1.5 hours. PS-trisamine (4 eq.) was added and the mixture was stirred for another 2 hours at room temperature, filtered, concentrated and purified by preparatory HPLC (without TFA) to afford the title compound as a white solid (0.0535 g). LC-MS: m/z, 454 (M+H). 1H NMR (400 MHz, CHLOROFORM-d) δ ppm 8.25 (1H, d, J=2.51 Hz) 7.62 (1H, d, J=8.28 Hz) 7.53-7.57 (1H, m, J=8.53, 2.51 Hz) 7.04 (1H, d, J=9.29 Hz) 3.94-4.02 (1H, m, J=7.25, 7.25, 5.33, 1.76 Hz) 3.83 (1H, dq, J=10.20, 2.29 Hz) 3.74 (1H, dd, J=10.79, 2.01 Hz) 3.53 (3H, s) 3.46 (1H, dd, J=10.79, 2.01 Hz) 3.42 (1H, dd, J=10.16, 5.14 Hz) 3.26 (1H, dt, J=10.29, 1.63 Hz) 2.34 (1H, td, J=7.03, 3.01 Hz) 1.89 (1H, dd, J=14.18, 2.38 Hz)

### EXAMPLE 11

### 2,5-Dichloro-N-{(3R,5R)-1-cyano-5-[(methyloxy)methyl]-3-pyrrolidinyl}benzenesulfonamide

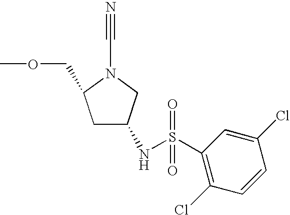

Example 10 was prepared using the general procedure described above for Example 11, replacing 1,1-dimethylethyl (2R,4R)-4-{[(2,5-dibromophenyl)sulfonyl]amino}-2-[(methyloxy)methyl]-1-pyrrolidinecarboxylate (0.159 g, 0.30 mmol) with 1,1-dimethylethyl (2R,4R)-4-{[(2,5-dichlorophenyl)sulfonyl]amino}-2-[(methyloxy)methyl]-1-pyrrolidinecarboxylate (0.144 g, 0.33 mmol) to afford the title compound as a colorless oil (0.0146 g). LC-MS: m/z, 365 (M+H). 1H NMR (400 MHz, CHLOROFORM-d) δ ppm 8.03 (1H, d, J=1.51 Hz) 7.46-7.49 (2H, m) 7.01 (1H, d, J=8.78 Hz) 3.93-4.00 (1H, m) 3.80 (1H, dd, J=10.04, 2.51 Hz) 3.66-3.73 (1H, m) 3.49 (3H, s) 3.38-3.46 (2H, m) 3.23 (1H, dd, J=10.29, 1.51 Hz) 2.30 (1H, ddd, J=13.99, 10.10, 7.28 Hz) 1.84 (1H, d, J=1.51 Hz)

### EXAMPLE 12

### 2-Bromo-N-{(3R,5R)-1-cyano-5-[(methyloxy)methyl]-3-pyrrolidinyl}-5-(trifluoromethyl)benzenesulfonamide

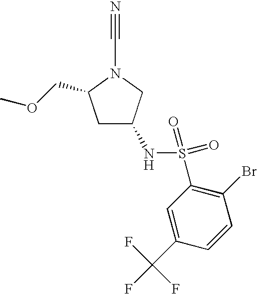

Example 12 was prepared using the general procedure described above for Example 10, replacing 1,1-dimethylethyl (2R,4R)-4-{[(2,5-dibromophenyl)sulfonyl]amino}-2-[(methyloxy)methyl]-1-pyrrolidinecarboxylate (0.159 g, 0.30 mmol) with 1,1-dimethylethyl (2R,4R)-4-({[2-bromo-5-(trifluoromethyl)phenyl]sulfonyl}amino)-2-[(methyloxy)methyl]-1-pyrrolidinecarboxylate (0.129 g, 0.25 mmol) to afford the title compound as a colorless liquid oil (0.0248 g). LC-MS: m/z, 443 (M+H). 1H NMR (400 MHz, CHLOROFORM-d) δ ppm 8.30 (1H, s) 7.84 (1H, d, J=8.03 Hz) 7.57-7.65 (1H, m) 7.06 (1H, d, J=9.29 Hz) 3.93 (1H, td, J=3.45, 1.63 Hz) 3.74-3.82 (1H, m) 3.68 (1H, dd, J=10.79, 2.01 Hz) 3.47 (3H, s) 3.40 (1H, dd, J=10.67, 1.88 Hz) 3.35 (1H, dd, J=10.16, 4.89 Hz) 3.17-3.24 (1H, m) 2.28 (1H, td, J=7.03, 3.26 Hz) 1.84 (1H, d, J=14.05 Hz)

### EXAMPLE 13

### 2,5-Dichloro-N-{(3R,5R)-1-cyano-5-[(phenyloxy)methyl]-3-pyrrolidinyl}benzenesulfonamide

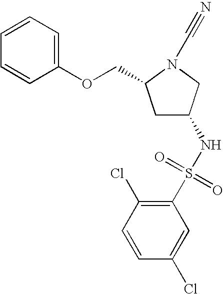

1,1-dimethylethyl (2R,4R)-4-{[(2,5-dichlorophenyl)sulfonyl]amino}-2-[(phenyloxy)methyl]-1-pyrrolidinecarboxylate (0.0545 g, 0.11 mmol, 1 eq.) was mixed for 1.5 hours in 4 N HCl in dioxane (3 mL). The solvent was removed under vacuum to yield a white solid which was diluted in DCM (˜1 mL) and DIEA (0.77 μL, 0.44 mmol). To the resultant mixture BrCN (0.11 mL, 0.33 mmol) was added and stirred at room temperature overnight. PS-trisamine (4 eq.) was added and the resultant mixture was stirred for another 2 hours at room temperature, filtered, concentrated and purified by preparatory HPLC without TFA to afford the title compound (0.00071 g). LC-MS: m/z, 427 (M+H). 1H NMR (400 MHz, CHLOROFORM-d) δ ppm 8.13 (1H, d, J=2.51 Hz) 7.46-7.53 (2H, m) 7.37 (2H, dd, J=8.78, 7.53 Hz) 7.03-7.11 (2H, m) 6.77 (1H, d, J=9.29 Hz) 4.35 (1H, dd, J=10.29, 2.26 Hz) 4.04-4.10 (2H, m, J=7.31, 4.75, 4.75, 2.26 Hz) 4.04-4.10 (1H, m) 4.02 (1H, d, J=2.51 Hz) 3.49 (1H, dd, J=10.29, 5.02 Hz) 3.36 (1H, ddd, J=10.48, 1.82, 1.51 Hz) 2.48 (1H, ddd, J=14.05, 9.91, 7.15 Hz) 2.01-2.10 (1H, m)

### EXAMPLE 14

### 2,5-Dibromo-N-{(3R,5R)-1-cyano-5-[(phenyloxy)methyl]-3-pyrrolidinyl}benzenesulfonamide

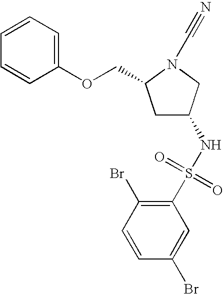

Example 14 was prepared using the general procedure described above for Example 13, replacing 1,1-dimethylethyl (2R,4R)-4-{[(2,5-dichlorophenyl)sulfonyl]amino}-2-[(phenyloxy)methyl]-1-pyrrolidinecarboxylate (0.0545 g, 0.11 mmol) with 11,1-dimethyl ethyl (2R,4R)-4-{[(2,5-dibromophenyl)sulfonyl]amino}-2-[(phenyloxy)methyl]-1-pyrrolidinecarboxylate (0.063 g, 0.11 mmol) to afford the title compound (0.0053 g). LC-MS: m/z, 516 (M+H). 1H NMR (400 MHz, CHLOROFORM-d) δ ppm 8.31 (1H, d, J=2.26 Hz) 7.55-7.63 (2H, m) 7.36 (1H, d, J=7.28 Hz) 7.34-7.41 (1H, m) 7.06 (3H, d, J=8.78 Hz) 6.83 (1H, d, J=9.03 Hz) 4.32-4.38 (1H, m) 4.02-4.09 (3H, m) 3.48 (1H, dd, J=10.29, 5.02 Hz) 3.33-3.39 (1H, m) 2.48 (1H, td, J=7.09, 2.89 Hz) 2.09 (1H, d, J=14.31 Hz)

### EXAMPLE 15

### ((2R,4R)-1-Cyano-4-{[(2,5-dibromophenyl)sulfonyl]amino}-2-pyrrolidinyl)methyl 2,2-dimethylpropanoate

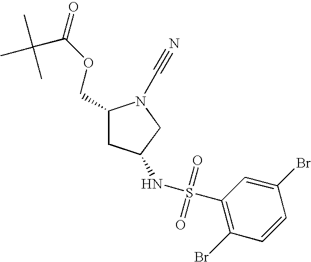

1,1-dimethylethyl (2R,4R)-4-{[(2,5-dibromophenyl)sulfonyl]amino}-2-{[(2,2-dimethylpropanoyl)oxy]methyl}-1-pyrrolidinecarboxylate (0.075 g, 0.12 mmol, 1 eq.) was mixed for 2 hours at room temperature with 4 N HCl in dioxane (2 mL). The solvent was removed under vacuum to yield a white powder which was diluted in DCM (˜5 mL) and DIPEA (0.087 mL, 0.50 mmol. BrCN (0.125 mL, 0.375 mmol) was added and the resultant mixture stirred at room temperature overnight. PS-trisamine (4 eq.) was added and the resultant mixture was stirred for another 2 hours at room temperature, filtered, concentrated in vacuum and purified by preparatory HPLC (without TFA) to afford the title compound (0.008 g). LC-MS: m/z, 524 (M+H). 1H NMR (400 MHz, CHLOROFORM-d) δ ppm 8.15 (1H, d, J=2.01 Hz) 7.45-7.52 (2H, m) 5.44 (1H, br. s.) 4.14 (1H, dd, J=11.92, 4.14 Hz) 3.96-4.02 (1H, m) 3.69-3.81 (2H, m) 3.42 (1H, dd, J=10.04, 6.53 Hz) 3.16 (1H, dd, J=10.04, 6.53 Hz) 2.15 (1H, t, J=7.28 Hz) 1.54 (1H, dt, J=13.30, 7.53 Hz) 1.11 (9H, s)

### EXAMPLE 16

### ((2R,4R)-1-Cyano-4-{[(2,5-dichlorophenyl)sulfonyl]amino}-2-pyrrolidinyl)methyl 2,2-dimethylpropanoate

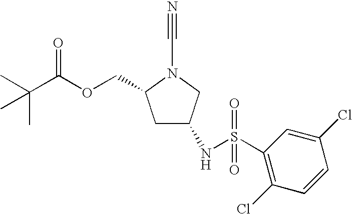

1,1-dimethylethyl (2R,4R)-4-{[(2,5-dichlorophenyl)sulfonyl]amino}-2-{[(2,2-dimethylpropanoyl)oxy]methyl}-1-pyrrolidinecarboxylate (0.157 g, 0.31 mmol, 1 eq.) was mixed for 1.5 hours at room temperature with 4 N HCl in dioxane (1 mL). The crude was concentrated under vacuum to yield a white solid which was diluted in DCM (3 mL) and DIEA (0.21 mL, 1.23 mmol). BrCN (0.31 mL, 0.92 mmol) was added and the resultant mixture was stirred at room temperature. After 16 hours, more BrCN (1 eq.) was added. After stirring for 2 more hours, PS-trisamine (5 eq.) was added and the mixture was stirred for another 1 hour at room temperature, filtered and purified by preparatory HPLC (without TFA) to afford the title compound (37.1 mg). LC-MS: m/z, 435 (M+H). 1H NMR (400 MHz, CHLOROFORM-d) δ ppm 8.09 (1H, d, J=2.51 Hz) 7.46-7.58 (2H, m) 5.94 (1H, d, J=7.28 Hz) 4.24 (1H, dd, J=11.80, 4.02 Hz) 4.07-4.16 (1H, m) 3.82-3.96 (2H, m) 3.55 (1H, dd, J=9.91, 6.90 Hz) 3.30 (1H, dd, J=10.04, 7.03 Hz) 2.28 (1H, t, J=13.93 Hz) 1.68 (1H, dt, J=13.30, 7.91 Hz) 1.23 (9H, s)

### EXAMPLE 17

### [(2R,4R)-4-({[5-Chloro-2-(methyloxy)phenyl]sulfonyl}amino)-1-cyano-2-pyrrolidinyl]methyl 2,2-dimethylpropanoate

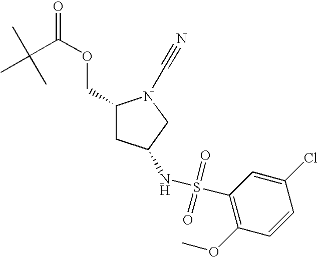

1,1-dimethylethyl (2R,4R)-4-{[(2,5-dichlorophenyl)sulfonyl]amino}-2-{[(2,2-dimethylpropanoyl)oxy]methyl}-1-pyrrolidinecarboxylate (0.133 g, 0.26 mmol, 1 eq.) was mixed with in 4 N HCl in dioxane (1 mL) for 1.5 hours at room temperature. The crude was concentrated under vacuum to yield a white solid which was diluted in DCM (3 mL) and DIEA (0.18 mL, 1.05 mmol). BrCN (0.26 mL, 0.79 mmol) was added and the resultant mixture stirred at room temperature. After stirring for 16 hours, BrCN (1 eq.) was added the resultant mixture. PS-trisamine (5 eq.) was added and stirred for another 1 hour at room temperature, then the mixture was filtered and purified by preparatory HPLC (without TFA) to afford the title compound (40.6 mg). LC-MS: m/z, 430 (M+H). 1H NMR (400 MHz, CHLOROFORM-d) δ ppm 7.81 (1H, d, J=2.76 Hz) 7.49 (1H, dd, J=8.91, 2.64 Hz) 6.98 (1H, d, J=8.78 Hz) 6.50 (1H, br. s.) 4.13 (1H, dd, J=11.80, 4.02 Hz) 4.05 (1H, dd, J=11.80, 6.78 Hz) 3.93 (3H, s) 3.75-3.85 (2H, m) 3.44 (1H, dd, J=9.91, 6.90 Hz) 3.15-3.20 (1H, m) 2.16 (1H, ddd, J=13.61, 7.28, 6.96 Hz) 1.56 (1H, dt, J=13.30, 7.78 Hz) 1.17 (9H, s)

### EXAMPLE 18

### 2-Bromo-N-[(3R,5S)-1-cyano-5-methyl-3-pyrrolidinyl]-5-nitrobenzenesulfonamide

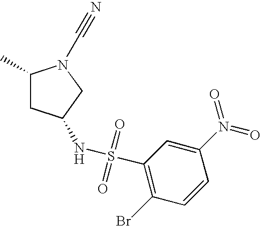

1,1-dimethylethyl (2S,4R)-4-{[(2-bromo-5-nitrophenyl)sulfonyl]amino}-2-methyl-1-pyrrolidinecarboxylate (51.3 mg, 0.11 mmol, 1 eq.) was mixed for 2 hours with 4 N HCl in dioxane (0.57 mL). The solvent was concentrated under vacuum to yield a brown solid which was diluted in DCM (2 mL) and DIEA (0.79 mL, 0.46 mmol). To the resultant mixture BrCN (0.073 mL, 0.22 mmol) was added and stirred at room temperature overnight. The reaction was quenched with PS-trisamine (4 eq.) and stirred for another 3 hours at room temperature, filtered, concentrated under vacuum and purified by preparatory HPLC (without TFA) to afford the title compound (20 mg). LC-MS: m/z, 390 (M+H). 1H NMR (400 MHz, CHLOROFORM-d) δ ppm 8.43 (1H, d, J=2.26 Hz) 7.90 (1H, dd, J=8.28, 2.26 Hz) 7.78-7.84 (1H, m) 5.45 (1H, d, J=7.78 Hz) 3.77-3.84 (1H, m) 3.53 (1H, dt, J=9.03, 6.27 Hz) 3.44 (1H, dd, J=10.04, 7.28 Hz) 3.19 (1H, dd, J=9.91, 6.90 Hz) 2.18 (1H, dt, J=12.86, 6.49 Hz) 1.40 (1H, dt, J=12.80, 9.03 Hz) 1.29 (3H, d, J=6.27 Hz)

### EXAMPLE 19

### ((2R,4R)-1-Cyano-4-{[(2,5-dibromophenyl)sulfonyl]amino}-2-pyrrolidinyl)methyl phenylcarbamate

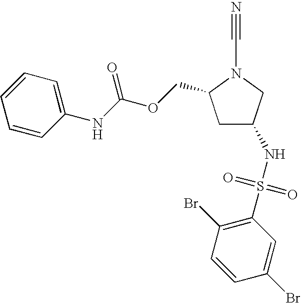

1,1-dimethylethyl (2R,4R)-4-{[(2,5-dibromophenyl)sulfonyl]amino}-2-({[(phenylamino)carbonyl]oxy}methyl)-1-pyrrolidinecarboxylate (0.06 g, 0.95 mmol, 1 eq.) was mixed for 3 hours with 4 N HCl (0.237 mL). The solvent was concentrated under vacuum to yield a white powder which was diluted in DCM (2 mL) and DIEA (66 μL, 0.38 mmol). BrCN (47 μL, 0.142 mmol) was added and the resultant mixture was stirred at room temperature overnight. PS-trisamine (4 eq.) was added and the resultant mixture was stirred for another 1.5 hours at room temperature then filtered, concentrated under vacuum and purified by preparatory HPLC (without TFA) to afford the title compound (0.0233 mg). LC-MS: m/z, 559 (M+H). 1H NMR (400 MHz, CHLOROFORM-d) δ ppm 8.28 (1H, d, J=2.26 Hz) 7.56-7.63 (2H, m) 7.43 (2H, d, J=7.78 Hz) 7.33 (2H, t, J=8.03 Hz) 7.28 (1H, s) 7.10 (1H, t, J=7.40 Hz) 6.11 (1H, br. s.) 4.46 (1H, dd, J=12.05, 3.26 Hz) 4.26 (1H, dd, J=12.05, 5.02 Hz) 3.92-3.99 (2H, m) 3.53 (1H, dd, J=10.16, 5.90 Hz) 3.39 (1H, dd, J=10.16, 4.39 Hz) 2.33-2.37 (1H, m) 1.93 (1H, ddd, J=13.55, 5.40, 5.14 Hz)

### EXAMPLE 20

### ((2R,4R)-1-Cyano-4-{[(2,5-dibromophenyl)sulfonyl]amino}-2-pyrrolidinyl)methyl (phenylmethyl)carbamate

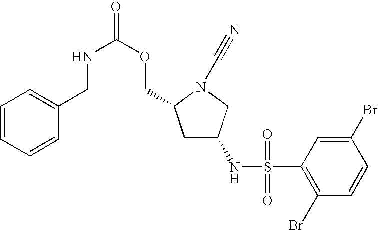

To 1,1-dimethylethyl (2R,4R)-4-{[(2,5-dibromophenyl)sulfonyl]amino}-2-[({[(phenylmethyl)amino]carbonyl}oxy)methyl]-1-pyrrolidinecarboxylate (Intermediate 45) (0.0723 g, 0.112 mmol) in dioxane (0.5 mL) was added 4 N HCl (0.70 mL). After stirring for 2 hours, the solvent was removed under vacuum to afford the crude secondary amine.

A mixture of the crude material previously obtained in DCM (2 mL) was neutralized by the addition of DIEA (0.072 g, 0.55 mmol, 4 eq). BrCN (0.139 mL, 0.42 mmol, 3 eq) was added and the resultant mixture was stirred at room temperature for 2 hours. PS-trisamine (4 eq) was added and the mixture was stirred overnight at room temperature then filtered, concentrated under vacuum and purified by preparatory HPLC (without TFA) to afford the title compound (0.0315 g). LC-MS: m/z, 573 (M+H). 1H NMR (400 MHz, CHLOROFORM-d) δ ppm 8.26 (d, J=2.26 Hz, 1H) 7.60-7.63 (m, 1H) 7.56-7.60 (m, 1H) 7.25-7.41 (m, 5H) 6.14 (d, J=6.27 Hz, 1H) 5.49 (t, J=5.65 Hz, 1H) 4.40 (dd, J=6.02, 2.26 Hz, 2H) 4.35 (dd, J=11.92, 3.64 Hz, 1H) 4.20 (dd, J=11.92, 5.40 Hz, 1H) 3.87-3.94 (m, 2H) 3.49 (dd, J=10.04, 6.02 Hz, 1H) 3.34 (dd, J=10.04, 4.77 Hz, 1H) 2.22-2.38 (m, 1H) 1.82 (ddd, J=13.36, 5.71, 5.52 Hz, 1H)

### EXAMPLE 21

### ((2R,4R)-1-Cyano-4-{[(2,5-dibromophenyl)sulfonyl]amino}-2-pyrrolidinyl)methyl {[2-(methyl oxy)phenyl]methyl}carbamate

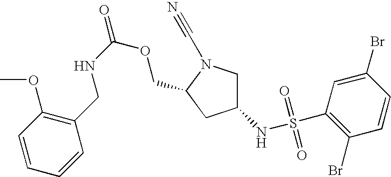

((2R,4R)-4-{[(2,5-Dibromophenyl)sulfonyl]amino}-2-pyrrolidinyl)methyl {[2-(methyloxy)phenyl]methyl}carbamate (0.0752 g, 0.13 mmol, 1 eq.) in DCM (2 mL) was neutralized by the addition of DIEA (0.072 g, 0.55 mmol). BrCN (0.139 mL, 0.41 mmol) was added and the resultant mixture was stirred at room temperature for 2 hours. PS-trisamine (4 eq.) was added and the mixture was stirred overnight at room temperature then filtered, concentrated under vacuum and purified by preparatory HPLC (without TFA) to afford the title compound (0.0315 g). LC-MS: m/z, 603 (M+H). 1H NMR (400 MHz, CHLOROFORM-d) δ ppm 8.26 (d, J=2.01 Hz, 1H) 7.59-7.63 (m, 1H) 7.54-7.59 (m, 1H) 7.25-7.32 (m, 2H) 6.94 (t, J=7.53 Hz, 1H) 6.89 (d, J=8.53 Hz, 1H) 6.20 (d, J=6.02 Hz, 1H) 5.60 (t, J=5.90 Hz, 1H) 4.38 (d, J=6.27 Hz, 2H) 4.31 (dd, J=12.05, 3.76 Hz, 1H) 4.16 (dd, J=12.05, 5.02 Hz, 1H) 3.80-3.92 (m, 5H) 3.47 (dd, J=10.16, 5.90 Hz, 1H) 3.32 (dd, J=10.04, 4.52 Hz, 1H) 2.28 (ddd, J=13.80, 8.41, 7.15 Hz, 1H) 1.75-1.83 (m, 1H)

### EXAMPLE 22

### ((2R,4R)-1-Cyano-4-{[(2,5-dibromophenyl)sulfonyl]amino}-2-pyrrolidinyl)methyl {[3-(methyloxy)phenyl]methyl}carbamate

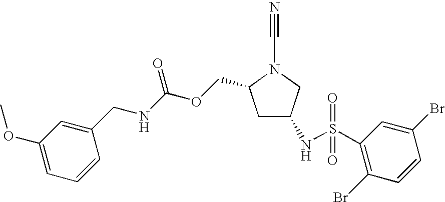

((2R,4R)-4-{[(2,5-dibromophenyl)sulfonyl]amino}-2-pyrrolidinyl)methyl {[3-(methyloxy)phenyl]methyl}carbamate (Intermediate 62) (0.08 g, 0.14 mmol, 1 eq.) in DCM (2 mL) was neutralized by the addition of DIEA (0.072 g, 0.55 mmol). BrCN (0.139 mL, 0.41 mmol) was added and the resultant mixture was stirred at room temperature for 2 hours. PS-trisamine (4 eq.) was added and the mixture was stirred overnight at room temperature then filtered, concentrated under vacuum and purified by preparatory HPLC (without TFA) to afford the title compound (0.0361 g). LC-MS: m/z, 603 (M+H). 1H NMR (400 MHz, CHLOROFORM-d) δ ppm 8.24 (d, J=2.26 Hz, 1H) 7.54-7.62 (m, 1H) 7.59 (t, J=8.78 Hz, 1H) 7.26 (br. s., 1H) 6.79-6.91 (m, 3H) 6.25-6.35 (m, 1H) 5.54-5.62 (m, 1H) 4.34 (d, J=6.02 Hz, 2H) 4.29 (d, J=3.26 Hz, 1H) 4.15-4.22 (m, 1H) 3.85-3.92 (m, 2H) 3.80 (s, 3H) 3.48 (dd, J=9.91, 6.40 Hz, 1H) 3.29-3.36 (m, 1H) 2.21-2.31 (m, 1H) 1.78 (br. s., 1H)

### EXAMPLE 23

### ((2R,4R)-1-Cyano-4-{[(2,5-dibromophenyl)sulfonyl]amino}-2-pyrrolidinyl)methyl {[4-(methyloxy)phenyl]methyl}carbamate

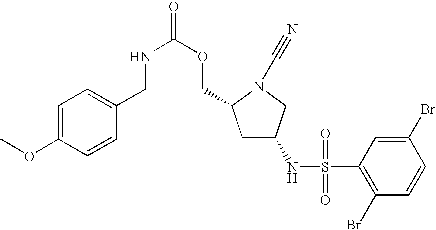

((2R,4R)-4-{[(2,5-dibromophenyl)sulfonyl]amino}-2-pyrrolidinyl)methyl {[4-(methyloxy)phenyl]methyl}carbamate (Intermediate 63) (0.0556 g, 0.096 mmol, 1 eq.) in DCM (2 mL) was neutralized by the addition DIEA (0.072 g, 0.55 mmol). BrCN (0.139 mL, 0.42 mmol) was added and the resultant mixture was stirred at room temperature for 2 hours. PS-trisamine (4 eq.) was added and the mixture was stirred overnight at room temperature then filtered, concentrated under vacuum and purified by preparatory HPLC (without TFA) to afford the title compound (0.0384 g). LC-MS: m/z, 603 (M+H). 1H NMR (400 MHz, CHLOROFORM-d) δ ppm 8.17 (d, J=2.26 Hz, 1H) 7.51-7.55 (m, 1H) 7.47-7.51 (m, 1H) 7.15 (m, J=8.53 Hz, 2H) 6.79 (m, J=8.53 Hz, 2H) 6.02 (br. s., 1H) 5.33 (t, J=5.52 Hz, 1H) 4.27 (d, J=3.51 Hz, 1H) 4.24 (d, J=2.01 Hz, 2H) 4.10 (dd, J=12.05, 5.27 Hz, 1H) 3.76-3.84 (m, 2H) 3.72 (s, 3H) 3.40 (dd, J=10.04, 6.02 Hz, 1H) 3.20-3.27 (m, 1H) 2.16-2.27 (m, 1H) 1.68-1.77 (m, 1H)

### EXAMPLE 24

### ((2R,4R)-1-Cyano-4-{[(2,5-dibromophenyl)sulfonyl]amino}-2-pyrrolidinyl)methyl [2-(methyloxy)phenyl]carbamate

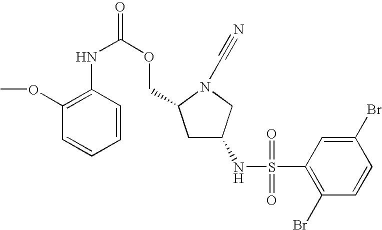

((2R,4R)-4-{[(2,5-Dibromophenyl)sulfonyl]amino}-2-pyrrolidinyl)methyl [2-(methyloxy)phenyl]carbamate (Intermediate 64) (0.0304 g, 0.054 mmol, 1 eq.) in DCM (2 mL) was neutralized by the addition DIEA (0.072 g, 0.55 mmol). BrCN (0.139 mL, 0.42 mmol) was added and the resultant mixture was stirred at room temperature for 2 hours. PS-trisamine (4 eq.) was added and the mixture was stirred overnight at room temperature then filtered, concentrated under vacuum and purified by preparatory HPLC (without TFA) to afford the title compound (0.0134 g). LC-MS: m/z, 589 (M+H). 1H NMR (400 MHz, CHLOROFORM-d) δ ppm 8.19 (d, J=2.26 Hz, 1H) 7.51-7.54 (m, 1H) 7.47-7.51 (m, 1H) 7.19 (s, 1H) 6.94-7.00 (m, 1H) 6.87-6.92 (m, 1H) 6.81 (dd, J=8.03, 1.25 Hz, 1H) 5.86 (br. s., 1H) 4.34 (dd, J=12.05, 4.02 Hz, 1H) 4.19-4.25 (m, 1H) 3.83-3.88 (m, 2H) 3.81 (s, 3H) 3.44 (dd, J=10.04, 6.02 Hz, 1H) 3.28 (dd, J=10.04, 4.77 Hz, 1H) 2.25 (ddd, J=13.68, 8.41, 7.03 Hz, 1H) 1.76 (ddd, J=13.61, 5.83, 5.65 Hz, 1H) 1.54 (s, 1H)

### EXAMPLE 25

### ((2R,4R)-1-Cyano-4-{[(2,5-dibromophenyl)sulfonyl]amino}-2-pyrrolidinyl)methyl [3-(methyloxy)phenyl]carbamate

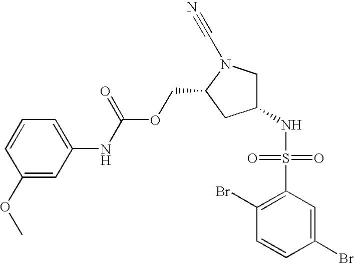

((2R,4R)-4-{[(2,5-Dibromophenyl)sulfonyl]amino}-2-pyrrolidinyl)methyl [3-(methyloxy)phenyl]carbamate (Intermediate 65) (0.0496 g, 0.88 mmol, 1 eq.) in DCM (2 mL) was neutralized by the addition of (0.072 g, 0.55 mmol). BrCN (0.139 mL, 0.41 mmol) was added and the resultant mixture was stirred at room temperature for 2 hours. PS-trisamine (4 eq.) was added and the mixture was stirred overnight at room temperature then filtered, concentrated under vacuum and purified by preparatory HPLC (without TFA) to afford the title compound (0.0397 g). LC-MS: m/z, 589 (M+H). 1H NMR (400 MHz, CHLOROFORM-d) δ ppm 8.19 (d, J=2.26 Hz, 1H) 7.51-7.54 (m, 1H) 7.46-7.50 (m, 1H) 7.19 (s, 1H) 7.13 (t, J=8.28 Hz, 1H) 7.03 (br. s., 1H) 6.85 (d, J=7.78 Hz, 1H) 6.56 (dd, J=8.28, 1.76 Hz, 1H) 6.00 (d, J=6.78 Hz, 1H) 4.37 (dd, J=12.05, 3.01 Hz, 1H) 4.16 (dd, J=12.05, 5.02 Hz, 1H) 3.81-3.89 (m, 2H) 3.72 (s, 3H) 3.44 (dd, J=10.29, 6.02 Hz, 1H) 3.29 (dd, J=10.04, 4.52 Hz, 1H) 2.20-2.30 (m, 1H) 1.83 (dt, J=13.55, 5.52 Hz, 1H)

### EXAMPLE 26

### ((2R,4R)-1-Cyano-4-{[(2,5-dibromophenyl)sulfonyl]amino}-2-pyrrolidinyl)methyl [4-(methyloxy)phenyl]carbamate

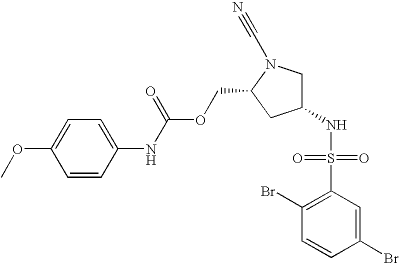

((2R,4R)-4-{[(2,5-Dibromophenyl)sulfonyl]amino}-2-pyrrolidinyl)methyl [4-(methyloxy)phenyl]carbamate (Intermediate 66) (0.0395 g, 0.070 mmol, 1 eq.) in DCM (2 mL) was neutralized by the addition of DIEA (0.072 g, 0.55 mmol). BrCN (0.139 mL, 0.42 mmol) was added and stirred at room temperature for 2 hours. PS-trisamine (4 eq.) was added and the resulting mixture was stirred overnight at room temperature then filtered, concentrated under vacuum and purified by preparatory HPLC (without TFA) to afford the title compound (0.0266 g). LC-MS: m/z, 589 (M+H). 1H NMR (400 MHz, CHLOROFORM-d) δ ppm 8.28 (d, J=2.26 Hz, 1H) 7.62-7.65 (m, 1H) 7.57-7.61 (m, 1H) 7.34 (m, J=8.53 Hz, 2H) 7.05 (br. s., 1H) 6.85-6.91 (m, 2H) 6.02 (br. s., 1H) 4.46 (dd, J=12.05, 3.26 Hz, 1H) 4.26 (dd, J=12.17, 4.89 Hz, 1H) 3.91-4.00 (m, 2H) 3.81 (s, 3H) 3.52 (dd, J=10.16, 5.65 Hz, 1H) 3.35-3.42 (m, 1H) 2.32-2.42 (m, 1H) 1.91-1.98 (m, 1H)

### EXAMPLE 27

### ((2R,4R)-1-Cyano-4-{[(2,5-dibromophenyl)sulfonyl]amino}-2-pyrrolidinyl)methyl [3-(trifluoromethyl)phenyl]carbamate

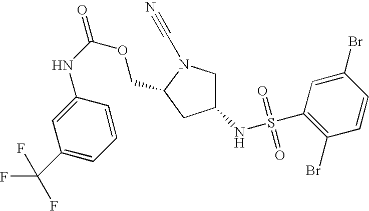

((2R,4R)-4-{[(2,5-Dibromophenyl)sulfonyl]amino}-2-pyrrolidinyl)methyl [3-(trifluoromethyl)phenyl]carbamate (Intermediate 68) (0.0746 g, 0.124 mmol, 1 eq.) in DCM (2 mL) was neutralized by the addition of DIEA (0.072 g, 0.55 mmol). BrCN (0.139 mL, 0.42 mmol) was added and the resulting mixture was stirred at room temperature for 2 hours. PS-trisamine (4 eq.) was added and the mixtures was stirred overnight at room temperature then filtered, concentrated under vacuum and purified by preparatory HPLC (without TFA) to afford the title compound (0.0462 g). LC-MS: m/z, 627 (M+H). 1H NMR (400 MHz, CHLOROFORM-d) δ ppm 8.26 (d, J=2.26 Hz, 1H) 7.76 (br. s., 1H) 7.70 (s, 1H) 7.58-7.61 (m, 1H) 7.54-7.58 (m, 2H) 7.40 (t, J=7.91 Hz, 1H) 7.27-7.34 (m, 1H) 6.27 (br. s., 1H) 4.45 (dd, J=12.05, 3.01 Hz, 1H) 4.26 (dd, J=11.92, 5.40 Hz, 1H) 3.89-4.00 (m, 2H) 3.55 (dd, J=10.04, 6.27 Hz, 1H) 3.39-3.46 (m, 1H) 2.28-2.38 (m, 1H) 1.87-1.96 (m, 1H)

### EXAMPLE 28

### ((2R,4R)-1-Cyano-4-{[(2,5-dibromophenyl)sulfonyl]amino}-2-pyrrolidinyl)methyl [4-(trifluoromethyl)phenyl]carbamate

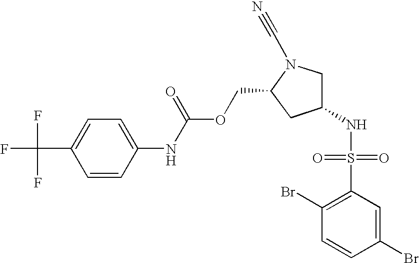

((2R,4R)-4-{[(2,5-Dibromophenyl)sulfonyl]amino}-2-pyrrolidinyl)methyl [4-(trifluoromethyl)phenyl]carbamate (Intermediate 69) (0.0829 g, 0.138 mmol, 1 eq.) in DCM (2 mL) was neutralized by the addition DIEA (0.102 g, 0.79 mmol). BrCN (0.198 mL, 0.59 mmol) was added and the reaction mixture was stirred at room temperature for 2 hours. PS-trisamine (4 eq.) was added and the mixture was stirred overnight at room temperature then filtered, concentrated under vacuum and purified by preparatory HPLC (without TFA) to afford the title compound (0.0570 g). LC-MS: m/z, 627 (M+H). 1H NMR (400 MHz, CHLOROFORM-d) δ ppm 8.27 (d, J=2.01 Hz, 1H) 7.67 (s, 1H) 7.61 (d, 1H) 7.59 (d, J=2.26 Hz, 1H) 7.55 (s, 4H) 6.16 (d, J=6.02 Hz, 1H) 4.48 (dd, J=12.05, 2.76 Hz, 1H) 4.27 (dd, J=12.05, 5.02 Hz, 1H) 3.93-4.00 (m, 2H) 3.56 (dd, J=10.16, 5.90 Hz, 1H) 3.43 (dd, J=10.16, 4.39 Hz, 1H) 2.37 (ddd, J=13.80, 8.78, 6.53 Hz, 1H) 1.98 (ddd, J=13.68, 5.40, 5.27 Hz, 1H)

### EXAMPLE 29

### ((2R,4R)-1-Cyano-4-{[(2,5-dibromophenyl)sulfonyl]amino}-2-pyrrolidinyl)methyl (2-methylphenyl)carbamate

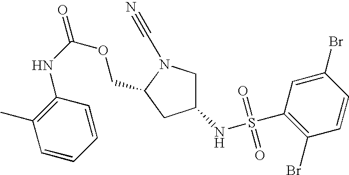

To 1,1-dimethylethyl (2R,4R)-4-{[(2,5-dibromophenyl)sulfonyl]amino}-2-[({[(2-methylphenyl)amino]carbonyl}oxy)methyl]-1-pyrrolidinecarboxylate (Intermediate 54) (0.0178 g, 0.027 mmol, 1 eq.) in dioxane (0.5 mL) was added 4 N HCl (0.9 mL). The resulting mixture was stirred for 2 hours then the solvent was removed under vacuum to a give a crude residue which was dissolved in DCM (2 mL) and neutralized by addition of DIEA (0.102 g, 0.79 mmol). BrCN (0.198 mL, 0.59 mmol) was added and the mixture was stirred at room temperature for 2 hours. PS-trisamine (4 eq.) was added and the resulting mixture was stirred overnight at room temperature then filtered, concentrated under vacuum and purified by preparatory HPLC (without TFA) to afford the title compound (0.0560 g). LC-MS: m/z, 573 (M+H). 1H NMR (400 MHz, CHLOROFORM-d) δ ppm 8.16 (d, J=2.01 Hz, 1H) 7.60 (br. s., 1H) 7.49-7.52 (m, 1H) 7.44-7.48 (m, 1H) 7.07-7.15 (m, 2H) 6.99 (t, J=7.15 Hz, 1H) 6.71 (s, 1H) 6.09 (br. s., 1H) 4.26-4.32 (m, 1H) 4.14-4.21 (m, 1H) 3.78-3.85 (m, 2H) 3.40 (dd, J=10.04, 6.27 Hz, 1H) 3.24 (br. s., 1H) 2.19 (s, 3H) 2.15-2.25 (m, 1H) 1.69-1.76 (m, 1H)

### EXAMPLE 30

### ((2R,4R)-1-Cyano-4-{[(2,5-dibromophenyl)sulfonyl]amino}-2-pyrrolidinyl)methyl (3-methylphenyl)carbamate

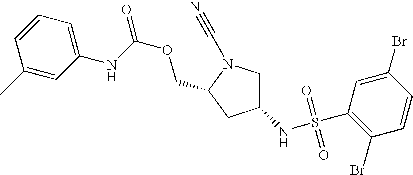

((2R,4R)-4-{[(2,5-Dibromophenyl)sulfonyl]amino}-2-pyrrolidinyl)methyl (3-methylphenyl)carbamate (Intermediate 70) (0.0697 g, 0.127 mmol, 1 eq.) in DCM (2 mL) was neutralized by the addition of DIEA (0.102 g, 0.79 mmol). BrCN (0.198 mL, 0.59 mmol) was added and stirred at room temperature for 2 hours. PS-trisamine (4 eq.) was added and the resulting mixture was stirred overnight at room temperature then filtered, concentrated under vacuum and purified by preparatory HPLC (without TFA) to afford the title compound (0.0622 g). LC-MS: m/z, 573 (M+H). 1H NMR (400 MHz, CHLOROFORM-d) δ ppm 8.12 (d, J=2.26 Hz, 1H) 7.44-7.48 (m, 1H) 7.40-7.43 (m, 1H) 7.10 (d, J=5.27 Hz, 2H) 7.04-7.07 (m, 2H) 6.76 (d, J=6.27 Hz, 1H) 6.07 (br. s., 1H) 4.28 (dd, J=12.05, 3.26 Hz, 1H) 4.10 (dd, J=11.92, 5.14 Hz, 1H) 3.74-3.82 (m, 2H) 3.38 (dd, J=10.04, 6.02 Hz, 1H) 3.24 (dd, J=10.04, 5.02 Hz, 1H) 2.19 (s, 3H) 2.15-2.21 (m, 1H) 1.70-1.79 (m, 1H)

### EXAMPLE 31

### ((2R,4R)-1-Cyano-4-{[(2,5-dibromophenyl)sulfonyl]amino}-2-pyrrolidinyl)methyl 1-naphthalenylcarbamate

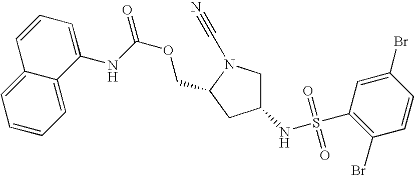

((2R,4R)-4-{[(2,5-dibromophenyl)sulfonyl]amino}-2-pyrrolidinyl)methyl naphthalenylcarbamate (Intermediate 71) (0.1157 g, 0.198 mmol, 1 eq.) in DCM (2 mL) was neutralized by the addition DIEA (0.102 g, 0.79 mmol). BrCN (0.198 mL, 0.59 mmol) was added and the mixture was stirred at room temperature for 2 hours. PS-trisamine (4 eq.) was added and the mixture was stirred overnight at room temperature then filtered, concentrated under vacuum and purified by preparatory HPLC (without TFA) to afford the title compound (0.0449 g). LC-MS: m/z, 609 (M+H). 1H NMR (400 MHz, CHLOROFORM-d) δ ppm 8.25 (d, J=1.76 Hz, 1H) 7.99 (d, J=7.78 Hz, 1H) 7.86-7.91 (m, 1H) 7.77-7.84 (m, 1H) 7.72 (d, J=8.03 Hz, 1H) 7.56 (d, J=4.27 Hz, 1H) 7.52-7.59 (m, 2H) 7.46-7.53 (m, 2H) 7.38 (br. s., 1H) 6.12 (br. s., 1H) 4.40-4.48 (m, 1H) 4.29 (br. s., 1H) 3.91 (br. s., 2H) 3.47 (br. s., 2H) 2.28 (br. s., 1H) 1.84 (br. s., 1H)

### EXAMPLE 32

### ((2R,4R)-1-Cyano-4-{[(2,5-dibromophenyl)sulfonyl]amino}-2-pyrrolidinyl)methyl 2-naphthalenylcarbamate

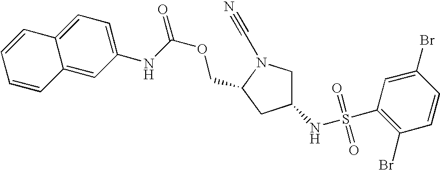

((2R,4R)-4-{[(2,5-dibromophenyl)sulfonyl]amino}-2-pyrrolidinyl)methyl 2-naphthalenylcarbamate (Intermediate 72) (0.0879 g, 0.150 mmol, 1 eq.) in DCM (2 mL) was neutralized by the addition of DIEA (0.102 g, 0.79 mmol). BrCN (0.198 mL, 0.59 mmol) was added and the mixture was stirred at room temperature for 2 hours. PS-trisamine (4 eq.) was added and the resulting mixture was stirred overnight at room temperature then filtered, concentrated under vacuum and purified by preparatory HPLC (without TFA) to afford the title compound (0.0409 g). LC-MS: m/z, 609 (M+H). 1H NMR (400 MHz, CHLOROFORM-d) δ ppm 8.13 (d, J=2.26 Hz, 1H) 7.86 (br. s., 1H) 7.60-7.66 (m, 3H) 7.40-7.45 (m, 1H) 7.36-7.40 (m, 1H) 7.23-7.35 (m, 4H) 6.00 (br. s., 1H) 4.33 (dd, J=12.05, 3.26 Hz, 1H) 4.13 (dd, J=12.05, 5.02 Hz, 1H) 3.74-3.83 (m, 2H) 3.37 (dd, J=10.04, 6.02 Hz, 1H) 3.25 (dd, J=10.04, 4.27 Hz, 1H) 2.17 (ddd, J=13.74, 8.60, 6.78 Hz, 1H) 1.78 (ddd, J=13.49, 5.58, 5.27 Hz, 1H)

### EXAMPLE 33

### ((2R,4R)-1-Cyano-4-{[(2,5-dibromophenyl)sulfonyl]amino}-2-pyrrolidinyl)methyl ethylcarbamate

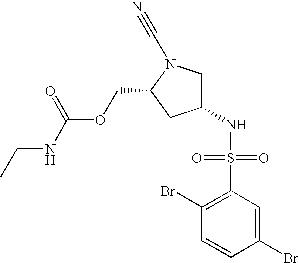

((2R,4R)-4-{[(2,5-Dibromophenyl)sulfonyl]amino}-2-pyrrolidinyl)methyl ethylcarbamate (Intermediate 73) (0.0871 g, 0.179 mmol, 1 eq.) in DCM (2 mL) was neutralized by the addition of DIEA (0.102 g, 0.79 mmol). BrCN (0.198 mL, 0.59 mmol) was added and the mixture was stirred at room temperature for 2 hours. PS-trisamine (4 eq.) was added and the mixture was stirred overnight at room temperature then filtered, concentrated under vacuum and purified by preparatory HPLC (without TFA) to afford the title compound (0.0430 g). LC-MS: m/z, 511 (M+H). 1H NMR (400 MHz, CHLOROFORM-d) δ ppm 8.27 (d, J=2.51 Hz, 1H) 7.61-7.66 (m, 1H) 7.56-7.61 (m, 1H) 6.26 (d, J=7.03 Hz, 1H) 5.10 (br. s., 1H) 4.28-4.35 (m, 1H) 4.17 (dd, J=12.05, 5.52 Hz, 1H) 3.86-3.95 (m, 2H) 3.50 (dd, J=10.16, 5.90 Hz, 1H) 3.36 (dd, J=10.04, 4.52 Hz, 1H) 3.21-3.30 (m, 2H) 2.32 (ddd, J=13.68, 8.66, 6.78 Hz, 1H) 1.83 (ddd, J=13.61, 5.46, 5.27 Hz, 1H) 1.17 (t, J=7.15 Hz, 3H)

### EXAMPLE 34

### ((2R,4R)-1-Cyano-4-{[(2,5-dibromophenyl)sulfonyl]amino}-2-pyrrolidinyl)methyl (1,1-dimethylethyl)carbamate

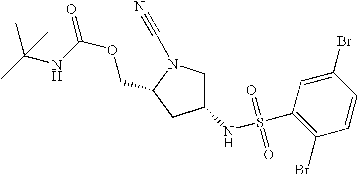

((2R,4R)-4-{[(2,5-Dibromophenyl)sulfonyl]amino}-2-pyrrolidinyl)methyl (1,1-dimethylethyl)carbamate (Intermediate 74) (0.253 g, 0.492 mmol, 1 eq.) in DCM (2 mL) was neutralized by the addition of DIEA (0.102 g, 0.79 mmol). BrCN (0.198 mL, 0.59 mmol) was added and the mixture was stirred at room temperature for 2 hours. PS-trisamine (4 eq.) was added and the mixtures was stirred overnight at room temperature then filtered, concentrated under vacuum and purified by preparatory HPLC (without TFA) to afford the title compound (0.0655 g). LC-MS: m/z, 539 (M+H). 1H NMR (400 MHz, CHLOROFORM-d) δ ppm 8.26 (d, J=2.26 Hz, 1H) 7.61-7.66 (m, 1H) 7.56-7.60 (m, 1H) 6.33 (br. s., 1H) 5.06 (s, 1H) 4.26 (dd, J=12.05, 3.51 Hz, 1H) 4.11 (dd, J=11.80, 4.02 Hz, 1H) 3.85-3.93 (m, 2H) 3.48 (dd, J=10.04, 5.77 Hz, 1H) 3.32-3.38 (m, 1H) 2.30 (ddd, J=13.87, 8.60, 6.90 Hz, 1H) 1.83 (dt, J=13.55, 5.27 Hz, 1H) 1.34 (s, 9H)

### EXAMPLE 35

### ((2R,4R)-1-Cyano-4-{[(2,5-dibromophenyl)sulfonyl]amino}-2-pyrrolidinyl)methyl [2-(trifluoromethyl)phenyl]carbamate

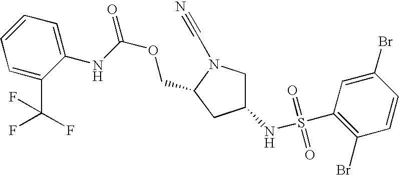

To ((2R,4R)-4-{[(2,5-Dibromophenyl)sulfonyl]amino}-2-pyrrolidinyl)methyl [2-(trifluoromethyl)phenyl]carbamate (Intermediate 67) (0.069 g, 0.114 mmol, 1 eq.) in DCM (2 mL) was neutralized by the addition of DIEA (0.072 g, 0.55 mmol). BrCN (0.139 mL, 0.41 mmol) was added and the mixture was stirred at room temperature for 2 hours. PS-trisamine (4 eq.) was added and the mixture was stirred overnight at room temperature then filtered, concentrated under vacuum and purified by preparatory HPLC (without TFA) to afford the title compound (0.0368 g). LC-MS: m/z, 627 (M+H). 1H NMR (400 MHz, CHLOROFORM-d) δ ppm 8.26 (d, J=2.26 Hz, 1H) 8.00 (d, J=8.03 Hz, 1H) 7.60-7.63 (m, 2H) 7.55-7.59 (m, 2H) 7.26 (t, J=7.65 Hz, 1H) 7.07 (br. s., 1H) 5.99 (br. s., 1H) 4.36-4.41 (m, 1H) 4.29-4.35 (m, 1H) 3.88-3.96 (m, 2H) 3.54 (dd, J=10.16, 6.65 Hz, 1H) 3.34 (dd, J=10.16, 6.15 Hz, 1H) 2.27-2.35 (m, 1H) 1.79 (ddd, J=13.55, 7.15, 6.90 Hz, 1H)

### EXAMPLE 36

### ((2R,4R)-1-Cyano-4-{[(2,5-dibromophenyl)sulfonyl]amino}-2-pyrrolidinyl)methyl cyclohexylcarbamate

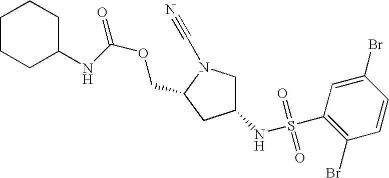

1,1-dimethylethyl (2S,4R)-4-{[(2,5-dibromo-3,6-difluorophenyl)sulfonyl]amino}-2-methyl-1-pyrrolidinecarboxylate (Intermediate 76) (0.084 g, 0.156 mmol, 1 eq.) in DCM (2 mL) was neutralized by the addition of DIEA (0.102 g, 0.79 mmol). BrCN (0.198 mL, 0.59 mmol) was added and the mixture was stirred at room temperature for 2 hours. PS-trisamine (4 eq.) was added and the mixture was stirred overnight at room temperature then filtered, concentrated under vacuum and purified by preparatory HPLC (without TFA) to afford the title compound (0.0378 g). LC-MS: m/z, 564 (M+H). 1H NMR (400 MHz, CHLOROFORM-d) δ ppm 8.19 (d, J=2.26 Hz, 1H) 7.53-7.57 (m, 1H) 7.48-7.52 (m, 1H) 6.23 (br. s., 1H) 4.94 (d, J=7.78 Hz, 1H) 4.23 (dd, J=12.05, 3.26 Hz, 1H) 4.07 (dd, J=12.05, 4.77 Hz, 1H) 3.78-3.87 (m, 2H) 3.37-3.45 (m, 2H) 3.27 (dd, J=10.04, 4.27 Hz, 1H) 2.24 (ddd, J=13.93, 8.66, 7.03 Hz, 1H) 1.82-1.90 (m, 2H) 1.77 (dt, J=13.55, 4.77 Hz, 1H) 1.61-1.68 (m, 2H) 1.49-1.56 (m, 1H) 1.21-1.33 (m, 2H) 1.08-1.15 (m, 3H)

### EXAMPLE 37

### 2,5-Dibromo-N-[(3R,5S)-1-cyano-5-methyl-3-pyrrolidinyl]-3,6-difluorobenzenesulfonamide

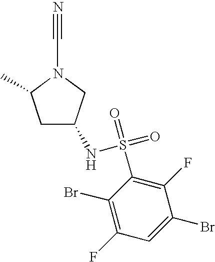

To a solution of 1,1-dimethylethyl (2S,4R)-4-{[(2,5-dibromo-3,6-difluorophenyl)sulfonyl]amino}-2-methyl-1-pyrrolidinecarboxylate (263 mg, 0.5 mmol) in dioxane (5 mL) was added 4 M HCl in dioxane (10 mL). The reaction mixture was stirred at room temperature for 1 hour followed by evaporation. The mixture was re-dissolved in DCM (10 mL). To the resultant mixture, DIEA (0.55 mL, 2 mmol) and CNBr solution (0.5 mL, 1.5 mmol) were added and stirred for an additional 1 hour at room temperature. PS-trisamine (1 g) was added and the mixture was stirred for 1 hour at room temperature. The resultant mixture was filtered, concentrated and purified on preparatory HPLC (without TFA) to afford the title compound as a brown oil (˜130 mg). LC-MS: m/z, 458 (M+H). 1H NMR (400 MHz, CHLOROFORM-d) δ ppm 1.39 (d, J=6.27 Hz, 3H) 1.51-1.57 (m, 1H) 2.37 (dt, J=12.86, 6.49 Hz, 1H) 3.35 (dd, J=9.91, 7.15 Hz, 1H) 3.62-3.69 (m, 1H) 3.64 (dd, J=9.79, 7.28 Hz, 1H) 4.06 (dd, J=8.78, 7.03 Hz, 1H) 5.19 (br. s., 1H) 7.64 (dd, J=6.78, 5.77 Hz, 1H)

### EXAMPLE 38

### 2,5-Dibromo-N-{(3R,5R)-1-cyano-5-[(1,3-dioxo-1,3-dihydro-2H-isoindol-2-yl)methyl]-3-pyrrolidinyl}benzenesulfonamide

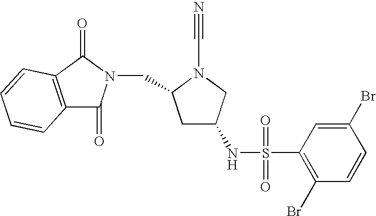

To a solution of 1,1-dimethylethyl (2R,4R)-4-{[(2,5-dibromophenyl)sulfonyl]amino}-2-[(1,3-dioxo-1,3-dihydro-2H-isoindol-2-yl)methyl]-1-pyrrolidinecarboxylate (50 mg, 0.08 mmol) in dioxane (5 mL) was added 4 M HCl in dioxane (10 mL). The reaction mixture was stirred at room temperature for 1 hour then evaporated and re-dissolved in DCM (10 mL). DIEA (70 μL, 0.4 mmol) and a 3 N BrCN solution in DCM (0.1 mL, 0.3 mmol) were added to the resultant mixture, which was then stirred for an additional 1 hour at room temperature. PS-trisamine (1 g) was added and the mixture was stirred for 1 hour at room temperature then filtered, concentrated and purified by preparatory HPLC (without TFA) to afford the title compound as a white solid (˜29 mg). LC-MS: m/z, 567 (M+H). 1H NMR (400 MHz, CHLOROFORM-d) δ ppm 1.85 (d, J=13.80 Hz, 1H) 2.30 (d, J=6.78 Hz, 1H) 3.46-3.51 (m, 1H) 3.52-3.57 (m, 1H) 3.91 (dd, J=10.16, 6.40 Hz, 2H) 4.04-4.10 (m, 2H) 6.15 (d, J=6.27 Hz, 1H) 7.58-7.62 (m, 1H) 7.63-7.66 (m, 1H) 7.78 (dd, J=5.40, 3.14 Hz, 2H) 7.90 (s, 1H) 7.92 (d, J=3.01 Hz, 1H) 8.29 (d, J=2.26 Hz, 1H)

### EXAMPLE 39

### N-[((2R,4R)-1-Cyano-4-{[(2,5-dibromophenyl)sulfonyl]amino}-2-pyrrolidinyl)methyl]benzamide

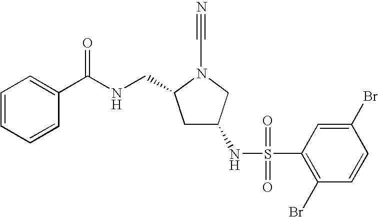

Example 39 was prepared using the general procedure described above for Example 37, replacing 1,1-dimethylethyl (2S,4R)-4-{[(2,5-dibromo-3,6-difluorophenyl)sulfonyl]amino}-2-methyl-1-pyrrolidinecarboxylate (263 mg, 0.5 mmol) with 1,1-dimethylethyl (2R,4R)-4-{[(2,5-dibromophenyl)sulfonyl]amino}-2-{[(phenylcarbonyl)amino]methyl}-1-pyrrolidinecarboxylate (62 mg, 0.1 mmol) to afford the title compound (˜31 mg). LC-MS: 541 m/z, (M+H). 1H NMR (400 MHz, CHLOROFORM-d) δ ppm 1.89 (d, J=13.55 Hz, 1H) 2.25 (d, J=7.03 Hz, 1H) 3.50-3.56 (m, 1H) 3.57-3.62 (m, 1H) 3.69 (dd, J=8.41, 6.40 Hz, 1H) 3.87 (dd, J=12.30, 5.52 Hz, 2H) 6.54 (d, J=5.77 Hz, 1H) 6.66 (br. s., 1H) 7.49 (t, J=7.40 Hz, 2H) 7.54-7.60 (m, 2H) 7.61-7.64 (m, 1H) 7.78-7.85 (m, 2H) 8.29 (d, J=2.01 Hz, 1H)

### EXAMPLE 40

### N-[((2R,4R)-1-Cyano-4-{[(2,5-dibromophenyl)sulfonyl]amino}-2-pyrrolidinyl)methyl]-2,2-dimethylpropanamide

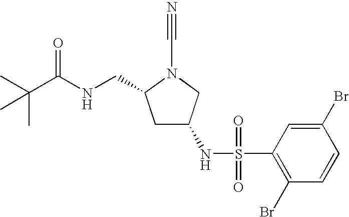

To a solution of 1,1-dimethylethyl (2R,4R)-4-{[(2,5-dibromophenyl)sulfonyl]amino}-2-{[(2,2-dimethylpropanoyl)amino]methyl}-1-pyrrolidinecarboxylate (119 mg, 0.2 mmol) in dioxane (5 mL) was added a 4 M HCl solution in dioxane (4 mL). The reaction mixture was stirred at room temperature for 1 hour then evaporated. The mixture was re-dissolved in DCM (10 mL). To the resultant mixture, DIEA (0.42 mL, 2.4 mmol) and a CNBr solution (0.6 mL, 1.8 mmol) were added and stirred for 1 hour at room temperature. PS-trisamine (1 g) was added and the mixture was stirred for 1 hour at room temperature. The resultant mixture was filtered, concentrated and purified by preparatory HPLC (without TFA) to afford the title compound (61 mg). 1H NMR (400 MHz, CHLOROFORM-d) δ ppm 8.19 (d, J=2.26 Hz, 1H), 7.52-7.56 (m, 1H), 7.49 (dd, J=8.28, 2.26 Hz, 1H), 6.59 (d, J=5.77 Hz, 1H), 6.01 (br. s., 1H), 3.65-3.72 (m, 1H), 3.65-3.72 (m, 1H), 3.57-3.64 (m, 1H), 3.45-3.51 (m, 1H), 3.38-3.43 (m, 1H), 3.28-3.35 (m, 1H), 2.10 (ddd, J=13.74, 7.15, 6.96 Hz, 1H), 1.65 (ddd, J=13.68, 5.14, 5.02 Hz, 1H), 1.15 (s, 9H)

### EXAMPLE 41

### N-[((2R,4R)-1-Cyano-4-{[(2,5-dibromophenyl)sulfonyl]amino}-2-pyrrolidinyl)methyl]-2-phenylacetamide

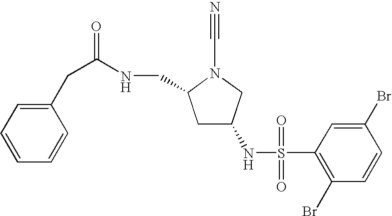

Example 41 was prepared using the general procedure described above for Example 37, replacing 1,1-dimethylethyl (2S,4R)-4-{[(2,5-dibromo-3,6-difluorophenyl)sulfonyl]amino}-2-methyl-1-pyrrolidinecarboxylate (263 mg, 0.5 mmol) with 1,1-dimethylethyl (2R,4R)-4-{[(2,5-dibromophenyl)sulfonyl]amino}-2-[(phenylacetyl)amino]methyl-1-pyrrolidinecarboxylate (126 mg, 0.2 mmol) to afford the title compound (74 mg). LC-MS: 555 m/z, (M+H). 1H NMR (400 MHz, CHLOROFORM-d) δ ppm 1.59 (d, J=13.55 Hz, 1H) 2.03 (d, J=6.53 Hz, 1H) 3.22 (dd, J=10.16, 4.89 Hz, 1H) 3.38-3.46 (m, 3H) 3.55-3.63 (m, 3H) 3.70 (d, J=5.77 Hz, 1H) 5.77 (br. s., 1H) 6.32 (d, J=6.02 Hz, 1H) 7.24 (d, J=1.51 Hz, 2H) 7.22 (s, 1H) 7.27 (d, J=7.03 Hz, 1H) 7.31 (d, J=7.28 Hz, 2H) 7.47-7.51 (m, 1H) 7.52-7.55 (m, 1H) 8.18 (d, J=2.26 Hz, 1H)

### EXAMPLE 42

### N-[((2R,4R)-1-Cyano-4-{[(2,5-dibromophenyl)sulfonyl]amino}-2-pyrrolidinyl)methyl]-3-methyl benzamide

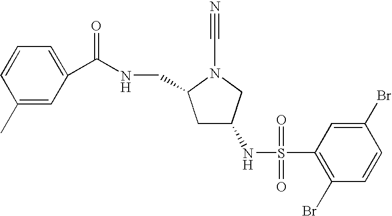

Example 42 was prepared using the general procedure described above for Example 37, replacing 1,1-dimethylethyl (2S,4R)-4-{[(2,5-dibromo-3,6-difluorophenyl)sulfonyl]amino}-2-methyl-1-pyrrolidinecarboxylate (263 mg, 0.5 mmol) with 1,1-dimethylethyl (2R,4R)-4-{[(2,5-dibromophenyl)sulfonyl]amino}-2-([(3-methylphenyl)carbonyl]aminomethyl)-1-pyrrolidinecarboxylate (126 mg, 0.2 mmol) to afford the title compound (65 mg). LC-MS: 555 m/z, (M+H). 1H NMR (400 MHz, CHLOROFORM-d) δ ppm 1.89 (d, J=13.80 Hz, 1H) 2.23 (s, 1H) 2.26 (d, J=6.78 Hz, 1H) 2.43 (s, 3H) 3.50-3.55 (m, 1H) 3.56-3.61 (m, 1H) 3.67 (s, 1H) 3.70 (dd, J=8.28, 1.76 Hz, 1H) 3.83-3.90 (m, 1H) 3.87 (dd, J=5.52, 4.27 Hz, 2H) 6.59 (d, J=5.77 Hz, 1H) 6.65 (s, 1H) 7.33-7.40 (m, 2H) 7.57-7.62 (m, 3H) 7.64 (s, 1H) 8.28 (d, J=2.01 Hz, 1H)

### EXAMPLE 43

### N-[((2R,4R)-1-Cyano-4-{[(2,5-dibromophenyl)sulfonyl]amino}-2-pyrrolidinyl)methyl]-2-(methyloxy)benzamide

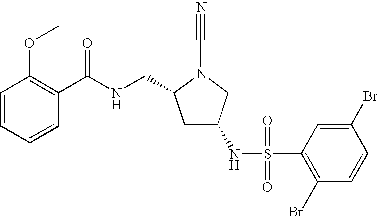

Example 43 was prepared using the general procedure described above for Example 37, replacing 1,1-dimethylethyl (2S,4R)-4-{[(2,5-dibromo-3,6-difluorophenyl)sulfonyl]amino}-2-methyl-1-pyrrolidinecarboxylate (263 mg, 0.5 mmol) with 1,1-dimethylethyl (2R,4R)-4-{[(2,5-dibromophenyl)sulfonyl]amino}-2-[({[2-(methyloxy)phenyl]carbonyl}amino)methyl]-1-pyrrolidinecarboxylate (129 mg, 0.2 mmol) to afford the title compound (82 mg). LC-MS: 571 m/z, (M+H). 1H NMR (400 MHz, CHLOROFORM-d) δ ppm 1.63 (s, 1H) 1.91 (d, J=13.80 Hz, 1H) 2.22 (d, J=7.03 Hz, 1H) 3.59 (d, J=5.02 Hz, 2H) 3.80 (d, J=1.76 Hz, 1H) 3.81-3.88 (m, 1H) 3.84 (t, J=5.52 Hz, 2H) 4.05 (s, 3H) 6.80 (d, J=6.02 Hz, 1H) 7.04 (d, J=8.53 Hz, 1H) 7.11 (dd, J=15.06, 1.00 Hz, 1H) 7.28 (s, 1H) 7.52 (ddd, J=8.60, 7.59, 2.13 Hz, 1H) 7.57 (d, J=6.53 Hz, 1H) 7.60 (s, 1H) 8.20 (dd, J=7.91, 1.88 Hz, 1H) 8.25 (d, J=2.26 Hz, 1H) 8.39 (br. s., 1H)

### EXAMPLE 44

### N-[((2R,4R)-1-Cyano-4-{[(2,5-dibromophenyl)sulfonyl]amino}-2-pyrrolidinyl)methyl]cyclohexanecarboxamide

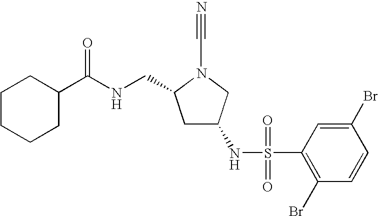

Example 44 was prepared using the general procedure described above for Example 37, replacing 1,1-dimethylethyl (2S,4R)-4-{[(2,5-dibromo-3,6-difluorophenyl)sulfonyl]amino}-2-methyl-1-pyrrolidinecarboxylate (263 mg, 0.5 mmol) with 1,1-dimethylethyl (2R,4R)-2-{[(cyclohexylcarbonyl)amino]methyl}-4-{[(2,5-dibromophenyl)sulfonyl]amino}-1-pyrrolidinecarboxylate (125 mg, 0.2 mmol) to afford the title compound (81 mg). LC-MS: 547 m/z, (M+H). 1H NMR (400 MHz, CHLOROFORM-d) δ ppm 1.25-1.30 (m, 2H) 1.32 (d, J=1.25 Hz, 1H) 1.43 (br. s., 1H) 1.46 (dd, J=9.29, 6.02 Hz, 2H) 1.73 (d, J=9.03 Hz, 1H) 1.78-1.85 (m, 3H) 1.90 (br. s., 2H) 2.18 (dd, J=13.05, 6.53 Hz, 1H) 2.15-2.20 (m, 1H) 3.43-3.50 (m, 2H) 3.56 (d, J=6.02 Hz, 1H) 3.58 (d, J=6.02 Hz, 1H) 3.66 (t, J=6.78 Hz, 1H) 3.71-3.77 (m, 1H) 3.81 (d, J=5.52 Hz, 1H) 5.88 (s, 1H) 6.64 (d, J=5.77 Hz, 1H) 7.57-7.60 (m, 1H) 7.62-7.65 (m, 1H) 8.28 (d, J=2.26 Hz, 1H)

### EXAMPLE 45

### N-[((2R,4R)-1-Cyano-4-{[(2,5-dibromophenyl)sulfonyl]amino}-2-pyrrolidinyl)methyl]-2-thiophenecarboxamide

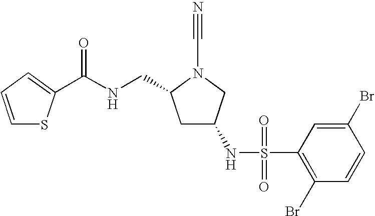

Example 45 was prepared using the general procedure described above for Example 37, replacing 1,1-dimethylethyl (2S,4R)-4-{[(2,5-dibromo-3,6-difluorophenyl)sulfonyl]amino}-2-methyl-1-pyrrolidinecarboxylate (263 mg, 0.5 mmol) with 1,1-dimethylethyl (2R,4R)-4-{[(2,5-dibromophenyl)sulfonyl]amino}-2-[(2-thienylcarbonyl)amino]methyl-1-pyrrolidinecarboxylate (125 mg, 0.2 mmol) to afford the title compound (67 mg). LC-MS: 547 m/z, (M+H). 1H NMR (400 MHz, CHLOROFORM-d) δ ppm 1.89 (d, J=13.80 Hz, 1H) 2.24 (d, J=7.03 Hz, 1H) 3.57 (dd, J=9.79, 5.52 Hz, 2H) 3.70 (d, J=6.53 Hz, 1H) 3.81-3.88 (m, 3H) 6.50 (d, J=5.77 Hz, 1H) 6.61 (s, 1H) 7.12 (dd, J=5.02, 3.76 Hz, 1H) 7.54-7.60 (m, 3H) 7.62 (s, 1H) 7.64 (s, 1H) 8.27 (d, J=2.26 Hz, 1H)

### EXAMPLE 46

### 2,5-Dibromo-N-[(3R,5R)-1-cyano-5-({[(phenylamino)carbonyl]amino}methyl)-3-pyrrolidinyl]benzenesulfonamide

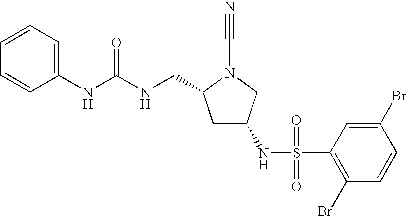

To 1,1-dimethylethyl (2R,4R)-2-(aminomethyl)-4-{[(2,5-dibromophenyl)sulfonyl]amino}-1-pyrrolidinecarboxylate (70 mg, 0.14 mmol) in DCM (3 mL) was added phenyl isocyanate (18 mg, 0.15 mmol). The reaction mixture was stirred at room temperature over the weekend then concentrated under vacuum to afford the crude 1,1-dimethylethyl (2R,4R)-4-[(2,5-dibromophenyl)sulfonyl]amino-2-({[(phenylamino)carbonyl]amino}methyl)-1-pyrrolidinecarboxylate. Following the general procedure described above in Example 37, replacing 1,1-dimethylethyl (2S,4R)-4-{[(2,5-dibromo-3,6-difluorophenyl)sulfonyl]amino}-2-methyl-1-pyrrolidinecarboxylate (263 mg, 0.5 mmol) with the crude 1,1-dimethylethyl (2R,4R)-4-{[(2,5-dibromophenyl)sulfonyl]amino}-2-({[(phenylamino)carbonyl]amino}methyl)-1-pyrrolidinecarboxylate previously obtained to afford the title compound (˜73 mg). LC-MS: 556 m/z, (M+H). 1H NMR (400 MHz, CHLOROFORM-d) δ ppm 1.92 (s, 1H) 3.50 (d, J=4.77 Hz, 1H) 3.51-3.58 (m, 1H) 3.54 (dd, J=6.15, 4.14 Hz, 2H) 3.77 (dt, J=5.52, 2.76 Hz, 2H) 5.62 (s, 1H) 6.77 (d, J=6.27 Hz, 1H) 7.12 (d, J=7.03 Hz, 1H) 7.20 (s, 1H) 7.28 (s, 2H) 7.31-7.37 (m, 4H) 7.53-7.61 (m, 2H) 8.26 (d, J=2.01 Hz, 1H)

### EXAMPLE 47

### 5-Chloro-N-[(3R,5S)-1-cyano-5-methyl-3-pyrrolidinyl]-2-(methyloxy)benzenesulfonamide

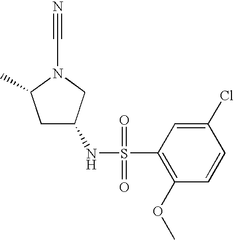

To 1,1-dimethylethyl (2S,4R)-4-({[5-chloro-2-(methyloxy)phenyl]sulfonyl}amino)-2-methyl-1-pyrrolidinecarboxylate (93.7 mg, 0.23 mmol) in DCM (20 mL) was added 4N HCl in dioxane (3 mL). The reaction mixture was stirred at room temperature for 3 hours and concentrated under vacuum to give a crude residue. To the crude material previously obtained in DCM (20 mL) was added DIEA (0.2 mL) then BrCN (0.16 mL) was added after checking the pH was basic. The resultant mixture was stirred overnight at room temperature. PS trisamine (4 eq) was added and the mixture was stirred for an additional 2 hours at room temperature, filtered, treated with water and DCM then purified by preparatory HPLC (without TFA) to afford the title compound (29.8 mg). LC-MS: m/z, 331 (M+H). 1H NMR (400 MHz, CHLOROFORM-d) δ ppm 7.89 (1H, d, J=2.51 Hz) 7.55 (1H, dd, J=8.91, 2.64 Hz) 7.03 (1H, d, J=8.78 Hz) 5.45 (1H, d, J=7.78 Hz) 4.01 (3H, s) 3.88 (1H, ddd, J=14.93, 7.65, 7.53 Hz) 3.60 (1H, dq, J=8.97, 6.38 Hz) 3.44 (1H, dd, J=9.79, 7.28 Hz) 3.18 (1H, dd, J=9.79, 7.03 Hz) 2.23 (1H, dd, J=12.92, 6.40 Hz) 1.44 (1H, dt, J=12.80, 9.03 Hz) 1.34 (3H, d, J=6.27 Hz)

### EXAMPLE 48

### N-[(3R,5S)-1-Cyano-5-methyl-3-pyrrolidinyl]-2,5-dimethylbenzenesulfonamide

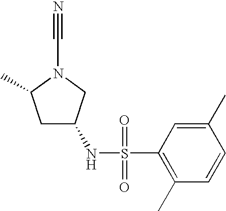

To 1,1-dimethylethyl (2S,4R)-4-{[(2,5-dimethylphenyl)sulfonyl]amino}-2-methyl-1-pyrrolidinecarboxylate (110.7 mg, 0.30 mmol, 1 eq.) in DCM (20 mL) was added slowly 4 N HCl in dioxane (3 mL). The reaction mixture was stirred at room temperature for 3 hours and concentrated under vacuum to give a crude residue. To the crude material previously obtained in DCM (20 mL) was added DIEA (0.21 mL). Then BrCN (0.16 mL) was added after checking that the pH was basic. The mixture was stirred overnight at room temperature then P-S trisamine (4 eq.) was added. After stirring for an additional 2 hours at room temperature, the mixture was filtered, extracted with water and DCM and purified on preparatory HPLC (without TFA) to afford the title compound (26.4 mg). LC-MS: m/z, 294 (M+H). 1H NMR (400 MHz, CHLOROFORM-d) δ ppm 7.80 (1H, s) 7.28-7.35 (1H, m) 7.22-7.27 (1H, m) 5.29 (1H, d, J=6.02 Hz) 3.79-3.86 (1H, m) 3.60 (1H, dq, J=9.32, 6.26 Hz) 3.46 (1H, dd, J=9.79, 7.53 Hz) 3.17 (1H, dd, J=9.79, 7.53 Hz) 2.59 (3H, s) 2.41 (3H, s) 2.26 (1H, ddd, J=12.67, 6.53, 6.40 Hz) 1.44 (1H, dt, J=12.80, 9.29 Hz) 1.34 (3H, d, J=6.02 Hz)

### EXAMPLE 49

### N-[(3R,5S)-1-Cyano-5-methyl-3-pyrrolidinyl]-3-(trifluoromethyl)benzenesulfonamide

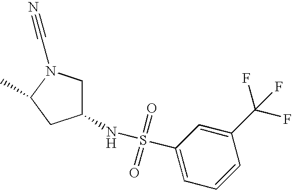

To 1,1-dimethylethyl (2S,4R)-2-methyl-4-({[3-(trifluoromethyl)phenyl]sulfonyl}amino)-1-pyrrolidinecarboxylate (104 mg, 0.25 mmol) in DCM (20 mL) was added slowly 4N HCl in dioxane (3 mL). The reaction mixture was stirred at room temperature for 3 hours and concentrated under vacuum to give a crude residue. To the crude material previously obtained in DCM (20 mL) was added DIEA (0.18 mL) then BrCN (0.17 mL) was added after checking that the pH was basic. The mixture was stirred overnight at room temperature then PS-trisamine (4 eq.) was added. After stirring for an additional 2 hours at room temperature, the mixture was filtered, extracted with water and DCM and purified on preparatory HPLC (without TFA) to afford the title compound (24.2 mg). LC-MS: m/z, 334 (M+H). 1H NMR (400 MHz, CHLOROFORM-d) δ ppm 8.16 (1H, s) 8.09 (1H, d, J=8.03 Hz) 7.90 (1H, d, J=7.78 Hz) 7.73 (1H, t, J=7.91 Hz) 5.57 (1H, d, J=7.78 Hz) 3.86-3.93 (1H, m) 3.64 (1H, dt, J=9.03, 6.40 Hz) 3.55 (1H, dd, J=9.79, 7.28 Hz) 3.25 (1H, dd, J=9.79, 7.03 Hz) 2.29 (1H, dt, J=12.86, 6.49 Hz) 1.47 (1H, dt, J=12.80, 8.91 Hz) 1.36 (3H, d, J=6.27 Hz)

### EXAMPLE 50

### 2,5-Dibromo-N-[(3R,5S)-1-cyano-5-methyl-3-pyrrolidinyl]-4-fluorobenzenesulfonamide

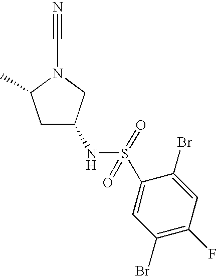

To a solution of 2,5-dibromo-4-fluoro-N-[(3R,5S)-5-methyl-3-pyrrolidinyl]benzenesulfonamide (0.625 mmol, 1 eq.) and triethylamine (210 μL, 1.87 mmol) in DCM (5 mL) was added BrCN (416 μL, 1.25 mmol, 2 eq.). The reaction mixture was stirred for 50 hours at room temperature. PS-trisamine resin (1.36 g, 2.5 mmol, 4 eq.) was added and the resulting mixture was stirred for one hour at room temperature, then filtered and evaporated to give crude material that was purified by preparatory HPLC to afford the title compound (45 mg). LC-MS: m/z, 441.9 (M+H). 1H NMR (400 MHz, CHLOROFORM-d) δ ppm 8.38 (1H, d, J=7.03 Hz) 7.56 (1H, d, J=7.28 Hz) 5.45 (1H, d, J=7.78 Hz) 3.89 (1H, dd, J=15.94, 7.91 Hz) 3.59-3.69 (1H, m, J=6.27, 3.51, 3.14, 3.14 Hz) 3.54 (1H, dd, J=9.79, 7.28 Hz) 3.29 (1H, dd, J=10.04, 7.03 Hz) 2.30 (1H, dt, J=12.86, 6.49 Hz) 1.50 (1H, dt, J=12.80, 9.03 Hz) 1.39 (3H, d, J=6.27 Hz)

### EXAMPLE 51

### N-[((2R,4R)-1-Cyano-4-{[(2,5-dichlorophenyl)sulfonyl]amino}-2-pyrrolidinyl)methyl]benzamide

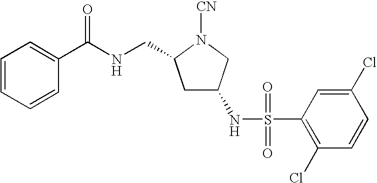

To 1,1-dimethylethyl (2R,4R)-2-(aminomethyl)-4-{[(2,5-dichlorophenyl)sulfonyl]amino}-1-pyrrolidinecarboxylate (100 mg, 0.24 mmol) in DCM (3 mL) was added TEA (48 mg, 0.48 mmol) and benzoyl chloride (36 mg, 0.25 mmol) at room temperature while stirring. The reaction mixture was stirred at room temperature for 1 hour, washed with 1 N HCl and the DCM layer was isolated and evaporated to give a crude oil that was treated with 4 N HCl in dioxane. The reaction mixture was stirred at room temperature for 1 hour and the solvent was evaporated. The mixture was re-dissolved in DCM (10 mL). To the resultant solution, DIEA (0.26 mL, 0.96 mmol) and CNBr solution (0.24 mL, 0.72 mmol) were added and stirred for an additional 1 hour at room temperature. PS-trisamine (1 g) was added and the mixture was stirred for 1 hour at room temperature. The resultant mixture was filtered, concentrated and purified on preparatory HPLC (without TFA) to afford the title compound. (91 mg, 83%). LC-MS: m/z, 455 (M+H), rt 1.90 min. 1H NMR (400 MHz, CHLOROFORM-d) δ ppm 1.89 (d, J=13.80 Hz, 1H) 2.22 (d, J=6.78 Hz, 1H) 3.56 (t, J=5.90 Hz, 2H) 3.66 (s, 1H) 3.67 (d, J=6.27 Hz, 1H) 3.87 (br. s., 1H) 3.85 (d, J=6.27 Hz, 2H) 3.88 (d, J=1.76 Hz, 1H) 6.88 (s, 1H) 7.43-7.48 (m, 2H) 7.50-7.57 (m, 3H) 7.81 (d, J=7.03 Hz, 1H) 7.80 (s, 1H) 8.10 (d, J=2.26 Hz, 1H)

### EXAMPLE 52

### N-[((2R,4R)-1-Cyano-4-{[(2,5-dichlorophenyl)sulfonyl]amino}-2-pyrrolidinyl)methyl]-3-methylbenzamide

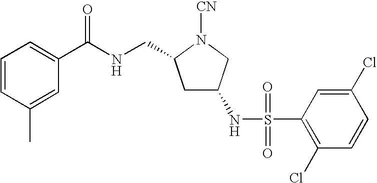

To 1,1-dimethylethyl (2R,4R)-2-(aminomethyl)-4-{[(2,5-dichlorophenyl)sulfonyl]amino}-1-pyrrolidinecarboxylate (100 mg, 0.24 mmol) in DCM (3 mL) was added TEA (48 mg, 0.48 mmol) and 3-methylbenzoyl chloride (39 mg, 0.25 mmol) at room temperature while stirring. The reaction mixture was stirred at room temperature for 1 hour, washed with 1 N HCl and the DCM layer was isolated and evaporated to give a crude oil that was treated with 4 N HCl in dioxane. The reaction mixture was stirred at room temperature for 1 hour after which the solvent was evaporated. The mixture was re-dissolved in DCM (10 mL). To the resultant mixture, DIEA (0.26 mL, 0.96 mmol) and CNBr solution (0.24 mL, 0.72 mmol) were added and stirred for an additional 1 hour at room temperature. PS-trisamine (1 g) was added and the mixture was stirred for 1 hour at room temperature. The resultant mixture was filtered, concentrated and purified on preparatory HPLC (without TFA) to afford the title compound. (47 mg, 42%). LC-MS: m/z, 469 (M+H), rt 2.01 min. 1H NMR (400 MHz, CHLOROFORM-d) δ ppm 1.66 (d, J=13.55 Hz, 1H) 2.00 (d, J=6.78 Hz, 1H) 2.21 (s, 3H) 3.35 (dd, J=11.92, 5.14 Hz, 1H) 3.45 (dd, J=8.53, 6.53 Hz, 1H) 3.62-3.69 (m, 3H) 6.40 (br. s., 1H) 6.46 (d, J=5.77 Hz, 1H) 7.09-7.17 (m, 2H) 7.25-7.32 (m, 2H) 7.36 (dd, J=4.89, 3.14 Hz, 1H) 7.41 (s, 1H) 7.88 (d, J=2.26 Hz, 1H)

### EXAMPLE 53

### N-[((2R,4R)-1-Cyano-4-{[(2,5-dichlorophenyl)sulfonyl]amino}-2-pyrrolidinyl)methyl]-2,2-dimethylpropanamide

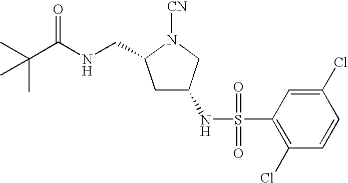

To 1,1-dimethylethyl (2R,4R)-2-(aminomethyl)-4-{[(2,5-dichlorophenyl)sulfonyl]amino}-1-pyrrolidinecarboxylate (100 mg, 0.24 mmol) in DCM (3 mL) was added TEA (48 mg, 0.48 mmol) and trimethylacetyl chloride (31 mg, 0.25 mmol) at room temperature while stirring. The reaction mixture was stirred at room temperature for 1 hour, washed with 1 N HCl and the DCM layer was isolated and evaporated to give a crude oil that was treated with 4 N HCl in dioxane. The reaction mixture was stirred at room temperature for 1 hour and the solvent evaporated. The mixture was re-dissolved in DCM (10 mL). To the resultant mixture, DIEA (0.26 mL, 0.96 mmol) and CNBr solution (0.24 mL, 0.72 mmol) were added and stirred for an additional 1 hour at room temperature. PS-trisamine (1 g) was added and the mixture was stirred for 1 hour at room temperature. The resultant mixture was filtered, concentrated and purified on preparatory HPLC (without TFA) to afford the title compound. (46 mg, 44%). LC-MS: m/z, 435 (M+H), rt 1.92 min. 1H NMR (400 MHz, CHLOROFORM-d) δ ppm 1.15 (s, 10H) 1.65 (d, J=13.55 Hz, 1H) 2.07 (d, J=6.78 Hz, 1H) 2.10 (s, 1H) 3.28-3.35 (m, 1H) 3.44 (d, J=4.02 Hz, 1H) 3.46-3.51 (m, 1H) 3.61 (d, J=7.03 Hz, 1H) 3.64-3.69 (m, 1H) 3.65 (t, J=6.40 Hz, 1H) 3.72 (d, J=5.77 Hz, 1H) 6.03 (br. s., 1H) 6.73 (d, J=5.77 Hz, 1H) 7.39-7.46 (m, 2H) 8.02 (d, J=2.26 Hz, 1H)

### EXAMPLE 54

### N-[((2R,4R)-1-Cyano-4-{[(2,5-dibromophenyl)sulfonyl]amino}-2-pyrrolidinyl)methyl]-3-fluorobenzamide

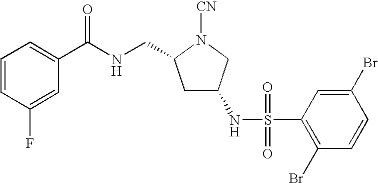

To 1,1-dimethylethyl (2R,4R)-2-(aminomethyl)-4-{[(2,5-dibromophenyl)sulfonyl]amino}-1-pyrrolidinecarboxylate (100 mg, 0.24 mmol) in DCM (3 mL) was added TEA (40 mg, 0.4 mmol) and 3-fluorobenzoyl chloride (32 mg, 0.20 mmol) at room temperature while stirring. The reaction mixture was stirred at room temperature for 1 hour, washed with 1 N HCl and the DCM layer was isolated and evaporated to give a crude oil that was treated with 4 N HCl in dioxane. The reaction mixture was stirred at room temperature for 1 hour followed by evaporation. The mixture was re-dissolved in DCM (10 mL). To the resultant mixture, DIEA (0.16 mL, 0.80 mmol) and CNBr solution (0.20 mL, 0.60 mmol) were added and stirred for an additional 1 hour at room temperature. PS-trisamine (1 g) was added and the mixture was stirred for 1 hour at room temperature. The resultant mixture was filtered, concentrated and purified on preparatory HPLC (without TFA) to afford the title compound. (61 mg, 57%). LC-MS: m/z, 561 (M+H), rt 2.00 min. 1H NMR (400 MHz, CHLOROFORM-d) δ ppm 1.87 (dt, J=13.55, 5.52 Hz, 1H) 2.26 (dt, J=13.87, 7.00 Hz, 1H) 3.50-3.55 (m, 1H) 3.56-3.62 (m, 1H) 3.68 (dd, J=8.03, 6.27 Hz, 1H) 3.82-3.89 (m, 1H) 3.86 (dd, J=13.30, 6.02 Hz, 2H) 6.56 (d, J=5.77 Hz, 1H) 6.88 (t, J=5.90 Hz, 1H) 7.44 (td, J=8.03, 5.52 Hz, 1H) 7.55-7.60 (m, 2H) 7.62 (s, 1H) 7.64 (s, 1H) 8.28 (d, J=2.26 Hz, 1H)

### EXAMPLE 55

### N-[((2R,4R)-1-Cyano-4-{[(2,5-dibromophenyl)sulfonyl]amino}-2-pyrrolidinyl)methyl]-4-fluorobenzamide

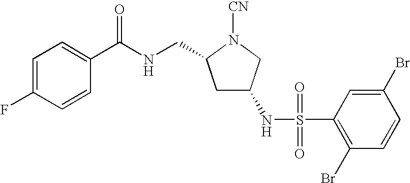

To 1,1-dimethylethyl (2R,4R)-2-(aminomethyl)-4-{[(2,5-dibromophenyl)sulfonyl]amino}-1-pyrrolidinecarboxylate (100 mg, 0.24 mmol) in DCM (3 mL) was added TEA (40 mg, 0.4 mmol) and 4-fluorobenzoyl chloride (32 mg, 0.20 mmol) at room temperature while stirring. The reaction mixture was stirred at room temperature for 1 hour, washed with 1 N HCl and the DCM layer was isolated and evaporated to give a crude oil that was treated with 4 N HCl in dioxane. The reaction mixture was stirred at room temperature for 1 hour followed by evaporation. The mixture was re-dissolved in DCM (10 mL). To the resultant mixture, DIEA (0.16 mL, 0.80 mmol) and CNBr solution (0.20 mL, 0.60 mmol) were added and stirred for an additional 1 hour at room temperature. PS-trisamine (1 g) was added and the mixture was stirred for 1 hour at room temperature. The resultant mixture was filtered, concentrated and purified on preparatory HPLC (without TFA) to afford the title compound. (˜70 mg, 66%). LC-MS: m/z, 561 (M+H), rt 1.99 min. 1H NMR (400 MHz, CHLOROFORM-d) δ ppm 1.88 (d, J=13.55 Hz, 1H) 2.25 (d, J=6.78 Hz, 1H) 3.51-3.55 (m, 1H) 3.56-3.61 (m, 1H) 3.67 (dd, J=8.03, 6.27 Hz, 1H) 3.83-3.90 (m, 3H) 6.60 (br. s., 1H) 6.75-6.82 (m, 1H) 7.10-7.17 (m, 2H) 7.61 (q, J=8.45 Hz, 1H) 7.57-7.65 (m, 1H) 7.83 (dd, J=8.78, 5.27 Hz, 2H) 7.80-7.87 (m, 1H) 8.28 (d, J=2.26 Hz, 1H)

### EXAMPLE 56

### 2,5-Dibromo-N-{(3R,5R)-1-cyano-5-[({[2-(methyloxy)phenyl)amino]carbonyl}amino)methyl]-3-pyrrolidinyl}benzene sulfonamide

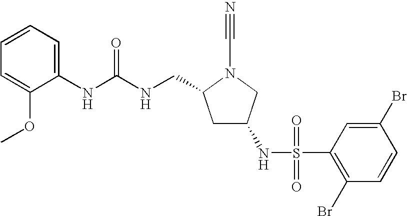

To 1,1-dimethylethyl (2R,4R)-2-(aminomethyl)-4-{[(2,5-dibromophenyl)sulfonyl]amino}-1-pyrrolidinecarboxylate (100 mg, 0.19 mmol) in DCM (3 mL) was added 1-isocyanato-2-(methyloxy)benzene (30 mg, 0.20 mmol). 3-fluorophenyl isocyanate (30 mg, 0.20 mmol). The reaction mixture was stirred at room temperature over the weekend then concentrated under vacuum to afford the crude, which was treated with 4N HCl in dioxane. The reaction mixture was stirred at room temperature for 1 hour followed by evaporation. The mixture was re-dissolved in DCM (10 mL). To the resultant mixture, DIEA (0.16 mL, 0.80 mmol) and CNBr solution (0.20 mL, 0.60 mmol) were added and stirred for an additional 1 hour at room temperature. PS-trisamine (1 g) was added and the mixture was stirred for 1 hour at room temperature. The resultant mixture was filtered, concentrated and purified on preparatory HPLC (without TFA) to afford the title compound. (74 mg, 66%). LC-MS: m/z, 588 (M+H), rt 2.02 min. 1H NMR (400 MHz, CHLOROFORM-d) δ ppm 1.80-1.88 (m, 1H) 2.10-2.18 (m, 1H) 3.40 (dd, J=10.04, 5.52 Hz, 1H) 3.47-3.54 (m, 3H) 3.72-3.79 (m, 1H) 3.78 (s, 4H) 5.85 (br. s., 1H) 6.61 (dd, J=8.16, 1.88 Hz, 1H) 6.84 (dd, J=8.03, 1.25 Hz, 1H) 7.06 (t, J=2.26 Hz, 1H) 7.18 (t, J=8.16 Hz, 1H) 7.49 (s, 1H) 7.52-7.59 (m, 2H) 8.25 (d, J=2.01 Hz, 1H)

### EXAMPLE 57

### 2,5-Dibromo-N-{(3R,5R)-1-cyano-5-[({[3-(methyloxy)phenyl)amino]carbonyl}amino)methyl]-3-pyrrolidinyl}benzene sulfonamide

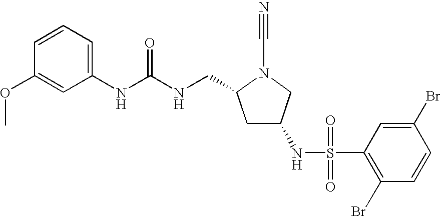

2,5-Dibromo-N-{(3R,5R)-1-cyano-5-[({[3-(methyloxy)phenyl)amino]carbonyl}amino)methyl]-3-pyrrolidinyl}benzene sulfonamide (82 mg, 73%) was prepared according to the general procedure of Example 56, substituting 1-isocyanato-3-(methyloxy)benzene (30 mg, 0.20 mmol) for 1-isocyanato-2-(methyloxy)benzene (30 mg, 0.20 mmol). LC-MS: 588 m/z, (M+H), rt 1.94 min. 1H NMR (400 MHz, CHLOROFORM-d) δ ppm 1.83 (t, J=12.92 Hz, 1H) 2.10-2.17 (m, 1H) 3.38-3.42 (m, 1H) 3.44-3.51 (m, 1H) 3.47 (dd, J=9.66, 3.64 Hz, 2H) 3.69 (d, J=6.78 Hz, 1H) 3.76 (s, 4H) 5.72 (br. s., 1H) 6.81 (m, 2H) 7.21 (m, 2H) 7.30 (d, J=13.30 Hz, 1H) 7.51-7.59 (m, 2H) 8.23 (d, J=2.01 Hz, 1H)

### EXAMPLE 58

### 2,5-Dibromo-N-{(3R,5R)-1-cyano-5-[({[4-(methyloxy)phenyl)amino]carbonyl}amino)methyl]-3-pyrrolidinyl}benzene sulfonamide

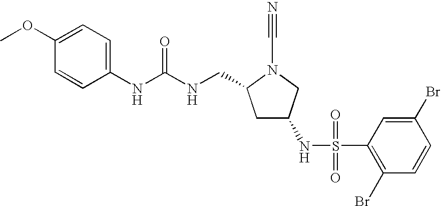

2,5-Dibromo-N-{(3R,5R)-1-cyano-5-[({[4-(methyloxy)phenyl)amino]carbonyl}amino)methyl]-3-pyrrolidinyl}benzene sulfonamide (112 mg, 100%) was prepared according to the general procedure of Example 56, substituting 1-isocyanato-4-(methyloxy)benzene (30 mg, 0.20 mmol) for 1-isocyanato-2-(methyloxy)benzene (30 mg, 0.20 mmol). LC-MS: 588 m/z, (M+H), rt 2.04 min. 1H NMR (400 MHz, CHLOROFORM-d) δ ppm 1.85 (dt, J=13.49, 6.68 Hz, 1H) 2.19 (ddd, J=13.80, 7.28, 7.03 Hz, 1H) 3.43 (dd, J=10.04, 5.52 Hz, 1H) 3.50-3.56 (m, 3H) 3.76 (t, J=6.15 Hz, 1H) 3.82 (t, J=6.27 Hz, 1H) 6.11 (br. s., 1H) 7.08 (br. s., 1H) 7.21 (t, J=7.65 Hz, 1H) 7.52-7.57 (m, 2H) 7.58-7.61 (m, 2H) 7.91 (d, J=8.28 Hz, 1H) 8.25 (d, J=2.01 Hz, 1H)

### EXAMPLE 59

### 2,5-Dibromo-N-((3R,5R)-1-cyano-5-{[({[2-(trifluoromethyl)phenyl]amino}carbonyl)amino]methyl}-3-pyrrolidinyl)benzene sulfonamide

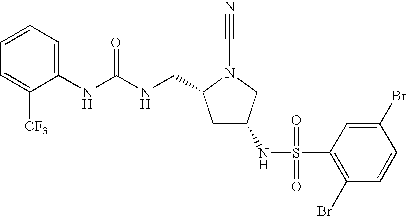

2,5-Dibromo-N-((3R,5R)-1-cyano-5-{[({[2-(trifluoromethyl)phenyl]amino}carbonyl)amino]methyl}-3-pyrrolidinyl)benzene sulfonamide (112 mg, 93%) was prepared according to the general procedure of Example 56, substituting 1-isocyanato-2-(trifluoromethyl)benzene (37 mg, 0.20 mmol) for 1-isocyanato-2-(methyloxy)benzene (30 mg, 0.20 mmol). LC-MS: 626 m/z, (M+H), rt 2.19 min. 1H NMR (400 MHz, CHLOROFORM-d) δ ppm 1.87 (d, J=7.03 Hz, 1H) 1.90 (s, 1H) 2.19 (d, J=7.03 Hz, 1H) 3.53-3.59 (m, 1H) 3.56 (dd, J=9.41, 5.65 Hz, 3H) 3.80 (t, J=12.55 Hz, 1H) 3.80 (d, J=11.80 Hz, 1H) 5.91 (br. s., 1H) 7.22-7.27 (m, 1H) 7.33 (t, J=7.91 Hz, 1H) 7.45 (d, J=8.28 Hz, 1H) 7.54-7.61 (m, 1H) 7.57 (d, J=3.01 Hz, 1H) 7.68 (d, J=13.05 Hz, 2H) 8.25 (d, J=2.01 Hz, 1H)

### EXAMPLE 60

### 2,5-Dibromo-N-((3R,5R)-1-cyano-5-{[({[3-(trifluoromethyl)phenyl]amino}carbonyl)amino]methyl}-3-pyrrolidinyl)benzene sulfonamide

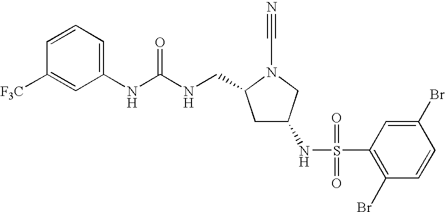

2,5-Dibromo-N-((3R,5R)-1-cyano-5-{[({[3-(trifluoromethyl)phenyl]amino}carbonyl)amino]methyl}-3-pyrrolidinyl)benzene sulfonamide (104 mg, 87%) was prepared according to the general procedure of Example 56, substituting 1-isocyanato-3-(trifluoromethyl)benzene (37 mg, 0.20 mmol) for 1-isocyanato-2-(methyloxy)benzene (30 mg, 0.20 mmol). LC-MS: 626 m/z, (M+H), rt 2.30 min. 1H NMR (400 MHz, CHLOROFORM-d) δ ppm 1.88 (d, J=13.80 Hz, 1H) 2.20 (d, J=7.03 Hz, 1H) 3.58 (dt, J=10.85, 5.49 Hz, 3H) 3.81 (td, J=11.04, 6.02 Hz, 2H) 5.98 (br. s., 1H) 7.41-7.49 (m, 4H) 7.54-7.61 (m, 2H) 7.72 (s, 1H) 8.25 (d, J=1.26 Hz, 1H)

### EXAMPLE 61

### 2,5-Dibromo-N-((3R,5R)-1-cyano-5-{[({4-(trifluoromethyl)phenyl]amino}carbonyl)amino]methyl}-3-pyrrolidinyl)benzene sulfonamide

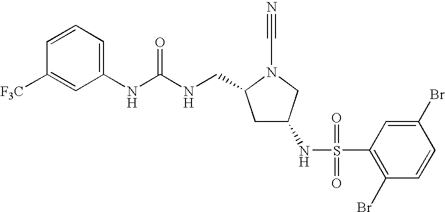

2,5-Dibromo-N-((3R,5R)-1-cyano-5-{[({4-(trifluoromethyl)phenyl]amino}carbonyl)amino]methyl}-3-pyrrolidinyl)benzene sulfonamide (111 mg, 93%) was prepared according to the general procedure of Example 56, substituting 1-isocyanato-4-(trifluoromethyl)benzene (37 mg, 0.20 mmol) for 1-isocyanato-2-(methyloxy)benzene (30 mg, 0.20 mmol). LC-MS: 626 m/z, (M+H), rt 2.32 min. 1H NMR (400 MHz, CHLOROFORM-d) δ ppm 1.88 (d, J=13.55 Hz, 1H) 2.14 (s, 1H) 2.17 (d, J=6.53 Hz, 1H) 3.38 (dd, J=10.04, 5.02 Hz, 1H) 3.50 (dd, J=10.04, 6.27 Hz, 1H) 3.56-3.62 (m, 1H) 3.59 (dd, J=5.14, 3.14 Hz, 1H) 3.72 (d, J=6.27 Hz, 1H) 3.79 (d, J=5.77 Hz, 1H) 3.89 (s, 3H) 5.48 (t, J=6.27 Hz, 1H) 6.92 (dd, J=8.03, 1.51 Hz, 1H) 6.94-7.00 (m, 1H) 7.05 (dd, J=7.91, 1.63 Hz, 1H) 7.20 (s, 1H) 7.53-7.60 (m, 2H) 7.99 (dd, J=7.91, 1.63 Hz, 1H) 8.26 (d, J=2.26 Hz, 1H)

### EXAMPLE 62

### 2,5-Dibromo-N-{(3R,5R)-1-cyano-5-[({[(3-fluorophenyl)amino]carbonyl}amino)methyl]-3-pyrrolidinyl}benzene sulfonamide

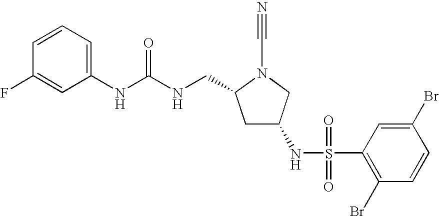

2,5-Dibromo-N-{(3R,5R)-1-cyano-5-[({[(3-fluorophenyl)amino]carbonyl}amino)methyl]-3-pyrrolidinyl}benzene sulfonamide (97 mg, 89%) was prepared according to the general procedure of Example 56, substituting with 3-fluorophenyl isocyanate (28 mg, 0.20 mmol) for 1-isocyanato-2-(methyloxy)benzene 3 (30 mg, 0.20 mmol). LC-MS: m/z, 576 (M+H), rt 2.06 min 1H NMR (400 MHz, CHLOROFORM-d) δ ppm 1.88 (t, J=6.53 Hz, 1H) 2.18 (t, J=14.18 Hz, 1H) 3.55 (d, J=17.82 Hz, 1H) 3.53 (dd, J=16.19, 5.65 Hz, 2H) 3.78 (qd, J=6.07, 5.90 Hz, 2H) 5.89 (t, J=5.65 Hz, 1H) 6.72 (td, J=8.28, 1.76 Hz, 1H) 6.97 (dd, J=8.28, 1.25 Hz, 1H) 7.19 (td, J=8.16, 6.53 Hz, 1H) 7.24-7.31 (m, 2H) 7.54-7.61 (m, 3H) 8.25 (d, J=2.26 Hz, 1H)

### EXAMPLE 63

### 2,5-Dibromo-N-{(3R,5R)-1-cyano-5-[({[(2-methylphenyl)amino]carbonyl}amino)methyl]-3-pyrrolidinyl}benzenesulfonamide

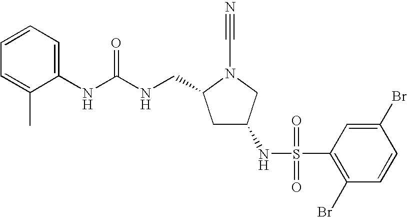

2,5-Dibromo-N-{(3R,5R)-1-cyano-5-[({[(2-methylphenyl)amino]carbonyl}amino)methyl]-3-pyrrolidinyl}benzenesulfonamide (84 mg, 77%) was prepared according to the general procedure of Example 56, substituting 1-isocyanato-2-methylbenzene (27 mg, 0.20 mmol) for 1-isocyanato-2-(methyloxy)benzene. LC-MS: m/z, 572 (M+H), rt 2.00 min. 1H NMR (400 MHz, CHLOROFORM-d) δ ppm 1.72 (dt, J=13.74, 5.55 Hz, 1H) 1.94-2.00 (m, 1H) 2.05 (s, 3H) 3.20-3.25 (m, 1H) 3.29 (dd, J=10.04, 6.02 Hz, 3H) 3.50 (dd, J=7.65, 5.65 Hz, 1H) 3.59 (t, J=5.90 Hz, 1H) 5.25 (br. s., 1H) 6.48 (s, 1H) 6.88-6.94 (m, 1H) 6.95-7.03 (m, 2H) 7.28-7.34 (m, 2H) 7.36 (s, 1H) 7.38 (s, 1H) 8.03 (d, J=2.26 Hz, 1H)

### EXAMPLE 64

### 2,5-Dibromo-N-{(3R,5R)-1-cyano-5-[({[(3-methylphenyl)amino]carbonyl}amino)methyl]-3-pyrrolidinyl}benzenesulfonamide

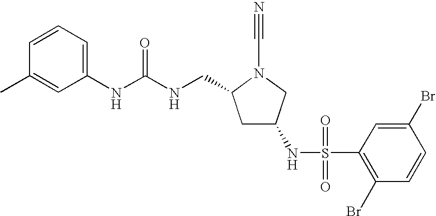

2,5-Dibromo-N-{(3R,5R)-1-cyano-5-[({[(3-methylphenyl)amino]carbonyl}amino)methyl]-3-pyrrolidinyl}benzenesulfonamide (93 mg, 86%) was prepared according to the general procedure of Example 56, substituting 1-isocyanato-3-methylbenzene (27 mg, 0.20 mmol) for 1-isocyanato-2-(methyloxy)benzene. LC-MS: m/z, 572 (M+H), rt 2.08 min. 1H NMR (400 MHz, CHLOROFORM-d) δ ppm 1.90 (t, J=6.02 Hz, 1H) 2.13-2.18 (m, 1H) 2.32 (s, 3H) 3.44-3.48 (m, 1H) 3.50-3.55 (m, 3H) 3.75 (dt, J=13.55, 6.78 Hz, 2H) 5.73 (br. s., 1H) 6.90 (d, J=7.28 Hz, 1H) 7.10-7.14 (m, 1H) 7.15-7.21 (m, 2H) 7.53-7.60 (m, 2H) 8.25 (d, J=2.01 Hz, 1H)

### EXAMPLE 65

### 2,5-Dibromo-N-{(3R,5R)-1-cyano-5-[({[(phenylmethyl)amino]carbonyl}amino)methyl]-3-pyrrolidinyl}benzenesulfonamide

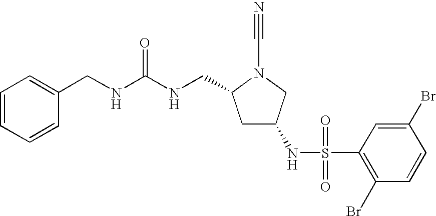

2,5-Dibromo-N-{(3R,5R)-1-cyano-5-[({[(phenyl methyl)amino]carbonyl}amino)methyl]-3-pyrrolidinyl}benzenesulfonamide (75 mg, 69%) was prepared according to the general procedure of Example 56, substituting (isocyanatomethyl)benzene (27 mg, 0.20 mmol) for 1-isocyanato-2-(methyloxy)benzene (30 mg, 0.20 mmol). LC-MS: m/z, 572 (M+H), rt 1.94 min. 1H NMR (400 MHz, CHLOROFORM-d) δ ppm 1.84 (ddd, J=13.68, 5.65, 5.52 Hz, 1H) 2.09-2.14 (m, 1H) 3.41 (d, J=4.52 Hz, 1H) 3.43-3.49 (m, 3H) 3.66 (t, J=5.52 Hz, 1H) 3.65 (q, J=5.52 Hz, 1H) 3.75 (t, J=5.77 Hz, 1H) 4.29 (d, J=5.77 Hz, 1H) 4.34 (dd, J=12.55, 5.77 Hz, 2H) 4.40 (d, J=6.02 Hz, 1H) 5.32 (t, J=6.15 Hz, 1H) 5.40 (t, J=5.77 Hz, 1H) 6.90 (br. s., 1H) 7.27-7.30 (m, 4H) 7.31-7.35 (m, 2H) 7.54-7.62 (m, 2H) 8.26 (d, J=2.26 Hz, 1H)

### EXAMPLE 66

### 2,5-Dibromo-N-[(3R,5R)-1-cyano-5-({[({[2-(methyloxy)phenyl]methyl}amino)carbonyl]amino}methyl)-3-pyrrolidinyl]benzene sulfonamide

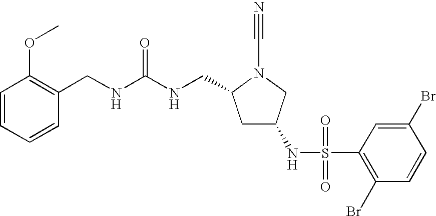

2,5-Dibromo-N-[(3R,5R)-1-cyano-5-({[({[2-(methyloxy)phenyl]methyl}amino)carbonyl]amino}methyl)-3-pyrrolidinyl]benzene sulfonamide (78 mg, 68%) was prepared according to the general procedure of Example 56, by substituting 1-(isocyanatomethyl)-2-(methyloxy)benzene (33 mg, 0.20 mmol) for 1-isocyanato-2-(methyloxy)benzene. LC-MS: m/z, 602 (M+H), rt 1.98 min. 1H NMR (400 MHz, CHLOROFORM-d) δ ppm 1.84 (t, J=6.02 Hz, 1H) 2.08 (s, 1H) 2.10 (d, J=6.53 Hz, 1H) 3.40 (d, J=5.02 Hz, 1H) 3.47 (d, J=8.03 Hz, 1H) 3.45 (dd, J=8.41, 6.15 Hz, 2H) 3.63 (d, J=6.78 Hz, 1H) 3.75 (t, J=5.90 Hz, 1H) 3.84 (s, 3H) 4.33 (dd, J=10.42, 5.90 Hz, 2H) 5.38 (t, J=6.27 Hz, 1H) 5.49 (t, J=6.15 Hz, 1H) 6.88 (d, J=8.03 Hz, 1H) 6.93 (t, J=7.53 Hz, 1H) 7.09 (br. s., 1H) 7.25 (d, J=7.53 Hz, 2H) 7.28 (s, 1H) 7.53-7.57 (m, 1H) 7.58-7.61 (m, 1H) 8.25 (d, J=2.26 Hz, 1H)

### EXAMPLE 67

### 2,5-Dibromo-N-[(3R,5R)-1-cyano-5-({[({[3-(methyloxy)phenyl]methyl}amino)carbonyl]amino}methyl)-3-pyrrolidinyl]benzene sulfonamide

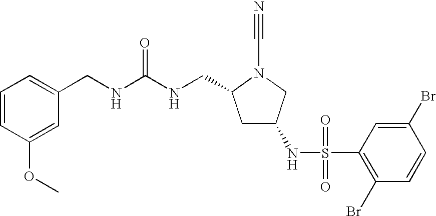

2,5-Dibromo-N-[(3R,5R)-1-cyano-5-({[({[3-(methyloxy)phenyl]methyl}amino)carbonyl]amino}methyl)-3-pyrrolidinyl]benzene sulfonamide (89 mg, 78%) was prepared according to the general procedure of Example 56 by substituting 1-(isocyanatomethyl)-3-(methyloxy)benzene (33 mg, 0.20 mmol) for 1-isocyanato-2-(methyloxy)benzene. LC-MS: m/z, 602 (M+H), rt 1.94 min. 1H NMR (400 MHz, CHLOROFORM-d) δ ppm 1.79 (s, 1H) 1.81 (d, J=6.53 Hz, 1H) 2.07 (s, 1H) 2.10 (d, J=6.53 Hz, 1H) 3.33 (dd, J=10.04, 5.27 Hz, 1H) 3.41 (t, J=5.65 Hz, 1H) 3.48 (dd, J=9.91, 6.40 Hz, 2H) 3.63 (d, J=6.02 Hz, 1H) 3.75 (t, J=6.15 Hz, 1H) 3.81 (s, 3H) 4.31 (d, J=5.77 Hz, 2H) 5.42 (br. s., 1H) 5.51 (t, J=5.90 Hz, 1H) 6.79 (d, J=2.51 Hz, 1H) 6.81-6.87 (m, 2H) 7.24 (t, J=7.91 Hz, 1H) 7.58 (q, J=8.45 Hz, 2H) 7.54-7.61 (m, 1H) 8.25 (d, J=2.26 Hz, 1H)

### EXAMPLE 68

### 2,5-Dibromo-N-[(3R,5R)-1-cyano-5-({[({[4-(methyloxy)phenyl]methyl}amino)carbonyl]amino}methyl)-3-pyrrolidinyl]benzene sulfonamide

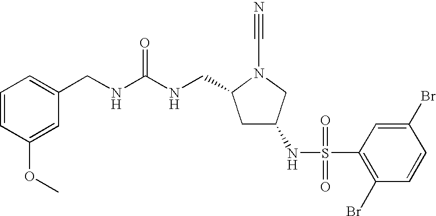

2,5-Dibromo-N-[(3R,5R)-1-cyano-5-({[({[4-(methyloxy)phenyl]methyl}amino)carbonyl]amino}methyl)-3-pyrrolidinyl]benzene sulfonamide (86 mg, 75%) was prepared according to the general procedure of Example 56 by substituting 1-(isocyanatomethyl)-4-(methyloxy)benzene (33 mg, 0.20 mmol) for 1-isocyanato-2-(methyloxy)benzene. LC-MS: m/z, 602 (M+H), rt 1.93 min. 1H NMR (400 MHz, CHLOROFORM-d) δ ppm 1.86 (d, J=13.80 Hz, 1H) 2.14 (d, J=7.28 Hz, 1H) 3.43 (d, J=4.52 Hz, 1H) 3.45-3.53 (m, 1H) 3.49 (d, J=5.77 Hz, 2H) 3.68 (dd, J=7.91, 5.40 Hz, 1H) 3.81 (s, 4H) 4.31 (s, 1H) 4.28 (d, J=6.27 Hz, 1H) 5.12 (s, 1H) 5.18 (t, J=5.65 Hz, 1H) 6.88 (d, J=8.78 Hz, 2H) 6.82-6.90 (m, 1H) 7.23 (d, J=8.78 Hz, 2H) 7.55-7.59 (m, 1H) 7.60-7.63 (m, 1H) 8.27 (d, J=2.26 Hz, 1H)

### EXAMPLE 69

### 2,5-Dibromo-N-[(3R,5R)-1-cyano-5-({[(cyclohexylamino) carbonyl]amino}methyl)-3-pyrrolidinyl]benzenesulfonamide

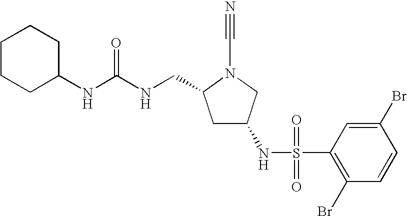

2,5-dibromo-N-[(3R,5R)-1-cyano-5-({[(cyclohexylamino) carbonyl]amino}methyl)-3-pyrrolidinyl]benzenesulfonamide (83 mg, 77%) was prepared according to the general procedure of Example 56, substituting isocyanatocyclohexane (25 mg, 0.20 mmol) for 1-isocyanato-2-(methyloxy)benzene. LC-MS: m/z, 564 (M+H), rt 1.98 min. 1H NMR (400 MHz, CHLOROFORM-d) δ ppm 1.12-1.19 (m, 3H) 1.36 (d, J=12.30 Hz, 2H) 1.71 (d, J=5.52 Hz, 2H) 1.74 (br. s., 1H) 1.94 (dt, J=13.55, 4.77 Hz, 3H) 2.16-2.22 (m, 1H) 3.47-3.55 (m, 5H) 3.74 (dd, J=8.16, 5.14 Hz, 1H) 3.80 (d, J=5.52 Hz, 1H) 4.76 (d, J=8.03 Hz, 1H) 5.07 (t, J=6.53 Hz, 1H) 7.13 (br. s., 1H) 7.55-7.59 (m, 1H) 7.60-7.64 (m, 1H) 8.27 (d, J=2.51 Hz, 1H)

### EXAMPLE 70

### 2,5-Dibromo-N-[(3R,5R)-1-cyano-5-({[4-(methyloxy)phenyl]oxy}methyl)-3-pyrrolidinyl]benzenesulfonamide

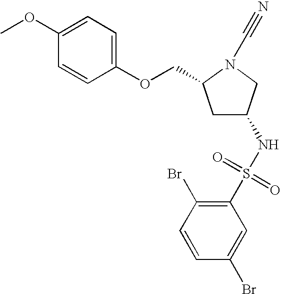

To a solution of 1,1-dimethylethyl (2R,4R)-4-amino-2-({[4-(methyloxy)phenyl]oxy}methyl)-1-pyrrolidinecarboxylate (140 mg, 0.434 mmol) (0.356 g, 1.1 mmol) in DCM (8 ml) was added DIEA (0.152 ml, 0.868 mmol) followed by 2,5-dibromobenzenesulfonyl chloride (145 mg, 0.434 mmol) at room temperature with stirring. The reaction mixture was kept stirring under argon for 2 h at room temperature then diluted with DCM and washed with 1 N HCl and saturated NaHCO₃. The DCM layer was dried (Na₂SO₄) and evaporated to give the crude as a pale yellow oil, which was mixed with 4 N HCl in dioxane (4 mL) for 1 hour followed by evaporation. The mixture was re-dissolved in DCM (10 mL) then DIEA (4 eq) and a CNBr solution (1.5 eq) were added and the resultant mixture was stirred for an additional 1 hour at room temperature. A PS-trisamine resin (0.5 g, 3.41 mmol/g) was added and the mixture was stirred for 1 hour at room temperature. The resultant mixture was filtered and the filtrate was concentrated and purified by preparatory HPLC (without TFA) to afford 2,5-dibromo-N-[(3R,5R)-1-cyano-5-({[4-(methyloxy)phenyl]oxy}methyl)-3-pyrrolidinyl]benzenesulfonamide (156 mg, 66%). LC-MS: m/z, 546 (M+H), rt2.14 min. 1H NMR (400 MHz, CHLOROFORM-d) δ ppm 1.93 (d, J=14.05 Hz, 1H) 2.32 (ddd, J=14.18, 10.04, 7.15 Hz, 1H) 3.20 (ddd, J=10.16, 1.88, 1.76 Hz, 1H) 3.33 (dd, J=10.04, 5.02 Hz, 1H) 3.76-3.97 (m, 4H) 4.15 (dd, J=10.54, 2.26 Hz, 1H) 6.69-6.81 (m, 4H) 6.81-6.93 (m, 3H) 7.44 (q, J=8.28 Hz, 2H) 8.15 (d, J=2.26 Hz, 1H)

### EXAMPLE 71

### 2,5-Dichloro-N-[(3R,5R)-1-cyano-5-({[4-(methyloxy)phenyl]oxy}methyl)-3-pyrrolidinyl]benzenesulfonamide

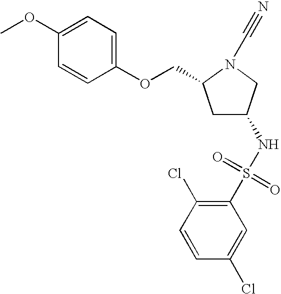

To a solution of 1,1-dimethylethyl (2R,4R)-4-amino-2-({[4-(methyloxy)phenyl]oxy}methyl)-1-pyrrolidinecarboxylate (140 mg, 0.434 mmol) (0.356 g, 1.1 mmol) in DCM (8 ml) was added DIEA (0.152 ml, 0.868 mmol) followed by 2,5-dichlorobenzenesulfonyl chloride (106 mg, 0.434 mmol) at room temperature with stirring. The reaction mixture was kept stirring under argon for 2 h at room temperature then diluted with DCM and washed with 1 N HCl and saturated NaHCO₃. The DCM layer was dried (Na₂SO₄) and evaporated to give the crude as a pale yellow oil, which was mixed with 4 N HCl in dioxane (4 mL) for 1 hour followed by evaporation. The mixture was re-dissolved in DCM (10 mL). To the resultant mixture, DIEA (4 eq) and CNBr solution (1.5 eq) were added and stirred for an additional 1 hour at room temperature. A PS-trisamine resin (0.5 g, 3.41 mmol/g) was added and the mixture was stirred for 1 hour at room temperature. The resultant mixture was filtered and the filtrate was concentrated and purified by preparatory HPLC (without TFA) to afford the 2,5-dichloro-N-[(3R,5R)-1-cyano-5-({[4-(methyloxy)phenyl]oxy}methyl)-3-pyrrolidinyl]benzenesulfonamide. (110 mg, 56%). LC-MS: m/z, 458 (M+H), rt 2.08 min. 1H NMR (400 MHz, CHLOROFORM-d) δ ppm 2.03 (s, 1H) 2.06 (d, J=14.05 Hz, 1H) 2.47 (td, J=7.03, 3.01 Hz, 1H) 3.31-3.38 (m, 1H) 3.49 (dd, J=10.04, 5.02 Hz, 1H) 3.81 (s, 3H) 3.96 (d, J=2.26 Hz, 1H) 3.98 (d, J=2.51 Hz, 1H) 4.01 (d, J=3.01 Hz, 1H) 4.03-4.09 (m, 1H) 4.29 (dd, J=10.54, 2.26 Hz, 1H) 6.85 (d, J=9.29 Hz, 1H) 6.90 (m, 2H) 6.99 (m, 2H) 7.49 (q, J=8.53 Hz, 1H) 7.46-7.53 (m, 1H) 8.12 (d, J=2.51 Hz, 1H)

### EXAMPLE 72

### 2,5-Dibromo-N-((3R,5R)-1-cyano-5-{[(4-fluorophenyl)oxy]methyl}-3-pyrrolidinyl)benzenesulfonamide

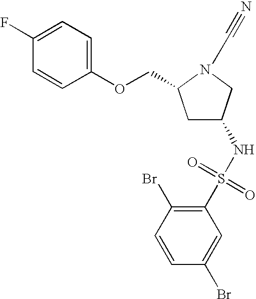

To a solution of 1,1-dimethylethyl (2R,4R)-4-amino-2-{[(4-fluorophenyl)oxy]methyl}-1-pyrrolidinecarboxylate (70 mg, 0.226 mmol) in dichloromethane (8 ml) was added DIEA (0.079 ml, 0.452 mmol followed by adding 2,5-dibromobenzenesulfonyl chloride (75 mg, 0.226 mmol) at room temperature with stirring. The reaction mixture was kept stirring under argon for 2 h at room temperature then diluted with DCM and washed with 1 N HCl and saturated NaHCO₃. The DCM layer was dried (Na₂SO₄) and evaporated to give the crude as a pale yellow oil, which was mixed with 4 N HCl in dioxane (4 mL) for 1 hour followed by evaporation. The mixture was re-dissolved in DCM (10 mL). To the resultant mixture, DIEA (4 eq) and a CNBr solution (1.5 eq) were added and stirred for 1 hour at room temperature. A PS-trisamine resin (0.5 g, 3.41 mmol/g) was added and the mixture was stirred for 1 hour at room temperature. The resultant mixture was filtered, concentrated and purified by preparatory HPLC (without TFA) to afford the 2,5-dibromo-N-((3R,5R)-1-cyano-5-{[(4-fluorophenyl)oxy]methyl}-3-pyrrolidinyl)benzenesulfonamide (45 mg, 37%). LC-MS: m/z, 534 (M+H), rt 2.24 min. 1H NMR (400 MHz, CHLOROFORM-d) δ ppm 2.06 (t, J=2.38 Hz, 1H) 2.47 (dt, J=9.98, 6.93 Hz, 1H) 3.35 (ddd, J=10.16, 2.38, 1.25 Hz, 1H) 3.49 (dd, J=10.29, 5.02 Hz, 1H) 3.99 (d, J=2.76 Hz, 1H) 4.01-4.07 (m, 3H) 4.31 (dd, J=10.29, 2.26 Hz, 1H) 6.73 (d, J=8.53 Hz, 1H) 6.99-7.07 (m, 4H) 7.56-7.63 (m, 2H) 8.30 (d, J=2.01 Hz, 1H)

### EXAMPLE 73

### 2,5-Dichloro-N-((3R,5R)-1-cyano-5-{[(4-fluorophenyl)oxy]methyl}-3-pyrrolidinyl)benzenesulfonamide

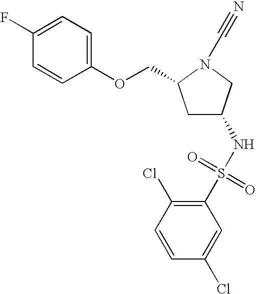

To a solution of 1,1-dimethylethyl (2R,4R)-4-amino-2-{[(4-fluorophenyl)oxy]methyl}-1-pyrrolidinecarboxylate (70 mg, 0.226 mmol) in dichloromethane (8 ml) was added DIEA (0.079 ml, 0.452 mmol followed by adding 2,5-dichlorobenzenesulfonyl chloride (55 mg, 0.226 mmol) at room temperature with stirring. The reaction mixture was kept stirring under argon for 2 h at room temperature then diluted with DCM and washed with 1 N HCl and saturated NaHCO₃. The DCM layer was dried (Na₂SO₄) and evaporated to give the crude as a pale yellow oil, which was mixed with 4 N HCl in dioxane (4 mL) for 1 hour followed by evaporation. The mixture was re-dissolved in DCM (10 mL). To the resultant mixture, DIEA (4 eq) and a CNBr solution (1.5 eq) were added and stirred for 1 hour at room temperature. A PS-trisamine resin (0.5 g, 3.41 mmol/g) was added and the mixture was stirred for 1 hour at room temperature. The resultant mixture was filtered, concentrated and purified on preparatory HPLC (without TFA) to afford 2,5-dichloro-N-((3R,5R)-1-cyano-5-{[(4-fluorophenyl)oxy]methyl}-3-pyrrolidinyl)benzenesulfonamide (56 mg, 56%). LC-MS: m/z, 446 (M+H), rt 2.18 min. 1H NMR (400 MHz, CHLOROFORM-d) δ ppm 2.05 (dd, J=17.07, 3.01 Hz, 1H) 2.47 (dt, J=9.85, 7.12 Hz, 1H) 3.35 (dd, J=9.41, 1.63 Hz, 1H) 3.50 (dd, J=10.29, 5.02 Hz, 1H) 3.98 (d, J=2.51 Hz, 1H) 4.01-4.08 (m, 2H) 4.30 (dd, J=10.29, 2.51 Hz, 1H) 6.67 (d, J=9.03 Hz, 1H) 6.98-7.02 (m, 2H) 7.04-7.08 (m, 2H) 7.47-7.54 (m, 1H) 7.50 (q, J=8.53 Hz, 1H) 8.12 (d, J=2.26 Hz, 1H)

### EXAMPLE 74

### 2-Bromo-5-chloro-N-[(3R,5S)-1-cyano-5-methyl-3-pyrrolidinyl]-4-fluorobenzenesulfonamide

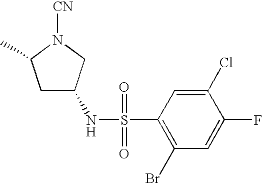

To 1,1-dimethylethyl (2S,4R)-4-amino-2-methyl-1-pyrrolidinecarboxylate (0.100 g, 0.50 mmol) in DCM (5 mL) was added TEA (0.139 mL, 1.0 mmol) and 5-bromo-2-chloro-4-fluorobenzenesulfonyl chloride (154 mg, 0.50 mmol). The resultant mixture was stirred at room temperature overnight followed by dilution with DCM. The mixture was washed with 1N HCl and saturated NaHCO₃, and the DCM layer was dried (Na₂SO₄) and evaporated. The residue was mixed with 4 N HCl in dioxane (4 mL) for 1 hour followed by evaporation. The mixture was re-dissolved in DCM (5 mL). To the resultant mixture, DIEA (0.35 mL, 2.0 mmol, 4 eq) and CNBr solution (0.25 mL, 0.75 mmol, 1.5 eq) were added and stirred for an additional 1 hour at room temperature. PS-trisamine (0.5 g, 3.41 mmol/g) was added and the mixture was stirred for 1 hour at room temperature. The resultant mixture was filtered, concentrated and purified by preparatory HPLC (without TFA) to afford 2-bromo-5-chloro-N-[(3R,5S)-1-cyano-5-methyl-3-pyrrolidinyl]-4-fluorobenzenesulfonamide (93 mg, 47%). LC-MS: m/z, 398 (M+H), rt 1.83 min. 1H NMR (400 MHz, CHLOROFORM-d) δ ppm 1.29 (d, J=6.27 Hz, 3H) 1.38-1.44 (m, 1H) 2.20 (dt, J=12.86, 6.49 Hz, 1H) 3.19 (dd, J=10.04, 7.03 Hz, 1H) 3.46 (dd, J=9.79, 7.28 Hz, 1H) 3.55 (dt, J=9.29, 6.27 Hz, 1H) 3.78-3.84 (m, 1H) 5.33 (d, J=8.03 Hz, 1H) 7.28 (d, J=7.53 Hz, 1H) 8.25 (d, J=7.03 Hz, 1H)

### EXAMPLE 75

### 2,5-Dichloro-N-[(3R,5S)-1-cyano-5-methyl-3-pyrrolidinyl]-4-fluorobenzenesulfonamide

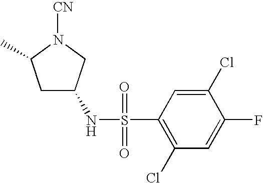

To 1,1-dimethylethyl (2S,4R)-4-amino-2-methyl-1-pyrrolidinecarboxylate (0.100 g, 0.50 mmol) in DCM (5 mL) was added TEA (0.139 mL, 1.0 mmol) and 2,5-dichloro-4-fluorobenzenesulfonyl chloride (132 mg, 0.50 mmol). The resultant mixture was stirred at room temperature overnight followed by dilution with DCM. The mixture was washed with 1N HCl and saturated NaHCO₃, and the DCM layer was dried (Na₂SO₄) and evaporated. The residue was mixed with 4 N HCl in dioxane (4 mL) for 1 hour followed by evaporation. The mixture was re-dissolved in DCM (5 mL). To the resultant mixture, DIEA (0.35 mL, 2.0 mmol, 4 eq) and CNBr solution (0.25 mL, 0.75 mmol, 1.5 eq) were added and stirred for an additional 1 hour at room temperature. PS-trisamine (0.5 g, 3.41 mmol/g) was added and the mixture was stirred for 1 hour at room temperature. The resultant mixture was filtered, concentrated and purified by preparatory HPLC (without TFA) to afford 2,5-dichloro-N-[(3R,5S)-1-cyano-5-methyl-3-pyrrolidinyl]-4-fluorobenzenesulfonamide (92 mg, 52%). LC-MS: m/z, 354 (M+H), rt 0.9 min. 1H NMR (400 MHz, CHLOROFORM-d) δ ppm 1.38 (d, J=6.27 Hz, 3H) 1.44-1.51 (m, 1H) 2.30 (dt, J=12.86, 6.49 Hz, 1H) 3.28 (dd, J=10.04, 7.03 Hz, 1H) 3.55 (dd, J=10.04, 7.28 Hz, 1H) 3.59-3.67 (m, J=6.27, 3.51, 3.14, 3.14 Hz, 1H) 3.89 (d, J=7.78 Hz, 1H) 5.41 (br. s., 1H) 7.41 (d, J=8.03 Hz, 1H) 8.21 (d, J=7.53 Hz, 1H)

### EXAMPLE 76

### 2-Bromo-5-chloro-N-[(3R,5S)-1-cyano-5-methyl-3-pyrrolidinyl]-4-fluorobenzenesulfonamide

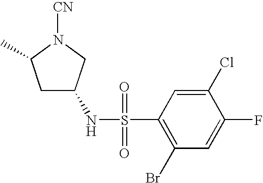

To 1,1-dimethylethyl (2S,4R)-4-amino-2-methyl-1-pyrrolidinecarboxylate (0.100 g, 0.50 mmol) in DCM (5 mL) was added TEA (0.139 mL, 1.0 mmol) and 2-bromo-5-chloro-4-fluorobenzenesulfonyl chloride (154 mg, 0.50 mmol). The resultant mixture was stirred at room temperature overnight followed by dilution with DCM. The mixture was washed with 1N HCl and saturated NaHCO₃, and the DCM layer was dried (Na₂SO₄) and evaporated. The residue was mixed with 4 N HCl in dioxane (4 mL) for 1 hour followed by evaporation. The mixture was re-dissolved in DCM (5 mL). To the resultant mixture, DIEA (0.35 mL, 2.0 mmol, 4 eq) and CNBr solution (0.25 mL, 0.75 mmol, 1.5 eq) were added and stirred for an additional 1 hour at room temperature. PS-trisamine (0.5 g, 3.41 mmol/g) was added and the mixture was stirred for 1 hour at room temperature. The resultant mixture was filtered, concentrated and purified by preparatory HPLC (without TFA) to afford 2-bromo-5-chloro-N-[(3R,5S)-1-cyano-5-methyl-3-pyrrolidinyl]-4-fluorobenzenesulfonamide (57 mg, 29%). LC-MS: m/z, 398 (M+H), rt 0.93 min. 1H NMR (400 MHz, CHLOROFORM-d) δ ppm 0.75 (t, J=7.40 Hz, 7H) 1.39-1.45 (m, 5H) 1.72 (dd, J=13.93, 2.13 Hz, 1H) 2.20 (ddd, J=10.10, 3.70, 3.51 Hz, 1H) 2.37 (d, J=2.51 Hz, 1H) 2.41 (d, J=2.26 Hz, 1H) 2.44-2.50 (m, 4H) 2.77 (dd, J=15.18, 3.14 Hz, 1H) 3.29 (d, J=4.27 Hz, 1H) 3.31 (d, J=4.52 Hz, 1H) 3.35-3.42 (m, 1H) 3.69 (dq, J=10.04, 2.68 Hz, 1H) 3.79 (dd, J=6.15, 4.64 Hz, 1H) 7.38 (d, J=2.26 Hz, 1H) 7.40 (d, J=2.51 Hz, 1H) 7.44-7.51 (m, 1H) 8.14 (d, J=2.51 Hz, 1H) 9.43 (br. s., 1H)

### EXAMPLE 77

### 2,5-Dibromo-N-{(3R,5R)-1-cyano-5-[(dipropylamino) methyl]-3-pyrrolidinyl}benzenesulfonamide

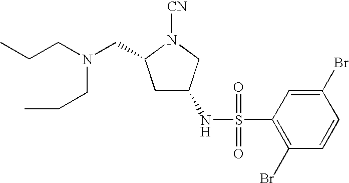

To a solution of 1,1-dimethylethyl (2R,4R)-2-(aminomethyl)-4-{[(2,5-dibromophenyl)sulfonyl]amino}-1-pyrrolidinecarboxylate (100 mg, 0.19 mmol) in methanol (4 ml), was added propanal (0.056 ml, 0.779 mmol). The reaction mixture was stirred at room temperature for 30 min followed by addition of sodium borohydride (29.5 mg, 0.779 mmol). After 30 min, more propanal (0.056 ml, 0.779 mmol) was added and the mixture was stirred at room temperature for 30 min followed by addition of sodium borohydride (29.5 mg, 0.779 mmol). The reaction mixture was kept stirring overnight. The reaction was quenched with water, and extracted with ethyl acetate. The ethyl acetate layer was dried (Na₂SO₄) and evaporated to give the crude as a pale yellow oil, which was purified by preparatory HPLC (without TFA). (35 mg, 30%). The product prepared above (35 mg, 0.059 mmol) was mixed with 4 N HCl in dioxane (4 mL) for 1 hour followed by evaporation. The mixture was re-dissolved in DCM (2 mL). To the resultant mixture, DIEA (0.04 mL, 0.234 mmol, 4 eq) and CNBr solution (0.03 mL, 0.09 mmol, 1.5 eq) were added and stirred for an additional 1 hour at room temperature. PS-trisamine (0.5 g, 3.41 mmol/g) was added and the mixture was stirred for 1 hour at room temperature. The resultant mixture was filtered, concentrated and purified by preparatory HPLC (without TFA) to afford 2,5-Dibromo-N-{(3R,5R)-1-cyano-5-[(dipropylamino) methyl]-3-pyrrolidinyl}benzenesulfonamide (14 mg, 46%). LC-MS: m/z, 523 (M+H), rt 0.78 min. 1H NMR (400 MHz, CHLOROFORM-d) δ ppm 1.39 (d, J=6.27 Hz, 3H) 1.45-1.52 (m, 1H) 2.30 (dt, J=13.05, 6.53 Hz, 1H) 3.28 (dd, J=10.04, 7.03 Hz, 1H) 3.54 (dd, J=9.79, 7.28 Hz, 1H) 3.64 (dt, J=9.03, 6.27 Hz, 1H) 3.88 (br. s., 1H) 5.45 (br. s., 1H) 7.60 (d, J=8.03 Hz, 1H) 8.25 (d, J=7.53 Hz, 1H)

### EXAMPLE 78

### 2,5-Dibromo-N-((3R,5R)-1-cyano-5-{[(phenylmethyl)oxy]methyl}-3-pyrrolidinyl)benzenesulfonamide

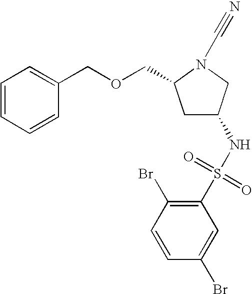

In a 20 ml flask was diluted 1,1-dimethylethyl (2R,4R)-4-amino-2-{[(phenylmethyl)oxy]methyl}-1-pyrrolidinecarboxylate (0.191 g, 0.625 mmol) in DCM (2 ml) to give a colorless solution. Then DIPEA (0.218 ml, 1.250 mmol) and dibromobenzenesulfonylchloride (0.209 g, 0.625 mmol) were added. The reaction mixture was stirred at room temperature for 16 hr followed by evaporation. The residue was loaded onto a SPE catridge (silica, 5 g) and eluted sequencially with DCM, EtOAc. The EtOAc fractions were recovered and concentrated to yield the desired product as an oil, which was mixed with 4 N HCl in dioxane (4 mL) for 1 hour followed by evaporation. The mixture was re-dissolved in DCM (10 mL). To the resultant mixture, DIEA (0.12 mL, 0.688 mmol, 4 eq) and CNBr solution (0.086 mL, 0.258 mmol, 1.5 eq) were added and stirred for 1 hour at room temperature. PS-trisamine (0.5 g, 3.43 mmol/g) was added and the mixture was stirred for 1 hour at room temperature. The resultant mixture was filtered, concentrated and purified on preparatory HPLC (without TFA) to afford 2,5-dibromo-N-((3R,5R)-1-cyano-5-{[(phenylmethyl)oxy]methyl}-3-pyrrolidinyl)benzenesulfonamide (44 mg, 46%). LC-MS: m/z, 530 (M+H), rt 1.15 min. 1H NMR (400 MHz, CHLOROFORM-d) δ ppm 1.95 (dd, J=14.05, 2.26 Hz, 1H) 2.23 (d, J=5.77 Hz, 1H) 2.21 (s, 1H) 2.35 (ddd, J=6.90, 3.64, 3.51 Hz, 1H) 3.25-3.33 (m, 1H) 3.42 (dd, J=10.16, 4.89 Hz, 1H) 3.54 (dd, J=10.79, 2.01 Hz, 1H) 3.80-3.87 (m, 3H) 3.87 (s, 1H) 3.96 (dt, J=4.71, 2.29 Hz, 1H) 4.09-4.16 (m, 2H) 6.92-6.99 (m, 3H) 7.04 (d, J=9.29 Hz, 1H) 7.27-7.35 (m, 3H) 7.52-7.57 (m, 1H) 7.58-7.63 (m, 1H) 8.28 (d, J=2.26 Hz, 1H)

### EXAMPLE 79

### 2,5-Dibromo-N-((3R,5R)-1-cyano-5-{[(phenylmethyl)oxy]methyl}-3-pyrrolidinyl)benzenesulfonamide

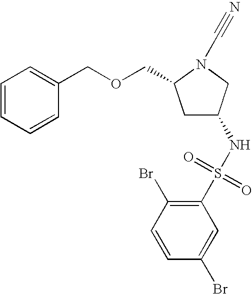

To a solution of 1,1-dimethylethyl (2R,4R)-4-amino-2-({[3-(phenyloxy)propyl]oxy}methyl)-1-pyrrolidinecarboxylate (0.092 g, 0.263 mmol) in dichloromethane (DCM) (1.5 ml) were added DIEA (0.092 ml, 0.525 mmol) and dibromobenzenesulphonylchloride (0.097 g, 0.289 mmol). The resulting mixture was stirred at room temperature for 16 h. The crude was then purified using SPE (silica, 5 g) eluting sequentially with hexane, DCM, ether and ethyl acetate. The corresponding fractions were recovered and concentrated to yield the desired product as an orange oil which was mixed with 4 N HCl in dioxane (4 mL) for 1 hour followed by evaporation. The mixture was re-dissolved in DCM (10 mL). To the resultant mixture, DIEA (0.10 mL, 0.58 mmol, 4 eq) and CNBr solution (0.072 mL, 0.217 mmol, 1.5 eq) were added and stirred for 1 hour at room temperature. PS-trisamine (0.5 g, 3.43 mmol/g) was added and the mixture was stirred for 1 hour at room temperature. The resultant mixture was filtered, concentrated and purified on preparatory HPLC (without TFA) to afford the 2,5-dibromo-N-((3R,5R)-1-cyano-5-{[(phenylmethyl)oxy]methyl}-3-pyrrolidinyl)benzenesulfonamide (43 mg, 52%). LC-MS: m/z, 574 (M+H), rt 1.22 min. 1H NMR (400 MHz, CHLOROFORM-d) δ ppm 1.94 (dd, J=14.05, 2.26 Hz, 1H) 2.36 (dd, J=7.03, 3.51 Hz, 1H) 3.29 (dt, J=10.23, 1.54 Hz, 1H) 3.43 (dd, J=10.29, 5.02 Hz, 1H) 3.54 (dd, J=10.54, 1.76 Hz, 1H) 3.84 (t, J=1.88 Hz, 1H) 3.84-3.90 (m, 1H) 4.03 (td, J=4.64, 2.26 Hz, 1H) 4.59-4.66 (m, 1H) 4.75 (d, J=11.54 Hz, 1H) 6.84 (d, J=9.29 Hz, 1H) 7.36-7.43 (m, 5H) 7.51-7.54 (m, 1H) 7.55-7.59 (m, 1H)

### EXAMPLE 80

### N-{[(2R,4R)-4-({[5-Chloro-2-(methyloxy)phenyl]sulfonyl}amino)-1-cyano-2-pyrrolidinyl]methyl}-2,2-dimethylpropanamide

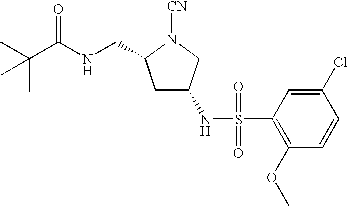

To a solution of 1,1-dimethylethyl (2R,4R)-2-(aminomethyl)-4-({[5-chloro-2-(methyloxy)phenyl]sulfonyl}amino)-1-pyrrolidinecarboxylate (280 mg, 0.667 mmol) in DCM (10 mL) was added TEA (0.186 mL, 1.344 mmol) followed by trimethylacetyl chloride (88 mg, 0.733 mmol) at room temperature while stirring. The reaction mixture was stirred at room temperature for 1 hour, washed with 1 N HCl and saturated NaHCO₃. The DCM layer was isolated and evaporated to give a crude oil that was purified by automated flash chromatography (12 g of silica cartridge) to afford a colorless oil (234 mg, 70%). This oily product (234 mg, 0.464 mmol) was treated with 4 N HCl in dioxane (4 mL). The reaction mixture was stirred at room temperature for 1 hour followed by evaporation. The mixture was re-dissolved in DCM (5 mL). To the resultant mixture, DIEA (0.324 mL, 1.857 mmol) and a CNBr solution (0.232 mL, 0.696 mmol) were added and stirred for an additional 1 hour at room temperature. PS-trisamine (0.5 g, 3.43 mmol/g) was added and the mixture was stirred for 1 hour at room temperature. The resultant mixture was filtered, concentrated and purified on preparatory HPLC (without TFA) to afford N-{[(2R,4R)-4-({[5-chloro-2-(methyloxy)phenyl]sulfonyl}amino)-1-cyano-2-pyrrolidinyl]methyl}-2,2-dimethylpropanamide (104 mg, 52%). LC-MS: m/z, 429 (M+H), rt 0.86 min. 1H NMR (400 MHz, CHLOROFORM-d) δ ppm 7.90 (d, J=2.51 Hz, 1H), 7.54 (dd, J=8.91, 2.64 Hz, 1H), 7.02 (d, J=8.78 Hz, 1H), 6.22 (d, J=5.27 Hz, 1H), 6.16 (br. s., 1H), 4.04 (s, 3H), 3.63-3.80 (m, 3H), 3.48-3.56 (m, 2H), 3.37-3.44 (m, 1H), 2.07-2.14 (m, 1H), 1.67-1.75 (m, 1H), 1.24 (s, 9H)

### EXAMPLE 81

### N-{[(2R,4R)-4-({[5-Bromo-2-(methyloxy)phenyl]sulfonyl}amino)-1-cyano-2-pyrrolidinyl]methyl}-2,2-dimethylpropanamide

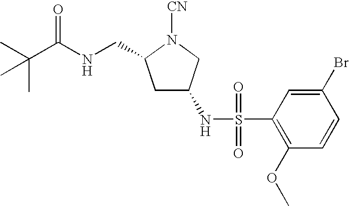

To a solution of 1,1-dimethylethyl (2R,4R)-2-(aminomethyl)-4-({[5-brom-2-(methyloxy)phenyl]sulfonyl}amino)-1-pyrrolidinecarboxylate (330 mg, 0.711 mmol) in DCM (10 mL) was added TEA (0.297 mL, 2.132 mmol) followed by trimethylacetyl chloride (171 mg, 1.421 mmol) at room temperature while stirring. The reaction mixture was stirred at room temperature for 1 hour, washed with 1 N HCl and saturated NaHCO₃. The DCM layer was isolated and evaporated to give a crude oil that was purified by automated flash chromatography (12 g of silica cartridge) to afford a colorless oil (300 mg, 77%). This oily product (300 mg, 0.547 mmol) was treated with 4 N HCl in dioxane (4 mL). The reaction mixture was stirred at room temperature for 1 hour followed by evaporation. The mixture was re-dissolved in DCM (4 mL). To the resultant mixture, DIEA (0.382 mL, 2.188 mmol) and CNBr solution (0.273 mL, 0.82 mmol) were added and stirred for an additional 1 hour at room temperature. PS-trisamine (0.5 g, 3.43 mmol/g) was added and the mixture was stirred for 1 hour at room temperature. The resultant mixture was filtered, concentrated and purified by preparatory HPLC (without TFA) to afford N-{[(2R,4R)-4-({[5-bromo-2-(methyloxy)phenyl]sulfonyl}amino)-1-cyano-2-pyrrolidinyl]methyl}-2,2-dimethylpropanamide (151 mg, 58%). LC-MS: m/z, 475 (M+H), rt 0.87 min. 1H NMR (400 MHz, CHLOROFORM-d) δ ppm 1.24 (s, 9H) 1.71 (d, J=13.55 Hz, 1H) 2.07-2.14 (m, 1H) 3.37-3.45 (m, 1H) 3.53 (dd, J=8.66, 5.14 Hz, 2H) 3.68 (d, J=6.78 Hz, 1H) 3.70-3.77 (m, 1H) 3.73 (dd, J=14.31, 6.27 Hz, 1H) 4.04 (s, 3H) 6.15 (br. s., 1H) 6.21 (d, J=5.02 Hz, 1H) 6.97 (d, J=8.78 Hz, 1H) 7.69 (dd, J=8.78, 2.51 Hz, 1H) 8.03 (d, J=2.51 Hz, 1H)

### EXAMPLE 82

### N-{[(2R,4R)-4-({[2,5-Bis(methyloxy)phenyl]sulfonyl}amino)-1-cyano-2-pyrrolidinyl]methyl}-2,2-dimethylpropanamide

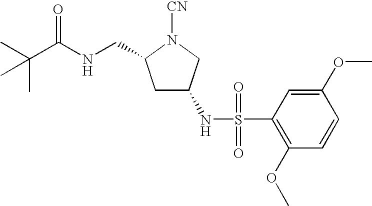

To a solution of 1,1-dimethylethyl (2R,4R)-2-(aminomethyl)-4-({[2,5-bis(methyloxy)phenyl]sulfonyl}amino)-1-pyrrolidinecarboxylate (280 mg, 0.674 mmol) in DCM (10 mL) was added TEA (0.282 mL, 2.022 mmol) followed by trimethylacetyl chloride (163 mg, 1.348 mmol) at room temperature while stirring. The reaction mixture was stirred at room temperature for 1 hour, washed with 1 N HCl and saturated NaHCO₃. The DCM layer was isolated and evaporated to give a crude oil that was purified by automated flash chromatography (12 g of silica cartridge) to afford a colorless oil (280 mg, 83%). This oily product (280 mg, 0.560 mmol) was treated with 4 N HCl in dioxane (4 mL). The reaction mixture was stirred at room temperature for 1 hour followed by evaporation. The mixture was re-dissolved in DCM (4 mL). To the resultant mixture, DIEA (0.392 mL, 2.242 mmol) and CNBr solution (0.280 mL, 0.841 mmol) were added and stirred for an additional 1 hour at room temperature. PS-trisamine (0.5 g, 3.43 mmol/g) was added and the mixture was stirred for 1 hour at room temperature. The resultant mixture was filtered, concentrated and purified by preparatory HPLC (without TFA) to afford N-{[(2R,4R)-4-({[2,5-bis(methyloxy)phenyl]sulfonyl}amino)-1-cyano-2-pyrrolidinyl]methyl}-2,2-dimethylpropanamide (151 mg, 64%). LC-MS: m/z, 425 (M+H), rt 0.79 min. 1H NMR (400 MHz, CHLOROFORM-d) δ ppm 1.21 (s, 9H) 1.65 (d, J=13.30 Hz, 1H) 2.08 (d, J=6.78 Hz, 1H) 3.42 (dd, J=16.06, 6.02 Hz, 1H) 3.46-3.51 (m, 1H) 3.63 (dt, J=14.05, 6.27 Hz, 1H) 3.74 (s, 1H) 3.72 (d, J=6.27 Hz, 1H) 3.83 (s, 3H) 3.97 (s, 3H) 6.08 (d, J=5.52 Hz, 1H) 6.24-6.32 (m, 1H) 7.00 (d, J=9.03 Hz, 1H) 7.10 (d, J=3.26 Hz, 1H) 7.13 (d, J=3.26 Hz, 1H) 7.43 (d, J=3.26 Hz, 1H)

### EXAMPLE 83

### N-({(2R,4R)-4-[{[5-Bromo-2-(methyloxy)phenyl]sulfonyl}(phenylmethyl)amino]-1-cyano-2-pyrrolidinyl}methyl)-2,2-dimethylpropanamide

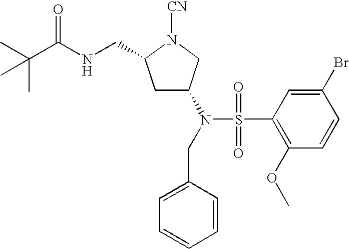

To a solution of 1,1-dimethylethyl (2R,4R)-2-(aminomethyl)-4-[{[5-bromo-2-(methyloxy)phenyl]sulfonyl}(phenylmethyl)amino]-1-pyrrolidinecarboxylate (280 mg, 0.505 mmol) in DCM (10 mL) was added TEA (0.211 mL, 1.515 mmol) followed by trimethylacetyl chloride (73 mg, 0.61 mmol) at room temperature while stirring. The reaction mixture was stirred at room temperature for 1 hour, washed with 1 N HCl and saturated NaHCO₃. The DCM layer was isolated and evaporated to give a crude oil that was purified by automated flash chromatography (12 g of silica cartridge) to afford colorless oil (280 mg, 87%). The oily product (280 mg, 0.438 mmol) prepared above was treated with 4 N HCl in dioxane (4 mL). The reaction mixture was stirred at room temperature for 1 hour followed by evaporation. The mixture was re-dissolved in DCM (4 mL). To the resultant mixture, DIEA (0.32 mL, 1.83 mmol) and CNBr solution (0.23 mL, 0.69 mmol) were added and stirred for an additional 1 hour at room temperature. PS-trisamine (0.5 g, 3.43 mmol/g) was added and the mixture was stirred for 1 hour at room temperature. The resultant mixture was filtered, concentrated and purified by preparatory HPLC (without TFA) to afford N-({(2R,4R)-4-[{[5-bromo-2-(methyloxy)phenyl]sulfonyl}(phenylmethyl)amino]-1-cyano-2-pyrrolidinyl}methyl)-2,2-dimethylpropanamide (159 mg, 64%). LC-MS: m/z, 565 (M+H), rt 1.11 min. 1H NMR (400 MHz, CHLOROFORM-d) δ ppm 1.18 (s, 9H) 1.68 (s, 1H) 1.88 (d, J=6.27 Hz, 1H) 1.90 (s, 1H) 3.15 (t, J=9.66 Hz, 1H) 3.24-3.29 (m, 1H) 3.31 (s, 1H) 3.53 (d, J=6.02 Hz, 1H) 3.64 (d, J=6.53 Hz, 1H) 3.98 (s, 3H) 4.37 (s, 1H) 4.39-4.45 (m, 1H) 4.63 (d, J=16.56 Hz, 1H) 6.06 (s, 1H) 6.93 (d, J=8.78 Hz, 1H) 7.28-7.35 (m, 5H) 7.64 (dd, J=8.78, 2.51 Hz, 1H) 8.00 (d, J=2.51 Hz, 1H)

### EXAMPLE 84

### N-({(2R,4R)-4-[{[2,5-Bis(methyloxy)phenyl]sulfonyl}(phenylmethyl)amino]-1-cyano-2-pyrrolidinyl}methyl)-2,2-dimethylpropanamide

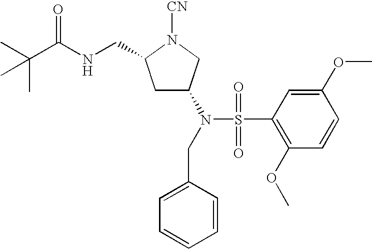

To a solution of 1,1-dimethylethyl (2R,4R)-2-(aminomethyl)-4-[{[2,5-bis(methyloxy)phenyl]sulfonyl}(phenylmethyl)amino]-1-pyrrolidinecarboxylate (270 mg, 0.534 mmol) in DCM (10 mL) was added TEA (0.149 mL, 1.068 mmol) followed by trimethylacetyl chloride (77 mg, 0.641 mmol) at room temperature while stirring. The reaction mixture was stirred at room temperature for 1 hour, washed with 1 N HCl and saturated NaHCO₃. The DCM layer was isolated and evaporated to give a crude oil that was purified by automated flash chromatography (12 g of silica cartridge) to afford colorless oil (270 mg, 86%). The oily product (270 mg, 0.458 mmol) prepared above was treated with 4 N HCl in dioxane (4 mL). The reaction mixture was stirred at room temperature for 1 hour followed by evaporation. The mixture was re-dissolved in DCM (4 mL). To the resultant mixture, DIEA (0.32 mL, 1.83 mmol) and CNBr solution (0.23 mL, 0.69 mmol) were added and stirred for an additional 1 hour at room temperature. PS-trisamine (0.5 g, 3.43 mmol/g) was added and the mixture was stirred for 1 hour at room temperature. The resultant mixture was filtered, concentrated and purified on preparatory HPLC (without TFA) to afford N-({(2R,4R)-4-[{[2,5-bis(methyloxy)phenyl]sulfonyl}(phenylmethyl)amino]-1-cyano-2-pyrrolidinyl}methyl)-2,2-dimethylpropanamide (192 mg, 81%). LC-MS: m/z, 515 (M+H), rt 1.00 min. 1H NMR (400 MHz, CHLOROFORM-d) δ ppm 1.18 (s, 9H) 1.64 (d, J=11.04 Hz, 1H) 1.86 (d, J=6.27 Hz, 1H) 1.88 (s, 1H) 3.13 (t, J=9.66 Hz, 1H) 3.18-3.25 (m, 1H) 3.27 (s, 1H) 3.54 (dd, J=14.31, 6.27 Hz, 1H) 3.61 (dd, J=6.53, 3.51 Hz, 1H) 3.82 (s, 3H) 3.94 (s, 3H) 4.43 (d, J=16.56 Hz, 2H) 4.63 (d, J=16.56 Hz, 1H) 6.05 (s, 1H) 6.98 (d, J=9.03 Hz, 1H) 7.09 (d, J=3.26 Hz, 1H) 7.11 (d, J=3.26 Hz, 1H) 7.28-7.35 (m, 5H) 7.45 (d, J=3.26 Hz, 1H)

### EXAMPLE 85

### 2,5-Dichloro-N-((3R,5R)-1-cyano-5-{[({[2-(methyloxy)ethyl]amino}carbonyl)amino]methyl}-3-pyrrolidinyl)benzenesulfonamide

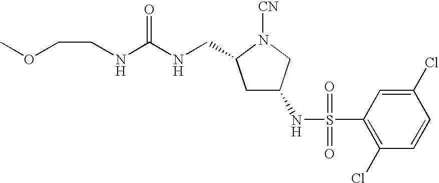

To a solution of 1,1-dimethylethyl (2R,4R)-2-(aminomethyl)-4-{[(2,5-dichlorophenyl)sulfonyl]amino}-1-pyrrolidinecarboxylate (80 mg, 0.189 mmol) in THF (3 ml), was added TEA (0.053 ml, 0.377 mmol) followed by phenyl chloroformate (0.026 ml, 0.207 mmol). The reaction mixture was stirred at room temperature overnight then diluted with DCM and washed with 1 N HCl and saturated NaHCO₃. The DCM layer was dried (Na₂SO₄) and evaporated to give the crude as a white solid, which was mixed with 2-(methyloxy)ethanamine (138 mg, 1.837 mmol) in 1,2-Dichloroethane (DCE) (3 ml) The resultant mixture was heated by microwave at 100° C. for 80 min and 120° C. for 2 hr. The mixture was diluted with DCM and 1 N NaOH. The organic layer was isolated via a hydrophobic frit and evaporated. The crude material was purified by loading it onto a NH₂ SPE (2 g) and eluting sequentially with DCM(3×) and ethyl acetate (3×)). The ethyl acetate fractions were combined and evaporated to give the desired product (73 mg, 76%).

The product described in the foregoing paragraph was treated with 4 N HCl in dioxane (2 mL). The reaction mixture was stirred at room temperature for 1 hour followed by evaporation. The mixture was re-dissolved in DCM (2 mL). To the resultant mixture, DIEA (0.097 mL, 0.556 mmol) and CNBr solution (0.070 mL, 0.21 mmol) were added and stirred for an additional 1 hour at room temperature. PS-trisamine (0.2 g, 3.43 mmol/g) was added and the mixture was stirred for 1 hour at room temperature. The resultant mixture was filtered, concentrated and purified by preparatory HPLC (without TFA) to afford 2,5-dichloro-N-((3R,5R)-1-cyano-5-{[({[2-(methyloxy)ethyl]amino}carbonyl)amino]-methyl}-3-pyrrolidinyl)benzenesulfonamide (15 mg, 24%). LC-MS: m/z, 452 (M+H), rt 0.72 min. 1H NMR (400 MHz, CHLOROFORM-d) δ ppm 1.89 (t, J=6.40 Hz, 1H) 2.13-2.19 (m, 1H) 3.36-3.41 (m, 5H) 3.48 (q, J=5.02 Hz, 2H) 3.47 (d, J=19.07 Hz, 1H) 3.54 (ddd, J=6.53, 3.14, 2.89 Hz, 2H) 3.71 (qd, J=6.02, 5.77 Hz, 1H) 3.86 (br. s., 1H) 5.48 (t, J=5.52 Hz, 1H) 5.62 (br. s., 1H) 7.35 (br. s., 1H) 7.46-7.53 (m, 1H) 7.50 (d, J=1.76 Hz, 1H) 8.09 (d, J=2.01 Hz, 1H)

### EXAMPLE 86

### 2,5-Dibromo-N-((3R,5R)-1-cyano-5-{[({[2-(methyloxy)ethyl]amino}carbonyl)amino]methyl}-3-pyrrolidinyl)benzenesulfonamide

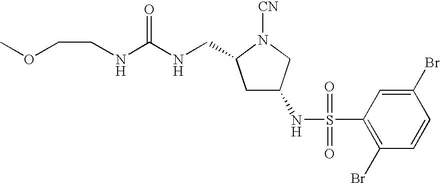

A mixture of 1,1-dimethylethyl (2R,4R)-4-{[(2,5-dibromophenyl)sulfonyl]amino}-2-({[(phenyloxy) carbonyl]amino}methyl)-1-pyrrolidinecarboxylate (150 mg, 0.237 mmol) and 2-(methyloxy)ethanamine (0.204 mL, 2.368 mmol) in 1,2-Dichloroethane (DCE) (3 ml) was heated by microwave at 120° C. for 1 h. The mixture was directly loaded onto a 5 g Si SPE cartridge eluting sequentially with DCM (3 volumes) and ethyl acetate (3 volumes). The ethyl acetate fractions were combined and evaporated to give the desired product (100 mg, 69%). This material was treated with 4 N HCl in dioxane (2 mL). The reaction mixture was stirred at room temperature for 1 hour followed by evaporation. The mixture was re-dissolved in DCM (2 mL). To the resultant mixture, DIEA (0.114 mL, 0.651 mmol) and CNBr solution (0.081 mL, 0.244 mmol) were added and stirred for an additional 1 hour at room temperature. PS-trisamine (0.2 g, 3.43 mmol/g) was added and the mixture was stirred for 1 hour at room temperature. The resultant mixture was filtered, concentrated and purified on preparatory HPLC (without TFA) to afford 2,5-dibromo-N-((3R,5R)-1-cyano-5-{[({[2-(methyloxy)ethyl]amino}carbonyl)amino]methyl}-3-pyrrolidinyl)benzenesulfonamide (57 mg, 65%). LC-MS: m/z, 452 (M+H), rt 0.83 min. 1H NMR (400 MHz, CHLOROFORM-d) δ ppm 1.90 (ddd, J=13.43, 5.90, 5.77 Hz, 1H) 2.17 (t, J=7.28 Hz, 1H) 3.36-3.42 (m, 1H) 3.41 (s, 5H) 3.48-3.55 (m, 5H) 3.72 (dd, J=7.15, 5.65 Hz, 1H) 3.85 (br. s., 1H) 5.39 (t, J=5.52 Hz, 1H) 7.55-7.59 (m, 1H) 7.60-7.64 (m, 1H) 8.27 (d, J=2.51 Hz, 1H)

### EXAMPLE 87

### N-[((2R,4R)-1-Cyano-4-{[(2,5-dibromophenyl)sulfonyl]amino}-2-pyrrolidinyl)methyl]-4-morpholinecarboxamide

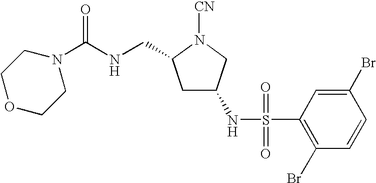

A mixture of 1,1-dimethylethyl (2R,4R)-4-{[(2,5-dibromophenyl)sulfonyl]amino}-2-({[(phenyloxy) carbonyl]amino}methyl)-1-pyrrolidinecarboxylate (150 mg, 0.237 mmol) and morpholine (0.103 mL, 1.184 mmol) in 1,2-Dichloroethane (DCE) (3 ml) was heated by microwave at 120° C. for 1 h. The mixture was directly loaded onto a 5 g Si SPE cartridge eluting sequentially with DCM (3 volumes) and EA (3 volumes). The ethyl acetate fractions were combined and evaporated to give the desired product (141 mg, 95%). This material was treated with 4 N HCl in dioxane (2 mL). The reaction mixture was stirred at room temperature for 1 hour followed by evaporation. The mixture was re-dissolved in DCM (2 mL). To the resultant mixture, DIEA (0.157 mL, 0.900 mmol) and CNBr solution (0.113 mL, 0.338 mmol) were added and stirred for an additional 1 hour at room temperature. PS-trisamine (0.2 g, 3.43 mmol/g) was added and the mixture was stirred for 1 hour at room temperature. The resultant mixture was filtered, concentrated and purified on preparatory HPLC (without TFA) to afford N-[((2R,4R)-1-cyano-4-{[(2,5-dibromophenyl)sulfonyl]amino}-2-pyrrolidinyl)methyl]-4-morpholinecarboxamide (50 mg, 40%). LC-MS: m/z, 552 (M+H), rt 1.55 min. 1H NMR (400 MHz, CHLOROFORM-d) δ ppm 1.80 (ddd, J=13.11, 6.46, 6.27 Hz, 1H) 2.04-2.11 (m, 1H) 3.34-3.40 (m, 5H) 3.44-3.52 (m, 5H) 3.68 (qd, J=6.07, 5.90 Hz, 1H) 3.80 (qd, J=6.07, 5.90 Hz, 1H) 3.99 (s, 3H) 5.43 (t, J=5.52 Hz, 1H) 5.62 (br. s., 1H) 6.67 (br. s., 1H) 7.01 (d, J=9.03 Hz, 1H) 7.52 (dd, J=8.91, 2.64 Hz, 1H) 7.88 (d, J=2.76 Hz, 1H)

### EXAMPLE 88

### 5-Chloro-N-((3R,5R)-1-cyano-5-{[({[2-(methyloxy)ethyl]amino}carbonyl)amino]methyl}-3-pyrrolidinyl)-2-(methyloxy)benzenesulfonamide

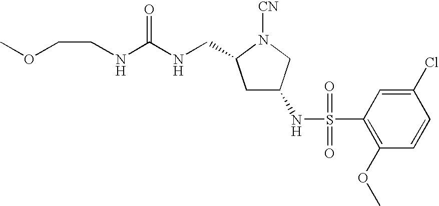

A mixture of 1,1-dimethylethyl (2R,4R)-4-({[5-chloro-2-(methyloxy)phenyl]sulfonyl}amino)-2-({[(phenyloxy)carbonyl]amino}methyl)-1-pyrrolidinecarboxylate (160 mg, 0.296 mmol) and 2-(methyloxy)ethanamine (0.255 mL, 2.96 mmol) in 1,2-dichloroethane (3 ml) was heated by microwave at 100° C. for 1 h. The mixture was diluted with DCM and 1 N NaOH. The organic layer was isolated via a hydrophobic frit. The solvent was removed in vacuo to give the desired product (150 mg, 97%). This material was treated with 4 N HCl in dioxane (2 mL). The reaction mixture was stirred at room temperature for 1 hour followed by evaporation. The mixture was re-dissolved in DCM (2 mL). To the resultant mixture, DIEA (0.201 mL, 1.152 mmol) and CNBr solution (0.144 mL, 0.432 mmol) were added and stirred for an additional 1 hour at room temperature. PS-trisamine (0.2 g, 3.43 mmol/g) was added and the mixture was stirred for 1 hour at room temperature. The resultant mixture was filtered, concentrated and purified on preparatory HPLC (without TFA) to afford 5-chloro-N-((3R,5R)-1-cyano-5-{[({[2-(methyloxy)ethyl]amino}carbonyl)amino]-methyl}-3-pyrrolidinyl)-2-(methyloxy)benzenesulfonamide (70 mg, 55%). LC-MS: m/z, 447 (M+H), rt 0.78 min. 1H NMR (400 MHz, CHLOROFORM-d) δ ppm 1.90 (d, J=13.80 Hz, 1H) 2.19 (s, 1H) 2.21 (d, J=6.78 Hz, 1H) 3.40 (s, 1H) 3.42 (d, J=5.02 Hz, 3H) 3.49-3.56 (m, 1H) 3.53 (d, J=4.27 Hz, 2H) 3.66 (d, J=14.81 Hz, 1H) 3.71-3.75 (m, 4H) 3.78 (t, J=8.16 Hz, 1H) 3.79 (d, J=6.27 Hz, 1H) 4.99 (s, 1H) 6.80 (d, J=5.77 Hz, 1H) 7.56-7.60 (m, 1H) 7.61-7.65 (m, 1H) 8.28 (d, J=2.26 Hz, 1H)

### EXAMPLE 89

### N-{[(2R,4R)-4-({[5-Chloro-2-(methyloxy)phenyl]sulfonyl}amino)-1-cyano-2-pyrrolidinyl]methyl}-4-morpholinecarboxamide

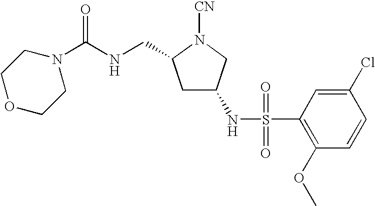

A mixture of 1,1-dimethylethyl (2R,4R)-4-({[5-chloro-2-(methyloxy)phenyl]sulfonyl}amino)-2-({[(phenyloxy)carbonyl]amino}methyl)-1-pyrrolidinecarboxylate (160 mg, 0.296 mmol) and morpholine (0.258 mL, 2.96 mmol) in 1,2-dichloroethane (3 ml) was heated by microwave at 120° C. for 2 h. The mixture was diluted with DCM and 1 N NaOH. The organic layer was isolated via a hydrophobic frit. The solvent was removed in vacuo to give the desired product (152 mg, 96%). This material was treated with 4 N HCl in dioxane (2 mL). The reaction mixture was stirred at room temperature for 1 hour followed by evaporation. The mixture was re-dissolved in DCM (2 mL). To the resultant mixture, DIEA (0.199 mL, 1.141 mmol) and CNBr solution (0.143 mL, 0.428 mmol) were added and stirred for an additional 1 hour at room temperature. PS-trisamine (0.2 g, 3.43 mmol/g) was added and the mixture was stirred for 1 hour at room temperature. The resultant mixture was filtered, concentrated and purified by preparatory HPLC (without TFA) to afford N-{[(2R,4R)-4-({[5-chloro-2-(methyloxy)phenyl]sulfonyl}amino)-1-cyano-2-pyrrolidinyl]methyl}-4-morpholinecarboxamide (83 mg, 64%). LC-MS: m/z, 552 (M+H), rt 1.42 min. 1H NMR (400 MHz, CHLOROFORM-d) δ ppm 1.82 (dt, J=13.55, 5.02 Hz, 1H) 2.08-2.14 (m, 1H) 3.36-3.40 (m, 4H) 3.44 (t, J=6.15 Hz, 1H) 3.47-3.54 (m, 1H) 3.50 (d, J=5.77 Hz, 1H) 3.59 (t, J=6.15 Hz, 1H) 3.70 (br. s., 1H) 3.68 (d, J=5.02 Hz, 3H) 3.74 (t, J=5.65 Hz, 1H) 3.95 (s, 1H) 4.00 (s, 3H) 5.37 (t, J=6.02 Hz, 1H) 6.46 (br. s., 1H) 7.01 (d, J=8.78 Hz, 1H) 7.53 (dd, J=8.78, 2.76 Hz, 1H) 7.87 (d, J=2.76 Hz, 1H)

### EXAMPLE 90

### N-((3R,5R)-5-{[(Aminocarbonyl)amino]methyl}-1-cyano-3-pyrrolidinyl)-5-chloro-2-(methyloxy)benzenesulfonamide

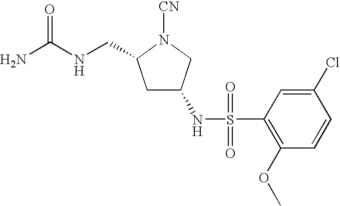

A mixture of 1,1-dimethylethyl (2R,4R)-4-({[5-chloro-2-(methyloxy)phenyl]sulfonyl}amino)-2-({[(phenyloxy)carbonyl]amino}methyl)-1-pyrrolidinecarboxylate (160 mg, 0.296 mmol) and ammonium hydroxide (30%, 0.39 mL, 2.96 mmol) in DMSO (3 ml) was heated by microwave at 100° C. for 2 h. The mixture was directly purified by preparatory HPLC (without TFA) to afford the desired compound. (38 mg, 28%). The product (38 mg, 0.082 mmol) prepared above was treated with 4 N HCl in dioxane (2 mL). The reaction mixture was stirred at room temperature for 1 hour followed by evaporation. The mixture was re-dissolved in DCM (2 mL). To the resultant mixture, DIEA (0.057 mL, 0.33 mmol) and CNBr solution (0.041 mL, 0.123 mmol) were added and stirred for an additional 1 hour at room temperature. PS-trisamine (0.2 g, 3.43 mmol/g) was added and the mixture was stirred for 1 hour at room temperature. The resultant mixture was filtered, concentrated and purified by preparatory HPLC (without TFA) to afford N-((3R,5R)-5-{[(aminocarbonyl)amino]methyl}-1-cyano-3-pyrrolidinyl)-5-chloro-2-(methyloxy)benzenesulfonamide. (19 mg, 59%). LC-MS: m/z, 390 (M+H), rt 0.75 min. 1H NMR (400 MHz, CHLOROFORM-d) δ ppm 1.76 (dt, J=13.36, 6.74 Hz, 1H) 2.01 (s, 1H) 2.08 (t, J=13.93 Hz, 1H) 2.08 (d, J=13.30 Hz, 1H) 3.33-3.41 (m, 6H) 3.43-3.49 (m, 1H) 3.45 (t, J=5.27 Hz, 3H) 3.67 (dd, J=12.30, 1.76 Hz, 1H) 3.73-3.79 (m, 1H) 3.82 (s, 3H) 3.94 (s, 3H) 5.34 (t, J=5.65 Hz, 1H) 5.55 (br. s., 1H) 6.26 (d, J=5.77 Hz, 1H) 6.99 (d, J=9.03 Hz, 1H) 7.09 (d, J=3.26 Hz, 1H) 7.11 (d, J=3.26 Hz, 1H) 7.42 (d, J=3.01 Hz, 1H)

### EXAMPLE 91

### N-((3R,5R)-1-Cyano-5-{[({[2-(methyloxy)ethyl]amino}carbonyl)amino]methyl}-3-pyrrolidinyl)-2,5-bis(methyloxy)benzenesulfonamide

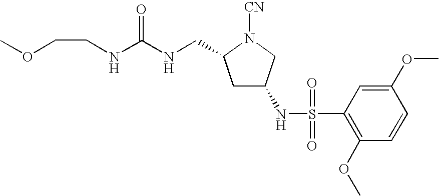

A mixture of 1,1-dimethylethyl (2R,4R)-4-({[2,5-bis(methyloxy)phenyl]sulfonyl}amino)-2-({[(phenyloxy)carbonyl]amino}methyl)-1-pyrrolidinecarboxylate (110 mg, 0.205 mmol)) and ammonium hydroxide (30%, 0.27 mL, 2.05 mmol) in 1,2-dichloroethane (3 ml) was heated by microwave at 120° C. for 2 h. The mixture was diluted with DCM and 1 N NaOH. The organic layer was isolated via a hydrophobic frit. The solvent was removed in vacuo to give the desired product (113 mg, 98%). The product (113 mg, 0.219 mmol) prepared above was treated with 4 N HCl in dioxane (2 mL). The reaction mixture was stirred at room temperature for 1 hour followed by evaporation. The mixture was re-dissolved in DCM (2 mL). To the resultant mixture, DIEA (0.153 mL, 0.875 mmol) and CNBr solution (0.11 mL, 0.33 mmol) were added and stirred for an additional 1 hour at room temperature. PS-trisamine (0.2 g, 3.41 mmol/g) was added and the mixture was stirred for 1 hour at room temperature. The resultant mixture was filtered, concentrated and purified on preparatory HPLC (without TFA) to afford N-((3R,5R)-1-Cyano-5-{[({[2-(methyloxy)ethyl]amino}carbonyl)amino]methyl}-3-pyrrolidinyl)-2,5-bis(methyloxy)-benzenesulfonamide (54 mg, 54%). LC-MS: m/z, 442 (M+H), rt 0.64 min. 1H NMR (400 MHz, CHLOROFORM-d) δ ppm 1.79 (d, J=6.78 Hz, 1H) 2.11 (d, J=6.78 Hz, 1H) 3.40-3.47 (m, 3H) 3.75 (dd, J=15.18, 6.65 Hz, 1H) 3.83 (s, 3H) 3.93 (s, 3H) 5.25 (br. s., 2H) 5.95 (t, J=6.15 Hz, 1H) 6.78 (d, J=6.02 Hz, 1H) 7.00 (d, J=9.29 Hz, 1H) 7.10 (d, J=3.26 Hz, 1H) 7.12 (d, J=3.26 Hz, 1H) 7.42 (d, J=3.01 Hz, 1H)

### EXAMPLE 92

### N-((3R,5R)-5-{[(Aminocarbonyl)amino]methyl}-1-cyano-3-pyrrolidinyl)-2,5-bis(methyloxy)benzenesulfonamide

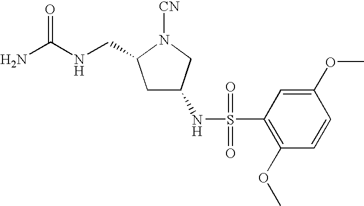

A mixture of 1,1-dimethylethyl (2R,4R)-4-({[2,5-bis(methyloxy)phenyl]sulfonyl}amino)-2-({[(phenyloxy)carbonyl]amino}methyl)-1-pyrrolidinecarboxylate (120 mg, 0.224 mmol) and 2-(methyloxy)ethanamine (0.193 mL, 2.240 mmol) in 1,2-dichloroethane (3 ml) was heated by microwave at 100° C. for 1 h. The mixture was directly purified by preparatory HPLC (without TFA) to afford the desired compound. (28 mg, 30%). The product (28 mg, 0.061 mmol) prepared above was treated with 4 N HCl in dioxane (2 mL). The reaction mixture was stirred at room temperature for 1 hour followed by evaporation. The mixture was re-dissolved in DCM (2 mL). To the resultant mixture, DIEA (0.043 mL, 0.244 mmol) and CNBr solution (0.031 mL, 0.092 mmol) were added and stirred for an additional 1 hour at room temperature. PS-trisamine (0.2 g, 3.41 mmol/g) was added and the mixture was stirred for 1 hour at room temperature. The resultant mixture was filtered, concentrated and purified on preparatory HPLC (without TFA) to afford N-((3R,5R)-5-{[(aminocarbonyl)amino]methyl}-1-cyano-3-pyrrolidinyl)-2,5-bis(methyloxy)benzenesulfonamide (10.3 mg, 44%). LC-MS: m/z, 442 (M+H), rt 0.66 min. 1H NMR (400 MHz, CHLOROFORM-d) δ ppm 1.83 (d, J=13.55 Hz, 1H) 2.13 (d, J=6.78 Hz, 1H) 2.16 (s, 1H) 3.49 (t, J=5.27 Hz, 3H) 3.52 (br. s., 1H) 3.75 (d, J=6.02 Hz, 1H) 3.76-3.83 (m, 1H) 3.99 (s, 3H) 5.09 (s, 2H) 5.77 (t, J=6.27 Hz, 1H) 6.80 (d, J=5.77 Hz, 1H) 7.02 (d, J=9.03 Hz, 1H) 7.54 (dd, J=8.78, 2.76 Hz, 1H) 7.87 (d, J=2.76 Hz, 1H)

### EXAMPLE 93

### ((2R,4R)-1-Cyano-4-{[(2,5-dibromophenyl)sulfonyl]amino}-2-pyrrolidinyl)methyl dimethylcarbamate

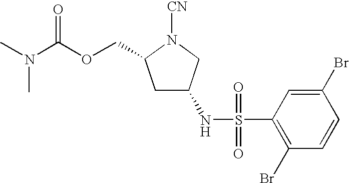

((2R,4R)-4-{[(2,5-Dibromophenyl)sulfonyl]amino}-1-{[(1,1-dimethylethyl)oxy]carbonyl}-2-pyrrolidinyl)methyl 1H-imidazole-1-carboxylate (59 mg, 0.096 mmol) was diluted in toluene (1 mL) and mixed with dimethylamine (0.194 mmol) at room temperature overnight. More dimethylamine (0.194 mmol) was added and the mixture was stirred for another 24 hours at room temperature. The reaction mixture was diluted with DCM and washed with brine. The organic layer was separated via a hydrophobic frit and concentrated under vacuum to give 1,1-dimethylethyl (2R,4R)-4-{[(2,5-dibromophenyl)sulfonyl]amino}-2-({[(dimethylamino)carbonyl]oxy}methyl)-1-pyrrolidinecarboxylate as a crude material (57 mg, 64% purity by LC/MS). This material was diluted in dioxane (0.5 mL) and mixed with 4 N HCl in dioxane (0.024 mL) for 16 hours. The reaction mixture was then concentrated under vacuum to give a white solid, which was diluted in DCM and neutralized with DIEA (0.067 mL). A BrCN solution (0.146 mmol) was added and the reaction mixture was stirred for 3 hours. A PS-trisamine resin (4 eq, 0.39 mmol, 3.41 mmol/g) was added and the mixture was stirred for 2 hour at room temperature. The resin was filtered off and the filtrate was concentrated under vacuum then purified by preparatory HPLC (without TFA) to afford ((2R,4R)-1-cyano-4-{[(2,5-dibromophenyl)sulfonyl]amino}-2-pyrrolidinyl)methyl dimethylcarbamate. (7.5 mg). LC-MS: m/z, 510.9 (M+), rt 1.82 min. 1H NMR (400 MHz, CHLOROFORM-d) δ ppm 8.20 (d, J=2.26 Hz, 1H) 7.53-7.57 (m, 1H) 7.49-7.53 (m, 1H) 5.96 (br. s., 1H) 4.19-4.24 (m, 1H) 4.07-4.13 (m, 1H) 3.80 (dd, J=6.78, 5.52 Hz, 1H) 3.80 (td, J=10.79, 4.27 Hz, 1H) 3.45 (dd, J=10.16, 6.40 Hz, 1H) 3.28 (dd, J=10.16, 5.65 Hz, 1H) 2.90 (s, 3H) 2.87 (s, 3H) 2.20 (ddd, J=13.30, 7.78, 7.53 Hz, 1H) 1.65 (ddd, J=13.43, 6.65, 6.53 Hz, 1H)

### EXAMPLE 94

### ((2R,4R)-1-Cyano-4-{[(2,5-dibromophenyl)sulfonyl]amino}-2-pyrrolidinyl)methyl ethyl(methyl)carbamate

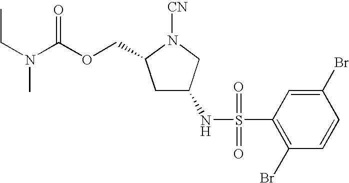

((2R,4R)-4-{[(2,5-Dibromophenyl)sulfonyl]amino}-1-{[(1,1-dimethylethyl)oxy]carbonyl}-2-pyrrolidinyl)methyl 1H-imidazole-1-carboxylate (420 mg, 0.697 mmol) was diluted in toluene (2 mL) then methylethylamine (2.09 mmol) was added and the reaction mixture was stirred at room temperature for 2.5 days. The reaction mixture was diluted with DCM and washed twice with brine. The organic layer was separated using a hydrophobic frit and concentrated under vacuum to give 1,1-dimethylethyl (2R,4R)-4-{[(2,5-dibromophenyl)sulfonyl]amino}-2-[({[ethyl (methyl)amino]carbonyl}oxy)methyl]-1-pyrrolidinecarboxylate as a crude material (303.2 mg, 75% purity by LC/MS). This material was treated with 4 N HCl in dioxane (1.7 mL) at room temperature for 4 hours. The solvent was then removed under vacuum to give a white solid, which was treated with DCM (1 mL) then DIEA (0.47 mL). A 1N BrCN solution (0.76 mmol) in DCM was added and the reaction mixture was stirred for 2 hours. A PS-trisamine resin (4 eq, 2.70 mmol, 3.41 mmol/g) was added and the mixture was stirred for 2 hour. The resin was filtered off and the filtrate was purified by preparatory HPLC (without TFA) to afford the title compound (120.5 mg). LC-MS: m/z, 525.2 (M+H), rt 1.91 min. 1H NMR (400 MHz, CHLOROFORM-d) δ ppm 8.19 (d, J=2.26 Hz, 1H) 7.53-7.57 (m, 1H) 7.49-7.52 (m, 1H) 6.00 (br. s., 1H) 4.18-4.25 (m, 1H) 4.06-4.12 (m, 1H) 3.76-3.84 (m, 2H) 3.46 (dd, J=9.03, 7.28 Hz, 1H) 3.23-3.30 (m, 1H) 3.29 (d, J=5.77 Hz, 2H) 2.86 (d, J=8.28 Hz, 3H) 2.20 (ddd, J=13.87, 7.40, 7.09 Hz, 1H) 1.64 (ddd, J=13.43, 6.65, 6.53 Hz, 1H) 1.06 (t, J=7.15 Hz, 3H)

### EXAMPLE 95

### ((2R,4R)-1-Cyano-4-{[(2,5-dibromophenyl)sulfonyl]amino}-2-pyrrolidinyl)methyl methyl(phenylmethyl)carbamate

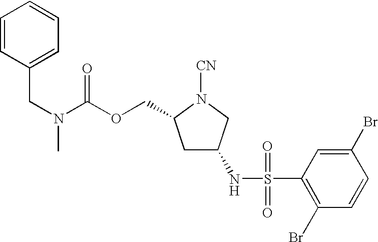

((2R,4R)-4-{[(2,5-Dibromophenyl)sulfonyl]amino}-1-{[(1,1-dimethylethyl)oxy]carbonyl}-2-pyrrolidinyl)methyl 1H-imidazole-1-carboxylate (420 mg, 0.697 mmol) was diluted in toluene (2 mL) then methyl(phenylmethyl)lamine (2.09 mmol) was added and the reaction mixture was stirred at room temperature for 2.5 days. The reaction mixture was diluted with DCM and washed twice with brine. The organic layer was separated using a hydrophobic frit and concentrated under vacuum to give 1,1-dimethylethyl (2R,4R)-4-{[(2,5-dibromophenyl)sulfonyl]amino}-2-[({[methyl(phenyl methyl)amino]carbonyl}-oxy)methyl]-1-pyrrolidinecarboxylate as a crude material (445.7 mg, 56% purity by LC/MS). This material was treated with 4 N HCl in dioxane (1.7 mL) at room temperature for 4 hours. The solvent was then removed under vacuum to give a white solid, which was treated with DCM (1 mL) then DIEA (0.47 mL). A 1N BrCN solution (0.76 mmol) in DCM was added and the reaction mixture was stirred for 2 hours. A PS-trisamine resin (4 eq, 2.70 mmol, 3.41 mmol/g) was added and the mixture was stirred for 2 hour. The resin was filtered off and the filtrate was purified by preparatory HPLC (without TFA) to afford the title compound (194 mg). LC-MS: m/z, 587 (M+H), rt 2.13 min. 1H NMR (400 MHz, CHLOROFORM-d) δ ppm 8.25 (d, J=4.27 Hz, 1H) 7.55-7.62 (m, 2H) 7.35 (t, J=7.15 Hz, 2H) 7.28-7.31 (m, 1H) 7.21-7.27 (m, 2H) 6.30 (br. s., 0.5H) 5.94 (br. s., 0.5H) 4.44-4.58 (m, 2H) 4.31 (td, J=10.98, 3.64 Hz, 1H) 4.17-4.24 (m, 1H) 3.85-3.94 (m, 1H) 3.81 (br. s., 1H) 3.43-3.56 (m, 1H) 3.35 (dd, J=9.66, 6.40 Hz, 0.5H) 3.10-3.18 (m, 0.5H) 2.95 (s, 1.5H) 2.91 (s, 1.5H) 2.22-2.32 (m, 0.5H) 2.15 (ddd, J=13.36, 7.15, 6.84 Hz, 0.5H) 1.72 (ddd, J=13.43, 6.90, 6.78 Hz, 0.5H) 1.54-1.64 (m, 0.5H)

### EXAMPLE 96

### ((2R,4R)-1-Cyano-4-{[(2,5-dibromophenyl)sulfonyl]amino}-2-pyrrolidinyl)methyl diethylcarbamate

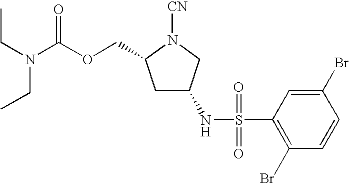

((2R,4R)-4-{[(2,5-Dibromophenyl)sulfonyl]amino}-1-{[(1,1-dimethylethyl)oxy]carbonyl}-2-pyrrolidinyl)methyl 1H-imidazole-1-carboxylate (420 mg, 0.697 mmol) was diluted in toluene (2 mL) then diethylamine (2.09 mmol) was added and the reaction mixture was stirred at room temperature for 2.5 days. The reaction mixture was diluted with DCM and washed twice with brine. The organic layer was separated using a hydrophobic frit and concentrated under vacuum to give 1,1-dimethylethyl (2R,4R)-4-{[(2,5-dibromophenyl)sulfonyl]amino}-2-([(diethylamino)carbonyl]oxy-methyl)-1-pyrrolidinecarboxylate as a crude material (404.5 mg, 72% purity by LC/MS). This material was treated with 4 N HCl in dioxane (1.7 mL) at room temperature for 4 hours. The solvent was then removed under vacuum to give a white solid, which was treated with DCM (1 mL) then DIEA (0.47 mL). A 1N BrCN solution (0.76 mmol) in DCM was added and the reaction mixture was stirred for 2 hours. A PS-trisamine resin (4 eq, 2.70 mmol, 3.41 mmol/g) was added and the mixture was stirred for 2 hour. The resin was filtered off and the filtrate was purified by preparatory HPLC (without TFA) to afford the title compound (56.5 mg). LC-MS: m/z, 539.0 (M+H), rt 1.95 min. 1H NMR (400 MHz, CHLOROFORM-d) δ ppm 8.19 (d, J=2.26 Hz, 1H) 7.53-7.57 (m, 1H) 7.49-7.52 (m, 1H) 6.07 (d, J=7.03 Hz, 1H) 4.22 (dd, J=11.54, 4.52 Hz, 1H) 4.05-4.12 (m, 1H) 3.75-3.85 (m, 2H) 3.47 (dd, J=10.16, 6.65 Hz, 1H) 3.29 (d, J=6.02 Hz, 1H) 3.24 (dd, J=14.30, 6.27 Hz, 4H) 2.15-2.23 (m, 1H) 1.65 (ddd, J=13.49, 7.03, 6.84 Hz, 1H) 1.07 (t, J=7.15 Hz, 6H)

### EXAMPLE 97

### 2,5-Dibromo-N-[(3R,5S)-1-cyano-5-methyl-3-pyrrolidinyl]-N-2-propen-1-ylbenzenesulfonamide

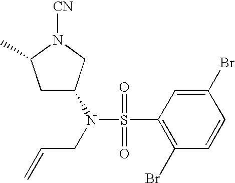

To a mixture of 1,1-dimethylethyl (2S,4R)-4-{[(2,5-dibromophenyl)sulfonyl]amino}-2-methyl-1-pyrrolidinecarboxylate (0.025 g, 0.050 mmol) in acetone (2.0 mL) and DMF (0.7 mL) were added potassium carbonate (0.069 g, 0.502 mmol) and allyl bromide (0.028 mL, 0.251 mmol) to give a yellow solution with a K₂CO₃ suspension. The reaction mixture was allowed to stir overnight. LC/MS showed the reaction was completed. A PS-triamine resin (0.118 g, 0.401 mmol, 3.41 mmol/g) was added to trap the excess allylbromide and the resulting mixture was stirred overnight. The resin was filtered off and the filtrate was concentrated to give a crude residue. To the residue previously obtained in DCM (2 mL) was added a 4N HCL aqueous solution (0.502 mL, 2.006 mmol). The reaction mixture was stirred for 2 hours at room temperature. LC/MS showed the reaction was completed. The solvent was evaporated and the residue was dissolved in DCM (2 mL). Triethylamine (0.028 mL, 0.201 mmol) was added and the vial was shaken for 5 minutes then BrCN (0.033 mL, 3N in DCM, 0.1 mmol) was added. The reaction mixture was shaken overnight. LC/MS indicated the reaction was complete. A PS-triamine resin (0.105 g, 0.2 mmol, 2.21 mmol/g) was added and the flask was shaken overnight to neutralize the excess BrCN. The resin was filtered off and the organic filtrate was concentrated to give a crude residue, which was purified by preparatory HPLC (without TFA) to give the title compound (13.7 mg). LC-MS: m/z, 463 (M+H), rt 2.23 min. 1H NMR (400 MHz, MeOD) δ ppm 8.19 (d, J=2.51 Hz, 1H) 7.66-7.70 (m, 1H) 7.63 (d, J=2.51 Hz, 1H) 5.72 (ddd, J=11.17, 5.52, 5.40 Hz, 1H) 5.18 (dd, J=17.07, 1.25 Hz, 1H) 5.03 (dd, J=10.29, 1.25 Hz, 1H) 4.54-4.61 (m, 1H) 4.01 (d, J=5.52 Hz, 2H) 3.54 (dt, J=10.54, 5.90 Hz, 1H) 3.33-3.40 (m, 2H) 2.08 (dt, J=12.36, 6.24 Hz, 1H) 1.61-1.68 (m, 1H) 1.25 (d, J=6.27 Hz, 3H)

### EXAMPLE 98

### N-(2-Biphenylylmethyl)-2,5-dibromo-N-[(3R,5S)-1-cyano-5-methyl-3-pyrrolidinyl]benzenesulfonamide

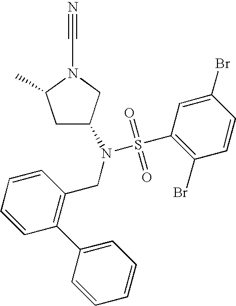

1,1-Dimethylethyl (2S,4R)-4-{[(2,5-dibromophenyl)sulfonyl]amino}-2-methyl-1-pyrrolidinecarboxylate (0.025 g, 0.050 mmol) was placed in a vial with potassium carbonate (0.104 g, 0.753 mmol) and 2-(bromomethyl)biphenyl (0.251 mmol) in acetone (2.0 mL) and DMF (0.7 mL) to give a clear solution containing a K₂CO₃ suspension. The reaction mixture was allowed to stir for 2.5 days. LC/MS sowed the reaction was complete. A PS-triamine resin (0.147 g, 0.501 mmol, 3.41 mmol/g) was added to trap the excess alkyl bromide and the resulting mixture was stirred overnight. The resin was filtered off and the filtrate was concentrated to give a crude residue. To the residue previously obtained in DCM (2 mL) was added a 4N HCL dioxane solution (0.5 mL, 2.0 mmol). The reaction mixture was stirred overnight. The solvent was evaporated to give a residue.

The residue previously obtained was taken up in DCM (3 mL) to which was added DIPEA (0.035 mL, 0.20 mmol) and the vial was shaken for 10 minutes. Then BrCN (0.033 mL, 3N in DCM, 0.1 mmol) was added to give a yellow solution. The reaction mixture was stirred for 2.5 days. LC/MS indicated the reaction was complete. A PS-triamine resin (0.059 g, 0.2 mmol, 3.41 mmol/g) was added and the flask was shaken for 2 hours to neutralize the excess BrCN. The resin was removed and the organic filtrate was concentrated to give a crude residue, which was purified by preparatory HPLC (without TFA) to give the title compound (19.41 mg). LC-MS: m/z, 587 (M+), rt 2.635 min. 1H NMR (500 MHz, DMSO-d₆) δ ppm 7.68-7.74 (m, 2H) 7.65 (m, 1H) 7.48 (m, 2H) 7.38-7.44 (m, 1H) 7.30-7.35 (m, 2H) 7.19-7.28 (m, 2H) 7.15 (s, 2H) 4.66-4.80 (m, 1H) 4.58 (dd, J=41.63, 16.66 Hz, 2H) 3.48-3.59 (m, 1H) 3.28 (m, 2H) 3.02 (t, J=8.82 Hz, 1H) 1.94 (s, 1H) 1.31 (q, J=11.01 Hz, 1H) 1.09 (d, J=6.16 Hz, 3H).

The compounds set out in the Examples shown in Table 1 were prepared according to the general procedure of Example 98, but substituting the appropriate alkyl bromide for the 2-(bromomethyl)biphenyl in Example 98.

TABLE 1

| | | Retention | LC-MS: |
|---|---|---|---|
| | | Time | m/z, |
| Example | Structure/NMR | (min) | #(M+) |
| 99 | 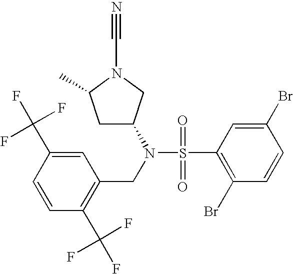 | 2.61 | 647 |
| | N-{[2,5-Bis(trifluoromethyl)phenyl]methyl}-2,5- | | |
| | dibromo-N-[(3R,5S)-1-cyano-5-methyl-3- | | |
| | pyrrolidinyl]benzenesulfonamide; | | |
| | 1 H NMR (500 MHz, DMSO-d₆) δ ppm 7.92-7.97 | | |
| | (m, 1 H) 7.86-7.90 (m, 1 H) 7.77-7.83 (m, 1 H) | | |
| | 7.74 (m, 1 H) 7.65-7.71 (m, 1 H) 7.57-7.62 (m, 1 | | |
| | H) 4.82-5.00 (m, 3 H) 3.59-3.67 (m, 1 H) 3.34- | | |
| | 3.43 (m, 2 H) 2.07-2.13 (m, 1 H) 1.53 (dd, | | |
| | J = 22.22, 11.29 Hz, 1 H) 1.16 (d, J = 6.23 Hz, 3 H) | | |
| 100 | 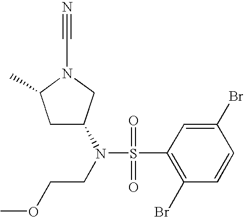 | 2.08 | 478.8 |
| | 2,5-Dibromo-N-[(3R, 5S)-1-cyano-5-methyl-3- | | |
| | pyrrolidinyl]-N-[2-(methyloxy)ethyl]- | | |
| | benzenesulfonamide | | |
| | 1 H NMR (500 MHz, DMSO-d₆) δ ppm 8.09 (m, 1 | | |
| | H) 7.80-7.82 (m, 1 H) 7.76-7.79 (m, 1 H) 4.45- | | |
| | 4.51 (m, 1 H) 3.56 (m, 1 H) 3.49-3.54 (m, 2 H) | | |
| | 3.40 (m, 2 H) 3.32 -3.37 (m, 2 H) 3.14 (s, 3 H) | | |
| | 2.00-2.06 (m, 1 H) 1.64 (dd, J = 22.46, 10.80 Hz, 1 | | |
| | H) 1.22 (d, J = 6.16 Hz, 3 H) | | |
| 101 | 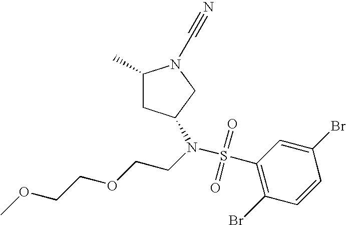 | 2.06 | 522.9 |
| | 2,5-Dibromo-N-[(3R,5S)-1-cyano-5-methyl-3- | | |
| | pyrrolidinyl]-N-(2-{[2-(methyloxy)ethyl]- | | |
| | oxy}ethyl)benzenesulfonamide | | |
| | 1 H NMR (500 MHz, DMSO-d₆) δ ppm 8.04-8.13 | | |
| | (m, 1 H) 7.80 (m, 1 H) 7.74-7.80 (m, 1 H) 4.40- | | |
| | 4.51 (m, 1 H) 3.51-3.61 (m, 1 H) 3.46 (m, 1 H) | | |
| | 3.42 (m, 4 H) 3.45 (m, 2 H) 3.39 (m, 3 H) 3.23 (s, | | |
| | 3 H) 1.95-2.06 (m, 1 H) 1.69 (q, J = 11.01 Hz, 1 H) | | |
| | 1.22 (d, J = 6.16 Hz, 3 H) | | |
| 102 | 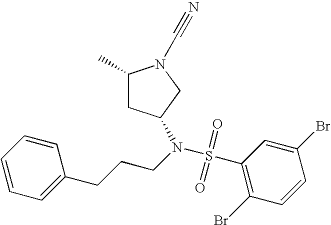 | 2.53 | 538.9 |
| | 2 2,5-Dibromo-N-[(3R,5S)-1-cyano-5-methyl-3- | | |
| | pyrrolidinyl]-N-(3-phenylpropyl)- | | |
| | benzenesulfonamide | | |
| | 1 H NMR (500 MHz, DMSO-d₆) δ ppm 8.05 (d, | | |
| | J = 2.20 Hz, 1 H) 7.76-7.82 (m, 2 H) 7.26 (m, 2 H) | | |
| | 7.17 (s, 1 H) 7.10 (s, 2 H) 4.46-4.52 (m, 1 H) | | |
| | 3.54-3.60 (m, 1 H) 3.33-3.39 (m, 6 H) 2.02-2.08 | | |
| | (m, 1 H) 1.68-1.72 (m, 2 H) 1.51 (dd, J = 22.16, | | |
| | 11.29 Hz, 1 H) 1.18 (d, J = 6.16 Hz, 3 H) | | |
| 103 | 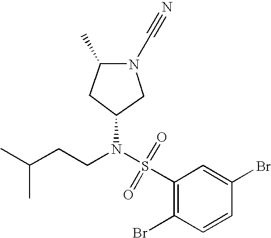 | 2.465 | 490.9 |
| | 2,5-Dibromo-N-[(3R,5S)-1-cyano-5-methyl-3- | | |
| | pyrrolidinyl]-N-(3-methylbutyl)benzenesulfonamide | | |
| | 1 H NMR (500 MHz, DMSO-d₆) δ ppm 8.09 (d, | | |
| | J = 2.26 Hz, 1 H) 7.77-7.83 (m, 2 H) 4.45 (m, 1 H) | | |
| | 3.55-3.61 (m, 1 H) 3.34 (m, 4 H) 2.03-2.09 (m, 1 | | |
| | H) 1.57 (q, J = 11.05 Hz, 1 H) 1.45-1.51 (m, 1 H) | | |
| | 1.27-1.35 (m, 2 H) 1.23 (d, J = 6.16 Hz, 3 H) 0.81 | | |
| | (dd, J = 6.59, 3.17 Hz, 6 H) | | |
| 104 | 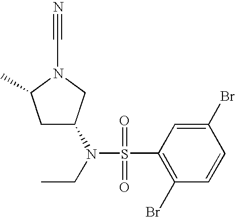 | 2.12 | 448.8 |
| | 2,5-Dibromo-N-[(3R,5S)-1-cyano-5-methyl-3- | | |
| | pyrrolidinyl]-N-ethylbenzenesulfonamide | | |
| | 1 H NMR (500 MHz, DMSO-d₆) δ ppm 8.10 (d, | | |
| | J = 2.26 Hz, 1 H) 7.76-7.82 (m, 2 H) 4.42-4.48 | | |
| | (m, 1 H) 3.56-3.61 (m, 1 H) 3.37-3.43 (m, 2 H) | | |
| | 3.34 (m, 2 H) 2.05 (d, J = 6.29 Hz, 1 H) 1.60 (q, | | |
| | J = 11.05 Hz, 1 H) 1.23 (d, J = 6.16 Hz, 3 H) 1.05 (t, | | |
| | J = 7.05 Hz, 3 H) | | |
| 105 | 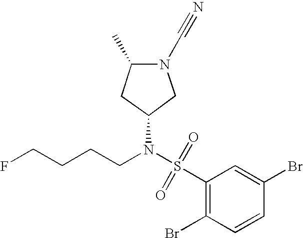 | 2.20 | 494.9 |
| | 2,5-Dibromo-N-[(3R,5S)-1-cyano-5-methyl-3- | | |
| | pyrrolidinyl]-N-(4-fluorobutyl)benzenesulfonamide | | |
| | 1 H NMR (500 MHz, DMSO-d₆) δ ppm 8.10 (d, | | |
| | J = 2.20 Hz, 1 H) 7.77-7.83 (m, 1 H) 4.43 (m, 2 H) | | |
| | 4.31-4.36 (m, 1 H) 3.58 (m, 1 H) 3.33 (m, 4 H) | | |
| | 2.03 (m, 1 H) 1.58-1.63 (m, 2 H) 1.54 (m, 3 H) | | |
| | 1.22 (d, J = 6.16 Hz, 3 H) | | |
| 106 | 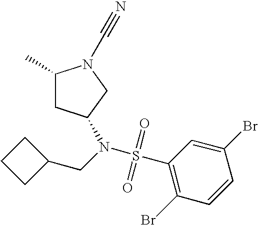 | 2.40 | 488.9 |
| | 2,5-Dibromo-N-[(3R,5S)-1-cyano-5-methyl-3- | | |
| | pyrrolidinyl]-N-(cyclobutylmethyl)- | | |
| | benzenesulfonamide | | |
| | 1 H NMR (500 MHz, DMSO-d₆) δ ppm 8.07 (d, | | |
| | J = 2.20 Hz, 1 H) 7.76-7.82 (m, 2 H) 4.40 (m, 1 H) | | |
| | 3.58 (m, 1 H) 3.35-3.41 (m, 4 H) 2.40 (m, 1 H) | | |
| | 2.03 (m, 2 H) 1.83 (m, 2 H) 1.70 (m, 2 H) 1.59 (m, | | |
| | 2 H) 1.23 (d, J = 6.16 Hz, 3 H) | | |
| 107 | 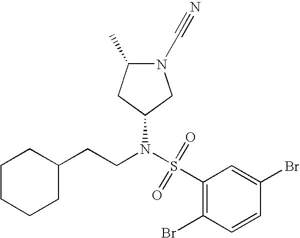 | 2.76 | 531 |
| | 2,5-Dibromo-N-[(3R,5S)-1-cyano-5-methyl-3- | | |
| | pyrrolidinyl]-N-(2-cyclohexylethyl)- | | |
| | benzenesulfonamide | | |
| | 1 H NMR (500 MHz, DMSO-d₆) δ ppm 8.09 (d, | | |
| | J = 2.20 Hz, 1 H) 7.77-7.83 (m, 2 H) 4.46 (m, 1 H) | | |
| | 3.57 (m, 1 H) 3.31-3.35 (m, 4 H) 2.06 (m, 1 H) | | |
| | 1.58 (m, 6 H) 1.30 (m, 2 H) 1.22 (d, J = 6.16 Hz, 3 | | |
| | H) 1.11 (m, 4 H) 0.82 (m, 2 H) | | |
| 108 | 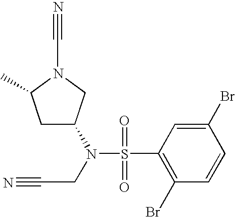 | 1.93 | 459.7 |
| | 2,5-Dibromo-N-(cyanomethyl)-N-[(3R,5S)-1- | | |
| | cyano-5-methyl-3-pyrrolidinyl]benzenesulfonamide | | |
| | 1 H NMR (500 MHz, DMSO-d₆) δ ppm 8.17 (s, 1 H) | | |
| | 7.84 (d, J = 0.73 Hz, 2 H) 4.64 (s, 2 H) 4.54-4.61 | | |
| | (m, 1 H) 3.59-3.64 (m, 1 H) 3.33-3.48 (m, 2 H) | | |
| | 2.07-2.13 (m, 1 H) 1.75 (q, J = 11.09 Hz, 1 H) 1.25 | | |
| | (d, J = 6.16 Hz, 3 H) | | |
| 109 | 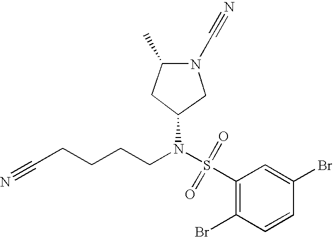 | 2.06 | 501.9 |
| | 2,5-Dibromo-N-(4-cyanobutyl)-N-[(3R,5S)-1- | | |
| | cyano-5-methyl-3-pyrrolidinyl]benzenesulfonamide | | |
| | 1 H NMR (500 MHz, DMSO-d₆) δ ppm 8.11 (d, | | |
| | J = 2.26 Hz, 1 H) 7.77-7.83 (m, 2 H) 4.39-4.44 | | |
| | (m, 1 H) 3.56 (m, 1 H) 3.26-3.32 (m, 6 H) 2.01 (m, | | |
| | 1 H) 1.51-1.57 (m, 5 H) 1.22 (d, J = 6.16 Hz, 3 H) | | |
| 110 | 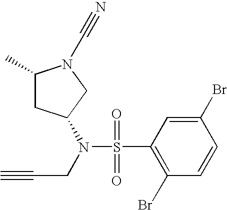 | 2.09 | 458.8 |
| | 2,5-Dibromo-N-[(3R,5S)-1-cyano-5-methyl-3- | | |
| | pyrrolidinyl]-N-2-propyn-1-ylbenzenesulfonamide | | |
| | 1 H NMR (500 MHz, DMSO-d₆) δ ppm 8.13 (d, | | |
| | J = 2.01 Hz, 1 H) 7.77-7.83 (m, 2 H) 4.50-4.56 | | |
| | (m, 1 H) 4.23-4.27 (m, 2 H) 3.57-3.63 (m, 1 H) | | |
| | 3.54 (t, J = 8.82 Hz, 1 H) 3.37 (t, J = 8.79 Hz, 1 H) | | |
| | 3.24 (t, 1 H) 2.05-2.10 (m, 1 H) 1.79 (dd, | | |
| | J = 22.71, 10.86 Hz, 1 H) 1.24 (d, J = 6.16 Hz, 3 H) | | |
| 111 | 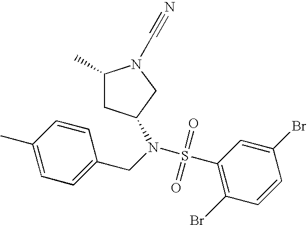 | 2.49 | 524.9 |
| | 2,5-Dibromo-N-[(3R,5S)-1-cyano-5-methyl-3- | | |
| | pyrrolidinyl]-N-[(4-methylphenyl)methyl]- | | |
| | benzenesulfonamide | | |
| | 1 H NMR (500 MHz, DMSO-d₆) δ ppm 7.78-7.81 | | |
| | (m, 3 H) 7.58 (d, J = 8.42 Hz, 1 H) 7.48 (d, J = 2.38 | | |
| | Hz, 3 H) 7.34-7.38 (m, 1 H) 4.82-4.87 (m, 1 H) | | |
| | 4.68 (dd, J = 25.64, 16.60 Hz, 2 H) 3.62-3.70 (m, 1 | | |
| | H) 3.59 (d, J = 8.24 Hz, 2 H) 2.26 (s, 3 H) 2.20- | | |
| | 2.24 (m, 1 H) 1.79 (dd, J = 22.46, 10.56 Hz, 1 H) | | |
| | 1.25 (d, J = 6.16 Hz, 3 H) | | |
| 112 | 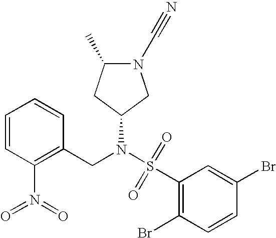 | 2.30 | 555.9 |
| | 2,5-Dibromo-N-[(3R,5S)-1-cyano-5-methyl-3- | | |
| | pyrrolidinyl]-N-[(2-nitrophenyl)methyl]- | | |
| | benzenesulfonamide | | |
| | 1 H NMR (500 MHz, DMSO-d₆) δ ppm 7.70-7.76 | | |
| | (m, 2 H) 7.65-7.66 (m, 1 H) 7.09 (d, J = 7.93 Hz, 2 | | |
| | H) 7.01 (d, J = 7.87 Hz, 2 H) 4.67-4.71 (m, 1 H) | | |
| | 4.54-4.58 (m, 2 H) 3.54-3.59 (m, 1 H) 3.33- | | |
| | 3.34 (m, 2 H) 2.22 (s, 3 H) 1.98-2.03 (m, 1 H) | | |
| | 1.60 (dd, J = 22.46, 10.80 Hz, 1 H) 1.16 (d, J = 6.16 | | |
| | Hz, 3 H) | | |
| 113 | 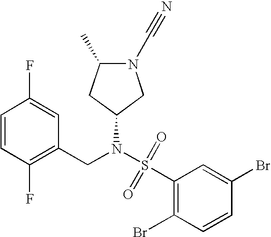 | 2.37 | 546.9 |
| | 2,5-Dibromo-N-[(3R,5S)-1-cyano-5-methyl-3- | | |
| | pyrrolidinyl]-N-[(2,5-difluorophenyl)methyl]- | | |
| | benzenesulfonamide | | |
| | 1 H NMR (500 MHz, DMSO-d₆) δ ppm 8.02-8.05 | | |
| | (m, 2 H) 7.79-7.84 (m, 1 H) 7.74-7.78 (m, 1 H) | | |
| | 7.66-7.73 (m, 1 H) 7.57-7.61 (m, 1 H) 7.47- | | |
| | 7.55 (m, 1 H) 4.99-5.16 (m, 2 H) 4.68-4.73 (m, 1 | | |
| | H) 3.52-3.58 (m, 1 H) 3.22-3.23 (m, 2 H) 1.86- | | |
| | 1.92 (m, 1 H) 1.50 (dd, J = 22.28, 11.35 Hz, 1 H) | | |
| | 1.13 (d, J = 6.16 Hz, 3 H) | | |
| 114 | 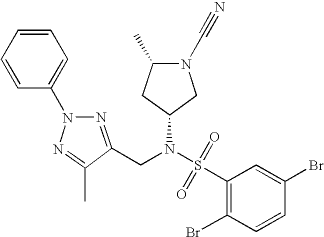 | 2.51 | 592 |
| | 2,5-Dibromo-N-[(3R,5S)-1-cyano-5-methyl-3- | | |
| | pyrrolidinyl]-N-[(5-methyl-2-phenyl-2H-1,2,3- | | |
| | triazol-4-yl)methyl]benzenesulfonamide | | |
| | 1 H NMR (500 MHz, DMSO-d₆) δ ppm 7.80-7.83 | | |
| | (m, 1 H) 7.74-7.77 (m, 1 H) 7.67-7.68 (m, 1 H) | | |
| | 7.07-7.11 (m, 2 H) 7.01-7.06 (m, 1 H) 4.76- | | |
| | 4.82 (m, 1 H) 4.65 (s, 2 H) 3.57-3.63 (m, 1 H) | | |
| | 3.35-3.46 (m, 2 H) 2.00-2.15 (m, 1 H) 1.62 (dd, | | |
| | J = 22.52, 10.80 Hz, 1 H) 1.19 (d, J = 6.16 Hz, 3 H) | | |

### EXAMPLE 115

### 2,5-Dibromo-N-[(3R,5S)-1-cyano-5-methyl-3-pyrrolidinyl]-N-cyclobutylbenzenesulfonamide

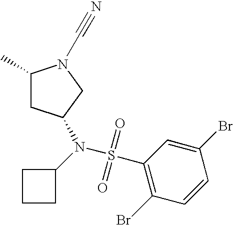

1,1-Dimethylethyl (2S,4R)-4-{[(2,5-dibromophenyl)sulfonyl]amino}-2-methyl-1-pyrrolidinecarboxylate (0.025 g, 0.050 mmol) was placed in a vial with potassium carbonate (0.104 g, 0.753 mmol) and bromocyclobutane (0.251 mmol) in acetone (2.0 mL) and DMF (0.7 mL) to give a clear solution containing a K₂CO₃ suspension. The reaction mixture was allowed to stir for 2.5 days. LC/MS showed the reaction was not complete. Sodium iodide (3 mg) was added and the mixture was heated at 75° C. for another 6 hr and then 120° C. overnight. A PS-triamine resin (0.147 g, 0.501 mmol, 3.41 mmol/g) was added to trap the excess alkyl bromide and the resulting mixture was stirred overnight. The resin was filtered off and the filtrate was concentrated to give a crude residue.

To the residue previously obtained in DCM (2 mL) was added a 4N HCL dioxane solution (0.5 mL, 2.0 mmol). The reaction mixture was stirred overnight. The solvent was evaporated to give a residue.

The residue previously obtained was taken up in DCM (3 mL), DIPEA (0.035 mL, 0.20 mmol) was added and the vial was shaken for 10 minutes. Then BrCN (0.033 mL, 3N in DCM, 0.1 mmol) was added to give a yellow solution. The reaction mixture was stirred for 2.5 days. LC/MS indicated the reaction was complete. A PS-triamine resin (0.059 g, 0.2 mmol, 3.41 mmol/g) was added and the flask was shaken for 2 hours to neutralize the excess BrCN. The resin was removed and the organic filtrate was concentrated to give a crude residue, which was purified by preparatory HPLC (without TFA) to give the title compound (4.14 mg). LC-MS: m/z, 474.8 (M+), rt 2.28 min; 1H NMR (500 MHz, DMSO-d₆) δ ppm 7.89-7.93 (m, 1H) 7.56-7.62 (m, 2H) 4.24-4.30 (m, 1H) 3.38-3.43 (m, 1H) 3.28-3.30 (m, 2H) 3.04-3.06 (m, 1H) 1.84-1.90 (m, 1H) 1.52 (dd, J=22.52, 11.17 Hz, 1H) 1.04 (d, J=6.23 Hz, 4H) 0.68-0.74 (m, 1H) 0.19-0.25 (m, 2H) −0.01-0.04 (m, 2H).

### EXAMPLE 116

### N-((3R,5R)-5-{[({[3,5-Bis(trifluoromethyl)phenyl]amino}-carbonyl)amino]methyl}-1-cyano-3-pyrrolidinyl)-2,5-dibromobenzenesulfonamide

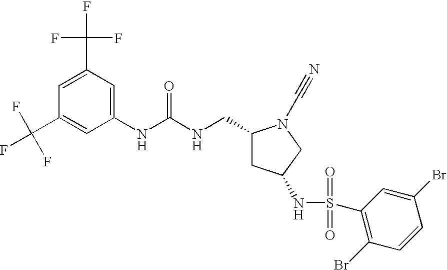

To a sample of 1-isocyanato-3,5-bis(trifluoromethyl)benzene 4.26 mg, 0.101 mmol) placed in a 5 mL vial was added 1,1-dimethylethyl (2R,4R)-2-(aminomethyl)-4-{[(2,5-dibromophenyl)sulfonyl]amino}-1-pyrrolidinecarboxylate (0.040 g, 0.078 mmol) in DCM (2.0 ml), giving a pale yellow solution. The reaction mixture was shaken overnight. LC/MS showed that the reaction was completed. A PS-Trisamine resin (0.047 g, 0.156 mmol) was added to trap the excess isocyanate. The mixture was shaken overnight and the PS-reagent was filtered off. The organic filtrate was collected and concentrated to give a crude residue.

To the crude residue previously obtained was added a 4N HCl dioxane solution (1.0 ml, 4.00 mmol) to give a pale yellow solution. The mixture was shaken overnight and then concentrated to give a crude mixture. To this mixture was added Et₃N (0.043 ml, 0.312 mmol) and DCM (2.0 ml) then the vial was shaken for 10 minutes. BrCN (0.052 ml, 0.156 mmol, 3N in DCM) was added and the mixture was shaken overnight. LC/MS showed the reaction was completed. A PS-Trisamine resin (0.093 g, 0.312 mmol, 3.34 mmol/g) was added to get rid of the excess BrCN. The mixture was concentrated to give a residue that was purified by preparatory HPLC (without TFA) to give the title compound (3.2 mg). LC-MS: m/z, 693 (M+), rt 2.39 min; 1H NMR (400 MHz, MeOD) δ ppm 8.26 (d, J=2.32 Hz, 1H) 8.03 (d, J=0.38 Hz, 2H) 7.72-7.76 (m, 1H) 7.67-7.70 (m, 1H) 7.52 (d, J=0.63 Hz, 1H) 3.91-3.98 (m, 1H) 3.75-3.83 (m, 1H) 3.65 (s, 2H) 3.56-3.60 (m, 2H) 2.16-2.23 (m, 1H) 1.64-1.71 (m, 1H)

The compounds shown in Table 2 were prepared according to the general procedure of Example 116, substituting the relevant isocyanate for the 1-isocyanato-3,5-bis(trifluoromethyl)benzene in Example 116.

TABLE 2

| | | Retention | LCMS: |
|---|---|---|---|
| | | Time | m/z, |
| Example | Structure | (min) | #(M+) |
| 117 | 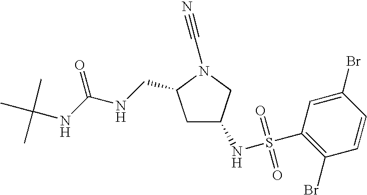 | 1.61 | 538 |
| | 2,5-Dibromo-N-{(3R,5R)-1-cyano-5-[({[(1,1- | | |
| | dimethylethyl)amino]carbonyl}amino)methyl]-3- | | |
| | pyrrolidinyl}benzenesulfonamide | | |
| | 1 H NMR (500 MHz, DMSO-d₆) δ ppm 8.06 (d, J = 2.26 | | |
| | Hz, 1 H) 7.73-7.79 (m, 2 H) 5.87 (br. s., 1 H) 5.73 (s, 2 | | |
| | H) 3.82-3.88 (m, 1 H) 3.51-3.57 (m, 1 H) 3.39-3.45 | | |
| | (m, 2 H) 3.11-3.17 (m, 1 H) 2.94-3.00 (m, 1 H) 1.91- | | |
| | 1.97 (m, 1 H) 1.49-1.54 (m, 1 H) 1.19 (s, 9 H) | | |
| 118 | 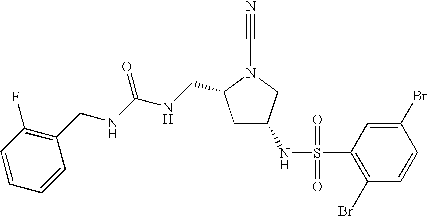 | 1.91 | 589 |
| | 2,5-Dibromo-N-((3R,5R)-1-cyano-5-{[({[(2- | | |
| | fluorophenyl)methyl]amino}carbonyl)amino]methyl}-3- | | |
| | pyrrolidinyl)benzenesulfonamide | | |
| | 1 H NMR (500 MHz, DMSO-d₆) δ ppm 8.06 (d, J = 2.32 | | |
| | Hz, 1 H) 7.69-7.84 (m, 2 H) 7.27-7.32 (m, 2 H) 7.10- | | |
| | 7.16 (m, 2 H) 6.43 (br. s., 2 H) 6.27 (br. s., 1 H) 4.21- | | |
| | 4.26 (m, 2 H) 3.82-3.88 (m, 1 H) 3.55-3.61 (m, 1 H) | | |
| | 3.38-3.43 (m, 2 H) 3.10-3.17 (m, 1 H) 3.01-3.09 (m, | | |
| | 1 H) 1.92-2.04 (m, 1 H) 1.47-1.58 (m, 1 H) | | |
| 119 | 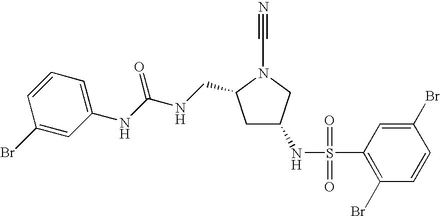 | 2.13 | 636 |
| | 2,5-Dibromo-N-{(3R,5R)-5-[({[(3- | | |
| | bromophenyl)amino]carbonyl}amino)methyl]-1-cyano-3- | | |
| | pyrrolidinyl}benzenesulfonamide | | |
| | 1 H NMR (400 MHz, MeOD) δ ppm 8.26 (d, J = 2.07 Hz, 1 | | |
| | H) 7.74 (dd, J = 8.16, 0.25 Hz, 2 H) 7.70 (d, J = 2.32 Hz, 1 | | |
| | H) 7.22-7.28 (m, 1 H) 7.10-7.20 (m, 2 H) 3.91-3.94 | | |
| | (m, 1 H) 3.73-3.80 (m, 1 H) 3.68 (s, 2 H) 3.50-3.56 | | |
| | (m, 2 H) 2.13-2.20 (m, 1 H) 1.63-1.74 (m, 1 H) | | |
| 120 | 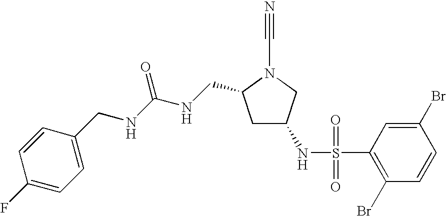 | 1.92 | 589 |
| | 2,5-Dibromo-N-((3R,5R)-1-cyano-5-{[({[(4- | | |
| | fluorophenyl)methyl]amino}carbonyl)amino]methyl}-3- | | |
| | pyrrolidinyl)benzenesulfonamide | | |
| | 1 H NMR (500 MHz, DMSO-d₆) δ ppm 8.06 (d, J = 2.26 | | |
| | Hz, 1 H) 7.69-7.83 (m, 2 H) 7.24-7.29 (m, 2 H) 7.04- | | |
| | 7.17 (m, 2 H) 6.43 (br. s., 2 H) 6.23 (br. s., 1 H) 4.15- | | |
| | 4.19 (m, 2 H) 3.78-3.92 (m, 1 H) 3.53-3.64 (m, 1 H) | | |
| | 3.28-3.34 (m, 2 H) 3.10-3.17 (m, 1 H) 3.01-3.09 (m, | | |
| | 1 H) 1.89-2.06 (m, 1 H) 1.43-1.59 (m, 1 H) | | |

### EXAMPLE 121

### 2,5-Dibromo-N-{(3R,5R)-1-cyano-5-[({[(3-cyanophenyl)amino]carbonyl}amino)methyl]-3-pyrrolidinyl}benzenesulfonamide

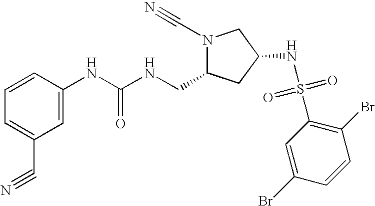

To a sample of 1-isocyanato-3,5-bis(trifluoromethyl)benzene 4.26 mg, 0.101 mmol) placed in a 5 mL vial was added 1,1-dimethylethyl (2R,4R)-2-(aminomethyl)-4-{[(2,5-dibromophenyl)sulfonyl]amino}-1-pyrrolidinecarboxylate (0.040 g, 0.078 mmol) in DCM (2.0 ml), giving a pale yellow solution. The reaction mixture was shaken overnight. LC/MS showed that the reaction was partially completed (30% product). The mixture was allowed to heat at 60° C. for 24 hours. LC/MS showed no additional product had formed. A PS-Trisamine resin (0.047 g, 0.156 mmol) was added to trap the excess isocyanate. The mixture was shaken overnight and the PS-reagent was filtered off. The organic filtrate was collected and concentrated to give a crude residue.

To the crude residue previously obtained was added a 4N HCl dioxane solution (1.0 ml, 4.00 mmol) to give a pale yellow solution. The mixture was shaken overnight and then concentrated to give a crude mixture. To this mixture was added Et₃N (0.043 ml, 0.312 mmol) and DCM (2.0 ml) then the vial was shaken for 10 minutes. BrCN (0.052 ml, 0.156 mmol, 3N in DCM) was added and the mixture was shaken overnight. LC/MS showed the reaction was completed. A PS-Trisamine resin (0.093 g, 0.312 mmol, 3.34 mmol/g) was added to get rid of the excess BrCN. The mixture was concentrated to give a residue that was purified by preparatory HPLC (without TFA) to give the title compound (1.3 mg). LC-MS: m/z, 583 (M+H), rt 0.95 min. 1H NMR (400 MHz, MeOD) δ ppm 8.26 (d, J=2.32 Hz, 1H) 7.91 (ddd, J=1.76, 1.07, 0.94 Hz, 1H) 7.74-7.78 (m, 1H) 7.66-7.71 (m, 1H) 7.58 (ddd, J=8.30, 2.24, 1.13 Hz, 1H) 7.44 (dd, J=15.94, 0.44 Hz, 1H) 7.33 (ddd, J=7.80, 1.30, 1.13 Hz, 1H) 3.91-3.98 (m, 1H) 3.73-3.82 (m, 1H) 3.68 (s, 2H) 3.50-3.54 (m, 2H) 2.14-2.22 (m, 1H) 1.67-1.73 (m, 1H).

### EXAMPLE 122

### 2,5-Dibromo-N-{(3R,5R)-1-cyano-5-[({[(4′-fluoro-3-biphenylyl)amino]carbonyl}amino)methyl]-3-pyrrolidinyl}benzenesulfonamide

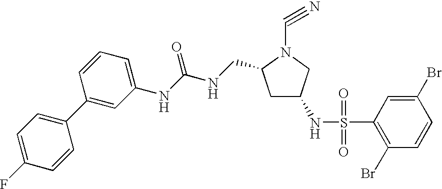

To a 4 mL vial containing 4′-fluoro-3-biphenylcarboxylic acid (9.00 mg, 0.2 mmol) were added Et₃N (0.033 ml, 0.240 mmol), toluene (1.0 ml) and then DPPA (0.051 ml, 0.240 mmol). The mixture was heated at 110° C. for 2 hr. After cooling down, a solution of 1,1-dimethylethyl (2R,4R)-2-(aminomethyl)-4-{[(2,5-dibromophenyl)sulfonyl]amino}-1-pyrrolidinecarboxylate (0.041 g, 0.080 mmol) in THF (1.0 ml) was added. The mixture was shaken overnight. LC/MS showed the reaction was completed. A PS-Trisamine resin (0.180 g, 0.600 mmol, 3.34 mmol/g) was added to trap the potential excess isocyanate. The mixture was shaken overnight. The polymer-supported reagent was filtered off, the filtrate was collected, concentrated to give a residue that was purified by preparatory HPLC (without TFA).

To the product previously obtained was added a HCl dioxane solution (1.0 ml, 3.00 mmol) to give a pale yellow solution. The mixture was shaken for 3 hr and then concentrated to give a crude mixture. To this mixture was added Et₃N (0.045 ml, 0.320 mmol) and DCM (2.0 ml) then the vial was shaken for 10 minutes. BrCN (0.053 ml, 0.160 mmol, 3N in DCM) was added and the mixture was shaken overnight. A PS-Trisamine resin (0.096 g, 0.320 mmol, 3.34 mmol/g) was added to get rid of the excess BrCN. The mixture was shaken overnight, the resin was removed and the solution was purified by preparatory HPLC (without TFA) to give the title compound (7.48 mg). LC-MS: m/z, 653 (M+), rt 1.92 min. 1H NMR (400 MHz, MeOD) δ ppm 8.26 (d, J=2.26 Hz, 1H) 7.60-7.72 (m, 5H) 7.29-7.35 (m, 2H) 7.22-7.26 (m, 1H) 7.18 (t, J=8.78 Hz, 1H) 7.14-7.21 (m, 1H) 3.93-3.99 (m, 1H) 3.74-3.82 (m, 1H) 3.52-3.59 (m, 2H) 3.38-3.42 (m, 2H) 2.18 (m, 1H) 1.68-1.75 (m, 1H)

The compounds shown in Table 3 were prepared according to the general procedure of Example 122, substituting the relevant carboxylic acid for the 4′-fluoro-3-biphenylcarboxylic acid in Example 122.

TABLE 3

| | | Retention | LCMS: |
|---|---|---|---|
| | | Time | m/z, |
| Example | Structure | (min) | #(M+) |
| 123 | 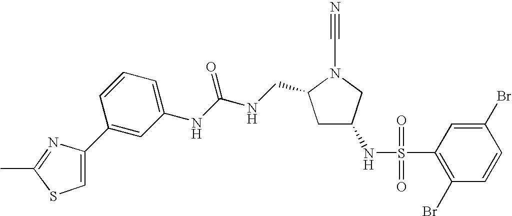 | 2.25 | 653 |
| | 2,5-Dibromo-N-((3R,5R)-1-cyano-5-{[({[3-(2-methyl-1,3- | | |
| | thiazol-4-yl)phenyl]amino}carbonyl)amino]methyl}-3- | | |
| | pyrrolidinyl)benzenesulfonamide | | |
| | 1 H NMR (400 MHz, MeOD) δ ppm 8.25 (d, J = 2.26 Hz, 1 | | |
| | H) 7.90-7.97 (m, 1 H) 7.70-7.74 (m, 1 H) 7.65-7.69 | | |
| | (m, 1 H) 7.60 (s, 1 H) 7.53 (dt, J = 7.03, 1.76 Hz, 1 H) | | |
| | 7.31-7.38 (m, 2 H) 3.92-3.99 (m, 1 H) 3.74-3.81 (m, | | |
| | 1 H) 3.55 (ddd, J = 9.60, 2.45, 2.26 Hz, 2 H) 3.40-3.42 | | |
| | (m, 2 H) 2.77 (s, 3 H) 2.17 (ddd, J = 13.11, 7.03, 6.71 Hz, | | |
| | 1 H) 1.69-1.75 (m, 1 H) | | |
| 124 | 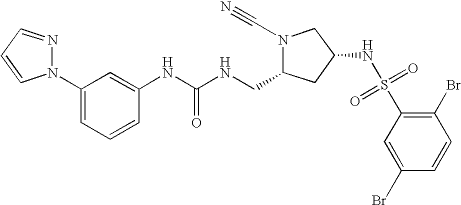 | 1.70 | 624 |
| | 2,5-Dibromo-N-((3R,5R)-1-cyano-5-{[({[3-(1H-pyrazol-1- | | |
| | yl)phenyl]amino}carbonyl)amino]methyl}-3- | | |
| | pyrrolidinyl)benzenesulfonamide | | |
| | 1 H NMR (400 MHz, MeOD) δ ppm 8.26 (d, J = 2.26 Hz, 1 | | |
| | H) 8.19 (d, J = 1.76 Hz, 1 H) 7.92 (t, J = 1.76 Hz, 1 H) 7.71- | | |
| | 7.75 (m, 2 H) 7.65-7.69 (m, 1 H) 7.34-7.41 (m, 2 H) | | |
| | 7.29 (t, J = 1.88 Hz, 1 H) 6.54 (d, J = 1.76 Hz, 1 H) 3.95 | | |
| | (dd, J = 8.78, 7.03 Hz, 1 H) 3.74-3.82 (m, 1 H) 3.52- | | |
| | 3.57 (m, 2 H) 3.39-3.42 (m, 2 H) 2.18 (ddd, J = 13.05, | | |
| | 6.90, 6.65 Hz, 1 H) 1.68-1.75 (m, 1 H) | | |
| 125 | 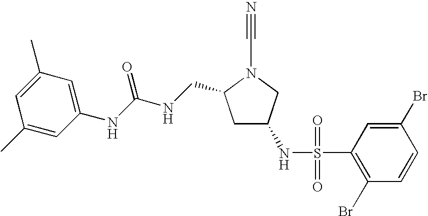 | 2.34 | 586 |
| | 2,5-Dibromo-N-{(3R,5R)-1-cyano-5-[({[(3,5- | | |
| | dimethylphenyl)amino]carbonyl}amino)methyl]-3- | | |
| | pyrrolidinyl}benzenesulfonamide | | |
| | 1 H NMR (400 MHz, MeOD) δ ppm 8.26 (d, J = 2.51 Hz, 1 | | |
| | H) 7.71-7.80 (m, 1 H) 7.63-7.71 (m, 1 H) 7.00 (s, 2 H) | | |
| | 6.67 (s, 1 H) 3.87-4.00 (m, 1 H) 3.69-3.82 (m, 1 H) | | |
| | 3.47-3.62 (m, 2 H) 3.36-3.40 (m, 2 H) 2.27 (s, 6 H) | | |
| | 2.16 (ddd, J = 13.11, 7.03, 6.71 Hz, 1 H) 1.69 (ddd, | | |
| | J = 12.92, 8.66, 8.53 Hz, 1 H) | | |

### EXAMPLE 126

### 2,5-Dibromo-N-((3R,5R)-1-cyano-5-{[methyl(4-pyridinylmethyl)amino]methyl}-3-pyrrolidinyl)benzenesulfonamide

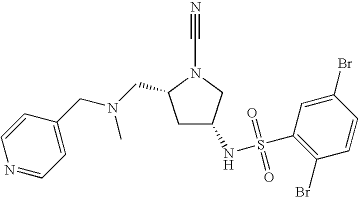

To a vial containing N-methyl-1-(4-pyridinyl)methanamine (0.600 mmol) was added 1,1-dimethylethyl (2R,4R)-4-{[(2,5-dibromophenyl)sulfonyl]amino}-2-formyl-1-pyrrolidine-carboxylate (0.061 g, 0.12 mmol) in DCM (2.0 ml), methanol (1.0 ml) and 2 drops of acetic acid. The mixture was shaken for 10 minutes before the addition of a PS-(OAc)₃BH resin (0.258 g, 0.600 mmol, 2.33 mmol/g). The mixture was shaken overnight. LC-MS indicated the consumption of all the starting material. The PS-reagent was then filtered off and the organic layer was collected, concentrated and the residue was purified by preparatory HPLC (without TFA) to give a secondary amine intermediate.

To the product previously prepared was added a HCl dioxane solution (1.0 ml, 4.00 mmol) to give a pale yellow solution. The mixture was shaken for 3 hr and then concentrated to give a crude mixture. To this mixture was added Et₃N (0.084 ml, 0.600 mmol) and DCM (2.0 ml) then the vial was shaken for 10 minutes. BrCN (0.080 ml, 0.240 mmol, 3N in DCM) was added and the mixture was shaken overnight. A PS-Trisamine resin (0.144 g, 0.480 mmol, 3.34 mmol/g) was added to get rid of the excess BrCN. The mixture was shaken overnight, the resin was removed and the solution was purified by preparatory HPLC (without TFA) to give the title compound (8.69 mg). LC-MS: m/z, 544 (M+), rt 1.30 min. 1H NMR (400 MHz, MeOD) δ ppm 8.47 (s, 1H) 8.44-8.51 (m, 1H) 8.26 (d, J=2.26 Hz, 1H) 7.74-7.78 (m, 1H) 7.68-7.72 (m, 1H) 7.50 (d, J=6.02 Hz, 2H) 3.98 (dt, J=8.66, 7.15 Hz, 1H) 3.80-3.87 (m, J=7.65, 7.65, 7.53, 4.77 Hz, 1H) 3.64 (dd, J=34.63, 14.56 Hz, 2H) 3.52 (dd, J=9.79, 7.28 Hz, 1H) 3.27-3.29 (m, 1H) 2.72 (dd, J=13.05, 7.53 Hz, 1H) 2.56 (dd, J=13.05, 4.77 Hz, 1H) 2.25 (s, 3H) 2.13-2.21 (m, 1H) 1.57-1.63 (m, 1H)

The compounds shown in Table 4 were prepared according to the general procedure of Example 126, substituting the relevant amine for N-methyl-1-(4-pyridinyl)methanamine.

TABLE 4

| | | Retention | LCMS: |
|---|---|---|---|
| | | Time | m/z, |
| Example | structure | (min) | #(M + H) |
| 127 | 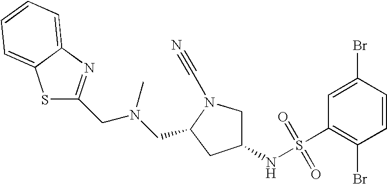 | 1.82 | 600 |
| | N-((3R,5R)-5-{[(1,3-Benzothiazol-2- | | |
| | ylmethyl)(methyl)amino]methyl}-1-cyano-3-pyrrolidinyl)- | | |
| | 2,5-dibromobenzenesulfonamide | | |
| | 1 H NMR (400 MHz, MeOD) δ ppm 8.26 (d, J = 2.26 Hz, | | |
| | 1 H) 8.00 (d, J = 7.28 Hz, 1 H) 7.93 (d, J = 7.53 Hz, 1 H) | | |
| | 7.71-7.76 (m, 1 H) 7.64-7.70 (m, 1 H) 7.52 (td, | | |
| | J = 7.72, 1.38 Hz, 1 H) 7.42-7.48 (m, 1 H) 4.05 (s, 2 H) | | |
| | 3.96-4.03 (m, 1 H) 3.86 (dt, J = 13.99, 6.93 Hz, 1 H) | | |
| | 3.52-3.57 (m, 1 H) 3.33 (m, 1H) 2.87 (dd, J = 13.05, | | |
| | 6.53 Hz, 1 H) 2.71 (dd, J = 13.05, 5.77 Hz, 1 H) 2.44 (s, | | |
| | 3 H) 2.25 (ddd, J = 13.11, 7.03, 6.71 Hz, 1 H) 1.69 (dt, | | |
| | J = 12.80, 8.53 Hz, 1 H) | | |
| 128 | 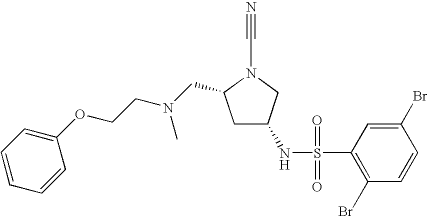 | 1.57 | 573 |
| | 2,5-Dibromo-N-[(3R,5R)-1-cyano-5-({methyl[2- | | |
| | (phenyloxy)ethyl]amino}methyl)-3- | | |
| | pyrrolidinyl]benzenesulfonamide | | |
| | 1 H NMR (400 MHz, MeOD) δ ppm 8.25 (d, J = 2.26 Hz, | | |
| | 1 H) 7.70-7.75 (m, 1 H) 7.65-7.69 (m, 1 H) 7.27 (dd, | | |
| | J = 8.78, 7.53 Hz, 2 H) 6.89-6.96 (m, 3 H) 4.13 (t, | | |
| | J = 5.65 Hz, 2 H) 3.95 (dq, J = 6.78, 6.53 Hz, 1 H) 3.79 | | |
| | (dd, J = 12.80, 1.76 Hz, 1 H) 3.50 (dd, J = 9.79, 6.53 Hz, 1 | | |
| | H) 3.28-3.31 (m, 1 H) 2.92 (td, J = 5.58, 2.64 Hz, 2 H) | | |
| | 2.77-2.83 (m, 2 H) 2.46 (s, 3 H) 2.23-2.30 (m, 1 H) | | |
| | 1.71 (ddd, J = 13.30, 7.03, 6.78 Hz, 1 H) | | |
| 129 | 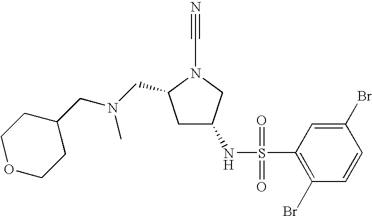 | 1.28 | 551 |
| | 2,5-Dibromo-N-((3R,5R)-1-cyano-5-{[methyl(tetrahydro- | | |
| | 2H-pyran-4-ylmethyl)amino]methyl}-3- | | |
| | pyrrolidinyl)benzenesulfonamide | | |
| | 1 H NMR (400 MHz, MeOD) δ ppm 8.26 (d, J = 2.26 Hz, | | |
| | 1 H) 7.74-7.79 (m, 1 H) 7.68-7.73 (m, 1 H) 3.91- | | |
| | 3.97 (m, 3 H) 3.73 (dt, J = 13.55, 6.78 Hz, 1 H) 3.49- | | |
| | 3.55 (m, 1 H) 3.39-3.46 (m, 2 H) 3.24-3.29 (m, 1 H) | | |
| | 2.54-2.60 (m, 1 H) 2.44-2.48 (m, 1 H) 2.21-2.27 (m, | | |
| | 6 H) 1.70-1.77 (m, 3 H) 1.61-1.67 (m, 1 H) 1.18- | | |
| | 1.24 (m, 2 H) | | |
| 130 | 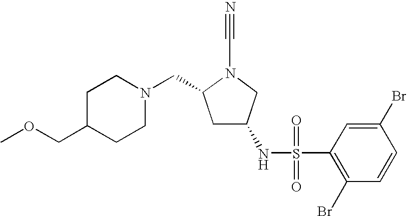 | 1.35 | 551 |
| | 2,5-Dibromo-N-[(3R,5R)-1-cyano-5-({4- | | |
| | [(methyloxy)methyl]-1-piperidinyl}methyl)-3- | | |
| | pyrrolidinyl]benzenesulfonamide | | |
| | 1 H NMR (400 MHz, MeOD) δ ppm 8.27 (d, J = 2.26 Hz, | | |
| | 1 H) 7.75-7.80 (m, 1 H) 7.67-7.74 (m, 1 H) 3.92 (ddd, | | |
| | J = 11.17, 5.77, 5.65 Hz, 1 H) 3.78-3.85 (m, 1 H) 3.48- | | |
| | 3.55 (m, 1 H) 3.37-3.42 (m, 1 H) 3.33 (s, 3 H) 3.24 (m, | | |
| | 2 H) 3.09 (d, J = 10.79 Hz, 1 H) 2.91 (d, J = 11.04 Hz, 1 | | |
| | H) 2.56-2.62 (m, 2 H) 2.23-2.24 (m, 3 H) 1.66-1.73 | | |
| | (m, 3 H) 1.54-1.64 (m, 1 H) 1.39-1.46 (m, 2 H) | | |
| 131 | 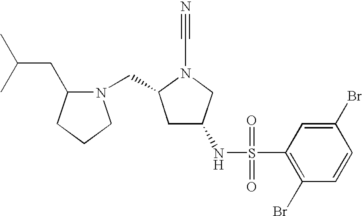 | 1.64 | 549 |
| | 2,5-Dibromo-N-((3R,5R)-1-cyano-5-{[2-(2- | | |
| | methylpropyl)-1-pyrrolidinyl]methyl}-3- | | |
| | pyrrolidinyl)benzenesulfonamide | | |
| | 1 H NMR (400 MHz, MeOD) δ ppm 8.25 (d, J = 2.51 Hz, | | |
| | 1 H) 7.74-7.78 (m, 1 H) 7.67-7.71 (m, 1 H) 3.95 (m, 1 | | |
| | H) 3.90 (m, 1 H) 3.50-3.55 (m, 1 H) 3.42-3.49 (m, 2 | | |
| | H) 2.92-2.95 (m, 1 H) 2.62 (dd, J = 14.31, 3.26 Hz, 1 H) | | |
| | 2.48-2.55 (m, 1 H) 2.36-2.44 (m, 2 H) 1.99 (m, 2 H) | | |
| | 1.78-1.82 (m, 1 H) 1.70 (m, 1 H) 1.57 (m, 1 H) 1.40 | | |
| | (m, 2 H) 1.19-1.33 (m, 1 H) 0.88-0.95 (m, 6 H) | | |
| 132 | 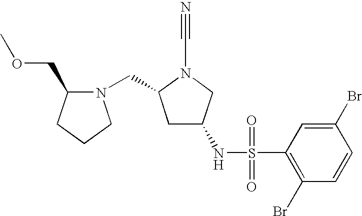 | 1.36 | 537 |
| | 2,5-Dibromo-N-[(3R,5R)-1-cyano-5-({(2S)-2- | | |
| | [(methyloxy)methyl]-1-pyrrolidinyl}methyl)-3- | | |
| | pyrrolidinyl]benzenesulfonamide | | |
| | 1 H NMR (400 MHz, MeOD) δ ppm 8.25 (d, J = 2.51 Hz, | | |
| | 1 H) 7.74-7.84 (m, 1 H) 7.62-7.69 (m, 1 H) 3.96- | | |
| | 4.02 (m, J = 5.90, 5.90, 4.02, 3.76 Hz, 1 H) 3.84 (dd, | | |
| | J = 8.78, 4.27 Hz, 1 H) 3.48-3.53 (m, 1 H) 3.36-3.43 | | |
| | (m, 1 H) 3.36-3.43 (m, 3 H) 3.29 (s, 3 H) 3.11 -3.18 | | |
| | (m, 1 H) 2.79-2.83 (m, 1 H) 2.71 (dd, J = 14.18, 3.39 | | |
| | Hz, 1 H) 2.41-2.47 (m, 1 H) 2.35 (td, J = 6.71, 2.38 Hz, | | |
| | 1 H) 1.86-1.93 (m, 3 H) 1.77-1.85 (m, 1 H) 1.48- | | |
| | 1.52 (m, 1 H) | | |
| 133 | 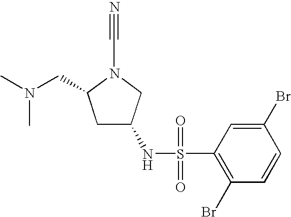 | 1.16 | 467 |
| | 2,5-Dibromo-N-{(3R,5R)-1-cyano-5- | | |
| | [(dimethylamino)methyl]-3- | | |
| | pyrrolidinyl}benzenesulfonamide | | |
| | 1 H NMR (400 MHz, MeOD) δ ppm 8.26 (d, J = 2.26 Hz, | | |
| | 1 H) 7.74-7.79 (m, 1 H) 7.67-7.73 (m, 1 H) 3.88- | | |
| | 3.95 (m, 1 H) 3.75-3.82 (m, J = 6.53, 6.53, 6.40, 5.14 | | |
| | Hz, 1 H) 3.50 (dd, J = 9.79, 6.27 Hz, 1 H) 3.32 (m, 1 H) | | |
| | 2.55-2.62 (m, 2 H) 2.34 (s, 6 H) 2.29 (dd, J = 7.40, 5.90 | | |
| | Hz, 1 H) 1.69 (dt, J = 12.99, 6.43 Hz, 1 H) | | |
| 134 | 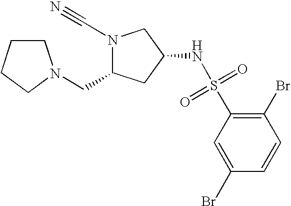 | 1.22 | 493 |
| | 2,5-Dibromo-N-[(3R,5R)-1-cyano-5-(1- | | |
| | pyrrolidinylmethyl)-3-pyrrolidinyl]benzenesulfonamide | | |
| | 1 H NMR (400 MHz, MeOD) δ ppm 8.26 (d, J = 2.26 Hz, | | |
| | 1 H) 7.75-7.82 (m, 1 H) 7.70 (d, J = 2.51 Hz, 1 H) 3.81- | | |
| | 3.88 (m, 2 H) 3.52 (dd, J = 9.79, 5.52 Hz, 1 H) 3.41- | | |
| | 3.44 (m, 1 H) 2.79-2.87 (m, 3 H) 2.76 (d, J = 4.52 Hz, 1 | | |
| | H) 2.66-2.73 (m, 2 H) 2.34 (td, J = 6.65, 2.26 Hz, 1 H) | | |
| | 1.87 (ddd, J = 6.59, 3.20, 3.01 Hz, 4 H) 1.75 (ddd, | | |
| | J = 9.47, 8.47, 4.39 Hz, 1 H) | | |
| 135 | 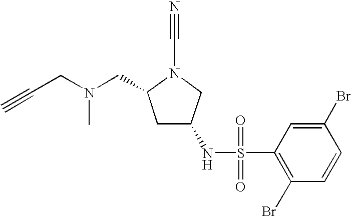 | 1.23 | 491 |
| | 2,5-Dibromo-N-((3R,5R)-1-cyano-5-{[methyl(2-propyn- | | |
| | 1-yl)amino]methyl}-3-pyrrolidinyl)benzenesulfonamide | | |
| | 1 H NMR (400 MHz, MeOD) δ ppm 8.26 (d, J = 2.26 Hz, | | |
| | 1 H) 7.74-7.79 (m, 1 H) 7.67-7.72 (m, 1 H) 3.93 (dq, | | |
| | J = 6.78, 6.61 Hz, 1 H) 3.73-3.80 (m, 1 H) 3.47-3.54 | | |
| | (m, 1 H) 3.41 (d, J = 2.51 Hz, 2 H) 3.30 (m, 1 H) 2.64- | | |
| | 2.71 (m, 3 H) 2.40 (s, 3 H) 2.21-2.29 (m, 1 H) 1.70 | | |
| | (ddd, J = 13.36, 7.15, 6.84 Hz, 1 H) | | |
| 136 | 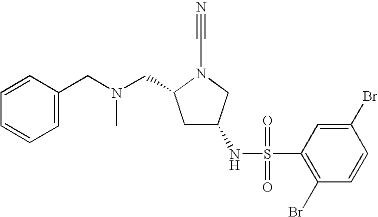 | 1.46 | 543 |
| | 2,5-Dibromo-N-((3R,5R)-1-cyano-5- | | |
| | {[methyl(phenylmethyl)amino]methyl}-3- | | |
| | pyrrolidinyl)benzenesulfonamide | | |
| | 1 H NMR (400 MHz, MeOD) δ ppm 8.25 (d, J = 2.51 Hz, | | |
| | 1 H) 7.74-7.79 (m, 1 H) 7.71 (d, J = 2.51 Hz, 1 H) 7.35 | | |
| | (s, 1 H) 7.37 (d, J = l.76 Hz, 1 H) 7.30 (t, J = 7.15 Hz, 1 | | |
| | H) 7.28-7.33 (m, 2 H) 3.93-4.00 (m, 1 H) 3.75-3.83 | | |
| | (m, 1 H) 3.57 (dd, J = 32.88, 13.05 Hz, 2 H) 3.47-3.55 | | |
| | (m, 1 H) 3.26 (dd, J = 9.79, 6.78 Hz, 1 H) 2.65-2.68 (m, | | |
| | 1 H) 2.48-2.52 (m, 1 H) 2.26 (s, 3 H) 2.14-2.22 (m, 1 | | |
| | H) 1.54-1.60 (m, 1 H) | | |

### EXAMPLE 137

### 2,5-Dibromo-N-[(3R,5R)-1-cyano-5-(4-morpholinylmethyl)-3-pyrrolidinyl]benzenesulfonamide

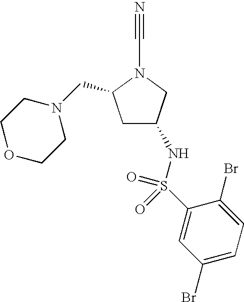

To a vial containing 1,1-dimethylethyl (2R,4R)-4-{[(2,5-dibromophenyl)sulfonyl]amino}-2-formyl-1-pyrrolidinecarboxylate (0.10 mmol) in DCM (2.0 ml) and methanol (1.0 ml) was added morpholine (0.035 g, 0.40 mmol) and 2 drops of acetic acid. The mixture was shaken for 5 minutes before the addition of a PS-(OAc)₃BH resin (0.5 mmol). The mixture was shaken overnight. LC-MS indicated that the composition of the mixture was 45% of the title compound and 55% of an alcohol by-product. The PS-reagent was filtered off and the organic layer was collected, concentrated and the residue was purified by preparatory HPLC (without TFA). All the fractions containing the title compound were collected and concentrated to give a crude material.

To the material previously prepared was added a 4M HCl dioxane solution (1.0 ml, 4.00 mmol). The mixture was shaken overnight. LC/MS indicated that the reaction was completed. The solution was concentrated to give a crude residue which was dissolved in DCM (2 mL). Then DIPEA (0.070 mL, 0.4 mmol) was added. The vial was shaken for 10 minutes, then BrCN (0.067 ml, 0.200 mmol, 3N in DCM) was added. The mixture was shaken for 3 hr. Then a PS-Trisamine resin (0.117 g, 0.400 mmol, 3.41 mmol/g) was added to get rid of the excess BrCN. The resin was filtered off and the filtrate was concentrated to give a residue that was purified by preparatory HPLC (without TFA) to give the title compound (5.5 mg). LC-MS: m/z, 508.9 (M+), rt 1.24 min. 1H NMR (400 MHz, MeOD) δ ppm 8.27 (d, J=2.26 Hz, 1H) 7.75-7.79 (m, 1H) 7.72 (d, J=2.26 Hz, 1H) 3.92 (dq, J=6.78, 6.53 Hz, 1H) 3.82 (t, J=5.65 Hz, 1H) 3.70-3.77 (m, 4H) 3.51 (dd, J=9.66, 6.65 Hz, 1H) 3.31 (m, 1H) 2.57-2.60 (m, 6H) 2.20-2.28 (m, 1H) 1.67 (ddd, J=13.49, 7.03, 6.84 Hz, 1H)

### EXAMPLE 138

### 2,5-Dibromo-N-{(3R,5R)-1-cyano-5-[(ethyloxy)methyl]-3-pyrrolidinyl}benzenesulfonamide

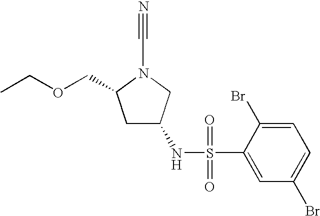

A flask was loaded with 1,1-dimethylethyl (2R,4R)-4-{[(2,5-dibromophenyl)sulfonyl]amino}-2-[(ethyloxy)methyl]-1-pyrrolidinecarboxylate (0.144 g, 0.266 mmol) and a 4M HCl dioxane solution (1.328 ml, 5.31 mmol). The mixture was stirred at room temperature for 3 hours, and then concentrated under vacuum to give crude orange oil. A flask was loaded with this material and DIPEA (0.139 mL, 0.798 mmol) in DCM (1.5 mL) to give an orange suspension. The pH was found to be 10.5. BrCN (0.177 ml, 0.532 mmol, 3N in DCM) was added and the resulting mixture was stirred at room temperature for 1.5 hr. A PS-Trisamine resin (0.296 g, 1.064 mmol, 3.6 mmol/g) was then added and the resulting mixture was stirred for another 1.5 hr. The resin was filtered off and the filtrate was concentrated to give a crude residue which was purified by preparatory HPLC (AcCN/water without TFA) to give the title compound (63.4 mg). LC-MS: m/z, 468 (M+H), rt 1.93 min. 1H NMR (400 MHz, CHLOROFORM-d) δ ppm 8.18 (1H, d, J=2.26 Hz) 7.52-7.57 (1H, m) 7.47 (1H, dd, J=8.53, 2.51 Hz) 7.18 (1H, d, J=9.29 Hz) 5.24 (1H, s) 3.82-3.90 (1H, m, J=6.84, 4.45, 4.27, 4.27 Hz) 3.77 (1H, dq, J=10.20, 2.12 Hz) 3.71 (1H, dd, J=10.79, 2.26 Hz) 3.56-3.67 (2H, m) 3.42 (1H, dd, J=10.79, 2.01 Hz) 3.36 (1H, d, J=4.77 Hz) 3.33 (1H, d, J=5.02 Hz) 3.23 (1H, dt) 2.27 (1H, ddd, J=14.05, 10.29, 7.03 Hz) 1.85 (1H, dd, J=14.05, 2.01 Hz) 1.28 (3H, t, J=7.03 Hz)

### EXAMPLE 139

### 2-Chloro-N-[(3R,5R)-1-cyano-5-(1-methylethyl)-3-pyrrolidinyl]-5-(trifluoromethyl)benzenesulfonamide

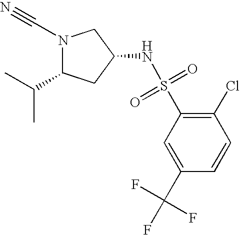

To 1,1-dimethylethyl (2R,4R)-4-({[(9H-fluoren-9-ylmethyl)oxy]carbonyl}amino)-2-(1-methylethyl)-1-pyrrolidinecarboxylate (121.5 mg, 0.27 mmol, 1 eq) in DCM (3 mL) is added a MP-trisamine resin (3 eq). After stirring overnight, the polymer is filtered off; the filtrate is recovered and dispensed into a vial containing 2-chloro-5-(trifluoromethyl)benzenesulfonyl chloride (83.7 mg, 0.30 mmol) after which Et₃N (0.109 g, 1.080 mmol) is added. The reaction mixture is shaken overnight. After LC/MS indicates the reaction is completed, 1.0 ml of water is added into the reaction mixture which is stirred for another 3 hr. The organic solution is separated using an hydrophobic separator. The organic solvent is evaporated and the residue obtained is dissolved in 1.0 mL of a 4.0M HCl in dioxane. After shaking overnight, the reaction mixture is evaporated to give a crude residue which is dissolved in DCM (2.0 mL). DIPEA (0.187 mL, 1.08 mmol) is added. The reaction mixture is shaken for 3 hr then CNBr (0.180 mL, 0.54 mmol) is added. After the CNBr is added, the mixture is shaken overnight until LC-MS indicates that the reaction is complete. A MP-Triamine resin (150 mg, 0.54 mmol) is added and the residue is shaken for 3 hours. After filtration and removal of the solvent, a crude residue is recovered and purified by preparatory HPLC (without TFA) to give the title compound (30 mgs). LC-MS: m/z, 395.9 (M+), rt 2.007 min.

The compounds shown in Table 5 can be prepared according to the general procedure of Example 139, substituting 2-chloro-5-(trifluoromethyl)benzenesulfonyl chloride with the relevant sulphonyl chloride.

TABLE 5

| | | Reten- | |
|---|---|---|---|
| Ex- | | tion | LCMS: |
| am- | | Time | m/z, # |
| ple | structure | (min) | (M + H) |
| 140 | 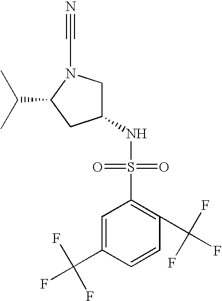 | 2.108 | 429 |
| | N-[(3R,5R)-1-Cyano-5-(1-methylethyl)- | | |
| | 3-pyrrolidinyl]- | | |
| | 2,5-bis(trifluoromethyl)benzenesulfonamide | | |
| 141 | 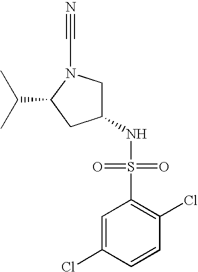 | 1.941 | 362.9 |
| | 2,5-Dichloro-N-[(3R,5R)-1-cyano-5- | | |
| | (1-methylethyl)-3- | | |
| | pyrrolidinyl]benzenesulfonamide | | |
| 142 | 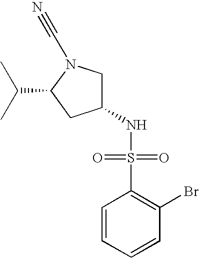 | 1.765 | 372.9 |
| | 2-Bromo-N-[(3R,5R)-1-cyano-5- | | |
| | (1-methylethyl)-3- | | |
| | pyrrolidinyl]benzenesulfonamide | | |
| 143 | 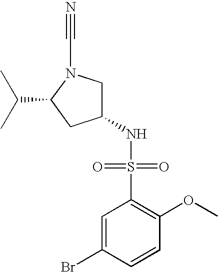 | 1.892 | 402.9 |
| | 5-Bromo-N-[(3R,5R)-1-cyano-5- | | |
| | (1-methylethyl)-3- | | |
| | pyrrolidinyl]-2- | | |
| | (methyloxy)benzenesulfonamide | | |
| 144 | 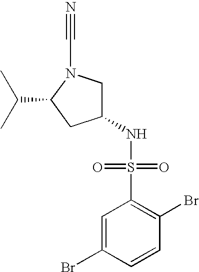 | 2.015 | 450.8 |
| | 2,5-Dibromo-N-[(3R,5R)-1-cyano-5- | | |
| | (1-methylethyl)-3- | | |
| | pyrrolidinyl]benzenesulfonamide | | |
| 145 | 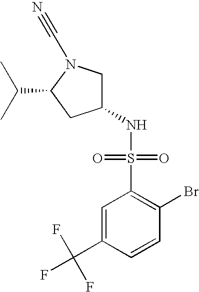 | 2.052 | 440.9 |
| | 2-Bromo-N-[(3R,5R)-1-cyano-5- | | |
| | (1-methylethyl)-3- | | |
| | pyrrolidinyl]-5-(trifluoro- | | |
| | methyl)benzenesulfonamide | | |
| 146 | 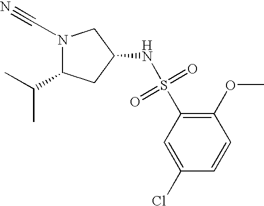 | 1.855 | 357.9 |
| | 5-Chloro-N-[(3R,5R)-1-cyano-5- | | |
| | (1-methylethyl)-3- | | |
| | pyrrolidinyl]-2- | | |
| | (methyloxy)benzenesulfonamide | | |

### EXAMPLE 147

### 2,5-Dichloro-N-[(3R,5R)-1-cyano-5-(cyanomethyl)-3-pyrrolidinyl]benzenesulfonamide

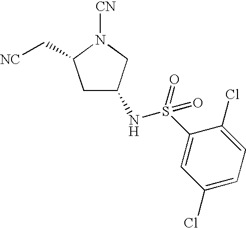

To a mixture of 1,1-dimethylethyl (2R,4R)-4-amino-2-(cyanomethyl)-1-pyrrolidinecarboxylate (140 mg, 0.621 mmol) and TEA (0.130 mL, 0.932 mmol) in DCM (10 mL) was added 2,5-dichorobenzenesulfonyl chloride (183 mg, 0.746 mmol) in 3 portions. The mixture was stirred at room temperature for 3 h then washed with water (2 mL). The organic layer was separated then concentrated under vacuum to give a crude material residue (270 mg, 0.622 mmol). This material was dissolved in 4M HCl in dioxane (5 ml), and then stirred for 15 hr at room temperature. The solvent was removed under reduced pressure, and the residue was suspended in DCM (5 ml), then DIEA (0.434 ml, 2.487 mmol) is added. A solution of BrCN (0.228 ml, 0.684 mmol, 3N in DCM) was added and the mixture was stirred for 1 h at room temperature then purified by automated flash chromatography to give the title compound (0.63 mg). LC/MS: m/z, 358.8 (M), rt 0.89 min. 1H NMR (400 MHz, CHLOROFORM-d) δ ppm 8.03 (1H, d, J=2.26 Hz) 7.43-7.50 (1H, m) 7.46 (1H, q, J=8.53 Hz) 5.42 (1H, d, J=7.78 Hz) 3.91-3.98 (1H, m) 3.75-3.82 (1H, m, J=6.09, 4.64, 3.54, 3.54 Hz) 3.51 (1H, dd, J=10.04, 7.03 Hz) 3.30 (1H, dd, J=10.16, 7.15 Hz) 2.79 (1H, dd, J=17.32, 6.02 Hz) 2.69 (1H, dd, J=17.32, 3.51 Hz) 2.40 (1H, ddd, J=13.68, 7.03, 6.90 Hz) 1.80 (1H, dt, J=13.30, 8.41 Hz)

### Biological Activity

The compounds according to Formula I are cathepsin C inhibitors, which indirectly inhibit the activity of serine proteases that are activated by cathepsin C, such as NE. The compounds according to Formula I, therefore, are useful in the treatment of COPD and other conditions involving cathepsin C and/or such serine proteases. The biological activity of the compounds according to Formula I can be determined using any suitable assay for determining the activity of a candidate compound as a cathepsin C inhibitor or for determining the ability of a candidate compound to prevent the cathepsin C mediated activation of certain serine proteases, as well as suitable tissue and in vivo models.

### A. Transpeptidation of Leucine-Leucine-O-Methyl (LLOM) Cell-Based Luminescence Viability Assay

### Principle:

Cathepsin C has been shown to catalyze the transpeptidation of dipeptidyl methyl-O-esters within the lysosome of cell form the monocytic lineage like HL60, U937 or THP1 causing a membranolythic effects that results in cell death (D L. Thiele P. Lipsky PNAS 1990 Vol. 87, pp. 83-87). This phenomenon was used to assess cellular Cathepsin C activity in the presence of our compounds.

### Reagents:

Leucine-Leucine-OMethyl (Bachem, G-2550)
Iscove's Modified Dulbecco's Medium (IMDM) with L-Glutamine with 25 mMolar HEPES buffer (GSK Media Prep Lab)
Heat Inactivated Fetal Bovine Serum (GSK media prep lab)
HL60 (ATCC, CCL-240)
Dimethyl Sulfoxide (DMSO) (Sigma, D8418)
1M HEPES buffer solution (Gibco, 15630-080)
CellTiter-Glo Luminescent Cell Viability Assay (Promega, G7572, G7573)

### Protocol:

Pre-warm IMDM with 20% FBS media.
Count HL60 cells with Hemocytometer.
Spin cells down at 1200K for 5 min.
Re-suspend cells at 200 K/ml in fresh pre-warmed media.
Dispense 100 ul/well of re-suspended cells in sterile black clear bottom polystyrene 96-well plate (Costar #3603).
Equilibrate cells for 30 min at 37° C., 5% CO2 incubator.
Add 1 ul of compound diluted at 100× concentrated DMSO. Compounds are ⅓ serial diluted in DMSO from 1 mM to 0.00 uM. Last row is DMSO only. Compounds are assayed in triplicate with final concentration 10 uM to 0.00 uM in wells.
Incubate plates at 37° C., 5% CO2 incubator for 30 min. Each plate is placed flat on shelf.
Add to each well 1 ul of 25 mM LLOM with 25 mM HEPES added in IMDM 20% FBS media to have 250 uM final in wells. LLOM solution is prepared fresh prior to addition.
Include a standard curve of compound GSK1473094A for QC.
Include a standard curve of LLOM for QC. LLOM is ½ serial diluted in IMDM with 20% FBS and 0.25 mM of HEPES media from 2 mM to 31.35 uM, Last row is only media.
Include three rows of cells without LLOM as 100% signal reference.
Incubate cells for 4 hours at 37° C., 5% CO2 incubator. Each plate is placed flat on shelf.
Thaw CellTiter-Glo buffer and substrate and equilibrate to room temperature.
Take the plates out of the incubator and lay flat on bench for 30 min to equilibrate to room temperature.
Add 100 ul of CellTiter-Glo to each well.
Rock plates for 5 min.
Read Luminescence (Wallace Envision reader and its software).
Plot and analyze data (using Graphpad Prism 4).

### B. Human Neutrophil Cathepsin C Assay

### Neutrophil Isolation:

### Reagents:

Ficol-Paque Plus (Amersham Biosciences #17-1440-03)
PBS without calcium and magnesium, room temperature
Dextran T-500 (Pharmacia # 17-0320-01)—a 6% (w/v) solution in PBS without calcium and magnesium, stored in 25 ml aliqouts in freezer
Sterile water
Trypan blue
10×PBS without calcium and magnesium

### Protocol:

At least 30 min. before blood is drawn, place 15 mL Ficol-Paque Plus into 50 mL Blue Max tubes.
Blood is collected, and each 25 mL of blood is layered over 15 mL Ficol-Paque Plus and centrifuged at 400 g at room temperature for 30 min. (brake is OFF).
Discard everything above the red cell fraction.
Red blood cell (RBC) pellets are resuspended to 35 mL in PBS w/o. Mix Dextran tubes by inversion, and add 12 mL to each blood tube. Mix RBC tubes by inversion and allow to stand undisturbed at room temperature for about 40 minutes (a clearly defined Richleau layer appears).
The layer above the RBC is collected, adjusted to 50 mL with PBS w/o, and centrifuged at 800 g at room temperature for 5 min. (brake can be on).
The sups are decanted and discarded down to about 3 mL, then the cells are gently dislodged and resuspended (in remaining supt.).
Lyse the RBC by adding 18 mL sterile water for 30 seconds at room temperature followed by 2 mL 10×PBS w/o.
The cell suspensions are adjusted to 50 mL with PBS w/o and centrifuged at 800 g for 5 min. at room temperature. Decant and discard sup.
Resuspend cells in PBS w/o (5 mL in each tube, pour two tubes together to make 10 mL). Remove 100 uL from tube of 10 mL, add it to 800 uL PBS w/o for counting, then bring the tubes up to 50 mL with PBS w/o. Centrifuge tubes once more at 800 g for 5 min.
To count cells, add 100 uL Trypan blue to the 900 uL tube. Place 10 uL on Hemacytometer. Count the cells in 5 different fields and average. This number ×106 is your total number of cells.

### Assay:

### Reagents:

PBS: Dulbecco's Phosphorus Buffered Saline without calcium and magnesium
PBS/gelatin: Dulbecco's Phosphorus Buffered Saline without calcium and magnesium with 0.1% gelatin made from a stock of 3% gelatin (Sigma) which is boiled and frozen in aliquots.
96-welled v-bottom plates (polypropylene)
96-welled flat-bottom tissue culture plates
PMN isolated from human blood

### Protocol:

In a 96-welled v-bottom plate (polypropylene): 20 uL stock compound solution (10 mM in DMSO) added to wells in top row. DMSO added to alternating rows at 20 and 23 uL. Dilutions are made by placing 10 uL to the row below, mixing then repeating serially until reaching the bottom row using a multichannel pipettor.
Resuspend PMN in PBS/gelatin to a final concentration of 200,000 cells per mL. Plate in a 96-welled flat-bottom tissue culture plate 100 uL per well, giving a final concentration of 20,000 cells per well.
Add 1 uL of compound per well in triplicate, and mix for 5 min on a plate shaker. Incubate at 37 C 5% CO2 for 30 min.
Add 5 uL of freshly diluted (H-Gly-Arg)₂ R110 Substrate (0.5 mM in PBS) and mix on a plate shaker for 5 min. Incubate at 37 C, 5% CO2 for 3 hrs.
Read plate using the Analyst HD reader and Criterion Host software Excitation=485 nm, Emmission=530 nm, dichrioc mirror=505 nm.
Graph data using Graph Pad Prism non-linear regression curve fit analysis.

### C. Recombinant Cathepsin C In Vitro Assay

The activity of recombinant human cathepsin C is measured by the cleavage of a fluorogenic substrate, H-Ser-Tyr-AMC. Briefly, 20 pM cathepsin C is incubated with test compound (e.g. inhibitor) in a buffer consisting of 50 mM sodium acetate, 30 mM sodium chloride, 1 mM CHAPS, 1 mM dithiothreitol, 1 mM EDTA, pH 5.5 at room temperature for one hour. After one hour of incubating test compound with cathepsin C, the activity assay is initiated by the addition of an equal volume of 0.010 mM H-Ser-Tyr-AMC in the same buffer. After one hour, the activity assay is stopped by the addition of 1/10 volume of 10 mM 2-Aldrithiol. The reaction product is measured on a fluorescence reader set at an excitation wavelength of 360 nm and emission wavelength of 460 nm and equipped with a 400 nm dichroic mirror.

### Biological Activity Results

All of the compounds exemplified above were tested for activity as cathepsin C inhibitors. Where the assay for a particular compound had been performed two or more times, the following conclusion regarding their activities is based on the average of individual experiments: All exemplified compounds were found to have a pIC₅₀ of 5.0 or greater.

The foregoing examples are being provided to illustrate the invention, not to limit it. Reference is made to the claims for what is reserved to the inventors as being their invention.

What is claimed is:

1. A compound according to Formula I: 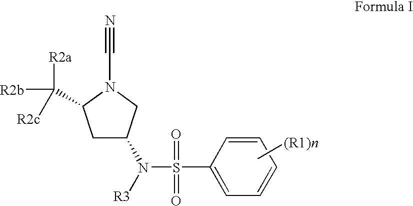
wherein:
each R1 is independently selected from the group consisting of: halo, C1-C4 alkyl, CF₃, CN, NO₂, —ORa, —OCF₃, —C(O)NHRa, —C(O)ORa, —NRaRa, —NHC(O)Ra, or —NHC(O)NHRa;
n is an integer from 0 to 4;
R2a is H, halo, —C(O)Rx, —C(O)ORy, —C(O)NRaRy, —OC(O)Rx, —OC(O)NRaRy, —NRaRy, —NRaC(O)Rx, NRaC(O)R22, —NRaC(O)ORy, —NRaC(O)NRaRy, R20, R21, R22, R23, R24, —OH, —OR20, —OR21, —OR22, —OR23, or —OR24; —CN
R2b is H or C1-C4 alkyl;
or R2a and R2b taken together with the carbon atom to which they are attached form a C3-C7 cycloalkyl group;
R2c is H or C1-C4 alkyl;
R20 is C1-C4 alkyl;
wherein said R20 is optionally substituted with one or more substituents independently selected from the group consisting of: halo, CF₃, CN, NO₂, R21, R22, R23, R24, —ORy, —C(O)Rx, —C(O)ORy, —C(O)NRaRy, —OC(O)Rx, —OC(O)NRaRy, —NRaRy, —NRaC(O)Rx, —NRaC(O)ORy, —NRaC(O)NRaRy;
R21 is C3-C6 cycloalkyl;
wherein said R21 is optionally substituted with one or more substituents independently selected from the group consisting of: CF₃, Rc, —ORa, —OCF₃, and —NRaRa;
R22 is heterocycloalkyl;
wherein said R22 is optionally substituted with one or more substituents independently selected from the group consisting of: CF₃, Rc, —ORa, —OCF₃, and —NRaRa;
R23 is phenyl;
wherein said R23 is optionally substituted with one or more substituents independently selected from the group consisting of: halo, CF₃, CN, NO₂, Rc, —ORa, —OCF₃, —C(O)Rb, —C(O)ORa, —C(O)NRaRa, —OC(O)Rb, —OC(O)NRaRa, —NRaRa, —NRaC(O)Rb, —NRaC(O)ORa, —NRaC(O)NRaRa;
R24 is monocyclic heteroaryl;
wherein said R24 is optionally substituted with one or more substituents independently selected from the group consisting of: halo, CF₃, CN, NO₂, Rc, —ORa, —OCF₃, —C(O)Rb, —C(O)ORa, —C(O)NRaRa, —OC(O)Rb, —OC(O)NRaRa, —NRaRa, —NRaC(O)Rb, —NRaC(O)ORa, —NRaC(O)NRaRa;
R3 is H, R30, or R31;
R30 is C1-C4 alkyl, C2-C4 alkenyl, or C2-C4 alkynyl;
wherein said R30 is optionally substituted with one or more substituents independently selected from the group consisting of: CF₃, Re, Rf, Rg, —ORa, CN, —OCF₃, —ORf, —ORg, —OR31, and —NRaRa;
R31 is C3-C6 cycloalkyl;
wherein said R31 is optionally substituted with one or more substituents independently selected from the group consisting of: Rb, —ORa, —OCF₃, and —NRaRa;
each Ra is independently H or C1-C4 alkyl;
each Rb is independently C1-C4 alkyl;
each Re is independently C1-C4 alkyl; wherein said C1-C4 alkyl is optionally substituted with one or more substituents independently selected from the group consisting of: CF₃, —ORa, OCF₃, and —NRaRa;
each Rd is independently C1-C4 alkyl; wherein said C1-C4 alkyl is optionally substituted with one or more substituents independently selected from the group consisting of: CF₃, —ORa, OCF₃, —NRaRa, Re, and Rf;
each Re is independently phenyl or heteroaryl optionally substituted with one or more substituents independently selected from the group consisting of: halo, CF₃, Rb, R23, R24, —ORa, OCF₃, and —NRaRa;
each Rf is independently monocyclic heteroaryl optionally substituted with one or more substituents independently selected from the group consisting of: halo, CF₃, Rb, R23, R24, —ORa, OCF₃, and —NRaRa;
each Rg is independently napthyl optionally substituted with one or more substituents independently selected from the group consisting of: halo, CF₃, Rb, —ORa, OCF₃, and —NRaRa;
each Rh is independently C3-C6 cycloalkyl optionally substituted with one or more substituents independently selected from the group consisting of: Rb, —ORa, —OCF₃, and —NRaRa;
each Rx is Rd, Re, Rf, Rg, or Rh; and
each Ry is H, Rd, Re, Rf, Rg, or Rh; or
a salt thereof.

2. A compound or salt of Formula I according to claim 1 wherein R2a is H.

3. A compound or salt of Formula I according to claim 1 wherein R2a is halo.

4. A compound or salt of Formula I according to claim 1 wherein R2a is —C(O)Rx, —C(O)ORy, or —C(O)NRaRy.

5. A compound or salt of Formula I according to claim 1 wherein R2a is —OC(O)Rx or —OC(O)NRaRy.

6. A compound or salt of Formula I according to claim 1 wherein R2a is —OC(O)Rx.

7. A compound or salt of Formula I according to claim 6 wherein Rx is Rd.

8. A compound or salt of Formula I according to claim 1 wherein R2a is —OC(O)NRaRy.

9. A compound or salt of Formula I according to claim 8 wherein Ry is phenyl optionally substituted with one or more substituents independently selected from the group consisting of: CF₃, Rb, and —ORa.

10. A compound or salt of Formula I according to claim 9 wherein Ra is H or C1-C4alkyl.

11. A compound or salt of Formula I according to claim 1 wherein R2a is —NRaRy, —NRaC(O)Rx, —NRaC(O)ORy, or —NRaC(O)NRaRy.

12. A compound or salt of Formula I according to claim 1 wherein R2a is —NRaC(O)Rx.

13. A compound or salt of Formula I according to claim 12 wherein Ra is H.

14. A compound or salt of Formula I according to claim 1 wherein R2a is —NRaC(O)NRaRy.

15. A compound or salt of Formula I according to claim 14 wherein each Ra is H.

16. A compound or salt of Formula I according to claim 1 wherein R2a is R20, R21, R22, R23, or R24.

17. A compound or salt of Formula I according to claim 1 wherein R2a is R20.

18. A compound or salt of Formula I according to claim 1 wherein R2a is —OH, —OR20, —OR21, —OR22, —OR23, or —OR24.

19. A compound or salt of Formula I according to claim 1 wherein R2a is —OR20.

20. A compound or salt of Formula I according to claim 1 wherein R2a is —OR23.

21. A compound or salt of Formula I according to claim 1 wherein each R1 is independently selected from the group consisting of: halo, C1-C4 alkyl, CF₃, CN, NO₂, —ORa, and —OCF₃.

22. A compound or salt of Formula I according to claim 1 wherein n is 0.

23. A compound or salt of Formula I according to claim 1 wherein n is 1.

24. A compound or salt of Formula I according to claim 1 wherein n is 2.

25. A compound or salt of Formula I according to claim 1 wherein n is 3.

26. A compound or salt of Formula I according to claim 1 wherein n is 4.

27. A compound or salt of Formula I according to claim 1 wherein R2b is H.

28. A compound or salt of Formula I according to any claim 1 wherein R2c is H.

29. A compound or salt of Formula I according to claim 1 wherein R3 is H.

30. A compound according to claim 1 which is:
2,5-dibromo-N-[(3R,5S)-1-cyano-5-methyl-3-pyrrolidinyl]benzenesulfonamide;
2,5-dichloro-N-[(3R,5S)-1-cyano-5-methyl-3-pyrrolidinyl]benzenesulfonamide
N-[(3R,5S)-1-cyano-5-methyl-3-pyrrolidinyl]-2,5-bis(methyloxy)benzenesulfonamide
3-bromo-N-[(3R,5S)-1-cyano-5-methyl-3-pyrrolidinyl]benzenesulfonamide
2-bromo-N-[(3R,5S)-1-cyano-5-methyl-3-pyrrolidinyl]-5-(trifluoromethyl)benzenesulfonamide
2-chloro-N-[(3R,5S)-1-cyano-5-methyl-3-pyrrolidinyl]-5-(trifluoromethyl)benzenesulfonamide
5-bromo-N-[(3R,5S)-1-cyano-5-methyl-3-pyrrolidinyl]-2-(methyloxy)benzenesulfonamide
5-bromo-N-[(3R,5S)-1-cyano-5-methyl-3-pyrrolidinyl]-2,4-difluorobenzenesulfonamide
N-[(3R,5S)-1-cyano-5-methyl-3-pyrrolidinyl]-2,3,5-trifluorobenzenesulfonamide
2,5-dibromo-N-{(3R,5R)-1-cyano-5-[(methyloxy)methyl]-3-pyrrolidinyl}benzenesulfonamide
2,5-dichloro-N-{(3R,5R)-1-cyano-5-[(methyloxy)methyl]-3-pyrrolidinyl}benzenesulfonamide
2-bromo-N-{(3R,5R)-1-cyano-5-[(methyloxy)methyl]-3-pyrrolidinyl}-5-(trifluoromethyl)benzenesulfonamide
2,5-dichloro-N-{(3R,5R)-1-cyano-5-[(phenyloxy)methyl]-3-pyrrolidinyl}benzenesulfonamide
2,5-dibromo-N-{(3R,5R)-1-cyano-5-[(phenyloxy)methyl]-3-pyrrolidinyl}benzenesulfonamide
((2R,4R)-1-cyano-4-{[(2,5-dibromophenyl)sulfonyl]amino}-2-pyrrolidinyl)methyl-2,2-dimethylpropanoate
((2R,4R)-1-cyano-4-{[(2,5-dichlorophenyl)sulfonyl]amino}-2-pyrrolidinyl)methyl-2,2-dimethylpropanoate
[(2R,4R)-4-({[5-chloro-2-(methyloxy)phenyl]sulfonyl}amino)-1-cyano-2-pyrrolidinyl]methyl 2,2-dimethylpropanoate
2-bromo-N-[(3R,5S)-1-cyano-5-methyl-3-pyrrolidinyl]-5-nitrobenzenesulfonamide
((2R,4R)-1-cyano-4-{[(2,5-dibromophenyl)sulfonyl]amino}-2-pyrrolidinyl)methyl phenylcarbamate
((2R,4R)-1-cyano-4-{[(2,5-dibromophenyl)sulfonyl]amino}-2-pyrrolidinyl)methyl (phenylmethyl)carbamate
((2R,4R)-1-cyano-4-{[(2,5-dibromophenyl)sulfonyl]amino}-2-pyrrolidinyl)methyl-{[2-(methyloxy)phenyl]methyl}carbamate
((2R,4R)-1-cyano-4-{[(2,5-dibromophenyl)sulfonyl]amino}-2-pyrrolidinyl)methyl {[3-(methyloxy)phenyl]methyl}carbamate
((2R,4R)-1-cyano-4-{[(2,5-dibromophenyl)sulfonyl]amino}-2-pyrrolidinyl)methyl-{[4-(methyloxy)phenyl]methyl}carbamate
((2R,4R)-1-cyano-4-{[(2,5-dibromophenyl)sulfonyl]amino}-2-pyrrolidinyl)methyl-[2-(methyloxy)phenyl]carbamate
((2R,4R)-1-cyano-4-{[(2,5-dibromophenyl)sulfonyl]amino}-2-pyrrolidinyl)methyl-[3-(methyloxy)phenyl]carbamate
((2R,4R)-1-cyano-4-{[(2,5-dibromophenyl)sulfonyl]amino}-2-pyrrolidinyl)methyl-[4-(methyloxy)phenyl]carbamate
((2R,4R)-1-cyano-4-{[(2,5-dibromophenyl)sulfonyl]amino}-2-pyrrolidinyl)methyl-[3-(trifluoromethyl)phenyl]carbamate
((2R,4R)-1-cyano-4-{[(2,5-dibromophenyl)sulfonyl]amino}-2-pyrrolidinyl)methyl-[4-(trifluoromethyl)phenyl]carbamate
((2R,4R)-1-cyano-4-{[(2,5-dibromophenyl)sulfonyl]amino}-2-pyrrolidinyl)methyl-(2-methylphenyl)carbamate
((2R,4R)-1-cyano-4-{[(2,5-dibromophenyl)sulfonyl]amino}-2-pyrrolidinyl)methyl-(3-methylphenyl)carbamate
((2R,4R)-1-cyano-4-{[(2,5-dibromophenyl)sulfonyl]amino}-2-pyrrolidinyl)methyl-1-naphthalenylcarbamate
((2R,4R)-1-cyano-4-{[(2,5-dibromophenyl)sulfonyl]amino}-2-pyrrolidinyl)methyl-2-naphthalenylcarbamate
((2R,4R)-1-cyano-4-{[(2,5-dibromophenyl)sulfonyl]amino}-2-pyrrolidinyl)methyl ethylcarbamate
((2R,4R)-1-cyano-4-{[(2,5-dibromophenyl)sulfonyl]amino}-2-pyrrolidinyl)methyl(1,1-dimethylethyl)carbamate
((2R,4R)-1-cyano-4-{[(2,5-dibromophenyl)sulfonyl]amino}-2-pyrrolidinyl)methyl[2-(trifluoromethyl)phenyl]carbamate
((2R,4R)-1-cyano-4-{[(2,5-dibromophenyl)sulfonyl]amino}-2-pyrrolidinyl)methyl cyclohexylcarbamate
2,5-dibromo-N-[(3R,5S)-1-cyano-5-methyl-3-pyrrolidinyl]-3,6-difluorobenzenesulfonamide
2,5-dibromo-N-{(3R,5R)-1-cyano-5-[(1,3-dioxo-1,3-dihydro-2H-isoindol-2-yl)methyl]-3-pyrrolidinyl}benzenesulfonamide
N-[((2R,4R)-1-cyano-4-{[(2,5-dibromophenyl)sulfonyl]amino}-2-pyrrolidinyl)methyl]benzamide
N-[((2R,4R)-1-cyano-4-{[(2,5-dibromophenyl)sulfonyl]amino}-2-pyrrolidinyl)methyl]-2,2-dimethylpropanamide
N-[((2R,4R)-1-cyano-4-{[(2,5-dibromophenyl)sulfonyl]amino}-2-pyrrolidinyl)methyl]-2-phenylacetamide
N-[((2R,4R)-1-cyano-4-{[(2,5-dibromophenyl)sulfonyl]amino}-2-pyrrolidinyl)methyl]-3-methylbenzamide
N-[((2R,4R)-1-cyano-4-{[(2,5-dibromophenyl)sulfonyl]amino}-2-pyrrolidinyl)methyl]-2-(methyloxy)benzamide
N-[((2R,4R)-1-cyano-4-{[(2,5-dibromophenyl)sulfonyl]amino}-2-pyrrolidinyl)methyl]cyclohexanecarboxamide
N-[((2R,4R)-1-cyano-4-{[(2,5-dibromophenyl)sulfonyl]amino}-2-pyrrolidinyl)methyl]-2-thiophenecarboxamide
2,5-dibromo-N-[(3R,5R)-1-cyano-5-({[(phenylamino)carbonyl]amino}methyl)-3-pyrrolidinyl]benzenesulfonamide
5-chloro-N-[(3R,5S)-1-cyano-5-methyl-3-pyrrolidinyl]-2-(methyloxy)benzenesulfonamide
N-[(3R,5S)-1-cyano-5-methyl-3-pyrrolidinyl]-2,5-dimethylbenzenesulfonamide
N-[(3R,5S)-1-cyano-5-methyl-3-pyrrolidinyl]-3-(trifluoromethyl)benzene-sulfonamide-2,5-dibromo-N-[(3R,5S)-1-cyano-5-methyl-3-pyrrolidinyl]-4-fluorobenzenesulfonamide
N-[((2R,4R)-1-cyano-4-{[(2,5-dichlorophenyl)sulfonyl]amino}-2-pyrrolidinyl)methyl]benzamide
N-[((2R,4R)-1-cyano-4-{[(2,5-dichlorophenyl)sulfonyl]amino}-2-pyrrolidinyl)methyl]-3-methylbenzamide
N-[((2R,4R)-1-cyano-4-{[(2,5-dichlorophenyl)sulfonyl]amino}-2-pyrrolidinyl)methyl]-2,2-dimethylpropanamide
N-[((2R,4R)-1-cyano-4-{[(2,5-dibromophenyl)sulfonyl]amino}-2-pyrrolidinyl)methyl]-3-fluorobenzamide
N-[((2R,4R)-1-cyano-4-{[(2,5-dibromophenyl)sulfonyl]amino}-2-pyrrolidinyl)methyl]-4-fluorobenzamide
2,5-dibromo-N-{(3R,5R)-1-cyano-5-[({[2-(methyloxy)phenyl)amino]carbonyl}amino)methyl]-3-pyrrolidinyl}benzene sulfonamide
2,5-dibromo-N-{(3R,5R)-1-cyano-5-[({[3-(methyloxy)phenyl)amino]carbonyl}amino)methyl]-3-pyrrolidinyl}benzene sulfonamide
2,5-dibromo-N-{(3R,5R)-1-cyano-5-[({[4-(methyloxy)phenyl)amino]carbonyl}amino)methyl]-3-pyrrolidinyl}benzene sulfonamide
2,5-dibromo-N-((3R,5R)-1-cyano-5-{[({[2-(trifluoromethyl)phenyl]amino}carbonyl)amino]methyl}-3-pyrrolidinyl)benzene sulfonamide
2,5-dibromo-N-((3R,5R)-1-cyano-5-{[({[3-(trifluoromethyl)phenyl]amino}carbonyl)amino]methyl}-3-pyrrolidinyl)benzene sulfonamide
2,5-dibromo-N-((3R,5R)-1-cyano-5-[({[4-(trifluoromethyl)phenyl]amino}carbonyl)amino]methyl}-3-pyrrolidinyl)benzene sulfonamide
2,5-dibromo-N-{(3R,5R)-1-cyano-5-[({[(3-fluorophenyl)amino]carbonyl}amino)methyl]-3-pyrrolidinyl}benzene sulfonamide
2,5-dibromo-N-{(3R,5R)-1-cyano-5-[({[(2-methyl phenyl)amino]carbonyl}amino)methyl]-3-pyrrolidinyl}benzenesulfonamide
2,5-dibromo-N-{(3R,5R)-1-cyano-5-[({[(3-methyl phenyl)amino]carbonyl}amino)methyl]-3-pyrrolidinyl}benzenesulfonamide
2,5-dibromo-N-{(3R,5R)-1-cyano-5-[({[(phenylmethyl)amino]carbonyl}amino)methyl]-3-pyrrolidinyl}benzenesulfonamide
2,5-dibromo-N-[(3R,5R)-1-cyano-5-({[({[2-(methyloxy)phenyl]methyl}amino)carbonyl]amino}methyl)-3-pyrrolidinyl]benzene sulfonamide
2,5-dibromo-N-[(3R,5R)-1-cyano-5-({[({[3-(methyloxy)phenyl]methyl}amino)carbonyl]amino}methyl)-3-pyrrolidinyl]benzene sulfonamide
2,5-dibromo-N-[(3R,5R)-1-cyano-5-({[({[4-(methyloxy)phenyl]methyl}amino)carbonyl]amino}methyl)-3-pyrrolidinyl]benzene sulfonamide
2,5-dibromo-N-[(3R,5R)-1-cyano-5-({[(cyclohexylamino) carbonyl]amino}methyl)-3-pyrrolidinyl]benzenesulfonamide
2,5-dibromo-N-[(3R,5R)-1-cyano-5-({[4-(methyloxy)phenyl]oxy}methyl)-3-pyrrolidinyl]benzenesulfonamide
2,5-dichloro-N-[(3R,5R)-1-cyano-5-({[4-(methyloxy)phenyl]oxy}methyl)-3-pyrrolidinyl]benzenesulfonamide
2,5-dibromo-N-((3R,5R)-1-cyano-5-{[(4-fluorophenyl)oxy]methyl}-3-pyrrolidinyl)benzenesulfonamide
2,5-dichloro-N-((3R,5R)-1-cyano-5-{[(4-fluorophenyl)oxy]methyl}-3-pyrrolidinyl)benzenesulfonamide
2-bromo-5-chloro-N-[(3R,5S)-1-cyano-5-methyl-3-pyrrolidinyl]-4-fluorobenzenesulfonamide
2,5-dichloro-N-[(3R,5S)-1-cyano-5-methyl-3-pyrrolidinyl]-4-fluorobenzenesulfonamide
2-bromo-5-chloro-N-[(3R,5S)-1-cyano-5-methyl-3-pyrrolidinyl]-4-fluorobenzenesulfonamide
2,5-dibromo-N-{(3R,5R)-1-cyano-5-[(dipropylamino) methyl]-3-pyrrolidinyl}benzenesulfonamide
2,5-dibromo-N-((3R,5R)-1-cyano-5-{[(phenylmethyl)oxy]methyl}-3-pyrrolidinyl)benzenesulfonamide
2,5-dibromo-N-((3R,5R)-1-cyano-5-{[(phenylmethyl)oxy]methyl}-3-pyrrolidinyl)benzenesulfonamide
N-{[(2R,4R)-4-({[5-chloro-2-(methyloxy)phenyl]sulfonyl}amino)-1-cyano-2-pyrrolidinyl]methyl}-2,2-dimethylpropanamide
N-{[(2R,4R)-4-({[5-bromo-2-(methyloxy)phenyl]sulfonyl}amino)-1-cyano-2-pyrrolidinyl]methyl}-2,2-dimethylpropanamide
N-{[(2R,4R)-4-({[2,5-bis(methyloxy)phenyl]sulfonyl}amino)-1-cyano-2-pyrrolidinyl]methyl}-2,2-dimethylpropanamide
N-({(2R,4R)-4-[{[5-bromo-2-(methyloxy)phenyl]sulfonyl}(phenylmethyl)amino]-1-cyano-2-pyrrolidinyl}methyl)-2,2-dimethylpropanamide
N-({(2R,4R)-4-[{[2,5-bis(methyloxy)phenyl]sulfonyl}(phenyl methyl)amino]-1-cyano-2-pyrrolidinyl}methyl)-2,2-dimethylpropanamide
2,5-dichloro-N-((3R,5R)-1-cyano-5-{[({[2-(methyloxy)ethyl]amino}carbonyl)-amino]methyl}-3-pyrrolidinyl)benzenesulfonamide
2,5-dibromo-N-((3R,5R)-1-cyano-5-{[({[2-(methyloxy)ethyl]amino}carbonyl)amino]methyl}-3-pyrrolidinyl)benzenesulfonamide
N-[((2R,4R)-1-cyano-4-{[(2,5-dibromophenyl)sulfonyl]amino}-2-pyrrolidinyl)methyl]-4-morpholinecarboxamide
5-chloro-N-((3R,5R)-1-cyano-5-{[({[2-(methyloxy)ethyl]amino}carbonyl)-amino]methyl}-3-pyrrolidinyl)-2-(methyloxy)benzenesulfonamide
N-{[(2R,4R)-4-({[5-chloro-2-(methyloxy)phenyl]sulfonyl}amino)-1-cyano-2-pyrrolidinyl]methyl}-4-morpholinecarboxamide
N-((3R,5R)-5-{[(aminocarbonyl)amino]methyl}-1-cyano-3-pyrrolidinyl)-5-chloro-2-(methyloxy)benzenesulfonamide
N-((3R,5R)-1-cyano-5-{[({[2-(methyloxy)ethyl]amino}carbonyl)amino]methyl}-3-pyrrolidinyl)-2,5-bis(methyloxy)benzenesulfonamide
N-((3R,5R)-5-{[(aminocarbonyl)amino]methyl}-1-cyano-3-pyrrolidinyl)-2,5-bis(methyloxy)benzenesulfonamide
((2R,4R)-1-cyano-4-{[(2,5-dibromophenyl)sulfonyl]amino}-2-pyrrolidinyl)methyl dimethylcarbamate
((2R,4R)-1-cyano-4-{[(2,5-dibromophenyl)sulfonyl]amino}-2-pyrrolidinyl)methyl ethyl(methyl)carbamate
((2R,4R)-1-cyano-4-{[(2,5-dibromophenyl)sulfonyl]amino}-2-pyrrolidinyl)methyl methyl(phenylmethyl)carbamate
((2R,4R)-1-cyano-4-{[(2,5-dibromophenyl)sulfonyl]amino}-2-pyrrolidinyl)methyl diethylcarbamate
2,5-dibromo-N-[(3R,5S)-1-cyano-5-methyl-3-pyrrolidinyl]-N-2-propen-1-ylbenzenesulfonamide
N-(2-biphenylylmethyl)-2,5-dibromo-N-[(3R,5S)-1-cyano-5-methyl-3-pyrrolidinyl]benzenesulfonamide
N-{[2,5-bis(trifluoromethyl)phenyl]methyl}-2,5-dibromo-N-[(3R,5S)-1-cyano-5-methyl-3-pyrrolidinyl]benzenesulfonamide
2,5-dibromo-N-[(3R,5S)-1-cyano-5-methyl-3-pyrrolidinyl]-N-[2-(methyloxy)ethyl]benzenesulfonamide
2,5-dibromo-N-[(3R,5S)-1-cyano-5-methyl-3-pyrrolidinyl]-N-(2-{[2-(methyloxy)ethyl]oxy}ethyl)benzenesulfonamide
2,5-dibromo-N-[(3R,5S)-1-cyano-5-methyl-3-pyrrolidinyl]-N-(3-phenylpropyl)benzenesulfonamide
2,5-dibromo-N-[(3R,5S)-1-cyano-5-methyl-3-pyrrolidinyl]-N-(3-methylbutyl)benzenesulfonamide
2,5-dibromo-N-[(3R,5S)-1-cyano-5-methyl-3-pyrrolidinyl]-N-ethylbenzenesulfonamide
2,5-dibromo-N-[(3R,5S)-1-cyano-5-methyl-3-pyrrolidinyl]-N-(4-fluorobutyl)benzenesulfonamide
2,5-dibromo-N-[(3R,5S)-1-cyano-5-methyl-3-pyrrolidinyl]-N-(cyclobutyl methyl)benzenesulfonamide
2,5-dibromo-N-[(3R,5S)-1-cyano-5-methyl-3-pyrrolidinyl]-N-(2-cyclohexylethyl)benzenesulfonamide
2,5-dibromo-N-(cyanomethyl)-N-[(3R,5S)-1-cyano-5-methyl-3-pyrrolidinyl]benzenesulfonamide
2,5-dibromo-N-(4-cyanobutyl)-N-[(3R,5S)-1-cyano-5-methyl-3-pyrrolidinyl]benzenesulfonamide
2,5-dibromo-N-[(3R,5S)-1-cyano-5-methyl-3-pyrrolidinyl]-N-2-propyn-1-ylbenzenesulfonamide
2,5-dibromo-N-[(3R,5S)-1-cyano-5-methyl-3-pyrrolidinyl]-N-[(4-methylphenyl)methyl]benzenesulfonamide
2,5-dibromo-N-[(3R,5S)-1-cyano-5-methyl-3-pyrrolidinyl]-N-[(2-nitrophenyl)methyl]benzenesulfonamide
2,5-dibromo-N-[(3R,5S)-1-cyano-5-methyl-3-pyrrolidinyl]-N-[(2,5-difluorophenyl)methyl]benzenesulfonamide
2,5-dibromo-N-[(3R,5S)-1-cyano-5-methyl-3-pyrrolidinyl]-N-[(5-methyl-2-phenyl-2H-1,2,3-triazol-4-yl)methyl]benzenesulfonamide
2,5-dibromo-N-[(3R,5S)-1-cyano-5-methyl-3-pyrrolidinyl]-N-cyclobutylbenzenesulfonamide
N-((3R,5R)-5-{[({[3,5-Bis(trifluoromethyl)phenyl]amino}-carbonyl)amino]methyl}-1-cyano-3-pyrrolidinyl)-2,5-dibromobenzenesulfonamide
2,5-dibromo-N-{(3R,5R)-1-cyano-5-[({[(1,1-dimethylethyl)amino]carbonyl}amino)methyl]-3-pyrrolidinyl}benzenesulfonamide
2,5-dibromo-N-((3R,5R)-1-cyano-5-{[({[(2-fluorophenyl)methyl]amino}-carbonyl)amino]methyl}-3-pyrrolidinyl)benzenesulfonamide
2,5-dibromo-N-{(3R,5R)-5-[({[(3-bromophenyl)amino]carbonyl}amino)methyl]-1-cyano-3-pyrrolidinyl}benzenesulfonamide
2,5-dibromo-N-((3R,5R)-1-cyano-5-{[({[(4-fluorophenyl)methyl]amino}carbonyl)-amino]methyl}-3-pyrrolidinyl)benzenesulfonamide
2,5-dibromo-N-{(3R,5R)-1-cyano-5-[({[(3-cyanophenyl)amino]carbonyl}-amino)methyl]-3-pyrrolidinyl}benzenesulfonamide
2,5-dibromo-N-{(3R,5R)-1-cyano-5-[({[(4′-fluoro-3-biphenylyl)amino]-carbonyl}amino)methyl]-3-pyrrolidinyl}benzenesulfonamide
2,5-dibromo-N-((3R,5R)-1-cyano-5-{[({[3-(2-methyl-1,3-thiazol-4-yl)phenyl]amino}carbonyl)amino]methyl}-3-pyrrolidinyl)benzenesulfonamide
2,5-dibromo-N-((3R,5R)-1-cyano-5-{[({[3-(1H-pyrazol-1-yl)phenyl]amino}carbonyl)amino]methyl}-3-pyrrolidinyl)benzenesulfonamide
2,5-dibromo-N-{(3R,5R)-1-cyano-5-[({[(3,5-dimethylphenyl)amino]carbonyl}-amino)methyl]-3-pyrrolidinyl}benzenesulfonamide
2,5-dibromo-N-((3R,5R)-1-cyano-5-{[methyl(4-pyridinylmethyl)amino]methyl}-3-pyrrolidinyl)benzenesulfonamide
N-((3R,5R)-5-{[(1,3-benzothiazol-2-ylmethyl)(methyl)amino]methyl}-1-cyano-3-pyrrolidinyl)-2,5-dibromobenzenesulfonamide
2,5-dibromo-N-[(3R,5R)-1-cyano-5-({methyl[2-(phenyloxy)ethyl]amino}methyl)-3-pyrrolidinyl]benzenesulfonamide
2,5-dibromo-N-((3R,5R)-1-cyano-5-{[methyl (tetrahydro-2H-pyran-4-ylmethyl)amino]methyl}-3-pyrrolidinyl)benzenesulfonamide
2,5-dibromo-N-[(3R,5R)-1-cyano-5-({4-[(methyloxy)methyl]-1-piperidinyl}methyl)-3-pyrrolidinyl]benzenesulfonamide
2,5-dibromo-N-((3R,5R)-1-cyano-5-{[2-(2-methylpropyl)-1-pyrrolidinyl]methyl}-3-pyrrolidinyl)benzenesulfonamide
2,5-dibromo-N-[(3R,5R)-1-cyano-5-({(2S)-2-[(methyloxy)methyl]-1-pyrrolidinyl}methyl)-3-pyrrolidinyl]benzenesulfonamide
2,5-dibromo-N-{(3R,5R)-1-cyano-5-[(dimethylamino)methyl]-3-pyrrolidinyl}benzenesulfonamide
2,5-dibromo-N-[(3R,5R)-1-cyano-5-(1-pyrrolidinylmethyl)-3-pyrrolidinyl]benzenesulfonamide
2,5-dibromo-N-((3R,5R)-1-cyano-5-{[methyl(2-propyn-1-yl)amino]methyl}-3-pyrrolidinyl)benzenesulfonamide
2,5-dibromo-N-((3R,5R)-1-cyano-5-{[methyl (phenylmethyl)amino]methyl}-3-pyrrolidinyl)benzenesulfonamide
2,5-dibromo-N-[(3R,5R)-1-cyano-5-(4-morpholinylmethyl)-3-pyrrolidinyl]benzenesulfonamide
2,5-dibromo-N-{(3R,5R)-1-cyano-5-[(ethyloxy)methyl]-3-pyrrolidinyl}benzenesulfonamide
2-chloro-N-[(3R,5R)-1-cyano-5-(1-methylethyl)-3-pyrrolidinyl]-5-(trifluoromethyl)benzenesulfonamide
N-[(3R,5R)-1-cyano-5-(1-methylethyl)-3-pyrrolidinyl]-2,5-bis(trifluoromethyl)benzenesulfonamide
2,5-dichloro-N-[(3R,5R)-1-cyano-5-(1-methylethyl)-3-pyrrolidinyl]benzenesulfonamide
2-bromo-N-[(3R,5R)-1-cyano-5-(1-methylethyl)-3-pyrrolidinyl]-benzenesulfonamide
5-bromo-N-[(3R,5R)-1-cyano-5-(1-methylethyl)-3-pyrrolidinyl]-2-(methyloxy)benzenesulfonamide
2,5-dibromo-N-[(3R,5R)-1-cyano-5-(1-methylethyl)-3-pyrrolidinyl]-benzenesulfonamide
2-bromo-N-[(3R,5R)-1-cyano-5-(1-methylethyl)-3-pyrrolidinyl]-5-(trifluoromethyl)benzenesulfonamide
5-chloro-N-[(3R,5R)-1-cyano-5-(1-methylethyl)-3-pyrrolidinyl]-2-(methyloxy)benzenesulfonamide
2,5-dichloro-N-[(3R,5R)-1-cyano-5-(cyanomethyl)-3-pyrrolidinyl]-benzenesulfonamide
or a salt thereof.

31. A pharmaceutical composition comprising a compound or pharmaceutically acceptable salt of Formula I according to claim 1 and one or more pharmaceutically-acceptable excipient.

32. A method for treating COPD comprising administering an effective amount of a compound or pharmaceutically acceptable salt of Formula I according to claim 1 to a patient in need thereof.
